SEQUENCE LISTING (269 sequences)

SEQ ID NO: 1 - protein, 133 aa, Homo sapiens [sequence omitted]

SEQ ID NO: 2 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 3 - protein, 524 aa, Homo sapiens
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 4 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 5 - protein, 133 aa, synthetic construct
  VARIANT 38: note = X can be any amino acid other than Arginine
APTSSSTKKTQLQLEHLLLDLQMILNGINNYKNPKLTXMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 6 - protein, 133 aa, synthetic construct
  VARIANT 42: note = X can be any amino acid other than Phenylalanine
APTSSSTKKTQLQLEHLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - protein, 133 aa, synthetic construct
  VARIANT 43: note = X can be any amino acid other than Lysine
APTSSSTKKTQLQLEHLLLDLQMILNGINNYKNPKLTRMLTFXFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 133 aa, synthetic construct
  VARIANT 45: note = X can be any amino acid other than Tyrosine
APTSSSTKKTQLQLEHLLLDLQMILNGINNYKNPKLTRMLTFKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 9 - protein, 133 aa, synthetic construct
  VARIANT 62: note = X can be any amino acid other than Glutamate
APTSSSTKKTQLQLEHLLLDLQMILNGINNYKNPKLTRMLTFKFYMPKKATELKHLQCLEEXLKPLEEVLNLAQSKNFHLRPRDLISNINVIVLELKGSETTFMCEYADETATIVEFLNRWITFCQSIISTLT

SEQ ID NO: 10 - protein, 133 aa, synthetic construct
  VARIANT 65: note = X can be any amino acid other than Proline
APTSSSTKKTQLQLEHLLLDLQMILNGINNYKNPKLTRMLTFKFYMPKKATELKHLQCLEEELKXLEEVLNLAQSKNFHLRPRDLISNINVIVLELKGSETTFMCEYADETATIVEFLNRWITFCQSIISTLT

SEQ ID NO: 11 - protein, 133 aa, synthetic construct
  VARIANT 68: note = X can be any amino acid other than Glutamate
APTSSSTKKTQLQLEHLLLDLQMILNGINNYKNPKLTRMLTFKFYMPKKATELKHLQCLEEELPKLEXVLNLAQSKNFHLRPRDLISNINVIVLELKGSETTFMCEYADETATIVEFLNRWITFCQSIISTLT

SEQ ID NO: 12 - protein, 133 aa, synthetic construct
  VARIANT 69: note = X can be any amino acid other than Valine
APTSSSTKKTQLQLEHLLLDLQMILNGINNYKNPKLTRMLTFKFYMPKKATELKHLQCLEEELPKLEEXLNLAQSKNFHLRPRDLISNINVIVLELKGSETTFMCEYADETATIVEFLNRWITFCQSIISTLT

SEQ ID NO: 13 - protein, 133 aa, synthetic construct
  VARIANT 72: note = X can be any amino acid other than Leucine
APTSSSTKKTQLQLEHLLLDLQMILNGINNYKNPKLTRMLTFKFYMPKKATELKHLQCLEEELPKLEEVLNXAQSKNFHLRPRDLISNINVIVLELKGSETTFMCEYADETATIVEFLNRWITFCQSIISTLT

SEQ ID NO: 14 - protein, 133 aa, synthetic construct
  VARIANT 15: note = X can be any amino acid other than Glutamate
APTSSSTKKTQLQLXHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 15 - protein, 133 aa, synthetic construct
  VARIANT 16: note = X can be any amino acid other than Histidine
APTSSSTKKTQLQLEXLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 16 - protein, 133 aa, synthetic construct
  VARIANT 19: note = X can be any amino acid other than Leucine
APTSSSTKKTQLQLEHLLXDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 17 - protein, 133 aa, synthetic construct
  VARIANT 20: note = X can be any amino acid other than Aspartate
APTSSSTKKTQLQLEHLLLXLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 18 - protein, 133 aa, synthetic construct
  VARIANT 84: note = X can be any amino acid other than Aspartate
APTSSSTKKTQLQLEHLLLDLQMILNGINNYKNPKLTRMLTFKFYMPKKATELKHLQCLEEELKPLEEVLNLAQSKNFHLRPRXLISNINVIVLELKGSETTFMCEYADETATIVEFLNRWITFCQSIISTLT

SEQ ID NO: 19 - protein, 133 aa, synthetic construct
  VARIANT 87: note = X can be any amino acid other than Serine
APTSSSTKKTQLQLEHLLLDLQMILNGINNYKNPKLTRMLTFKFYMPKKATELKHLQCLEEELKPLEEVLNLAQSKNFHLRPRDLIXNINVIVLELKGSETTFMCEYADETATIVEFLNRWITFCQSIISTLT

SEQ ID NO: 20 - protein, 133 aa, synthetic construct
  VARIANT 88: note = X can be any amino acid other than Asparagine
APTSSSTKKTQLQLEHLLLDLQMILNGINNYKNPKLTRMLTFKFYMPKKATELKHLQCLEEELKPLEEVLNLAQSKNFHLRPRDLISXINVIVLELKGSETTFMCEYADETATIVEFLNRWITFCQSIISTLT

SEQ ID NO: 21 - protein, 133 aa, synthetic construct
  VARIANT 91: note = X can be any amino acid other than Valine
APTSSSTKKTQLQLEHLLLDLQMILNGINNYKNPKLTRMLTFKFYMPKKATELKHLQCLEEELKPLEEVLNLAQSKNFHLRPRDLISNINXIVLELKGSETTFMCEYADETATIVEFLNRWITFCQSIISTLT

SEQ ID NO: 22 - protein, 133 aa, synthetic construct
  VARIANT 92: note = X can be any amino acid other than Isoleucine
APTSSSTKKTQLQLEHLLLDLQMILNGINNYKNPKLTRMLTFKFYMPKKATELKHLQCLEEELKPLEEVLNLAQSKNFHLRPRDLISNINVXVLELKGSETTFMCEYADETATIVEFLNRWITFCQSIISTLT

SEQ ID NO: 23 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 227 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 32 - protein, 276 aa, Homo sapiens
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 33 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 34 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 35 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 5 aa, synthetic construct
  VARIANT 4: note = X is any amino acid other than Proline
VPGXG

SEQ ID NO: 37 - protein, 223 aa, synthetic construct
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKCTPPVLDSDGSFFLYSRLTVDKSRWQEGNVFSCSVMHEALHNHYTQKSLSLSPG

SEQ ID NO: 38 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 107 aa, synthetic construct [sequence omitted]

SEQ ID NO: 41 - protein, 120 aa, synthetic construct [sequence omitted]

SEQ ID NO: 42 - protein, 50 aa, synthetic construct
  VARIANT 1..50: note = wherein up to 9 copies of GGGGS may be deleted
GGGGSGGGGSGGGGSGGGGSGGGGSGGGGSGGGGSGGGGSGGGGSGGGGS

SEQ ID NO: 43 - protein, 11 aa, synthetic construct
RASQDVNTAVA

SEQ ID NO: 44 - protein, 6 aa, synthetic construct
SASFLY

SEQ ID NO: 45 - protein, 8 aa, synthetic construct
QQHYTTPP

SEQ ID NO: 46 - protein, 8 aa, synthetic construct
GFNIKDTY

SEQ ID NO: 47 - protein, 8 aa, synthetic construct
IYPTNGYT

SEQ ID NO: 48 - protein, 13 aa, synthetic construct
SRWGGDGFYAMDY

SEQ ID NO: 49 - protein, 242 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 50 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 448 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 107 aa, synthetic construct [sequence omitted]

SEQ ID NO: 53 - protein, 119 aa, synthetic construct [sequence omitted]

SEQ ID NO: 54 - protein, 11 aa, synthetic construct
KASQDVSIGVA

SEQ ID NO: 55 - protein, 6 aa, synthetic construct
SASYRY

SEQ ID NO: 56 - protein, 8 aa, synthetic construct
QQYYIYPY

SEQ ID NO: 57 - protein, 10 aa, synthetic construct
GFTFTDYTMD

SEQ ID NO: 58 - protein, 18 aa, synthetic construct
ADVNPNSGGSIYNQRFKG

SEQ ID NO: 59 - protein, 12 aa, synthetic construct
ARNLGPSFYFDY

SEQ ID NO: 60 - protein, 15 aa, synthetic construct
KASQSVDYDGDSYLN

SEQ ID NO: 61 - protein, 7 aa, synthetic construct
DASNLVS

SEQ ID NO: 62 - protein, 9 aa, synthetic construct
QQSTEDPWT

SEQ ID NO: 63 - protein, 5 aa, synthetic construct
SYWMN

SEQ ID NO: 64 - protein, 17 aa, synthetic construct
QIWPGDGDTNYNGKFKG

SEQ ID NO: 65 - protein, 15 aa, synthetic construct
RETTTVGRYYYAMDY

SEQ ID NO: 66 - protein, 249 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVS

SEQ ID NO: 67 - protein, 250 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 68 - protein, 217 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTESS

SEQ ID NO: 69 - protein, 450 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 107 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTK

SEQ ID NO: 71 - protein, 120 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 72 - protein, 14 aa, synthetic construct
TGTSSDIGGYNSVS

SEQ ID NO: 73 - protein, 11 aa, synthetic construct
LMIYGVNNRPS

SEQ ID NO: 74 - protein, 10 aa, synthetic construct
SSYDIESATP

SEQ ID NO: 75 - protein, 10 aa, synthetic construct
GYSFTSYWIG

SEQ ID NO: 76 - protein, 16 aa, synthetic construct
WMGIIDPGDSRTRYSP

SEQ ID NO: 77 - protein, 11 aa, synthetic construct
GQLYGGTYMDG

SEQ ID NO: 78 - protein, 244 aa, synthetic construct
QVQLQQSGAEVKKPGASVKVSCKASGYTFTGYYMHWVRQAPGQGLEWMGRINPNSGGTNYAQKFQGRVTMTRDTSISTAYMELSRLRSEDTAVYYCARGRYYGMDVWGQGTMVTVSSGGGGSGGGGSGGGGSGGGGSEIVLTQSPATLSLSPGERATISCRASQSVSSNFAWYQQRPGQAPRLLIYDASNRATGIPPRFSGSGSGTDFTLTISSLEPEDFAAYYCHQRSNWLYTFGQGTKVDIK

SEQ ID NO: 79 - protein, 253 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDLRRTVVTPRAYYGMDVWGQGTTVTVSSGGGGSGGGGSGGGGSGGGGSDIQLTQSPSTLSASVGDRVTITCQASQDISNSLNWYQQKAGKAPKLLIYDASTLETGVPSRFSGSGSGTDFSFTISSLQPEDIATYYCQQHDNLPLTFGQGTKVEIK

SEQ ID NO: 80 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPIFTFGPGTKVDIK

SEQ ID NO: 81 - protein, 124 aa, synthetic construct
QMQLVESGGGVVQPGRSLRLSCTASGFTFSNNGMHWVRQAPGKGLEWVAVIWFDGMNKFYVDSVKGRFTISRDNSKNTLYLEMNSLRAEDTAIYYCAREGDGSGIYYYYGMDVWGQGTTVTVSS

SEQ ID NO: 82 - protein, 15 aa, synthetic construct
GGGGSGGGGSGGGGS

SEQ ID NO: 83 - protein, 106 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 84 - protein, 119 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 85 - protein, 112 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLG

SEQ ID NO: 86 - protein, 106 aa, synthetic construct
DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK

SEQ ID NO: 87 - protein, 119 aa, synthetic construct
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSS

SEQ ID NO: 88 - protein, 248 aa, synthetic construct
QMQLVESGGGVVQPGRSLRLSCTASGFTFSNNGMHWVRQAPGKGLEWVAVIWFDGMNKFYVDSVKGRFTISRDNSKNTLYLEMNSLRAEDTAIYYCAREGDGSGIYYYYGMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPIFTFGPGTKVDIK

SEQ ID NO: 89 - protein, 248 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPIFTFGPGTKVDIKGGGGSGGGGSGGGGSQMQLVESGGGVVQPGRSLRLSCTASGFTFSNNGMHWVRQAPGKGLEWVAVIWFDGMNKFYVDSVKGRFTISRDNSKNTLYLEMNSLRAEDTAIYYCAREGDGSGIYYYYGMDVWGQGTTVTVSS

SEQ ID NO: 90 - protein, 240 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 91 - protein, 240 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKGGGGSGGGGSGGGGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 92 - protein, 247 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSGGGGSGGGGSGGGGSDIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLG

SEQ ID NO: 93 - protein, 247 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGGGGGSGGGGSGGGGSQVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 94 - protein, 240 aa, synthetic construct
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSGGGGSGGGGSGGGGSDIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK

SEQ ID NO: 95 - protein, 240 aa, synthetic construct
DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSS

SEQ ID NO: 96 - protein, 10 aa, synthetic construct
GDSVSSNSAA

SEQ ID NO: 97 - protein, 9 aa, synthetic construct
TYYRSKWYN

SEQ ID NO: 98 - protein, 14 aa, synthetic construct
AREVTGDLEDAFDI

SEQ ID NO: 99 - protein, 6 aa, synthetic construct
QTIWSY

SEQ ID NO: 100 - protein, 9 aa, synthetic construct
QQSYSIPQT

SEQ ID NO: 101 - protein, 11 aa, synthetic construct
KASQDVSIAVA

SEQ ID NO: 102 - protein, 7 aa, synthetic construct
SASYRYT

SEQ ID NO: 103 - protein, 9 aa, synthetic construct
QQHYITPLT

SEQ ID NO: 104 - protein, 5 aa, synthetic construct
NYGMN

SEQ ID NO: 105 - protein, 17 aa, synthetic construct
WINTYTGEPTYTDDFKG

SEQ ID NO: 106 - protein, 12 aa, synthetic construct
GGFGSSYWYFDV

SEQ ID NO: 107 - protein, 5 aa, synthetic construct
TAGMQ

SEQ ID NO: 108 - protein, 17 aa, synthetic construct
WINTHSGVPKYAEDFKG

SEQ ID NO: 109 - protein, 12 aa, synthetic construct
SGFGSSYWYFDV

SEQ ID NO: 110 - protein, 11 aa, synthetic construct
KASQDVSTAVA

SEQ ID NO: 111 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 112 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 113 - protein, 107 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 114 - protein, 121 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 115 - protein, 217 aa, synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLGQPKAAPSVTLFPPSSEELQANKATLVCLISDFYPGAVTVAWKADSSPVKAGVETTTPDSKQSNNKYAASSYLSLTPEQWKSHRSYSCQVTHEGSTVEKTVAPTECS

SEQ ID NO: 116 - protein, 453 aa, synthetic construct
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 111 aa, synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLG

SEQ ID NO: 118 - protein, 131 aa, synthetic construct
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSV

SEQ ID NO: 119 - protein, 8 aa, synthetic construct
SSNIGSNT

SEQ ID NO: 120 - protein, 11 aa, synthetic construct
AAWDDSLNGWV

SEQ ID NO: 121 - protein, 8 aa, synthetic construct
GFTFGDYA

SEQ ID NO: 122 - protein, 10 aa, synthetic construct
SRSKAYGGTT

SEQ ID NO: 123 - protein, 14 aa, synthetic construct
ASSGYSSGWTPFDY

SEQ ID NO: 124 - protein, 249 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 125 - protein, 249 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRGGGGSGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 126 - protein, 11 aa, synthetic construct
SASQDISNYLN

SEQ ID NO: 127 - protein, 7 aa, synthetic construct
YTSNLHS

SEQ ID NO: 128 - protein, 9 aa, synthetic construct
QQYRKLPWT

SEQ ID NO: 129 - protein, 5 aa, synthetic construct
NYWMH

SEQ ID NO: 130 - protein, 17 aa, synthetic construct
ATYRGHSDTYYNQKFKG

SEQ ID NO: 131 - protein, 12 aa, synthetic construct
GAIYNGYDVLDN

SEQ ID NO: 132 - protein, 108 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 133 - protein, 121 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 134 - protein, 20 aa, synthetic construct
VTSAPDTRPAPGSTAPPAHG

SEQ ID NO: 135 - protein, 43 aa, synthetic construct
SNIKFRPGSVVVQLTLAFREGTINVHDVETQFNQYKTEAASRY

SEQ ID NO: 136 - protein, 35 aa, synthetic construct
SVVVQLTLAFREGTINVHDVETQFNQYKTEAASRY

SEQ ID NO: 137 - protein, 20 aa, synthetic construct
LAFREGTINVHDVETQFNQY

SEQ ID NO: 138 - protein, 25 aa, synthetic construct
SNIKFRPGSVVVQLTLAAFREGTIN

SEQ ID NO: 139 - protein, 5 aa, synthetic construct
RYGMS

SEQ ID NO: 140 - protein, 17 aa, synthetic construct
TISGGGTYIYYPDSVKG

SEQ ID NO: 141 - protein, 13 aa, synthetic construct
DNYGRNYDYGMDY

SEQ ID NO: 142 - protein, 10 aa, synthetic construct
SATSSVSYIH

SEQ ID NO: 143 - protein, 7 aa, synthetic construct
STSNLAS

SEQ ID NO: 144 - protein, 9 aa, synthetic construct
QQRSSSPFT

SEQ ID NO: 145 - protein, 5 aa, synthetic construct
GYAMS

SEQ ID NO: 146 - protein, 17 aa, synthetic construct
TISSGGTYIYYPDSVKG

SEQ ID NO: 147 - protein, 12 aa, synthetic construct
LGGDNYYEYFDV

SEQ ID NO: 148 - protein, 15 aa, synthetic construct
RASKSVSTSGYSYMH

SEQ ID NO: 149 - protein, 7 aa, synthetic construct
LASNLES

SEQ ID NO: 150 - protein, 9 aa, synthetic construct
QHSRELPFT

SEQ ID NO: 151 - protein, 5 aa, synthetic construct
DYAMN

SEQ ID NO: 152 - protein, 17 aa, synthetic construct
VISTFSGNINFNQKFKG

SEQ ID NO: 153 - protein, 10 aa, synthetic construct
SDYYGPYFDY

SEQ ID NO: 154 - protein, 16 aa, synthetic construct
RSSQTIVHSNGNTYLE

SEQ ID NO: 155 - protein, 7 aa, synthetic construct
KVSNRFS

SEQ ID NO: 156 - protein, 9 aa, synthetic construct
FQGSHVPFT

SEQ ID NO: 157 - protein, 9 aa, synthetic construct
LGGDNYYEY

SEQ ID NO: 158 - protein, 15 aa, synthetic construct
TASKSVSTSGYSYMH

SEQ ID NO: 159 - protein, 7 aa, synthetic construct
LVSNLES

SEQ ID NO: 160 - protein, 10 aa, synthetic construct
QHIRELTRSE

SEQ ID NO: 161 - protein, 8 aa, synthetic construct
GFTFSNYY

SEQ ID NO: 162 - protein, 8 aa, synthetic construct
ISGRGSTI

SEQ ID NO: 163 - protein, 10 aa, synthetic construct
VKDRGGYSPY

SEQ ID NO: 164 - protein, 6 aa, synthetic construct
QSISTY

SEQ ID NO: 165 - protein, 10 aa, synthetic construct
QQSYSTPPIT

SEQ ID NO: 166 - protein, 15 aa, synthetic construct
TEDEVQSYPSKHDYV

SEQ ID NO: 167 - protein, 12 aa, synthetic construct
EVQSYPSKHDYV

SEQ ID NO: 168 - protein, 8 aa, synthetic construct
GYTFTDYS

SEQ ID NO: 169 - protein, 8 aa, synthetic construct
INTETGVP

SEQ ID NO: 170 - protein, 8 aa, synthetic construct
ARRTGFDY

SEQ ID NO: 171 - protein, 11 aa, synthetic construct
KNLLHSDGITY

SEQ ID NO: 172 - protein, 9 aa, synthetic construct
VQVLELPFT

SEQ ID NO: 173 - protein, 8 aa, synthetic construct
GFTFSSYA

SEQ ID NO: 174 - protein, 8 aa, synthetic construct
ISDGGSYS

SEQ ID NO: 175 - protein, 14 aa, synthetic construct
ARDSYYDNSYVRDY

SEQ ID NO: 176 - protein, 6 aa, synthetic construct
QDINTF

SEQ ID NO: 177 - protein, 9 aa, synthetic construct
LQYDEFPLT

SEQ ID NO: 178 - protein, 213 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

SEQ ID NO: 179 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 5 aa, synthetic construct
NYGVH

SEQ ID NO: 181 - protein, 16 aa, synthetic construct
VIWSGGNTDYNTPFTS

SEQ ID NO: 182 - protein, 11 aa, synthetic construct
ALTYYDYEFAY

SEQ ID NO: 183 - protein, 11 aa, synthetic construct
RASQSIGTNIH

SEQ ID NO: 184 - protein, 7 aa, synthetic construct
YASESIS

SEQ ID NO: 185 - protein, 9 aa, synthetic construct
QQNNNQPTT

SEQ ID NO: 186 - protein, 5 aa, synthetic construct
SYWIE

SEQ ID NO: 187 - protein, 17 aa, synthetic construct
EILPGSKKTNYNEKFKG

SEQ ID NO: 188 - protein, 12 aa, synthetic construct
YYYRNDDYGMDT

SEQ ID NO: 189 - protein, 11 aa, synthetic construct
SASQDIRNYLN

SEQ ID NO: 190 - protein, 7 aa, synthetic construct
YTSTLHS

SEQ ID NO: 191 - protein, 9 aa, synthetic construct
QQYSKIPYT

SEQ ID NO: 192 - protein, 5 aa, synthetic construct
NYDMS

SEQ ID NO: 193 - protein, 16 aa, synthetic construct
YIGNGGNTYSPDTVKG

SEQ ID NO: 194 - protein, 8 aa, synthetic construct
HYGYDGRF

SEQ ID NO: 195 - protein, 16 aa, synthetic construct
RSSQSLEHSNGDTYLH

SEQ ID NO: 196 - protein, 7 aa, synthetic construct
KVSNRFS

SEQ ID NO: 197 - protein, 9 aa, synthetic construct
CQSTHVPWT

SEQ ID NO: 198 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 5 aa, synthetic construct
  VARIANT >5: note = The entire sequence of amino acids 1-5 may be repeated one or more times
GSGGS

SEQ ID NO: 201 - protein, 5 aa, synthetic construct
  VARIANT >5: note = The entire sequence of amino acids 1-5 may be repeated one or more times
GGGGS

SEQ ID NO: 202 - protein, 4 aa, synthetic construct
  VARIANT >4: note = The entire sequence of amino acids 1-4 may be repeated one or more times
GGGS

SEQ ID NO: 203 - protein, 4 aa, synthetic construct
GGSG

SEQ ID NO: 204 - protein, 5 aa, synthetic construct
GGSGG

SEQ ID NO: 205 - protein, 5 aa, synthetic construct
GSGSG

SEQ ID NO: 206 - protein, 5 aa, synthetic construct
GSGGG

SEQ ID NO: 207 - protein, 5 aa, synthetic construct
GGGSG

SEQ ID NO: 208 - protein, 5 aa, synthetic construct
GSSSG

SEQ ID NO: 209 - protein, 50 aa, synthetic construct
  VARIANT 1..50: note = wherein up to 9 copies of GSSSS may be deleted
GSSSSGSSSSGSSSSGSSSSGSSSSGSSSSGSSSSGSSSSGSSSSGSSSS

SEQ ID NO: 210 - protein, 20 aa, synthetic construct
GSSSSGSSSSGSSSSGSSSS

SEQ ID NO: 211 - protein, 25 aa, synthetic construct
GSSSSGSSSSGSSSSGSSSSGSSSS

SEQ ID NO: 212 - protein, 10 aa, synthetic construct
GGGGSGGGGS

SEQ ID NO: 213 - protein, 20 aa, synthetic construct
GGGGSGGGGSGGGGSGGGGS

SEQ ID NO: 214 - protein, 25 aa, synthetic construct
GGGGSGGGGSGGGGSGGGGSGGGGS

SEQ ID NO: 215 - protein, 5 aa, synthetic construct
AAAGG

SEQ ID NO: 216 - protein, 100 aa, synthetic construct
  VARIANT 1..100: note = wherein up to 19 copies of EAAAK may be deleted
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 217 - protein, 101 aa, synthetic construct
  VARIANT 2..101: note = wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 218 - protein, 102 aa, synthetic construct
  VARIANT 2..101: note = wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 219 - protein, 206 aa, synthetic construct
  VARIANT 2..101: note = wherein up to 19 copies of EAAAK may be deleted
  VARIANT 106..205: note = wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKALEAEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 220 - protein, 40 aa, synthetic construct
  VARIANT 1..40: note = wherein up to 19 copies of KP may be deleted
KPKPKPKPKPKPKPKPKPKPKPKPKPKPKPKPKPKPKPKP

SEQ ID NO: 221 - protein, 40 aa, synthetic construct
  VARIANT 1..40: note = wherein up to 19 copies of GP may be deleted
GPGPGPGPGPGPGPGPGPGPGPGPGPGPGPGPGPGPGPGP

SEQ ID NO: 222 - protein, 60 aa, synthetic construct
  VARIANT 1..60: note = wherein up to 19 copies of TPR may be deleted
TPRTPRTPRTPRTPRTPRTPRTPRTPRTPRTPRTPRTPRTPRTPRTPRTPRTPRTPRTPR

SEQ ID NO: 223 - protein, 40 aa, synthetic construct
  VARIANT 1..40: note = wherein up to 19 copies of AP may be deleted
APAPAPAPAPAPAPAPAPAPAPAPAPAPAPAPAPAPAPAP

SEQ ID NO: 224 - protein, 5 aa, synthetic construct
EAAAK

SEQ ID NO: 225 - protein, 10 aa, synthetic construct
EAAAKEAAAK

SEQ ID NO: 226 - protein, 15 aa, synthetic construct
EAAAKEAAAKEAAAK

SEQ ID NO: 227 - protein, 46 aa, synthetic construct
AEAAAKEAAAKEAAAKEAAAKALEAEAAAKEAAAKEAAAKEAAAKA

SEQ ID NO: 228 - protein, 12 aa, synthetic construct
AEAAAKEAAAKA

SEQ ID NO: 229 - protein, 5 aa, synthetic construct
PAPAP

SEQ ID NO: 230 - protein, 8 aa, synthetic construct
APAPAPAP

SEQ ID NO: 231 - protein, 12 aa, synthetic construct
APAPAPAPAPAP

SEQ ID NO: 232 - protein, 16 aa, synthetic construct
APAPAPAPAPAPAPAP

SEQ ID NO: 233 - protein, 20 aa, synthetic construct
APAPAPAPAPAPAPAPAPAP

SEQ ID NO: 234 - protein, 4 aa, synthetic construct
KPKP

SEQ ID NO: 235 - protein, 8 aa, synthetic construct
KPKPKPKP

SEQ ID NO: 236 - protein, 12 aa, synthetic construct
KPKPKPKPKPKP

SEQ ID NO: 237 - protein, 16 aa, synthetic construct
KPKPKPKPKPKPKPKP

SEQ ID NO: 238 - protein, 20 aa, synthetic construct
KPKPKPKPKPKPKPKPKPKP

SEQ ID NO: 239 - protein, 4 aa, synthetic construct
EPEP

SEQ ID NO: 240 - protein, 8 aa, synthetic construct
EPEPEPEP

SEQ ID NO: 241 - protein, 12 aa, synthetic construct
EPEPEPEPEPEP

SEQ ID NO: 242 - protein, 16 aa, synthetic construct
EPEPEPEPEPEPEPEP

SEQ ID NO: 243 - protein, 20 aa, synthetic construct
EPEPEPEPEPEPEPEPEPEP

SEQ ID NO: 244 - protein, 5 aa, synthetic construct
GCGGS

SEQ ID NO: 245 - protein, 5 aa, synthetic construct
CGGGS

SEQ ID NO: 246 - protein, 55 aa, synthetic construct
  VARIANT 6..55: note = wherein up to 9 copies of GGGGS may be deleted
CGGGSGGGGSGGGGSGGGGSGGGGSGGGGSGGGGSGGGGSGGGGSGGGGSGGGGS

SEQ ID NO: 247 - protein, 5 aa, synthetic construct
GGCGS

SEQ ID NO: 248 - protein, 55 aa, synthetic construct
  VARIANT 6..55: note = wherein up to 9 copies of GGGGS may be deleted
GGCGSGGGGSGGGGSGGGGSGGGGSGGGGSGGGGSGGGGSGGGGSGGGGSGGGGS

SEQ ID NO: 249 - protein, 5 aa, synthetic construct
GGGCS

SEQ ID NO: 250 - protein, 55 aa, synthetic construct
  VARIANT 6..55: note = wherein up to 9 copies of GGGGS may be deleted
GGGCSGGGGSGGGGSGGGGSGGGGSGGGGSGGGGSGGGGSGGGGSGGGGSGGGGS

SEQ ID NO: 251 - protein, 5 aa, synthetic construct
GGGGC

SEQ ID NO: 252 - protein, 55 aa, synthetic construct
  VARIANT 6..55: note = wherein up to 9 copies of GGGGS may be deleted
GGGGCGGGGSGGGGSGGGGSGGGGSGGGGSGGGGSGGGGSGGGGSGGGGSGGGGS

SEQ ID NO: 253 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 254 - protein, 133 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 255 - protein, 160 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSDYKDHDGDYKDHDIDYKDDDDK

SEQ ID NO: 256 - protein, 160 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSDYKDHDGDYKDHDIDYKDDDDK

SEQ ID NO: 257 - protein, 533 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 532 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 259 - protein, 380 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 374 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 261 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 262 - protein, 243 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 263 - protein, 243 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 264 - protein, 243 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSGGGGSGGGGSGGGGSDIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 265 - protein, 243 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKGGGGSGGGGSGGGGSQVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 266 - protein, 782 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 267 - protein, 532 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 268 - protein, 379 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 269 - protein, 9 aa, synthetic construct
NLVPMVATV

(19) United States
(12) Patent Application Publication
(10) Pub. No.: US 2025/0243254 A1
(43) Pub. Date: Jul. 31, 2025

(54) Interleukin-2 Polypeptides, Fusion Polypeptides, and Methods of Use Thereof

(71) Applicant: Cue Biopharma, Inc., Boston, MA (US)

(72) Inventors: Anish Suri, Boston, MA (US); Raymond J. Moniz, Boston, MA (US); Chee Meng Low, Boston, MA (US)

(21) Appl. No.: 19/075,563

(22) Filed: Mar. 10, 2025

Related U.S. Application Data
(63) Continuation of application No. PCT/US2023/073870, filed on Sep. 11, 2023.
(60) Provisional application No. 63/405,751, filed on Sep. 12, 2022.

(51) Int. Cl. C07K 14/55; A61K 38/00

(52) U.S. Cl. CPC C07K 14/55; A61K 38/00; C07K 2319/30

(57) ABSTRACT

IL-2 variant polypeptides and compositions comprising one or more IL-2 variant polypeptides, e.g., fusion polypeptides, having reduced affinity to IL-2Rα and IL-2Rβ are disclosed, as well as method for their use, e.g., in treatments involving cancer vaccines, TCR-T cell therapy and CAR-T cell therapy. Such polypeptides do not systemically activate multiple immune cell subsets, as native IL-2 delivered in high doses would, but rather predominantly activate only T cells whose T cell receptors (TCRs) are engaged with a peptide-MHC complex (pMHC) presented by an antigen presenting cell, which can thus provide a useful therapeutic index for pharmaceutical compositions comprising such polypeptides.

### CROSS-REFERENCE

This application claims the benefit of U.S. Provisional Patent Application No. 63/405,751, filed Sep. 12, 2022, which application is incorporated herein by reference in its entirety.

### INCORPORATION-BY-REFERENCE OF MATERIAL ELECTRONICALLY SUBMITTED

A Sequence Listing is provided herewith as a Sequence Listing XML, “CUEB-144WO_SEQ_LIST” created on Sep. 11, 2023 and having a size of 272,239 bytes. The contents of the Sequence Listing XML are incorporated by reference herein in their entirety.

### INTRODUCTION

Interleukin-2 (IL-2) is a cytokine that is produced by helper T cells to amplify immune responses. IL-2 has many immunostimulatory and immunoregulatory functions, including the proliferation of T cells, the activation of the cytotoxic function of natural killer (NK) cells, T lymphocytes and monocytes, and the activation of regulatory T cells (Tregs).

The known ability of IL-2 to activate T cells led to the development of the recombinant IL-2 product aldesleukin, which is marketed under the tradename Proleukin®. Aldesleukin is approved for use in treating metastatic melanoma and renal cell cancers, where it is hoped that the IL-2 will activate a sufficient number of T cells specific to the patient's cancer that it can reduce or kill the cancer. Unfortunately, however, Aldesleukin is such a powerful stimulator of T cells that it is associated with a range of potentially severe adverse effects that can include coma or even death. It is therefore recommended that aldesleukin be administered only in hospital settings where cardiopulmonary or intensive care medicine specialists are readily available.

Over the years, numerous variants of IL-2 have been investigated in an attempt to obtain the beneficial activity of IL-2, while reducing its known adverse effects. Because the IL-2 receptor on T cells (IL-2R) comprises three polypeptide chains, i.e., IL-2Rα (also referred to as CD25), IL-2Rβ (also referred to as CD122) and IL-2Rγ (also referred to as CD132), such variants have typically increased the affinity of IL-2 polypeptides to one or more of the IL-2R receptor chains and/or reduced the affinity of IL-2 polypeptides to one or more of the IL-2R receptor chains. For example, binding of IL-2 to IL-2Rα is known to upregulate the activity of Tregs. Hence, numerous “not-alpha” IL-2 variants that have a substantially reduced ability to bind to IL-2Rα have been developed for applications where the activation of Tregs is not desired.

IL-2 variants have been described that reduce the affinity of the IL-2 to bind to both IL-2Rα and IL-2Rβ. See, e.g., WO2018/119114 (Cue Biopharma, Inc.) and Quayle et al., Clin Cancer Res 2020; 26:1953-64, which disclosed an IL-2 variants comprising mutations that substantially eliminate binding to IL-2Rα and also significantly decrease the binding affinity to IL-2Rβ.

It has now been discovered that IL-2 variant polypeptides and compositions comprising one or more IL-2 variant polypeptides, e.g., fusion polypeptides, having such reduced affinity to IL-2Rα and IL-2Rβ have unexpected properties that can provide benefits in a number of different medical applications. More specifically, it has been discovered that the IL-2 variant polypeptides and compositions comprising one or more IL-2 variant polypeptides, e.g., fusion polypeptides, do not systemically activate multiple immune cell subsets, as native IL-2 delivered in high doses would, but rather predominantly activate only T cells whose T cell receptors (TCRs) are engaged with a peptide-MHC complex (pMHC) presented by an antigen presenting cell, which can thus provide a useful therapeutic index for pharmaceutical compositions comprising such IL-2 variant polypeptides or compositions comprising one or more IL-2 variant polypeptide, e.g., fusion polypeptides. Further, it has been discovered that such IL-2 variant polypeptides and compositions comprising one or more IL-2 variant polypeptide, e.g., fusion polypeptides, when engaged with IL-2 receptors on chimeric antigen receptor-T cells (CAR-T cells), TCR-T cells, and other cytotoxic cells (e.g., macrophages and NK cells) that have exogenous activation receptors such as chimeric antigen receptors (CARs) and TCRs, can provide homeostatic signals that can prolong the survival of such cells and/or provide activating signals to the cells that cause them to proliferate and retain their cytotoxic function.

### SUMMARY

Disclosed herein are methods for using IL-2 variants, and fusion polypeptides comprising such variants, wherein the variants have reduced binding to IL-2Rα and IL-2Rβ.

### BRIEF DESCRIPTION OF THE DRAWINGS

FIGS. 1A-1C are schematic depictions of higher-order forms of IL-2 variant polypeptides of this disclosure.

FIGS. 2A-2C are schematic depictions of fusion polypeptides comprising variant IL-2 polypeptides in the N-terminal position.

FIGS. 3A-3C are schematic depictions of fusion polypeptides comprising variant IL-2 polypeptides in the C-terminal position.

FIGS. 4A-4C are schematic depictions of fusion polypeptides comprising variant IL-2 polypeptides in the N-terminal and C-terminal positions.

FIGS. 5A-5B are schematic depictions of fusion polypeptide homodimers comprising variant IL-2 polypeptides in the N-terminal position.

FIGS. 6A-6B are schematic depictions of fusion polypeptide homodimers comprising variant IL-2 polypeptides in the C-terminal position.

FIGS. 7A-7B are schematic depictions of fusion polypeptide homodimers comprising variant IL-2 polypeptides in the N-terminal and C-terminal positions.

FIGS. 8A-8F are schematic depictions of fusion polypeptide heterodimers comprising variant IL-2 polypeptides in the N-terminal and C-terminal positions.

FIGS. 9A-9M provide amino acid sequences of immunoglobulin Fc polypeptides (SEQ ID NOs:23-35, respectively).

FIGS. 10A-10C are schematic depictions of fusion polypeptides comprising variant IL-2 polypeptides in the N-terminal position, and one or more additional proteins (designated “A”) in the C-terminal position.

FIGS. 11A-11C are schematic depictions of fusion polypeptides comprising variant IL-2 polypeptides in the C-terminal position, and one or more additional proteins (designated “A”) in the N-terminal position.

FIGS. 12A-12B are schematic depictions of fusion polypeptide homodimers comprising variant IL-2 polypeptides in the N-terminal position, and one or more additional proteins (designated “A”) in the C-terminal position.

FIGS. 13A-13B are schematic depictions of fusion polypeptide homodimers comprising variant IL-2 polypeptides in the C-terminal position, and one or more additional proteins (designated “A”) in the N-terminal position.

FIGS. 14A-14N are schematic depictions of fusion polypeptide heterodimers comprising variant IL-2 polypeptides in the N-terminal and/or C-terminal positions, and one or more additional proteins (designated “A” and “A′”) in the N-terminal and/or C-terminal positions.

FIGS. 15A-15D provide a comparison of binding signal for CUE-1646 vs CUE-1647 on human IL-2Rα and IL-2Rβ (see Example 1).

FIGS. 16A-16D provide representative sensorgrams and corresponding fits for CUE-1646 and CUE-1647 binding to human IL-2Rα and IL-2Rβ (see Example 1).

FIG. 17 provides Table 4, which shows the average affinity and kinetic measurements of CUE-1646 and CUE-1647 binding to human TL-2Rα and IL-2Rβ (see Example 1).

FIGS. 18A-18H provide amino acid sequences of exemplary anti-mesothelin scFv (SEQ ID NOs:88-95, respectively).

FIGS. 19A-19D provide amino acid sequences of exemplary anti-TROP-2 scFv (SEQ ID NOs:262-265, respectively).

FIG. 20 provides the amino acid sequence of an exemplary variant IL-2 fusion polypeptide (SEQ ID NO:266).

FIG. 21 provides the amino acid sequence of the polypeptide referred to as “1646” (SEQ ID NO:255).

FIG. 22 provides the amino acid sequence of the polypeptide referred to as “1647” (SEQ ID NO:256).

FIGS. 23A-23B provide the amino acid sequences of the variant IL-2/Fc fusion polypeptide referred to as “2657” or “Rgt-2657” (FIG. 23A; SEQ ID NO:257) and the variant IL-2/Fc fusion polypeptide designated “2657A” (FIG. 23B; SEQ ID NO:267) which lacks a C-terminal Lys.

FIG. 24 provides the amino acid sequence of the IL-2/Fc fusion polypeptide referred to as “3151” or “Rgt-3151” (SEQ ID NO:258).

FIGS. 25A-25B provide the amino acid sequences of the variant IL-2/Fc fusion polypeptide referred to as “2656” or “Rgt-2656” (FIG. 25A; SEQ ID NO:259) and the variant IL-2/Fc fusion polypeptide designated “2656A” (FIG. 25B; SEQ ID NO:268) which lacks a C-terminal Lys.

FIGS. 26A-26B provide amino acid sequences of the polypeptides referred to as “4123” (FIG. 26A; SEQ ID NO:260) and “4124” (FIG. 26B; SEQ ID NO:261).

FIG. 27 depicts the effect of the IL-2/Fc fusion polypeptide Rgt-2657 on proliferation of NK cells.

FIGS. 28A-28B depict the effect of the IL-2/Fc fusion polypeptide Rgt-2657 antigen-specific CD8⁺ T cells in vivo.

FIG. 29 depicts the effect of IL-2 valency on proliferation of CTLL-2 cells.

FIGS. 30A-30H are schematic depictions of heterodimeric fusion polypeptides comprising variant IL-2 polypeptides and, in some cases, one or more additional proteins (designated “A”).

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways, e.g., sequences can be aligned using various convenient methods and computer programs such as BLAST (Basic Local Alignment Search Tool) available over the world wide web at sites including https://blast.nebi.nlm.nih.gov/Blast.cgi. Unless otherwise stated, “sequence identity” as referred to herein is determined by BLAST with the default settings selected.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

“T cell” includes all types of immune cells expressing CD3, including T-helper cells (CD4⁺ cells), cytotoxic T-cells (CD8⁺ cells), T-regulatory cells (Treg), and NK-T cells.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The term “recombinant expression vector” is used herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (1996) Nature Structural Biol. 3:803; and Desmyter et al. (2015) Curr. Opin. Struct. Biol. 32:1). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

As used herein, the term “CDR” or “complementarity determining region” is intended to mean the non-contiguous antigen combining sites found within the variable region of both heavy and light chain polypeptides. CDRs have been described by Kabat et al (1977) J. Biol. Chem. 252:6609; Kabat et al., U.S. Dept. of Health and Human Services, “Sequences of proteins of immunological interest” (1991) (also referred to herein as Kabat 1991); by Chothia et al. (1987) J. Mol. Biol. 196:901 (also referred to herein as Chothia 1987); and MacCallum et al. (1996) J. Mol. Biol. 262:732 (also referred to herein as MacCallum 1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs, as defined by each of the above cited references are set forth below in Table 1 as a comparison.

TABLE 1
CDR Definitions

| | Kabat¹ | Chothia² | MacCallum³ |
|---|---|---|---|
| V_{H} CDR-1 | 31-35 | 26-32 | 30-35 |
| V_{H} CDR-2 | 50-65 | 53-55 | 47-58 |
| V_{H} CDR-3 | 95-102 | 96-101 | 93-101 |
| V_{L} CDR-1 | 24-34 | 26-32 | 30-36 |
| V_{L} CDR-2 | 50-56 | 50-52 | 46-55 |
| V_{L} CDR-3 | 89-97 | 91-96 | 89-96 |

¹Residue numbering follows the nomenclature of Kabat et al., 1991, supra
²Residue numbering follows the nomenclature of Chothia et al. (1987), supra
³Residue numbering follows the nomenclature of MacCallum et al. (1996), supra

As used herein, the terms “CDR-H1”, “CDR-H2”, and “CDR-H3” refer, respectively, to the first, second, and third CDRs in a heavy chain variable region. The terms “CDR-H1”, “CDR-H2”, and “CDR-H3” may be used interchangeably with “VH CDR1,” “VH CDR2,” and “VH CDR3,” respectively. As used herein, the terms “CDR-1”, “CDR-2”, and “CDR-3” refer, respectively, to the first, second and third CDRs of either chain's variable region.

As used herein, the term “affinity” refers to the equilibrium constant for the reversible binding of two agents (e.g., an antibody and an antigen) and is expressed as a dissociation constant (K_{D}).

The term “binding,” as used herein, refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. “Covalent binding” or “covalent bond,” as used herein, refers to the formation of one or more covalent chemical binds between two different molecules.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting, i.e., eliminating or reducing the disease or one or more symptoms; and/or (c) relieving the disease, i.e., causing regression or substantially eliminating the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc.

Unless indicated otherwise, the term “substantially” is intended to encompass both “wholly” and “largely but not wholly”. For example, an Ig Fc that “substantially does not induce ADCC” means an Ig Fc that induces no ADCC at all or that largely does not induce ADCC.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein and in the appended claims, the singular forms “a,” “an,” and “the” include plural referents unless the context clearly dictates otherwise. Thus, for example, reference to a “variant IL-2 polypeptide” includes a plurality of such polypeptides. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present disclosure. Nothing herein is to be construed as an admission that the present disclosure is not entitled to antedate such publication. Further, the dates of publication provided may be different from the actual publication dates which may need to be independently confirmed.

### DETAILED DESCRIPTION

This disclosure provides IL-2 variant polypeptides and compositions comprising IL-2 variant polypeptides, e.g., fusion polypeptides, wherein the variants have reduced binding to IL-2Rα and IL-2Rβ, and methods for use of such IL-2 variant polypeptides and compositions comprising IL-2 variant polypeptides.

In some cases, the methods include administering immunomodulatory proteins such as IL-2 variant polypeptides and compositions comprising one or more IL-2 variant polypeptides, e.g., fusion polypeptides, to an individual who also receives a therapy involving administration one of the following:
(a) modified or unmodified T cells, e.g., TCR-T therapy or therapy with tumor infiltrating lymphocytes (TILs);
(b) a product (e.g., a cancer vaccine) that can engage with the TCR of a T cell or be processed by an immune system to be presented by a pMHC complex to the TCR of a T cell;
(c) a precursor of a product (e.g., a nucleic acid vaccine such as an mRNA vaccine) that can engage with the TCR of a T cell or be processed by an immune system to be presented by an MHC to the TCR of a T cell;
(d) CAR-T cells; or
(e) modified cells comprising one or more exogenous activation receptors that can interact with target cells.

In some cases, the methods include administering immunomodulatory proteins such as IL-2 variant polypeptides and compositions comprising one or more IL-2 variant polypeptides, e.g., fusion polypeptides, to an individual who also receives a therapy involving administration one of the following:
(a) modified or unmodified T cells having a TCR, e.g., TCR-T therapy or therapy with TILs; or
(b) modified cells that comprise a chimeric antigen receptor (CAR), wherein the CAR binds to a target antigen and the modified cell comprises an intracellular signaling domain that is activated by interaction of the modified cell with IL-2; or
(c) modified cells comprising one or more exogenous activation receptors; or
(d) a product (e.g., a vaccine) that can engage with the TCR of a T cell or be processed by an immune system into one or more antigens that can be presented by a major histocompatibility complex (MHC) to the TCR of a T cell, optionally wherein the one or more antigens are cancer-associated antigens; or
(e) one or more nucleic acids (e.g., a nucleic acid vaccine such as an mRNA vaccine) encoding one or more polypeptides that can engage with the TCR of a T cell or can be processed by an immune system into one or more antigens that can be presented by a major histocompatibility complex (MHC) to the TCR of a T cell, optionally wherein the one or more antigens are cancer-associated antigens; or
(f) at least one immune checkpoint inhibitor (CPI). In cases where an individual is administered a first composition comprising (a), (b), (c), (d) or (e), the individual also may be administered a CPI.

As noted above, it has now been discovered that IL-2 variant polypeptides and compositions comprising one or more IL-2 variant polypeptides, e.g., fusion polypeptides, having such reduced affinity to IL-2Rα and IL-2Rβ have unexpected properties that can provide benefits in a number of different medical applications. More specifically, it has been discovered that the IL-2 variant polypeptides and compositions comprising one or more IL-2 variant polypeptides, e.g., fusion polypeptides, do not systemically activate multiple immune cell subsets, as native IL-2 does, but rather predominantly (i.e., mostly) or preferentially (i.e., more often) activate T cells whose T cell receptors (TCRs) are engaged with a peptide-MHC complex (pMHC) presented by an antigen presenting cell, as opposed to activating T cells whose T cell receptors (TCRs) are not engaged with a peptide-MHC complex (pMHC) presented by an antigen presenting cell. For example, a variant IL-2 polypeptide, or a fusion polypeptide comprising a variant IL-2 polypeptide of the present disclosure can activate a T cell whose TCRs are engaged with a pMHC to an extent that is at least 5%, at least 10%, at least 20%, at least 30%, at least 40%, at least 50%, at least 75%, at least 100% (or 2-fold), or more than the extent to which a T cell whose TCR are not engaged with a pMHC is activated by the same variant IL-2 polypeptide. This property can thus provide a useful therapeutic index for pharmaceutical compositions comprising such IL-2 variant polypeptides or compositions comprising one or more IL-2 variant polypeptides, e.g., fusion polypeptides comprising one or more IL-2 variant polypeptides. That is, the pharmaceutical compositions comprising such IL-2 variant polypeptides or compositions comprising one or more IL-2 variant polypeptide, e.g., fusion polypeptides, can be administered in a range of doses at which such compositions are therapeutically effective without unacceptable adverse events or toxicity. Further, it has been discovered that such IL-2 variant polypeptides and compositions comprising one or more IL-2 variant polypeptides, e.g., fusion polypeptides, when engaged with IL-2 receptors on CAR-T cells, TCR-T cells, and other cytotoxic cells (e.g., macrophages and NK cells) that have exogenous activation receptors such as CARs and TCRs, can provide homeostatic signals that can prolong the survival of such cells and/or provide activating signals to the cells that cause them to proliferate and retain their cytotoxic function. The term “exogenous activation receptors” includes receptors that are not normally present in a cell but instead are introduced into the cell to provide a desired functionality, e.g., by introducing into the cell a nucleic acid comprising a nucleotide sequence encoding the activation receptor.

The variant IL-2 polypeptides, fusion proteins and methods are described below.

### Variant IL-2 Polypeptides

As noted above, the IL-2 variants comprise mutations that substantially eliminate binding to IL-2Rα and also decrease but not substantially eliminate the binding affinity to IL-2Rβ. By substantially eliminating the binding to IL-2Rα, the IL-2 variants do not markedly upregulate the production of Tregs, which are undesirable in applications where the cytotoxic activity of T cells is desired. The reduced binding affinity to IL-2Rβ, in combination with the substantially eliminated binding to IL-2Rα, largely reduces the ability of such IL-2 variants to systemically activate T cells.

A wild-type IL-2 amino acid sequence can be as
follows:
(SEQ ID NO: 1)
APTSSSTKKT QLQLEHLLLD LQMILNGINN YKNPKLTRML
TFKFYMPKKA TELKHLQCLEEELKPLEEVLNLAQSKNFHL
RPRDLISNIN VIVLELKGSE TTFMCEYADE
TATIVEFLNRWITFCQSIIS TLT.

As noted above, IL-2 receptor comprises IL-2Rα, IL-2Rβ, and IL-2Rγ chains. Amino acid sequences of these three chains are as follows.

Human IL-2Rα:
(SEQ ID NO: 2)
ELCDDDPPE IPHATFKAMA YKEGTMLNCE CKRGFRRIKS
GSLYMLCTGN SSHSSWDNQC QCTSSATRNT TKQVTPQPEE
QKERKTTEMQ SPMQPVDQAS LPGHCREPPP WENEATERIY
HFVVGQMVYY QCVQGYRALH RGPAESVCKM THGKTRWTQP
QLICTGEMET SQFPGEEKPQ ASPEGRPESE TSCLVTTTDF
QIQTEMAATM ETSIFTTEYQ VAVAGCVFLL ISVLLLSGLT
WQRRQRKSRR TI.
Human IL-2Rβ:
(SEQ ID NO: 3)
VNG TSQFTCFYNS RANISCVWSQ DGALQDTSCQ
VHAWPDRRRW NQTCELLPVS QASWACNLIL GAPDSQKLTT
VDIVTLRVLC REGVRWRVMA IQDFKPFENL RLMAPISLQV
VHVETHRCNI SWEISQASHY FERHLEFEAR TLSPGHTWEE
APLLTLKQKQ EWICLETLTP DTQYEFQVRV KPLQGEFTTW
SPWSQPLAFR TKPAALGKDT IPWLGHLLVG LSGAFGFIIL
VYLLINCRNT GPWLKKVLKC NTPDPSKFFS QLSSEHGGDV
QKWLSSPFPS SSFSPGGLAP EISPLEVLER DKVTQLLLQQ
DKVPEPASLS SNHSLTSCFT NQGYFFFHLP DALEIEACQV
YFTYDPYSEE DPDEGVAGAP TGSSPQPLQP LSGEDDAYCT
FPSRDDLLLF SPSLLGGPSP PSTAPGGSGA GEERMPPSLQ
ERVPRDWDPQ PLGPPTPGVP DLVDFQPPPE LVLREAGEEV
PDAGPREGVS FPWSRPPGQG EFRALNARLP LNTDAYLSLQ
ELQGQDPTHL V.
Human IL-2Rγ:
(SEQ ID NO: 4)
LNTTILTP NGNEDTTADF FLTTMPTDSL SVSTLPLPEV
QCFVFNVEYM NCTWNSSSEP QPTNLTLHYW YKNSDNDKVQ
KCSHYLFSEE ITSGCQLQKK EIHLYQTFVV QLQDPREPRR
QATQMLKLQN LVIPWAPENL TLHKLSESQL ELNWNNRFLN
HCLEHLVQYR TDWDHSWTEQ SVDYRHKFSL PSVDGQKRYT
FRVRSRFNPL CGSAQHWSEW SHPIHWGSNT SKENPFLFAL
EAVVISVGSM GLIISLLCVY FWLERTMPRI PTLKNLEDLV
TEYHGNFSAW SGVSKGLAES LQPDYSERLC LVSEIPPKGG
ALGEGPGASP CNQHSPYWAP PCYTLKPET.

Mutations that can reduce binding of IL-2 to IL-2Rα include substitutions, e.g., including (but not limited to) conservative amino acid substitutions, at one or more of amino acids R38, F42, K43, Y45, E62, P65, E68, V69, and L72. For example, an IL-2 variant can comprise substitutions at one, two, three, four, five or more of the foregoing amino acids. Exemplary substitutions include the following from Table 2:

TABLE 2

| | |
|---|---|
| R38 | Any amino acid other than Arg, e.g., Ala, Asp or Glu |
| F42 | Any amino acid other than Phe, e.g., Ala or Lys, as |
| | well as Met, Pro, Ser, Thr, Trp, Tyr or Val |
| K43 | Any amino acid other than Lys, e.g., Glu |
| Y45 | Any amino acid other than Tyr |
| E62 | Any amino acid other than Glu, e.g., Gln |
| P65 | Any amino acid other than Pro |
| E68 | Any amino acid other than Glu |
| V69 | Any amino acid other than Val |
| L72 | Any amino acid other than Leu |

In some cases, a variant IL-2 polypeptide exhibits reduced binding affinity to IL-2Rα, compared to the binding affinity for IL-2Rα of an IL-2 polypeptide comprising the amino acid sequence set forth in SEQ ID NO:1. For example, in some cases, a variant IL-2 polypeptide binds IL-2Rα with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, at least 98% less, or at least 99% less than the binding affinity of an IL-2 polypeptide comprising the amino acid sequence set forth in SEQ ID NO:1 for an IL-2Rα. In some cases, the binding affinity is at least 75% less. In some cases, the binding affinity is at least 80% less. In some cases, the binding affinity is at least 85% less. In some cases, the binding affinity is at least 90% less. In some cases, the binding affinity is at least 95% less. In some cases, the binding affinity is at least 98% less. In some cases, the binding affinity is at least 99% less. In some cases, the binding affinity is reduced by at least 50-fold. In some cases, the binding affinity is reduced by at least 75-fold. In some cases, the binding affinity is reduced by at least 100-fold. In some cases, the binding affinity is reduced by about 110-fold.

In some cases, a variant IL-2 polypeptide retains at least some minimal binding to IL-2Rα. For example, in some cases, a variant IL-2 polypeptide exhibits from 2% to 50% of the binding of wild-type IL-2 (e.g., an IL-2 polypeptide comprising the amino acid sequence of SEQ ID NO:1) to an IL-2Rα polypeptide. For example, in some cases, a variant IL-2 polypeptide exhibits from 2% to 5%, from 5% to 10%, from 10% to 15%, from 15% to 20%, from 20% to 25%, from 25% to 30%, from 30% to 35%, from 35% to 40%, from 40% to 45%, or from 45% to 50%, of the binding of wild-type IL-2 to an IL-2Rα polypeptide.

As noted above, a substitution of F42 with an amino acid other than Phe, e.g., Ala, has been shown to substantially reduce the binding of an TL-2 variant to IL-2Rα. See Quayle et al., Clin Cancer Res 2020; 26:1953-64, which reported that an F42A substitution caused a 110-fold decrease in the binding to IL-2Rα. The binding to IL-2Rα is thus substantially abrogated by this mutation.

Mutations that can reduce binding of IL-2 to IL-2Rβ include substitutions, e.g., including (but not limited to) conservative amino acid substitutions, at one or more of amino acids E15, H16, L19, D20, D84, S87, N88, V91, I92. For example, an IL-2 variant can comprise substitutions at one, two, three, four, five or more of the foregoing amino acids. Exemplary substitutions include the following from Table 3:

TABLE 3

| | |
|---|---|
| E15 | Any amino acid other than Glu |
| H16 | Any amino acid other than His, e.g., Ala, Glu, Thr, or Asp, |
| | as well as Gly, Val, Leu, Ile, Arg, Asn, Cys, Glu, Gln, Ile, |
| | Lys, Met, Phe, Pro, Ser, Tyr, Trp or Val |
| L19 | Any amino acid other than Leu |
| D20 | Any amino acid other than Asp, e.g., Asn |
| D84 | Any amino acid other than Asp, e.g., His, Lys, Arg or Lys |
| S87 | Any amino acid other than Ser |
| N88 | Any amino acid other than Asn, e.g., Ser, Ala, Gly, |
| | Arg, Thr or Asp |
| V91 | Any amino acid other than Val, e.g., Glu, Ala or Thr |
| I92 | Any amino acid other than Ile, e.g., Ala |

In some cases, a variant IL-2 polypeptide exhibits reduced binding affinity to IL-2R, compared to the binding affinity for IL-2Rβ of an IL-2 polypeptide comprising the amino acid sequence set forth in SEQ ID NO:1. For example, in some cases, a variant IL-2 polypeptide binds IL-2Rβ with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, at least 98% less, or at least 99% less than the binding affinity of an IL-2 polypeptide comprising the amino acid sequence set forth in SEQ ID NO:1 for an IL-2Rβ. In some cases, the binding affinity is at least 25% less. In some cases, the binding affinity is at least 30% less. In some cases, the binding affinity is at least 35% less. In some cases, the binding affinity is at least 40% less. In some cases, the binding affinity is at least 45% less. In some cases, the binding affinity is at least 50% less. In some cases, the binding affinity is reduced about 2-fold. In some cases, the binding affinity is reduced about three-fold.

In some cases, a variant IL-2 polypeptide retains binding to IL-2Rβ. For example, in some cases, a variant IL-2 polypeptide exhibits from 2% to 50% of the binding of wild-type IL-2 (e.g., an IL-2 polypeptide comprising the amino acid sequence of SEQ ID NO:1) to an IL-2Rβ polypeptide. For example, in some cases, a variant IL-2 polypeptide exhibits from 2% to 5%, from 5% to 10%, from 10% to 15%, from 15% to 20%, from 20% to 25%, from 25% to 30%, from 30% to 35%, from 35% to 40%, from 40% to 45%, or from 45% to 50%, of the binding of wild-type IL-2 to an IL-2Rβ polypeptide.

As noted above, a substitution of F42 with an amino acid other than His, e.g., Ala, has been shown to reduce the binding of an IL-2 variant to IL-2Rβ. See Quayle et al., Clin Cancer Res 2020; 26:1953-64, which reported that an H16A substitution caused a 3-fold decrease in the binding to IL-2Rβ.

Some exemplary combinations of mutations that reduce binding of an IL-2 variant polypeptide to IL-2Rα and IL-2Rβ include the following from Table 4:

TABLE 4

| Mutation(s) to | Mutation(s) to | |
|---|---|---|
| decrease binding | decrease binding | |
| to IL-2Rα | to IL-2Rβ | Exemplary combinations |
| R38 with any | E15 with any | E15A with R38A, |
| amino acid other | amino acid other | R38D or R38E |
| than Arg, e.g., | than Glu | |
| Ala, Asp, Glu | | |
| R38 with any | H16, with any | H16A with R38A, |
| amino acid other | amino acid other | R38D or R38E |
| than Arg, e.g., | than His, e.g., Ala, | H16T with R38A, |
| Ala, Asp, Glu | Glu, Thr, or Asp, | R38D or R38E |
| | | H16E with R38A, |
| | | R38D or R38E |
| | | H16D with R38A, |
| | | R38D or R38E |
| R38 with any | D84, with any | D84H with R38A, |
| amino acid other | amino acid other | R38D or R38E |
| than Arg, e.g., | than Asp, e.g., | D84K with R38A, |
| Ala, Asp, Glu | His, Lys or Arg | R38D or R38E |
| | | D84R with R38A, |
| | | R38D or R38E |
| R38 with any | N88, with any | R38A with N88S, N88A, N88G, |
| amino acid other | amino acid other | N88R, N88T, or N88D |
| than Arg, e.g., | than Asn, e.g., | R38D with N88S, N88A, N88G, |
| Ala, Asp, Glu | Ser, Ala, Gly, | N88R, N88T, or N88D |
| | Arg, Thr or Asp | R38E with N88S, N88A, N88G, |
| | | N88R, N88T, or N88D |
| R38 with any | V91 with any | R38A with V91E, |
| amino acid other | amino acid other | V91A or V91T |
| than Arg, e.g., | than Val, e.g., | R38D with V91E, |
| Ala, Asp, Glu | Glu, Ala or Thr | V91A or V91T |
| | | R38E with V91E, |
| | | V91A or V91T |
| R38 with any | I92 with any | R38A, I92A |
| amino acid other | amino acid other | R38D, I92A |
| than Arg, e.g., | than Ile, e.g., Ala | R38E, I92A |
| Ala, Asp, Glu | | |
| F42, with any | E15 with any | E15A, F42A |
| amino acid other | amino acid other | E15A, F42K |
| than Phe, e.g., | than Glu | |
| Ala or Lys,, as | | |
| well as Met, Pro, | | |
| Ser, Thr, Trp, | | |
| Tyr, and Val | | |
| F42, with any | H16, with any | H16A, F42A; H16T, F42A; |
| amino acid other | amino acid other | H16E, F42A; H16D, F42A |
| than Phe, e.g., | than His, e.g., Ala, | H16A, F42K; H16T, F42K; |
| Ala or Lys, as | Glu, Thr, or Asp, | H16E, F42K; H16D, F42K |
| well as Met, Pro, | | |
| Ser, Thr, Trp, | | |
| Tyr, and Val | | |
| F42, with any | D84, with any | F42A with D84H, |
| amino acid other | amino acid other | D84K or D84R |
| than Phe, e.g., | than Asp, e.g., | F42K with D84H, |
| Ala or Lys, as | His, Lys or Arg | D84K or D84R |
| well as Met, Pro, | | |
| Ser, Thr, Trp, | | |
| Tyr, and Val | | |
| F42, with any | N88, with any | F42A with N88S, N88A, N88G, |
| amino acid other | amino acid other | N88R, N88T, or N88D |
| than Phe, e.g., | than Asn, e.g., | F42K with N88S, N88A, N88G, |
| Ala or Lys, as | Ser, Ala, Gly, | N88R, N88T, or N88D |
| well as Met, Pro, | Arg, Thr or Asp | |
| Ser, Thr, Trp, | | |
| Tyr, and Val | | |
| F42, with any | V91 with any | F42A with V91E, |
| amino acid other | amino acid other | V91A, or V91T |
| than Phe, e.g., | than Val, e.g., | F42K with V91E, |
| Ala or Lys, as | Glu, Ala or Thr | V91A, or V91T |
| well as Met, Pro, | | |
| Ser, Thr, Trp, | | |
| Tyr, and Val | | |
| F42, with any | I92 with any | F42A with I92A |
| amino acid other | amino acid other | F42K with I92A |
| than Phe, e.g., | than Ile, e.g., Ala | |
| Ala or Lys, as | | |
| well as Met, Pro, | | |
| Ser, Thr, Trp, | | |
| Tyr, and Val | | |
| K43, with any | E15 with any | E15A, K43E |
| amino acid | amino acid other | |
| other than Lys, | than Glu | |
| e.g., Glu | | |
| K43, with any | H16, with any | H16A, K43E; H16T, K43E; |
| amino acid | amino acid other | H16E, K43E; H16D, K43E |
| other than Lys, | than His, e.g., Ala, | |
| e.g., Glu | Glu, Thr, or Asp, | |
| K43, with any | D84, with any | K43E with D84H, |
| amino acid | amino acid other | D84K or D84R |
| other than Lys, | than Asp, e.g., | |
| e.g., Glu | His, Lys or Arg | |
| K43, with any | N88, with any | K43E with N88S, N88A, N88G, |
| amino acid | amino acid other | N88R, N88T, or N88D |
| other than Lys, | than Asn, e.g., | |
| e.g., Glu | Ser, Ala, Gly, | |
| | Arg, Thr or Asp | |
| K43, with any | V91 with any | K43E with V91E, |
| amino acid | amino acid other | V91A, or V91T |
| other than Lys, | than Val, e.g., | |
| e.g., Glu | Glu, Ala or Thr | |
| K43, with any | I92 with any | K43E, I92A |
| amino acid | amino acid other | |
| other than Lys, | than Ile, e.g., Ala | |
| e.g., Glu | | |
| E62, with any | E15 with any | E15A, E62Q |
| amino acid | amino acid other | |
| other than Glu, | than Glu | |
| e.g., Gln | | |
| E62, with any | H16, with any | H16A, E62Q; H16T, E62Q; |
| amino acid | amino acid other | H16E, E62Q; H16D, E62Q |
| other than Glu, | than His, e.g., Ala, | |
| e.g., Gln | Glu, Thr, or Asp, | |
| E62, with any | D84, with any | E62Q with D84H, |
| amino acid | amino acid other | D84K or D84R |
| other than Glu, | than Asp, e.g., | |
| e.g., Gln | His, Lys or Arg | |
| E62, with any | N88, with any | E62Q with N88S, N88A, N88G, |
| amino acid | amino acid other | N88R, N88T, or N88D |
| other than Glu, | than Asn, e.g., | |
| e.g., Gln | Ser, Ala, Gly, | |
| | Arg, Thr or Asp | |
| E62, with any | V91 with any | E62Q with V91E, |
| amino acid | amino acid other | V91A, or V91T |
| other than Glu, | than Val, e.g., | |
| e.g., Gln | Glu, Ala or Thr | |
| E62, with any | I92 with any | E62Q, I92A |
| amino acid | amino acid other | |
| other than Glu, | than Ile, e.g., Ala | |
| e.g., Gln | | |
| F42, with any | E15 with any | E15A, F42A with D84H, |
| amino acid other | amino acid other | D84K or D84R |
| than Phe, e.g., | than Glu | E15A, F42K with D84H, |
| Ala or Lys, as | D84, with any | D84K or D84R |
| well as Met, Pro, | amino acid other | |
| Ser, Thr, Trp, | than Asp, e.g., | |
| Tyr, and Val | His, Lys or Arg | |
| F42, with any | E15 with any | E15A, F42A with N88S, N88A, |
| amino acid other | amino acid other | N88G, N88R, N88T, or N88D |
| than Phe, e.g., | than Glu | E15A, F42K with N88S, N88A, |
| Ala or Lys, as | N88, with any | N88G, N88R, N88T, or N88D |
| well as Met, Pro, | amino acid other | |
| Ser, Thr, Trp, | than Asn, e.g., | |
| Tyr, and Val | Ser, Ala, Gly, | |
| | Arg, Thr or Asp | |
| F42, with any | E15 with any | E15A, F42A with V91E, |
| amino acid other | amino acid other | V91A, or V91T |
| than Phe, e.g., | than Glu | E15A, F42K with V91E, |
| Ala or Lys, as | V91 with any | V91A, or V91T |
| well as Met, Pro, | amino acid other | |
| Ser, Thr, Trp, | than Val, e.g., | |
| Tyr, and Val | Glu, Ala or Thr | |
| F42, with any | E15 with any | E15A, F42A with I92A |
| amino acid other | amino acid other | E15A, F42K with I92A |
| than Phe, e.g., | than Glu | |
| Ala or Lys, as | I92 with any | |
| well as Met, Pro, | amino acid other | |
| Ser, Thr, Trp, | than Ile, e.g., Ala | |
| Tyr, and Val | | |
| F42, with any | H16, with any | H16A, F42A with D84H, |
| amino acid other | amino acid other | D84K or D84R |
| than Phe, e.g., | than His, e.g., Ala, | H16A, F42K with D84H, |
| Ala or Lys, as | Glu, Thr, or Asp | D84K or D84R |
| well as Met, Pro, | D84, with any | H16T, F42A with D84H, |
| Ser, Thr, Trp, | amino acid other | D84K or D84R |
| Tyr, and Val | than Asp, e.g., | H16T, F42K with D84H, |
| | His, Lys or Arg | D84K or D84R |
| | | H16E, F42A with D84H, |
| | | D84K or D84R |
| | | H16E, F42K with D84H, |
| | | D84K or D84R |
| | | H16D, F42A with D84H, |
| | | D84K or D84R |
| | | H16D, F42K with D84H, |
| | | D84K or D84R |
| F42, with any | H16, with any | H16A, F42A with N88S, N88A, |
| amino acid other | amino acid other | N88G, N88R, N88T, or N88D |
| than Phe, e.g., | than His, e.g., Ala, | H16A, F42K with N88S, N88A, |
| Ala or Lys, as | Glu, Thr, or Asp | N88G, N88R, N88T, or N88D |
| well as Met, Pro, | N88, with any | H16T, F42A with N88S, N88A, |
| Ser, Thr, Trp, | amino acid other | N88G, N88R, N88T, or N88D |
| Tyr, and Val | than Asn, e.g., | H16T, F42K with N88S, N88A, |
| | Ser, Ala, Gly, | N88G, N88R, N88T, or N88D |
| | Arg, Thr or Asp | H16E, F42A with N88S, N88A, |
| | | N88G, N88R, N88T, or N88D |
| | | H16E, F42K with N88S, N88A, |
| | | N88G, N88R, N88T, or N88D |
| | | H16D, F42A with N88S, N88A, |
| | | N88G, N88R, N88T, or N88D |
| | | H16D, F42K with N88S, N88A, |
| | | N88G, N88R, N88T, or N88D |
| F42, with any | H16, with any | H16A, F42A with V91E, |
| amino acid other | amino acid other | V91A, or V91T |
| than Phe, e.g., | than His, e.g., Ala, | H16A, F42K with V91E, |
| Ala or Lys, as | Glu, Thr, or Asp | V91A, or V91T |
| well as Met, Pro, | V91 with any | H16T, F42A with V91E, |
| Ser, Thr, Trp, | amino acid other | V91A, or V91T |
| Tyr, and Val | than Val, e.g., | H16T, F42K with V91E, |
| | Glu, Ala or Thr | V91A, or V91T |
| | | H16E, F42A with V91E, |
| | | V91A, or V91T |
| | | H16E, F42K with V91E, |
| | | V91A, or V91T |
| | | H16D, F42A with V91E, |
| | | V91A, or V91T |
| | | H16D, F42K with V91E, |
| | | V91A, or V91T |
| F42, with any | H16, with any | H16A, F42A with I92A |
| amino acid other | amino acid other | H16A, F42K with I92A |
| than Phe, e.g., | than His, e.g., Ala, | H16T, F42A with I92A |
| Ala or Lys, as | Glu, Thr, or Asp | H16T, F42K with I92A |
| well as Met, Pro, | I92 with any | H16E, F42A with I92A |
| Ser, Thr, Trp, | amino acid other | H16E, F42K with I92A |
| Tyr, and Val | than Ile, e.g., Ala | H16D, F42A with I92A |
| | | H16D, F42K with I92A |

Exemplary IL-2 variant amino acid sequences comprise an amino acid sequence having at least 85%, at least 90%, at least 95%, at least 97%, at least 98%, at least 99%, or 100%, amino acid sequence identity to any one of the following IL-2 variant amino acid sequences (1)-(XVIII):

(I) APTSSSTKKT QLQLEHLLLD LQMILNGINN YKNPKLTX₁ML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:5), wherein X₁ is any amino acid other than Arg, e.g., Ala, Asp or Glu, wherein the amino acid sequence comprises one or more additional mutations that reduce the binding of the IL-2 variant to IL-2Rβ (as compared to the binding affinity of wild-type IL-2 to IL-2Rβ) by at least 25%, at least 50%, or at least 75%, by at least about 2-fold, or by at least about 3-fold, and wherein the IL-2 variant exhibits a reduced binding affinity to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more mutations that reduce the binding of the IL-2 variant to IL-2Rα.

(II) APTSSSTKKT QLQLEHLLLD LQMILNGINN YKNPKLTRML TX₁KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:6), wherein X₁ is any amino acid other than Phe, e.g., Ala or Lys, wherein the amino acid sequence comprises one or more additional mutations that reduce the binding of the IL-2 variant to IL-2Rβ (as compared to the binding affinity of wild-type IL-2 to IL-2Rβ) by at least 25%, at least 50%, or at least 75%, by at least about 2-fold, or by at least about 3-fold, and wherein the IL-2 variant exhibits a reduced binding affinity to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more mutations that reduce the binding of the IL-2 variant to IL-2Rα. In some cases, X₁ instead may be Gly, Val, Leu, Ile, Arg, Asn, Cys, Glu, Gln, Ile, Lys, Met, Phe, Pro, Ser, Tyr, Trp or Val.

(III) APTSSSTKKT QLQLEHLLLD LQMILNGINN YKNPKLTRML TFX₁FYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:7), wherein X₁ is any amino acid other than Lys, e.g., Glu, wherein the amino acid sequence comprises one or more additional mutations that reduce the binding of the IL-2 variant to IL-2Rβ (as compared to the binding affinity of wild-type IL-2 to IL-2Rβ) by at least 25%, at least 50%, or at least 75%, by at least about 2-fold, or by at least about 3-fold, and wherein the IL-2 variant exhibits a reduced binding affinity to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more mutations that reduce the binding of the IL-2 variant to IL-2Rα.

(IV) APTSSSTKKT QLQLEHLLLD LQMILNGINN YKNPKLTRML TFKFX₁MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:8), wherein X₁ is any amino acid other than Tyr, wherein the amino acid sequence comprises one or more additional mutations that reduce the binding of the IL-2 variant to IL-2Rβ (as compared to the binding affinity of wild-type IL-2 to IL-2Rβ) by at least 25%, at least 50%, or at least 75%, by at least about 2-fold, or by at least about 3-fold, and wherein the IL-2 variant exhibits a reduced binding affinity to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more mutations that reduce the binding of the IL-2 variant to IL-2Rα.

(V) APTSSSTKKT QLQLEHLLLD LQMILNGINN YKNPKLTRML TFKFYMPKKA TELKHLQCLE EX₁LKPLEEVL NLAQSKNFHL RPRDLISNIN VIVLELKGSE TTFMCEYADE TATIVEFLNR WITFCQSIIS TLT (SEQ ID NO:9), wherein X₁ is any amino acid other than Glu, e.g., Gln, wherein the amino acid sequence comprises one or more additional mutations that reduce the binding of the IL-2 variant to IL-2Rβ (as compared to the binding affinity of wild-type IL-2 to IL-2Rβ) by at least 25%, at least 50%, or at least 75%, by at least about 2-fold, or by at least about 3-fold, and wherein the IL-2 variant exhibits a reduced binding affinity to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more mutations that reduce the binding of the IL-2 variant to IL-2Rα.

(VI) APTSSSTKKT QLQLEHLLLD LQMILNGINN YKNPKLTRML TFKFYMPKKA TELKHLQCLE EELKX₁LEEVL NLAQSKNFHL RPRDLISNIN VIVLELKGSE TTFMCEYADE TATIVEFLNR WITFCQSIIS TLT (SEQ ID NO:10), wherein X₁ is any amino acid other than Pro, wherein the amino acid sequence comprises one or more additional mutations that reduce the binding of the IL-2 variant to IL-2Rβ (as compared to the binding affinity of wild-type IL-2 to IL-2Rβ) by at least 25%, at least 50%, or at least 75%, by at least about 2-fold, or by at least about 3-fold, and wherein the IL-2 variant exhibits a reduced binding affinity to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more mutations that reduce the binding of the IL-2 variant to IL-2Rα.

(VII) APTSSSTKKT QLQLEHLLLD LQMILNGINN YKNPKLTRML TFKFYMPKKA TELKHLQCLE EELPKLEX₁VL NLAQSKNFHL RPRDLISNIN VIVLELKGSE TTFMCEYADE TATIVEFLNR WITFCQSIIS TLT (SEQ ID NO:11), wherein X₁ is any amino acid other than Glu, wherein the amino acid sequence comprises one or more additional mutations that reduce the binding of the IL-2 variant to IL-2Rβ (as compared to the binding affinity of wild-type IL-2 to IL-2R3) by at least 25%, at least 50%, or at least 75%, by at least about 2-fold, or by at least about 3-fold, and wherein the IL-2 variant exhibits a reduced binding affinity to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more mutations that reduce the binding of the IL-2 variant to IL-2Rα.

(VIII) APTSSSTKKT QLQLEHLLLD LQMILNGINN YKNPKLTRML TFKFYMPKKA TELKHLQCLE EELPKLEEX₁L NLAQSKNFHL RPRDLISNIN VIVLELKGSE TTFMCEYADE TATIVEFLNR WITFCQSIIS TLT (SEQ ID NO:12), wherein X₁ is any amino acid other than Val, wherein the amino acid sequence comprises one or more additional mutations that reduce the binding of the IL-2 variant to IL-2Rβ (as compared to the binding affinity of wild-type IL-2 to IL-2Rβ) by at least 25%, at least 50%, or at least 75%, by at least about 2-fold, or by at least about 3-fold, and wherein the IL-2 variant exhibits a reduced binding affinity to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more mutations that reduce the binding of the IL-2 variant to IL-2Rα.

(IX) APTSSSTKKT QLQLEHLLLD LQMILNGINN YKNPKLTRML TFKFYMPKKA TELKHLQCLE EELPKLEEVL NX₁AQSKNFHL RPRDLISNIN VIVLELKGSE TTFMCEYADE TATIVEFLNR WITFCQSIIS TLT (SEQ ID NO:13), wherein X₁ is any amino acid other than Leu, wherein the amino acid sequence comprises one or more additional mutations that reduce the binding of the IL-2 variant to IL-2Rβ (as compared to the binding affinity of wild-type IL-2 to IL-2Rβ) by at least 25%, at least 50%, or at least 75%, by at least about 2-fold, or by at least about 3-fold, and wherein the IL-2 variant exhibits a reduced binding affinity to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more mutations that reduce the binding of the IL-2 variant to IL-2Rα.

(X) APTSSSTKKT QLQLX₁HLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:14), wherein X₁ is any amino acid other than Glu, e.g., Ala, wherein the binding of the IL-2 variant to IL-2Rβ (as compared to the binding affinity of wild-type IL-2 to IL-2Rβ) is reduced by at least 2-fold or at least 3-fold as compared to the binding of wild-type IL-2 to IL-2R3, wherein the IL-2 comprises one or more mutations that reduce the binding affinity of the IL-2 variant to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more additional mutations that reduce the binding of the IL-2 variant to IL-2Rβ.

(XI) APTSSSTKKT QLQLEX₁LLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:15), wherein X₁ is any amino acid other than His, e.g., Ala, Glu, Thr, or Asp, wherein the binding of the IL-2 variant to IL-2Rβ (as compared to the binding affinity of wild-type IL-2 to IL-2Rβ) is reduced by at least 2-fold or at least 3-fold as compared to the binding of wild-type IL-2 to IL-2Rβ, wherein the IL-2 comprises one or more mutations that reduce the binding affinity of the IL-2 variant to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more additional mutations that reduce the binding of the IL-2 variant to IL-2Rβ. In some cases, X₁ is Gly, Val, Len, Ile, Arg, Asn, Cys, Glu, Gln, Ile, Lys, Met, Phe, Pro, Ser, Tyr, Trp or Val.

(XII) APTSSSTKKT QLQLEHLLX₁D LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:16), wherein X₁ is any amino acid other than Len, wherein the binding of the IL-2 variant to IL-2Rβ(as compared to the binding affinity of wild-type IL-2 to IL-2Rβ) is reduced by at least 2-fold or at least 3-fold as compared to the binding of wild-type IL-2 to IL-2R3, wherein the IL-2 comprises one or more mutations that reduce the binding affinity of the IL-2 variant to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more additional mutations that reduce the binding of the IL-2 variant to IL-2R$.

(XIII) APTSSSTKKT QLQLEHLLLX₁ LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:17), wherein X₁ is any amino acid other than Asp, e.g., Asn, wherein the binding of the IL-2 variant to IL-2Rβ (as compared to the binding affinity of wild-type IL-2 to IL-2Rβ) is reduced by at least 2-fold or at least 3-fold as compared to the binding of wild-type IL-2 to IL-2R3, wherein the IL-2 comprises one or more mutations that reduce the binding affinity of the IL-2 variant to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more additional mutations that reduce the binding of the IL-2 variant to IL-2Rβ.

(XIV) APTSSSTKKT QLQLEHLLLD LQMILNGINN YKNPKLTRML TFKFYMPKKA TELKHLQCLE EELKPLEEVL NLAQSKNFHL RPRX₁LISNIN VIVLELKGSE TTFMCEYADE TATIVEFLNR WITFCQSIIS TLT (SEQ ID NO:18), wherein X₁ is any amino acid other than Asp, e.g., His, Lys or Arg, wherein the binding of the IL-2 variant to IL-2Rβ (as compared to the binding affinity of wild-type IL-2 to IL-2Rβ) is reduced by at least 2-fold or at least 3-fold as compared to the binding of wild-type IL-2 to IL-2Rβ, wherein the IL-2 comprises one or more mutations that reduce the binding affinity of the IL-2 variant to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more additional mutations that reduce the binding of the IL-2 variant to IL-2Rβ.

(XV) APTSSSTKKT QLQLEHLLLD LQMILNGINN YKNPKLTRML TFKFYMPKKA TELKHLQCLE EELKPLEEVL NLAQSKNFHL RPRDLIX₁NIN VIVLELKGSE TTFMCEYADE TATIVEFLNR WITFCQSIIS TLT (SEQ ID NO:19), wherein X₁ is any amino acid other than Ser, wherein the binding of the IL-2 variant to IL-2Rβ (as compared to the binding affinity of wild-type IL-2 to IL-2Rβ) is reduced by at least 2-fold or at least 3-fold as compared to the binding of wild-type IL-2 to IL-2R3, wherein the IL-2 comprises one or more mutations that reduce the binding affinity of the IL-2 variant to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more additional mutations that reduce the binding of the IL-2 variant to IL-2Rβ.

(XVI) APTSSSTKKT QLQLEHLLLD LQMILNGINN YKNPKLTRML TFKFYMPKKA TELKHLQCLE EELKPLEEVL NLAQSKNFHL RPRDLISX₁N VIVLELKGSE TTFMCEYADE TATIVEFLNR WITFCQSIIS TLT (SEQ ID NO:20), wherein X₁ is any amino acid other than Asn, e.g., Ser, Ala, Gly, Arg, Thr or Asp, wherein the binding of the IL-2 variant to IL-2Rβ (as compared to the binding affinity of wild-type TL-2 to IL-2Rβ) is reduced by at least 2-fold or at least 3-fold as compared to the binding of wild-type IL-2 to IL-2Rβ, wherein the IL-2 comprises one or more mutations that reduce the binding affinity of the IL-2 variant to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more additional mutations that reduce the binding of the IL-2 variant to IL-2R3.

(XVII) APTSSSTKKT QLQLEHLLLD LQMILNGINN YKNPKLTRML TFKFYMPKKA TELKHLQCLE EELKPLEEVL NLAQSKNFHL RPRDLISNIN X₁IVLELKGSE TTFMCEYADE TATIVEFLNR WITFCQSIIS TLT (SEQ ID NO:21), wherein X₁ is any amino acid other than Val, e.g., Glu, Ala or Thr, wherein the binding of the IL-2 variant to IL-2Rβ (as compared to the binding affinity of wild-type IL-2 to IL-2Rβ) is reduced by at least 2-fold or at least 3-fold as compared to the binding of wild-type IL-2 to IL-2Rβ, wherein the IL-2 comprises one or more mutations that reduce the binding affinity of the IL-2 variant to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more additional mutations that reduce the binding of the IL-2 variant to IL-2Rβ.

(XVIII) APTSSSTKKT QLQLEHLLLD LQMILNGINN YKNPKLTRML TFKFYMPKKA TELKHLQCLE EELKPLEEVL NLAQSKNFHL RPRDLISNIN VX₁VLELKGSE TTFMCEYADE TATIVEFLNR WITFCQSIIS TLT (SEQ ID NO:22), wherein X₁ is any amino acid other than Ile, e.g., Ala, wherein the binding of the IL-2 variant to IL-2Rβ (as compared to the binding affinity of wild-type IL-2 to IL-2Rβ) is reduced by at least 2-fold or at least 3-fold as compared to the binding of wild-type IL-2 to IL-2Rβ, wherein the IL-2 comprises one or more mutations that reduce the binding affinity of the IL-2 variant to IL-2Rα (as compared to the binding affinity of wild-type IL-2 to IL-2Rα) by at least 50-fold, by at least 75-fold, by at least 100-fold or by about 110-fold, and wherein the IL-2 variant may include one or more additional mutations that reduce the binding of the IL-2 variant to IL-2Rβ.

The determination of binding affinity of the IL-2 variants to IL-2Rα and IL-2Rβ is carried out as described below in Example 1.

### Higher-Order Forms of IL-2 Variant Polypeptides

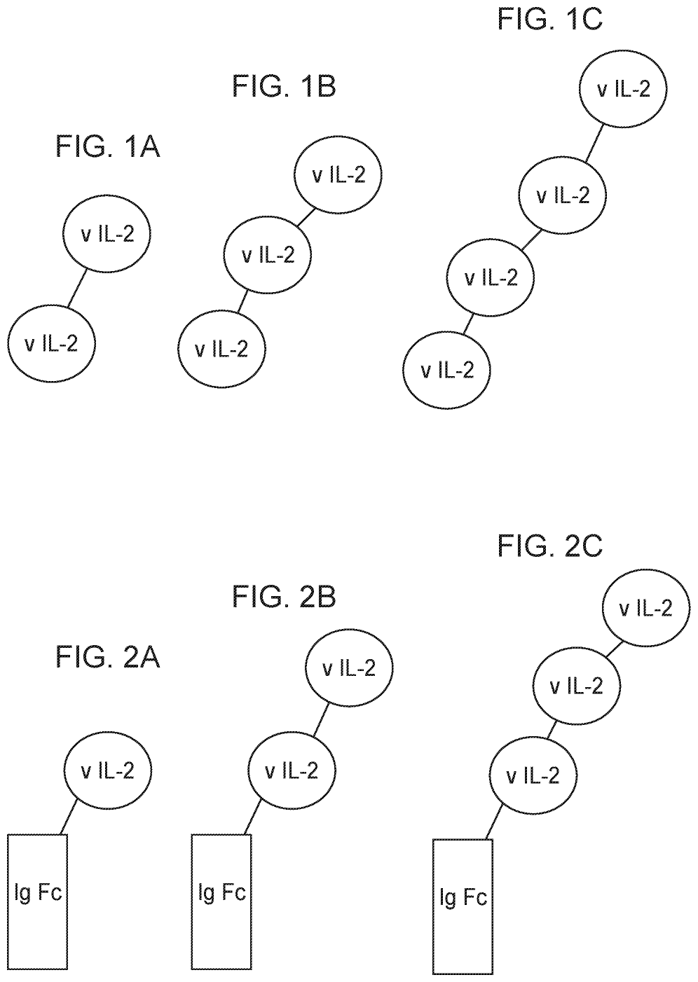

As illustrated in FIGS. 1A-1C, the IL-2 variant polypeptides of this disclosure may be linked together to form higher-order chains, e.g., dimers, trimers, tetramers, etc. The IL-2 variant polypeptides may be joined by one or more independently selected linkers (discussed below). The linkers for such higher order forms may include linkers, e.g., rigid linkers or short flexible linkers that enhance the avidity of the IL-2 variant polypeptides to their target cells and/or improve the yield of such higher order IL-2 variant polypeptides.

### Compositions Comprising IL-2 Variant Polypeptides

Compositions comprising one or more IL-2 variant polypeptides include fusion polypeptides on Ig Fc or other scaffold polypeptides, as well as compositions in which the one or more IL-2 variant polypeptides, or a fusion polypeptide comprising one or more IL-2 variant polypeptides, are associated with a carrier.

### Fusion Polypeptides

As noted above, this disclosure also provides compositions comprising IL-2 variant polypeptides, e.g., fusion polypeptides.

The heterologous fusion partner may comprise, e.g., an Ig Fc or other scaffold polypeptide, as well as one or more additional heterologous polypeptides such as the functional proteins described below. The incorporation of Ig Fe polypeptides or other scaffold polypeptide into a fusion polypeptide can provide increased stability, manufacturability and/or in vivo half-life to the resulting fusion polypeptide. Typically, a fusion polypeptide does not include an MHC class I polypeptide or an MHC class II polypeptide. For example, a fusion polypeptide typically does not include an MHC class I heavy chain polypeptide, a 02-microglobulin (02M) polypeptide, an MHC class II alpha chain polypeptide, or an MHC class II beta chain polypeptide. In some cases, a fusion polypeptide comprises: a) at least one IL-2 variant polypeptide; and b) an immunoglobulin (Ig) Fc polypeptide, but does not include any other heterologous polypeptide. some cases, a fusion polypeptide comprises: a) at least one IL-2 variant polypeptide; b) a peptide linker; and c) an Ig Fc polypeptide, but does not include any other heterologous polypeptide. In some cases, a fusion polypeptide comprises: a) at least one IL-2 variant polypeptide; b) optionally a peptide linker; c) an Ig Fc polypeptide, and at least one other heterologous polypeptide such as a functional protein described below.

Suitable scaffold polypeptides include antibody-based scaffold polypeptides and non-antibody-based scaffolds. Non-antibody-based scaffolds include, e.g., fibronectin-based scaffold proteins, albumin, an XTEN (extended recombinant) polypeptide, transferrin, an Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO:36), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

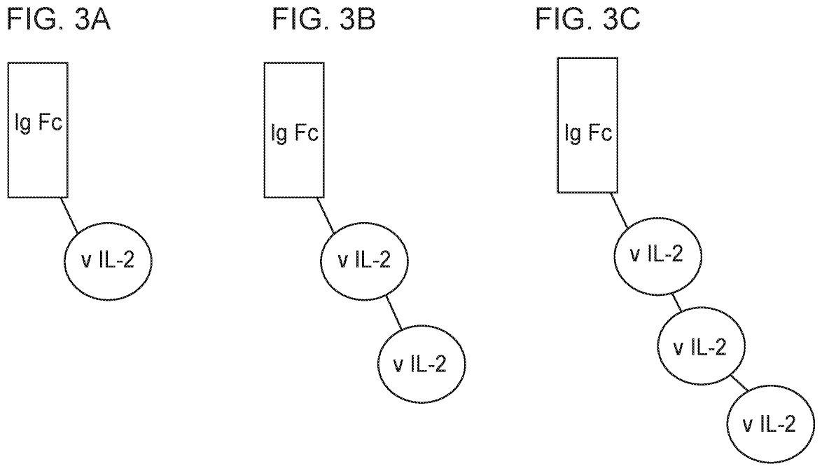

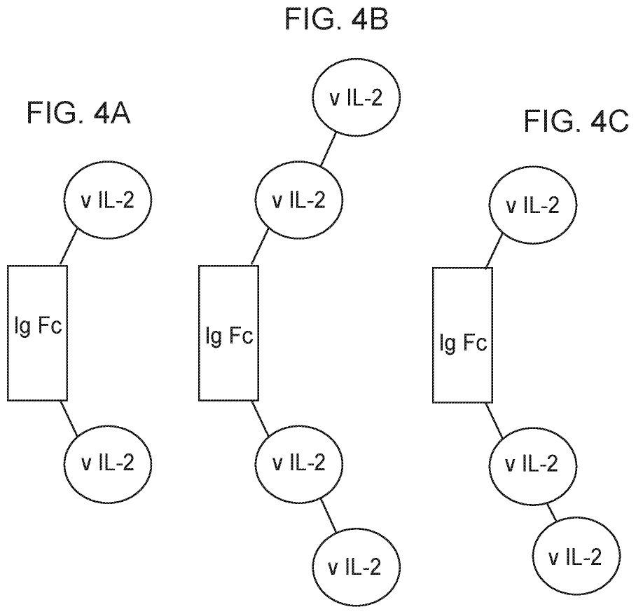

In some cases, the fusion polypeptide comprises at least one IL-2 variant polypeptide fused to the N-terminus of an Ig Fe fusion partner (see, e.g., FIGS. 2A-2C). In some cases, the fusion polypeptide comprises at least one IL-2 variant polypeptide fused to the C-terminus of an Ig Fc fusion partner (see, e.g., FIGS. 3A-3C). In some cases, the fusion polypeptide comprises at least one IL-2 variant polypeptide fused to the N-terminus of an Ig Fc fusion partner, and at least one IL-2 variant polypeptide fused to the C-terminus of an Ig Fc fusion partner (see, e.g., FIGS. 4A-4C). In FIGS. 2A-2C, 3A-3C and 4A-4C, the Ig Fe polypeptide of the fusion polypeptide has been altered using known methods to prevent dimerization with another fusion protein comprising an Ig Fc. See, e.g., Wang et al., Front. Immunol. 8:1545 (November 2017), Ying et al., mAbs 6:5, 1201-1210; September/October 2014, and Ying et al., J. Biol. Chem., vol. 287, no. 23, pp. 19399-19408, Jun. 1, 2012.

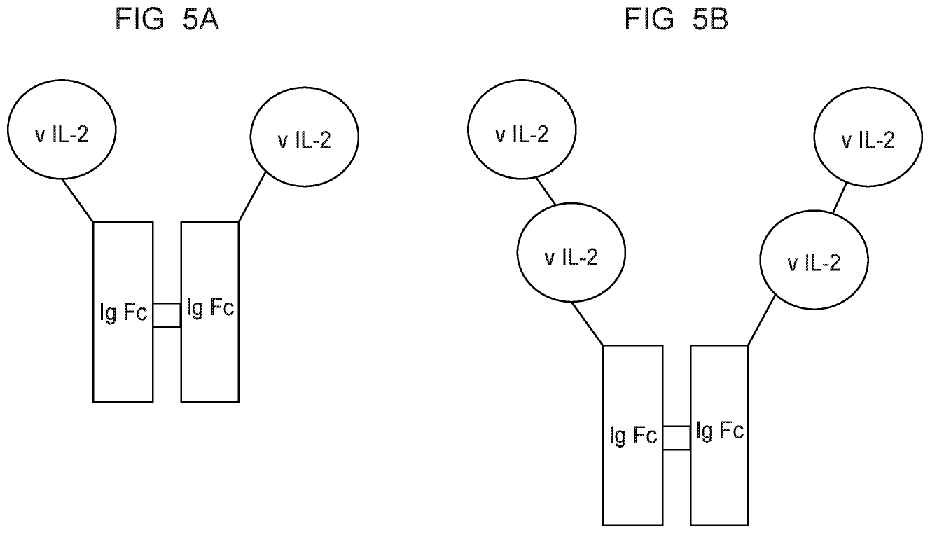

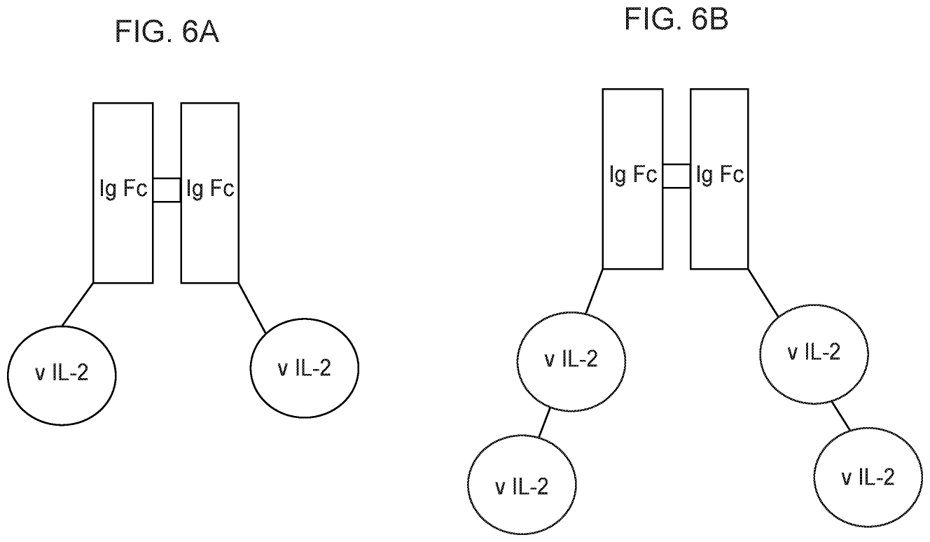

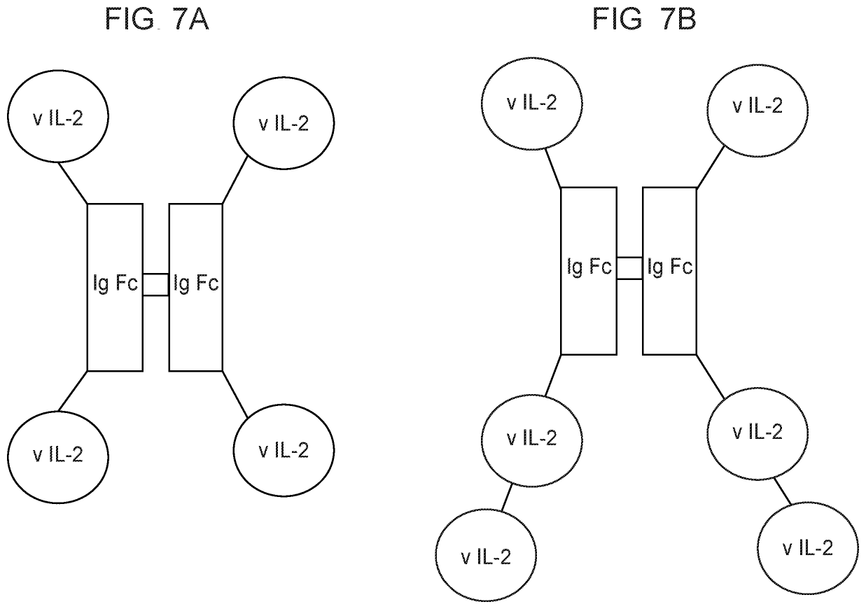

In some cases, the fusion polypeptides can form dimers or higher-order fusion polypeptides. In some cases, the dimers are homodimers in which each fusion polypeptide comprises the same amino acid sequence (see, e.g., FIGS. 5A-5B, 6A-6B, 7A and 7B). When the fusion partner is an Ig Fc, e.g., an IgG1 Fc, disulfide bonds (typically two) can spontaneously form to link the two fusion polypeptides and thereby form a homodimer.

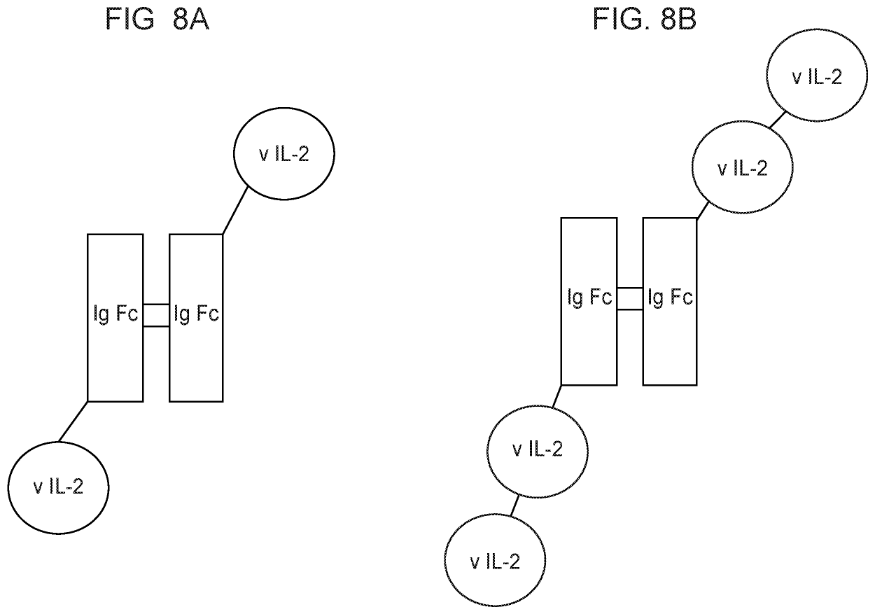

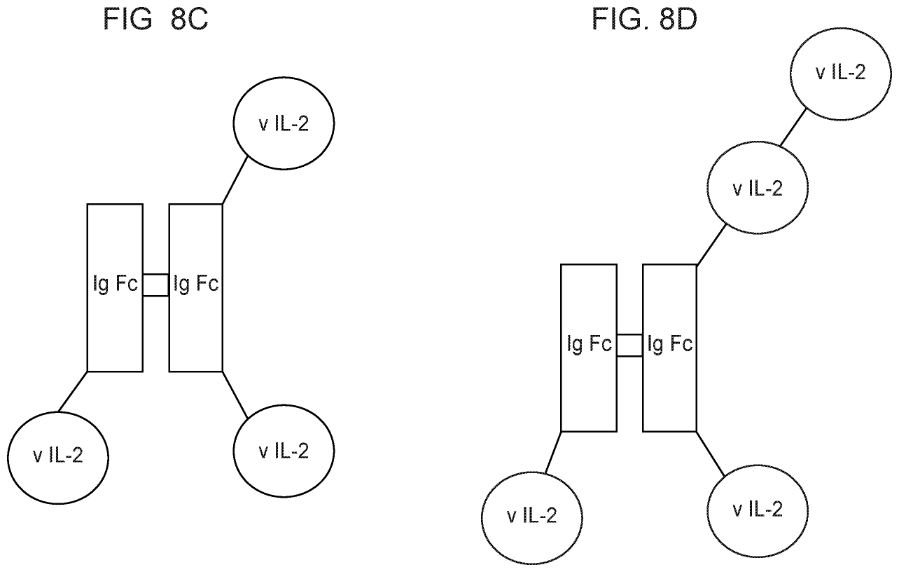

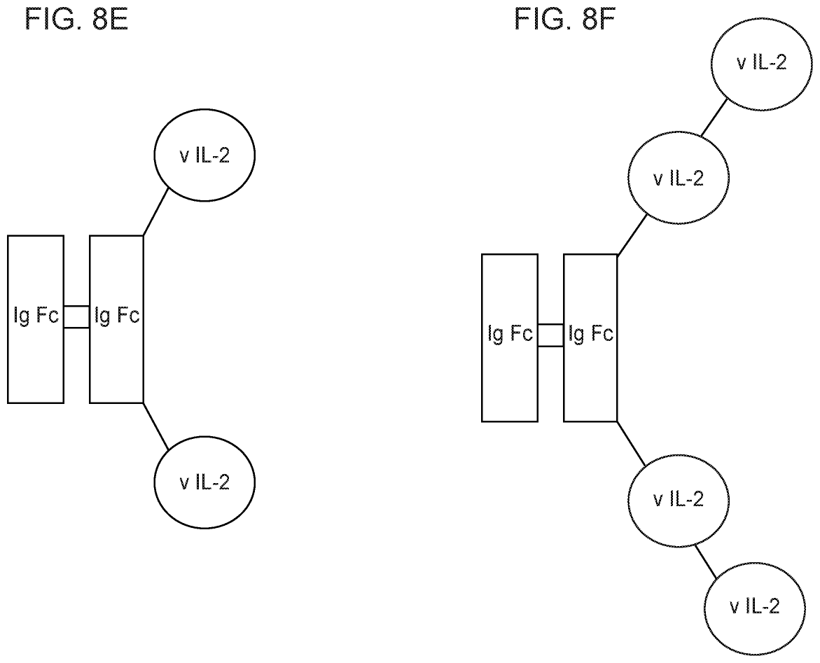

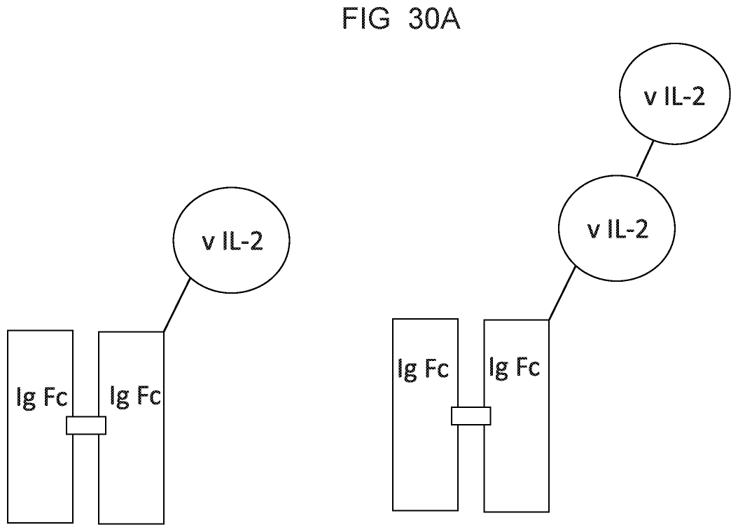

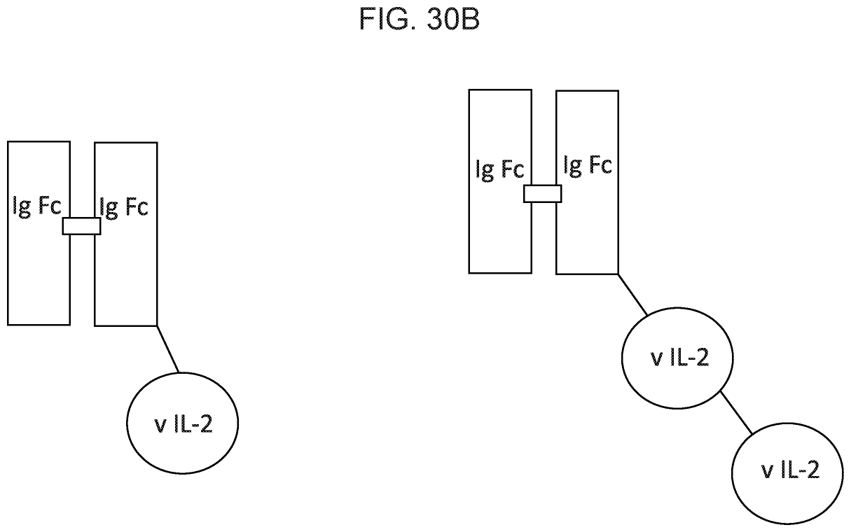

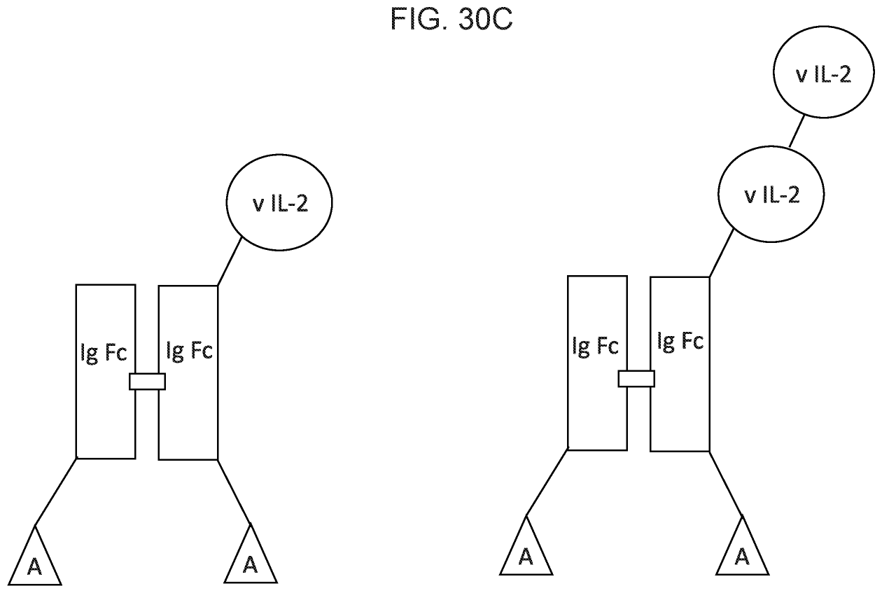

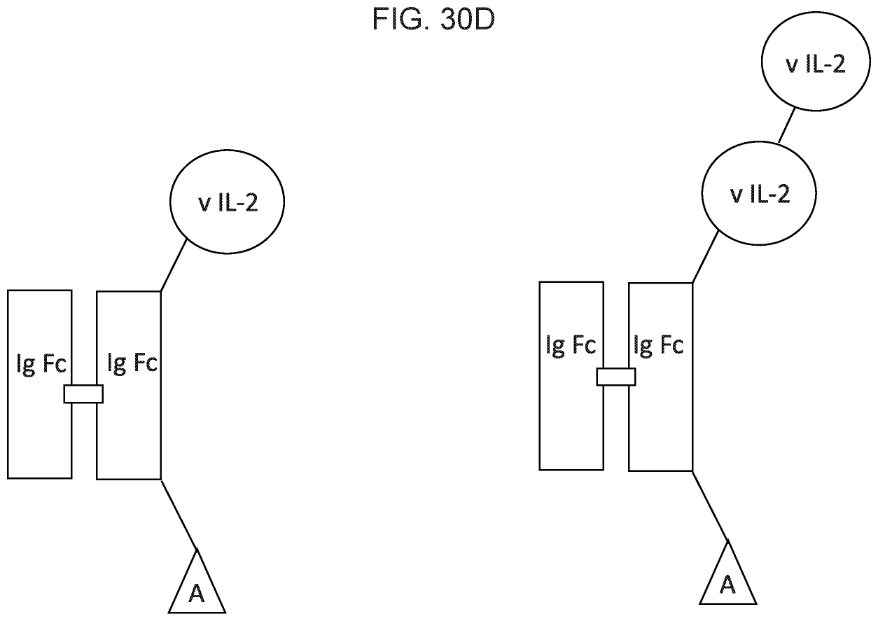

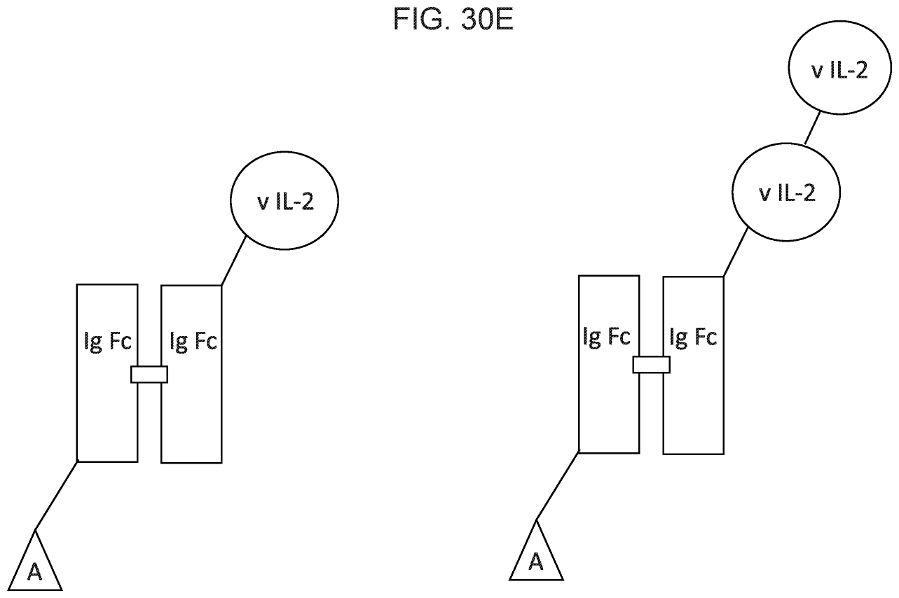

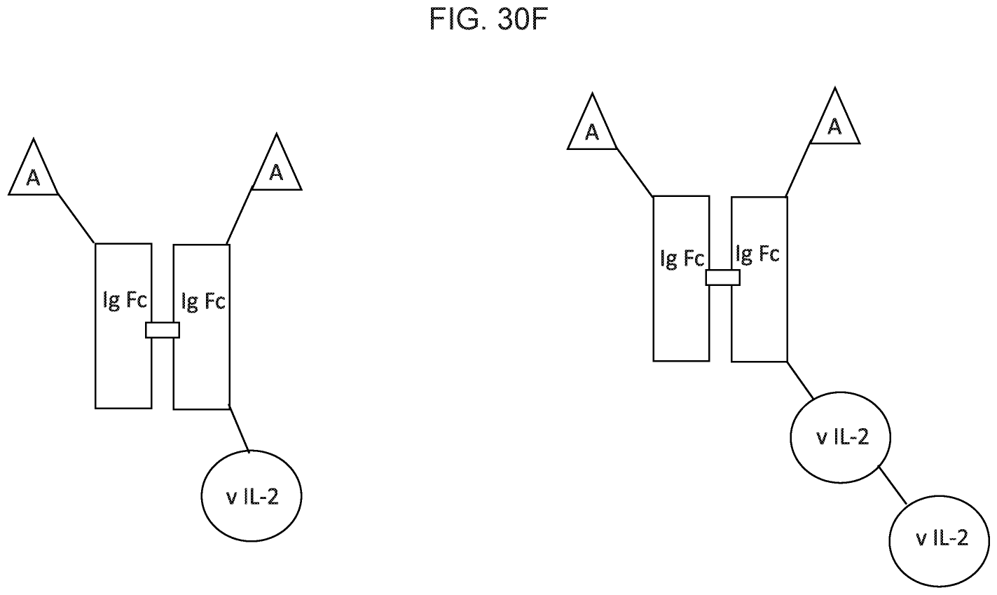

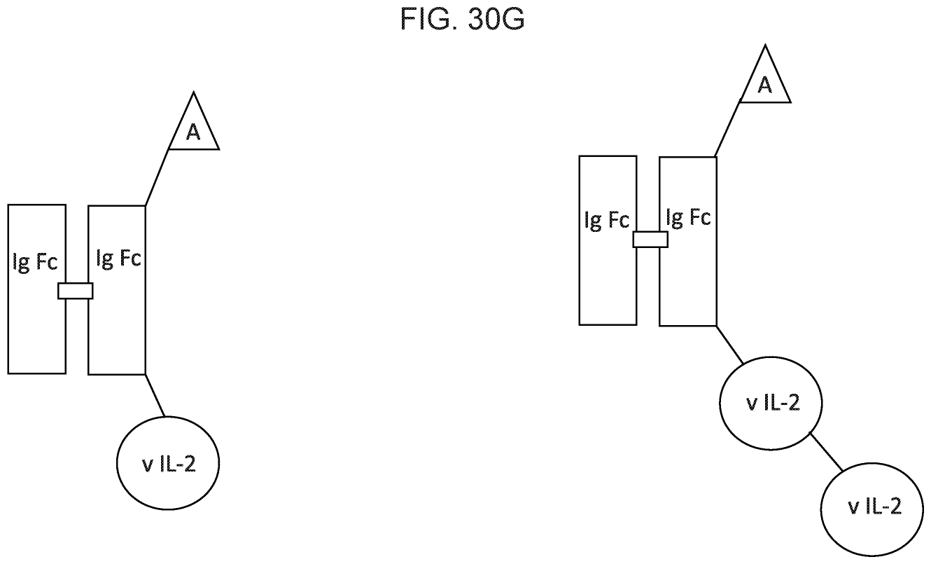

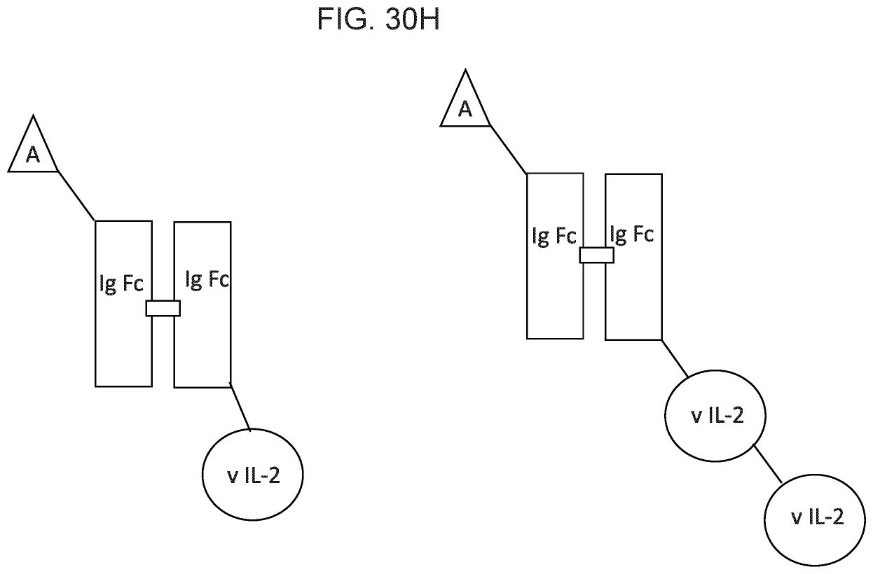

In some cases, it may be desired to form a heterodimeric fusion polypeptide in which each fusion polypeptide comprises a different amino acid sequence (see, e.g., FIGS. 8A-8F and FIGS. 30A-30G). In such instances, it is desirable to employ Ig Fc polypeptides comprising interspecific binding sequences.

Interspecific dimerization sequences, e.g., “Knob-in-Hole” sequences that permit two different fusion polypeptides that differ from one another in amino acid sequence to selectively dimerize. Interspecific binding sequences favor formation of heterodimers with their cognate polypeptide sequence (i.e., the interspecific sequence and its counterpart interspecific sequence), particularly those based on Ig Fe sequence variants. Such interspecific polypeptide sequences include Knob-in-Hole, and Knob-in-Hole sequences that facilitate the formation of one or more disulfide bonds. For example, one interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fe sequence. See Xu et al. mAbs 7:1, 231-242 (2015). Another interspecific binding pair has a first Fe polypeptide with Y349C, T366S, L368A, and Y407V substitutions and a second Ig Fc polypeptide with S354C, and T366W substitutions (disulfide bonds can form between the Y349C and the S354C). See, e.g., Brinkmann and Konthermann, mAbs 9:2, 182-212 (2015). Ig Fe polypeptide sequences, either with or without knob-in-hole modifications, can be stabilized by the formation of disulfide bonds between the Ig Fe polypeptides (e.g., the hinge region disulfide bonds). Thus, in some cases, a dimerized fusion polypeptide can be a heterodimeric fusion polypeptide comprising two fusion polypeptides that are not identical in amino acid sequence; such a dimerized fusion polypeptide can be referred to as a “heterodimeric fusion polypeptide”.

Interspecific dimerization sequences also may be employed to enable a fusion polypeptide to be linked to molecule that does not comprise an IL-2 variant. For example, a fusion polypeptide comprising an IL-2 variant could be linked to a molecule that comprise polypeptides (e.g., antibodies or binding fragments thereof such as single-chain Fe polypeptides (scFvs) or nanobodies) that bind to cancer-associated antigens, thereby enabling the fusion polypeptide comprising an IL-2 variant to localize to tissues comprising the cancer-associated antigen. As discussed below, the fusion polypeptide comprising at least one IL-2 variant could be coupled to a different fusion polypeptide comprising a protein having a different activity.

### Carriers Comprising IL-2 Variant Polypeptides

In some instances, it may be desirable to associate one or more IL-2 variant polypeptides with a carrier such as lipid vesicles (e.g., liposomes) or micelles, nanoparticles, PEGylated proteins (including site-specific PEGylation; i.e., comprising one or more poly(ethylene glycol) (PEG) moieties), or artificial antigen presenting cells, such as engineered erythroid cells and enucleated cells (see, e.g., US2019/0290686). Such carriers and their methods of manufacture are well known in the art and may provide desirable features such as increased stability, manufacturability, in vivo half-life, and/or targeting of the IL-2 variant(s) to target tissues in the body.

### Ig Fc Polypeptides

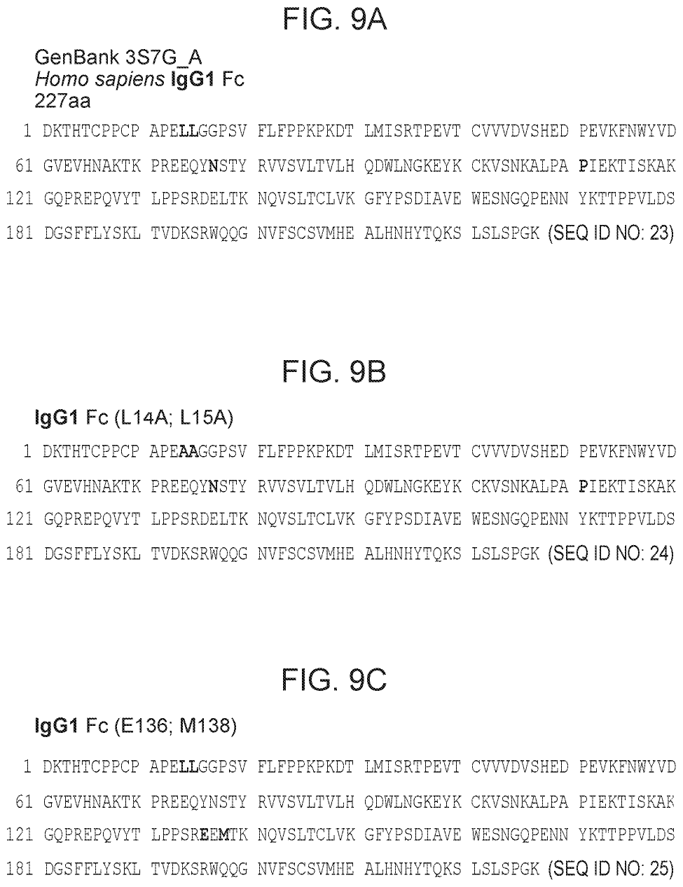

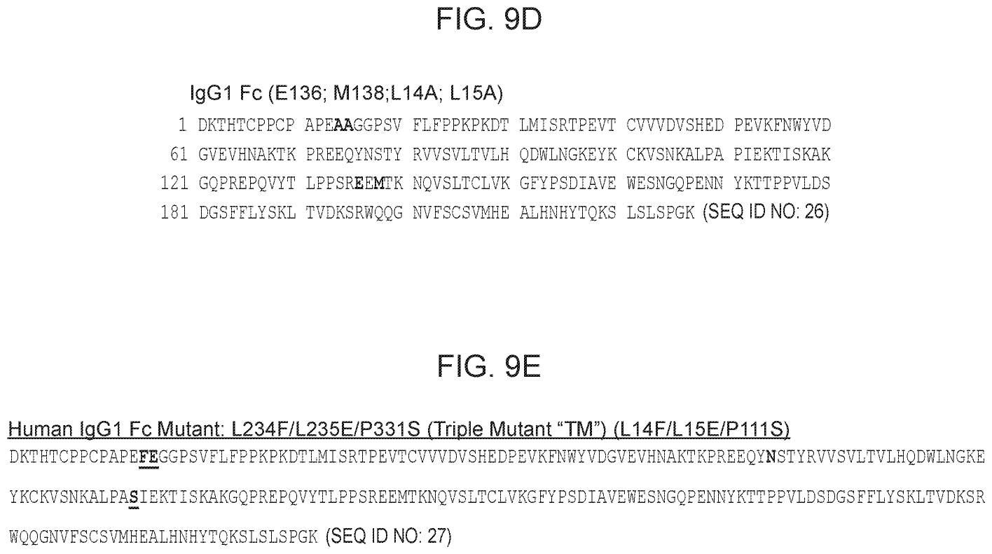

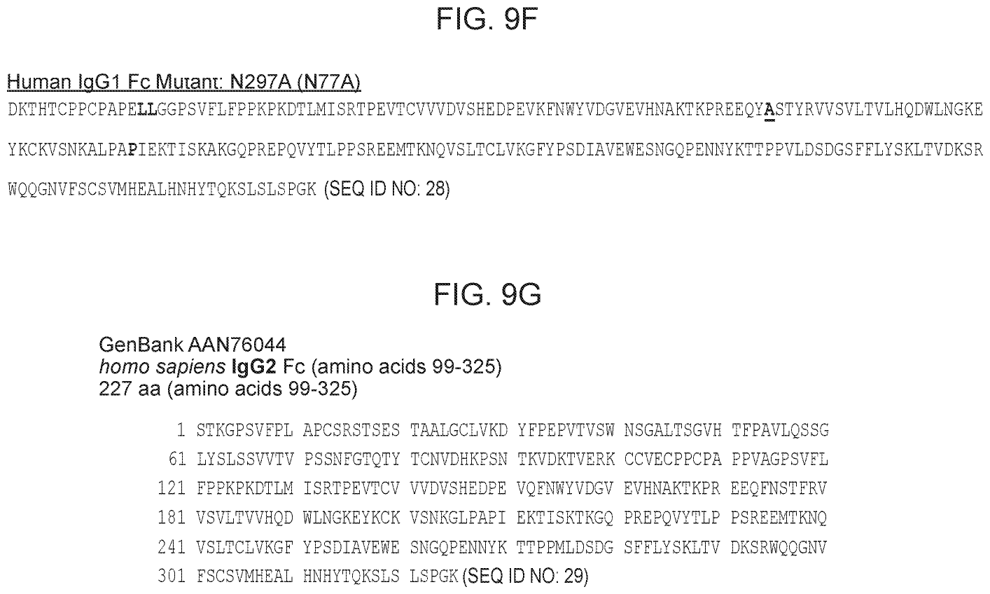

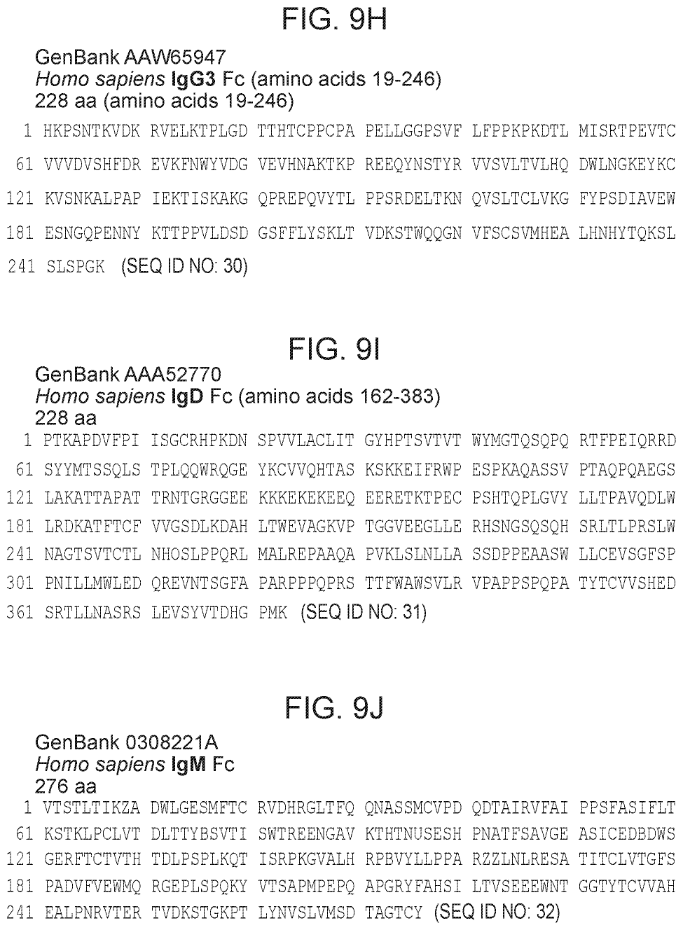

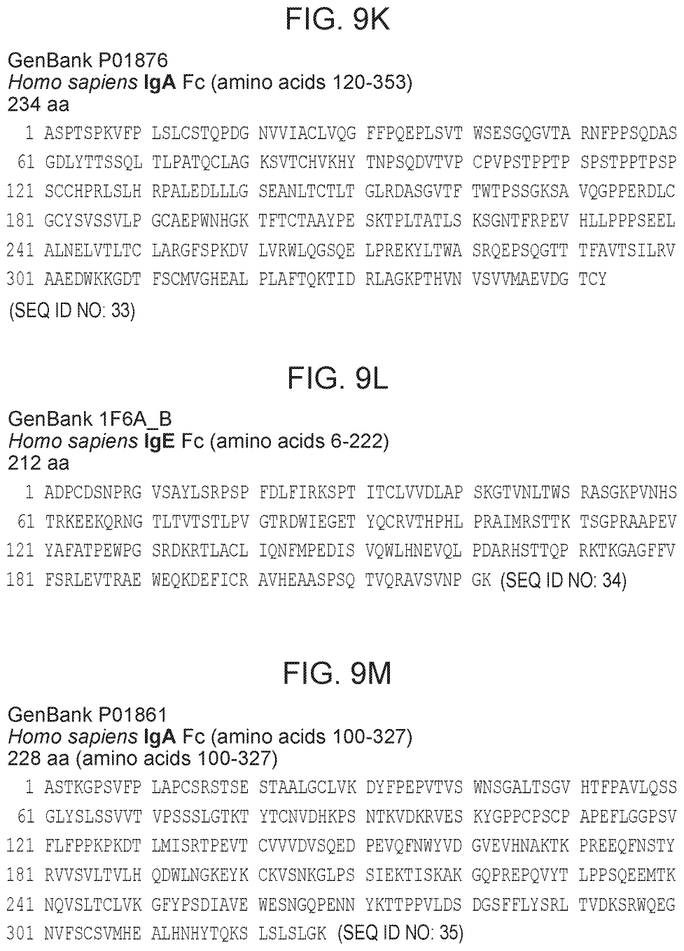

As noted above, fusion polypeptides comprising one or more IL-2 variants can comprise an Ig Fc polypeptide. The Ig Fc polypeptide can be, e.g., a human IgG1 Fe, a human IgG2 Fc, a human IgG3 Fc, a human IgG4 Fc, etc., or a variant of a wild-type Ig Fc polypeptide. Variants include naturally occurring variants, non-naturally occurring variants, and combinations thereof. For example, the Ig Fc can be a variant of a Fc polypeptide such as a human IgG1 Fc, which variant has a substantially reduced ability to effect complement-dependent cytotoxicity (CDC) or antibody-dependent cell cytotoxicity (ADCC). See, e.g., the variant human IgG1 Fc polypeptides of FIG. 9B and FIG. 9D).

In some cases, the Ig Fc polypeptide comprises an amino acid sequence having at least 85%, at least 90%, at least 95%, at least 97%, at least 98%, at least 99%, or 100%, amino acid sequence identity to the Ig Fc amino acid sequence depicted in any one of FIG. 9A-9M. The C-terminal Lys in any of the Ig Fc polypeptides depicted in FIGS. 9A-9I and 9L-9M may be deleted.

In some cases, the Ig Fc polypeptide is an IgG1 Fc polypeptide, or a variant of an IgG1 Fc polypeptide. For example, in some cases, the Ig Fc polypeptide comprises an amino acid sequence having at least 85%, at least 90%, at least 95%, at least 97%, at least 98%, at least 99%, or 100%, amino acid sequence identity to the human IgG1 Fe polypeptide depicted in FIG. 9A, either with or without a C-terminal Lys. As another example, in some cases, the Ig Fc polypeptide comprises an amino acid sequence having at least 85%, at least 90%, at least 95%, at least 97%, at least 98%, at least 99%, or 100%, amino acid sequence identity to the Ig Fc polypeptide depicted in FIG. 9B, either with or without a C-terminal Lys; where the Ig Fc polypeptide comprises an Ala at position 14 and an Ala at position 15, which together are known as the “LALA” mutation. In any of the above embodiments, the Ig Fc polypeptide can have an N77 substitution; i.e., the Ig Fc polypeptide can have an amino acid other than Asn at position 77, where in some cases, the Ig Fc polypeptide has an Ala at position 77. In some cases, an Ig Fc polypeptide comprises the amino acid sequence depicted in FIG. 9A, either with or without a C-terminal Lys. In some cases, an Ig Fc polypeptide comprises the amino acid sequence depicted in FIG. 9B, either with or without a C-terminal Lys.

In some cases, the Ig Fc polypeptide is an IgG1 Fc polypeptide, or a variant of an IgG1 Fc polypeptide, where variants include naturally occurring variants, non-naturally occurring variants, and combinations thereof. For example, in some cases, the Ig Fc polypeptide comprises an amino acid sequence having at least 85%, at least 90%, at least 95%, at least 97%, at least 98%, at least 99%, or 100%, amino acid sequence identity to the human IgG1 Fe polypeptide depicted in FIG. 9C, either with or without a C-terminal Lys; where the Ig Fc polypeptide comprises a Glu at position 136 and a Met at position 138. As another example, in some cases, the Ig Fc polypeptide comprises an amino acid sequence having at least 85%, at least 90%, at least 95%, at least 97%, at least 98%, at least 99%, or 100%, amino acid sequence identity to the human IgG1 Fe polypeptide depicted in FIG. 9D, either with or without a C-terminal Lys; where the Ig Fc polypeptide has Ala at positions 14 and 15; and where the Ig Fc polypeptide comprises a Glu at position 136 and a Met at position 138. In any of the above embodiments, the Ig Fc polypeptide can have an N77 substitution; i.e., the Ig Fc polypeptide can have an amino acid other than Asn at position 77, where in some cases, the Ig Fe polypeptide has an Ala at position 77. In some cases, an Ig Fe polypeptide comprises the amino acid sequence depicted in FIG. 9C, either with or without a C-terminal Lys. In some cases, an Ig Fc polypeptide comprises the amino acid sequence depicted in FIG. 9D, either with or without a C-terminal Lys.

In some cases, the Ig Fc polypeptide comprises the amino acid sequence depicted in FIG. 9E, either with or without a C-terminal Lys (human IgG1 Fc comprising an L234F substitution, an L235E substitution, and a P331S substitution; where L234 corresponds to amino acid 14 of the amino acid sequence depicted in FIG. 9A; L235 corresponds to amino acid 15 of the amino acid sequence depicted in FIG. 9E; and P331 corresponds to amino acid 111 of the amino acid sequence depicted in FIG. 9E). In some cases, the Ig Fc polypeptide comprises the amino acid sequence depicted in FIG. 9F, either with or without a C-terminal Lys, comprising an N279A substitution (N77A of the amino acid sequence depicted in FIG. 9F). Substitutions at N297 lead to the removal of carbohydrate modifications and result antibody sequences with reduced complement component 1q (“C1q”) binding compared to the wild-type protein, and accordingly a reduction in complement-dependent cytotoxicity (CDC). In some cases, the Ig Fc polypeptide comprises a substitution at K322. K322 (e.g., K322A) substitutions show a substantial reduction in reduction in FcγR binding affinity and a reduction in antibody-dependent cell-mediated cytotoxicity (ADCC), with the C1q binding and CDC functions substantially or completely eliminated.

In some cases, the Ig Fc polypeptide comprises an amino acid sequence having at least 85%, at least 90%, at least 95%, at least 97%, at least 98%, at least 99%, or 100%, amino acid sequence identity to the human IgG2 Fc polypeptide depicted in FIG. 9G, either with or without a C-terminal Lys; e.g., the Ig Fc polypeptide comprises an amino acid sequence having at least 85%, at least 90%, at least 95%, at least 97%, at least 98%, at least 99%, or 100%, amino acid sequence identity to amino acids 99-325 of the human IgG2 Fc polypeptide depicted in FIG. 9G (e.g., where the Ig Fc polypeptide has a length of about 227 amino acids). In some cases, the Ig Fc polypeptide comprises an amino acid sequence having at least 85%, at least 90%, at least 95%, at least 97%, at least 98%, at least 99%, or 100%, amino acid sequence identity to the human IgG3 Fe polypeptide depicted in FIG. 911, either with or without a C-terminal Lys; e.g., the Ig Fc polypeptide comprises an amino acid sequence having at least 85%, at least 90%, at least 95%, at least 97%, at least 98%, at least 99%, or 100%, amino acid sequence identity to amino acids 19-246 of the human IgG3 Fe polypeptide depicted in FIG. 9H (e.g., where the Ig Fc polypeptide has a length of about 228 amino acids). In some cases, the Ig Fc polypeptide comprises an amino acid sequence having at least 85%, at least 90%, at least 95%, at least 97%, at least 98%, at least 99%, or 100%, amino acid sequence identity to the human IgM Fc polypeptide depicted in FIG. 9J; e.g., the Ig Fc polypeptide comprises an amino acid sequence having at least 85%, at least 90%, at least 95%, at least 97%, at least 98%, at least 99%, or 100%, amino acid sequence identity to amino acids 1-276 to the human IgM Fe polypeptide depicted in FIG. 9J. In some cases, the Ig Fe polypeptide comprises an amino acid sequence having at least 85%, at least 90%, at least 95%, at least 97%, at least 98%, at least 99%, or 100%, amino acid sequence identity to the human IgA Fc polypeptide depicted in FIG. 9K; e.g., the Ig Fc polypeptide comprises an amino acid sequence having at least 85%, at least 90%, at least 95%, at least 97%, at least 98%, at least 99%, or 100%, amino acid sequence identity to amino acids 1-234 to the human IgA Fc polypeptide depicted in FIG. 9K.

In some cases, the Ig Fc polypeptide comprises an amino acid sequence having at least 85%, at least 90%, at least 95%, at least 97%, at least 98%, at least 99%, or 100%, amino acid sequence identity to the human IgE Fc polypeptide depicted in FIG. 9L, either with or without a C-terminal Lys. In some cases, the Ig Fc polypeptide comprises an amino acid sequence having at least 85%, at least 90%, at least 95%, at least 97%, at least 98%, at least 99%, or 100%, amino acid sequence identity to the human IgE Fc polypeptide depicted in FIG. 9L.

In some cases, the Ig Fc polypeptide comprises an amino acid sequence having at least 85%, at least 90%, at least 95%, at least 97%, at least 98%, at least 99%, or 100%, amino acid sequence identity to the human IgG4 Fc polypeptide depicted in FIG. 9M, either with or without a C-terminal Lys. In some cases, the Ig Fc polypeptide comprises an amino acid sequence having at least 85%, at least 90%, at least 95%, at least 97%, at least 98%, at least 99%, or 100%, amino acid sequence identity to amino acids 100 to 327 of the human IgG4 Fc polypeptide depicted in FIG. 9M (e.g., where the Ig Fc polypeptide has a length of about 228 amino acids, or 227 if the C-terminal Lys is omitted).

In some cases, the IG4 Fc polypeptide comprises the following amino acid sequence:

(SEQ ID NO: 37)
PPCPSCPAPEFLGGPSVFLFPPKPKDTLMISRTPEVTCVVVDVSQEDPEV
QFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFY
PSDIAVEWESNGQPENNYKCTPPVLDSDGSFFLYSRLTVDKSRWQEGNVF
SCSVMHEALHNHYTQKSLSLSPG.

As noted above, disulfide bonds (typically two) may spontaneously form between two Ig Fc polypeptides, e.g., between the Ig Fe heavy chain CH2 and CH3 domains, to form a dimer comprising two fusion polypeptides.

### Fusion Polypeptides Comprising IL-2 Variant Polypeptides and Functional Proteins

In some cases, it may be desirable for the fusion polypeptide comprising the one or more IL-2 variant polypeptides to include one or more proteins (“functional proteins”) that are not IL-2 variant polypeptides. Such functional proteins can include, for example, immunomodulatory polypeptides, scFv's or nanobodies that target cancer-associated antigens, T cell receptors (TCRs), TCR-like antibody fragments, proteins that target NK cells, e.g., anti-NK antibodies, and receptor-trap proteins, e.g., TGF-β trap proteins.

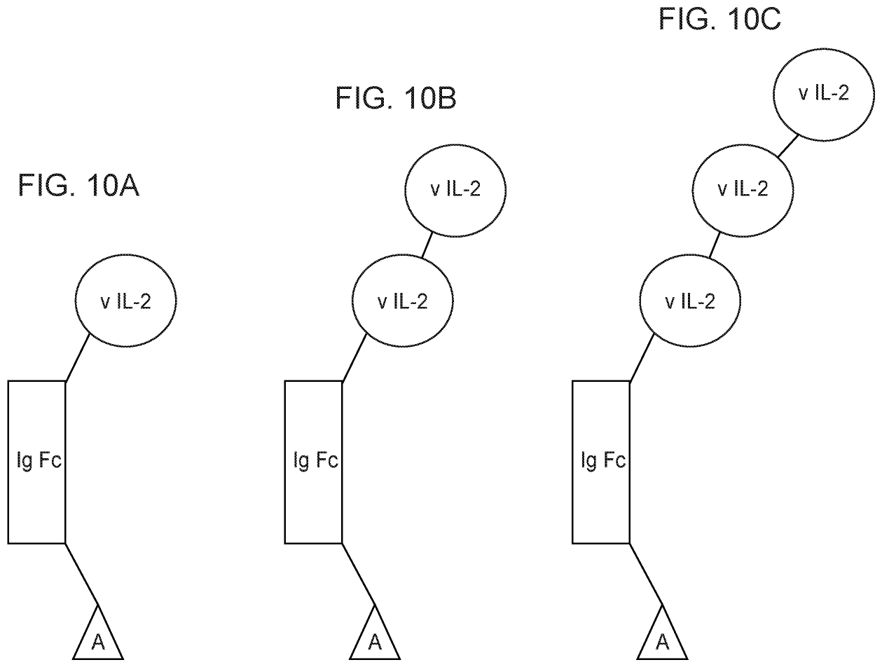

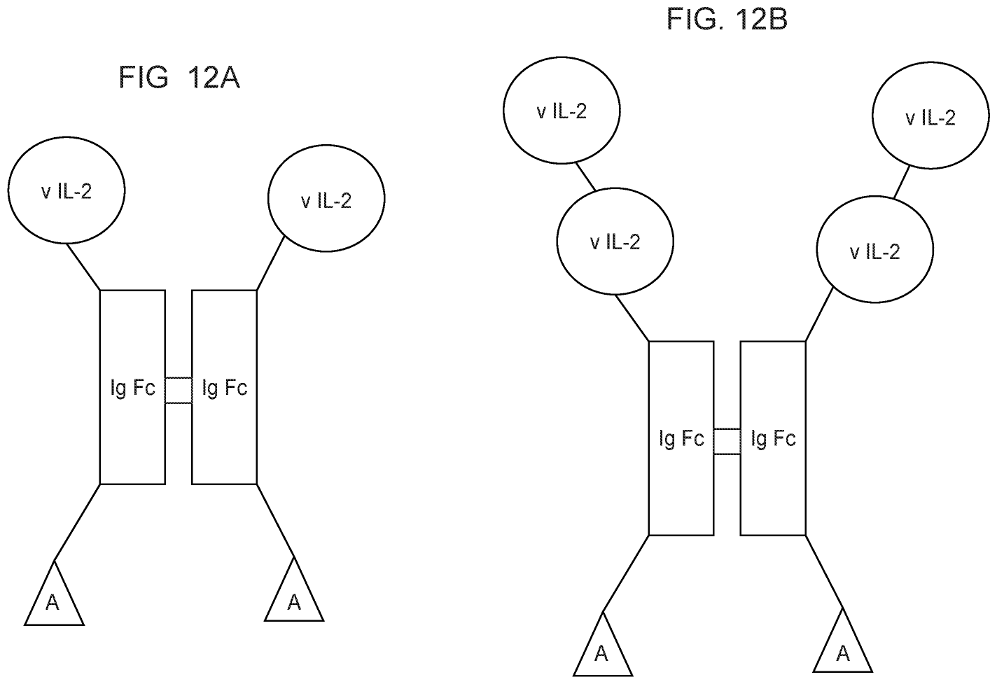

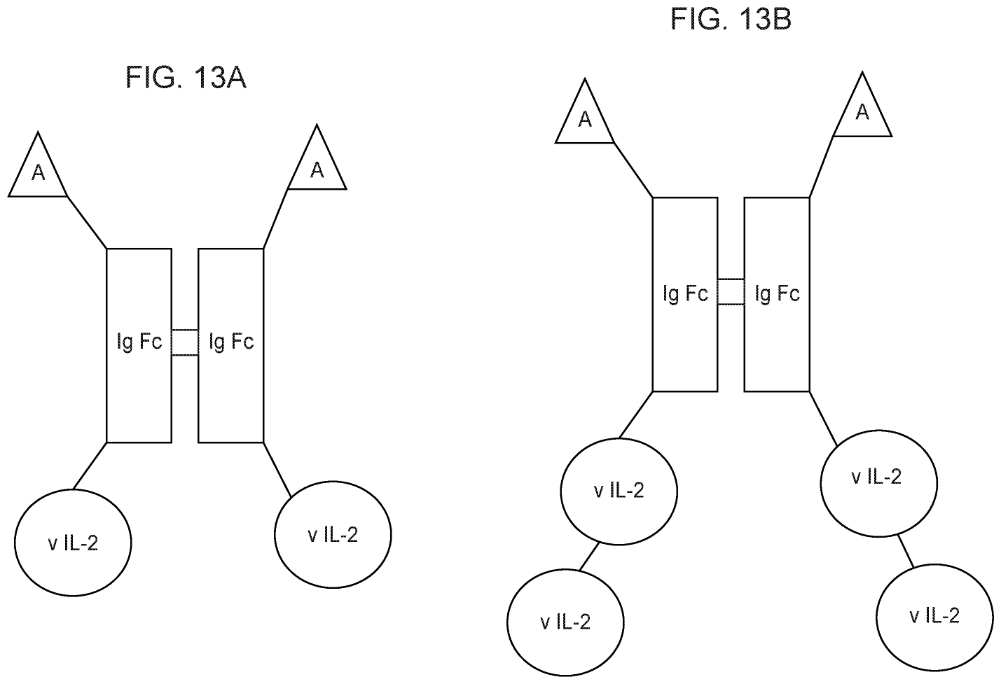

Fusion polypeptides comprising the one or more IL-2 variant polypeptides and one or more functional proteins can be arranged in a variety of configurations. FIGS. 10A-10C and FIGS. 11A-11C and FIGS. 30A-30G illustrate fusion polypeptides comprising one or more IL-2 variant polypeptides and a heterologous fusion partner comprising both an Ig Fe polypeptide and a functional protein. FIGS. 12A-12B and FIGS. 13A-13B illustrate homodimers comprising individual fusion polypeptides, each of which comprises one or more IL-2 variant polypeptides, and a heterologous fusion partner comprising both an Ig Fe polypeptide and a functional protein. FIGS. 30A-30G illustrate heterodimers comprising one or more variant IL-2 polypeptides, IgFc polypeptides, and, in some cases, a functional protein. As illustrated in FIGS. 30A-30G, in some cases, a heterodimer comprises a first Ig Fe polypeptide and a second Ig Fe polypeptide, where the first Ig Fe polypeptide is not fused to a variant IL-2 polypeptide, and where the second Ig Fe polypeptide is fused to a variant IL-2 polypeptide. The first and/or the second polypeptide may also be fused to one or more functional proteins (designated “A”). When the first and second polypeptides of a heterodimer each comprise a functional protein, the functional proteins may be the same or different (i.e., the functional proteins may have the same amino acid sequence, or may differ in amino acid sequence).

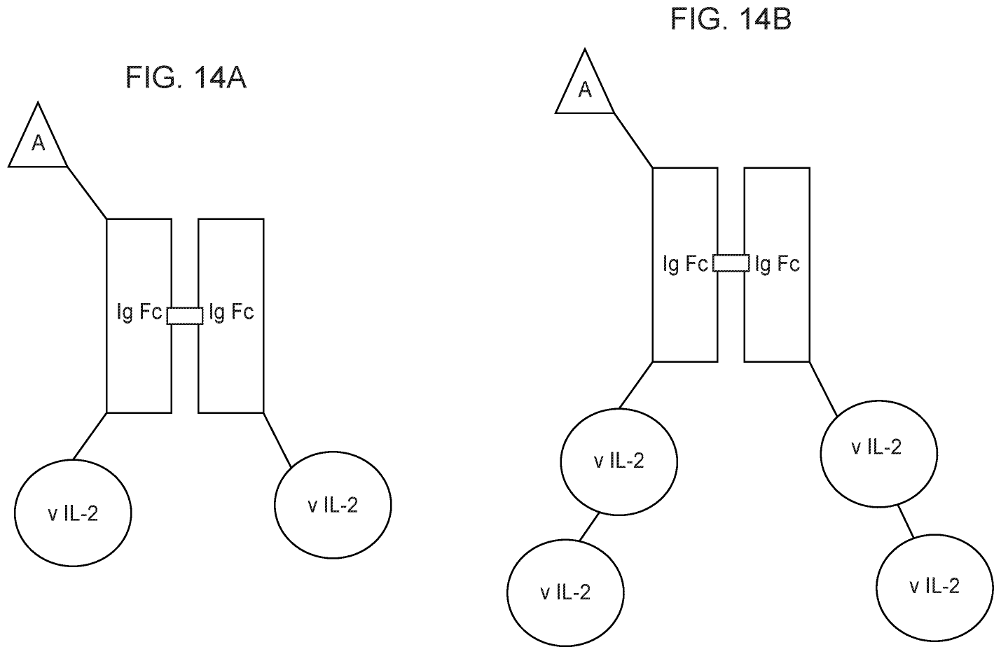

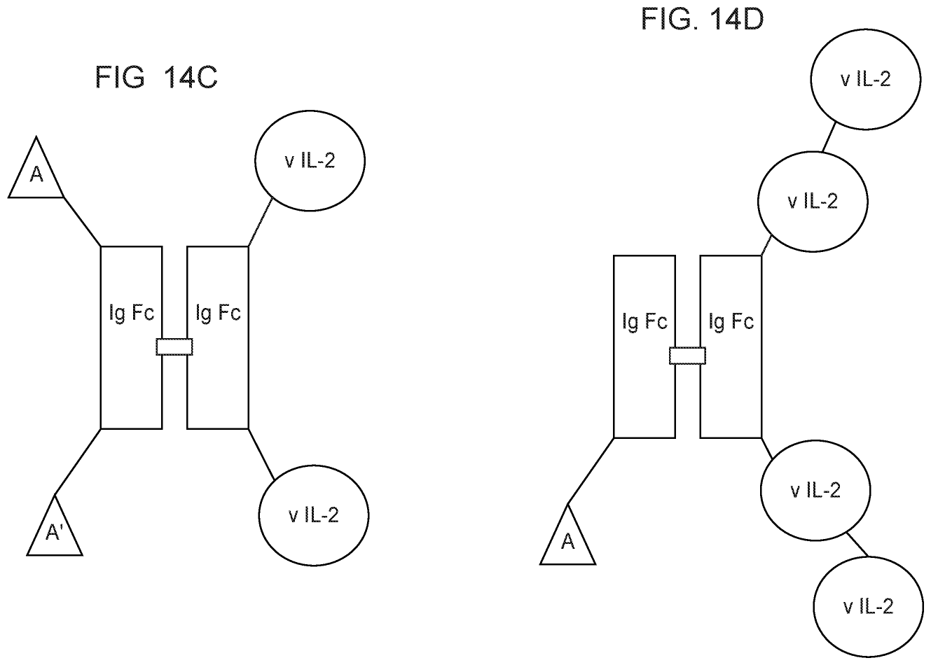

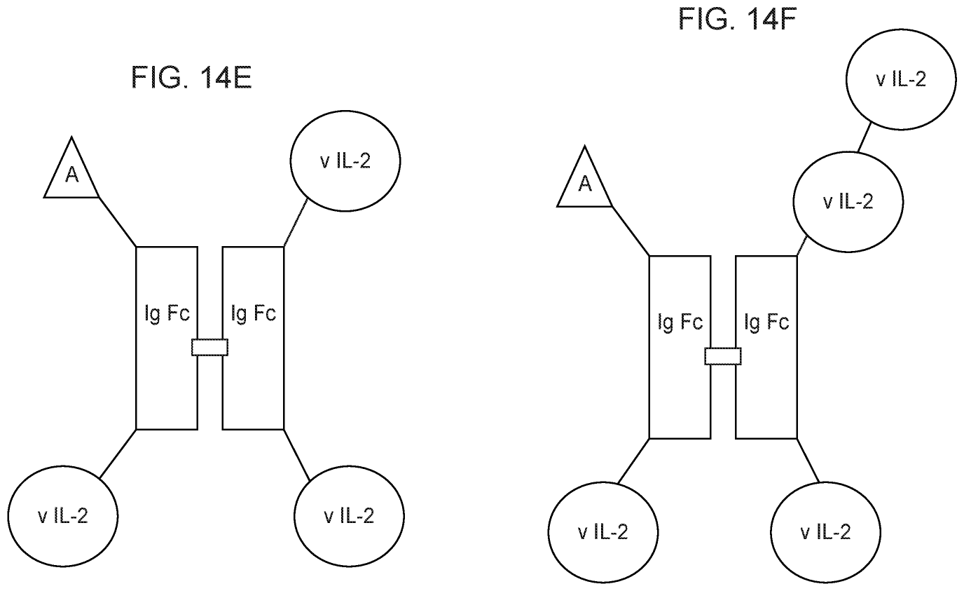

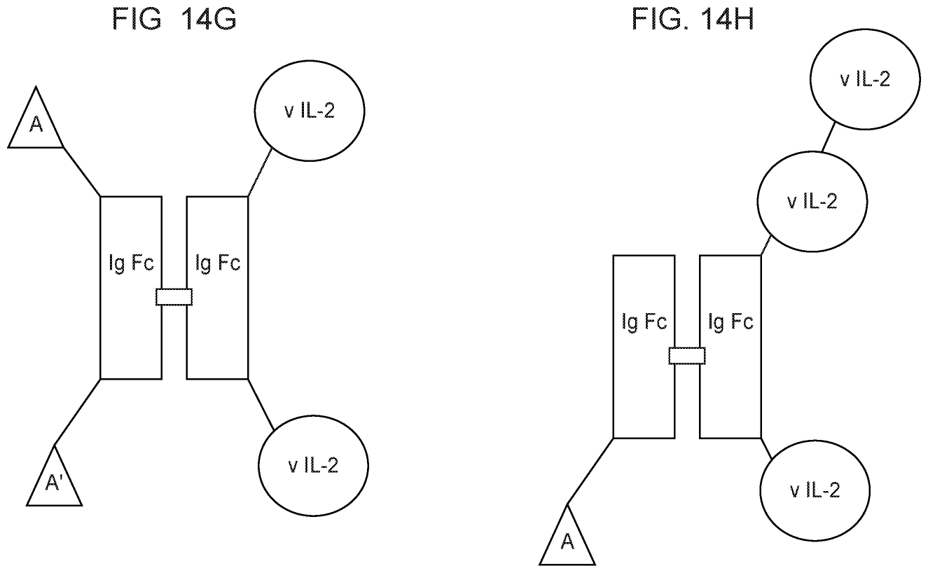

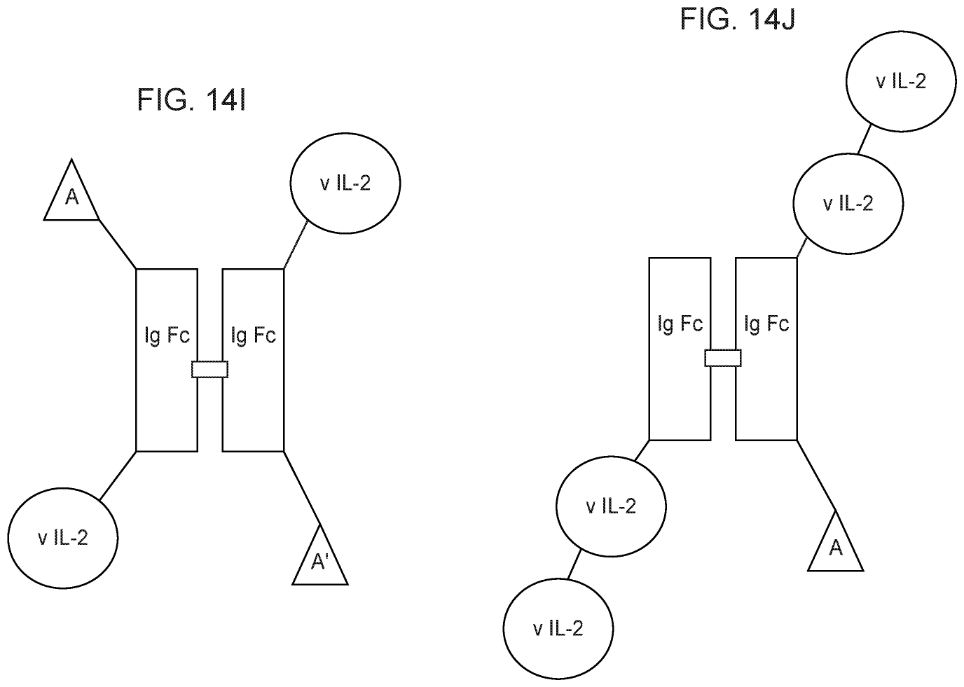

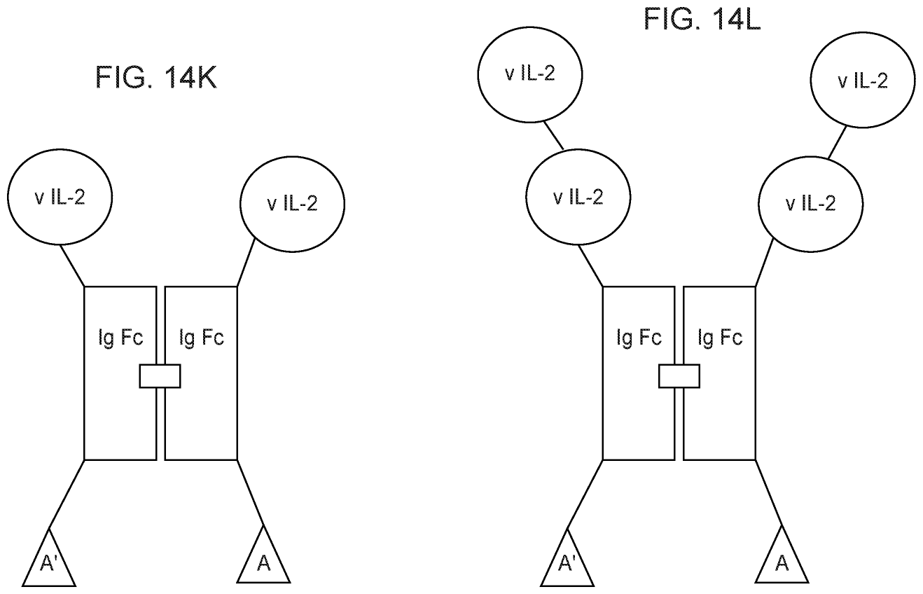

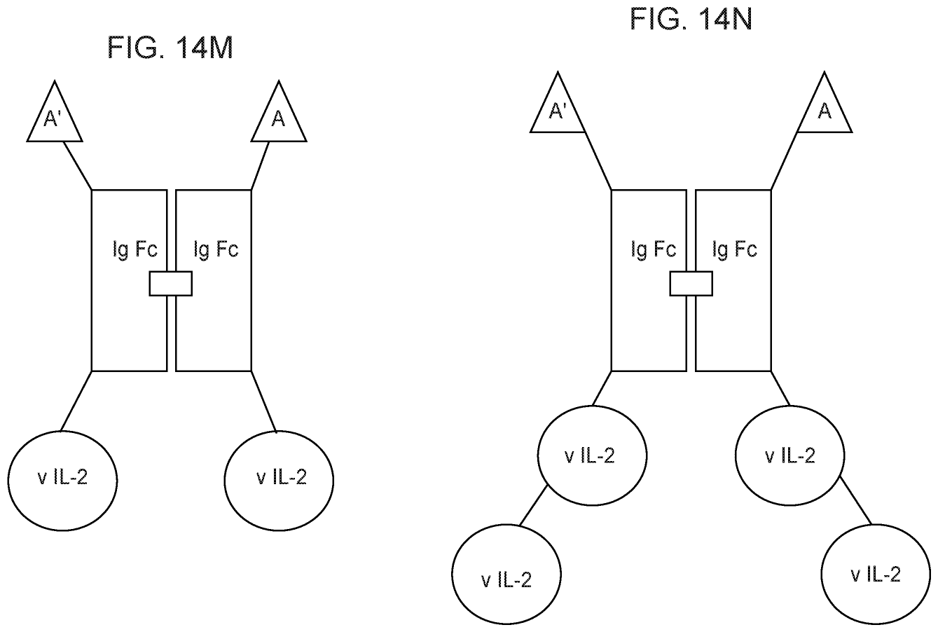

FIGS. 14A-14N illustrate heterodimeric polypeptides comprising individual fusion polypeptides, each of which comprises one or more IL-2 variant polypeptides and one or more functional proteins. Such proteins can be prepared using the interspecific binding sequences discussed above. In cases where a heterodimeric polypeptide comprises more than one functional protein, the functional proteins can be the same or different. See, e.g., FIG. 14C, FIG. 14G,FIG. 14I, FIG. 14K, FIG. 14L, FIG. 14M, and FIG. 14N, where the functional proteins A and A′ can be the same or different.

As noted above, non-antibody polypeptide scaffolds may be used in place of Ig Fc polypeptides.

As also noted above, the IL-2 variant polypeptide instead may be provided on a carrier such as a lipid vesicle (e.g., liposome) or micelle, a nanoparticle, a PEGylated protein (including site-specific PEGylation), a fibronectin-based scaffold protein, or an artificial antigen presenting cell, such as engineered erythroid cells and enucleated cells (see, e.g., US2019/0290686). In such instances, the functional protein is likewise associated with the carrier through means that are known in the art.

Some exemplary functional proteins are described below.

### Cancer-Targeting Polypeptides (CTPs)

Functional proteins can include cancer-targeting polypeptides (CTPs) that are specific for a cancer-associated epitope. A “cancer-associated” epitope is an epitope that is present in a cancer-associated antigen. In some cases, a CTP is an antibody. In some cases, a CTP is a single-chain T-cell receptor (scTCR). In some cases, the target of a CTP is a peptide/HLA (pHLA) complex on the surface of a cancer cell, where the peptide can be a cancer-associated peptide (e.g., a peptide fragment of a cancer-associated antigen).

Cancer-Associated Antigens that can be Targeted by CTPs

Cancer-associated antigens that can be targeted with a cancer-targeting polypeptide include, e.g., NY-ESO (New York Esophageal Squamous Cell Carcinoma 1), MART-1 (melanoma antigen recognized by T cells 1, also known as Mclan-A), HPV (human papilloma virus) E6, BCMA (B-cell maturation antigen), CD123, CD133, CD171, CD19, CD20, CD22, CD30, CD33, CEA (carcinoembryonic antigen), EGFR (epidermal growth factor receptor), EGFRvIII (epidermal growth factor receptor variant III), EpCAM (epithelial cell adhesion molecule), EphA2 (ephrin type-A receptor 2), disialoganglioside GD2, GPC3 (glypican-3), HER2, IL13Ralpha2 (Interleukin 13 receptor subunit alpha-2), LeY (a difucosylated type 2 blood group-related antigen), MAGE-A3 (melanoma-associated antigen 3), melanoma glycoprotein, mesothelin, MUC1 (mucin 1), MUC16 (mucin-16), myelin, NKG2D (Natural Killer Group 2D) ligands, PSMA (prostate specific membrane antigen), and ROR1 (type I receptor tyrosine kinase-like orphan receptor).

Cancer-associated antigens that can be targeted with a CTP include, but are not limited to, 17-1A-antigen, alpha-fetoprotein (AFP), alpha-actinin-4, A3, antigen specific for A33 antibody, ART-4, B7, Ba 733, BAGE, bcl-2, bcl-6, BCMA, BrE3-antigen, CA125, CAMEL, CAP-1, carbonic anhydrase IX (CAIX), CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CEA, CEACAM5, CEACAM6, claudin (e.g., claudin-1, claudin-10, claudin-18 (e.g., claudin-18, isoform 2)), complement factors (such as C3, C3a, C3b, C5a and C5), colon-specific antigen-p (CSAp), c-Met, CTLA-4, CXCR4, CXCR7, CXCL12, DAM, Dickkopf-related protein (DKK), ED-B fibronectin, epidermal growth factor receptor (EGFR), EGFRvIII, EGP-1 (TROP-2), EGP-2, ELF2-M, Ep-CAM, EphA2, EphA3, fibroblast activation protein (FAP), fibroblast growth factor (FGF), Flt-1, Flt-3, folate binding protein, folate receptor, G250 antigen, gangliosides (such as GC2, GD3 and GM2), GAGE, GD2, gp100, GPC3, GRO-13, HLA-DR, HM1.24, human chorionic gonadotropin (HCG) and its subunits, HER2, HER3, HMGB-1, hypoxia inducible factor (HIF-1), HIF-1a, HSP70-2M, HST-2, Ia, IFN-gamma, IFN-alpha, IFN-beta, IFN-X, IL-4R, IL-6R, IL-13R, IL13Ralpha2, IL-15R, IL-17R, IL-18R, IL-2, IL-6, IL-8, IL-12, IL-15, IL-17, IL-18, IL-23, IL-25, ILGF, ILGF-1R, insulin-like growth factor-1 (IGF-1), IGF-1R, integrin αVβ3, integrin α5β1, KC4-antigen, killer-cell immunoglobulin-like receptor (KIR), Kras, KS-1-antigen, KS1-4, LDR/FUT, Le^{gamma}, macrophage migration inhibitory factor (MIF), MAGE, MAGE-3, MART-1, MART-2, mCRP, MCP-1, melanoma glycoprotein, mesothelin, MIP-1A, MIP-1B, MIF, mucins (such as MUC1, MUC2, MUC3, MUC4, MUC5ac, MUC13, MUC16, MUM-1/2 and MUM-3), NCA66, NCA95, NCA90, Nectin-4, NY-ESO-1, PAM4 antigen, pancreatic cancer mucin, PD-1, PD-L1, PD-1 receptor, placental growth factor, p53, PLAGL2, prostatic acid phosphatase, PSA, PRAME, PSMA, P1GF, RSS, RANTES, SAGE, 5100, survivin, survivin-2B, T101, TAC, TAG-72, tenascin, Thomson-Friedenreich antigens, Tn antigen, TNF-alpha, tumor necrosis antigens, TRAG-3, TRAIL receptors, vascular endothelial growth factor (VEGF), VEGF receptor (VEGFR), WT-1, and COL6A3 exon 6.

In some cases, the cancer-associated antigen is an antigen associated with a hematological cancer. Examples of such antigens include, but are not limited to, BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, LGF-R1, 1L6, KIR, PD-1, PD-L1, TRAILR1, TRAILR2, transferrin receptor, and VEGF. In some cases, the cancer-associated antigen is an antigen expressed by malignant B cells, such as CD19, CD20, CD22, CD25, CD38, CD40, CD45, CD74, CD80, CTLA-4, IGF-R1, IL6, PD-1, TRAILR2, or VEGF.

In some cases, the cancer-associated antigen is an antigen associated with a solid tumor. Examples of such antigens include, but are not limited to, CAIX, cadherins, CEA, c-MET, CTLA-4, EGFR family members, EpCAM, EphA3, FAP, folate-binding protein, FR-alpha, gangliosides (such as GC2, GD3 and GM2), HER2, HER3, IGF-1R, integrin αVβ3, integrin α5β1, Le^{gamma}, Liv1, mesothelin, mucins, NaPi2b, PD-1, PD-L1, PD-1 receptor, pgA33, PSMA, RANKL, ROR1, TAG-72, tenascin, TRAILR1, TRAILR2, VEGF, VEGFR, and others listed above.

CTPs that Target Peptide/HLA Complexes

In some cases, the target of a CTP is a peptide-HLA (pHLA) complex on the surface of a cancer cell, where the peptide can be a cancer-associated peptide (e.g., a peptide fragment of a cancer-associated antigen). In such cases, the functional protein can be a T cell receptor or other polypeptide that binds to the pHLA complex on the surface of a cancer cell. Such pHLA complexes are known in the art. Cancer-associated peptides are known in the art. In some cases, a cancer-associated peptide is bound to an HLA complex comprising an HLA-A*0201 heavy chain and a β2M polypeptide.

In some cases, the epitope present in the pHLA on the surface of a cancer cell is bound to an HLA complex comprising an HLA heavy chain such as HLA-A*0101, A*0201, A*0301, A*1101, A*2301, A*2402, A*2407, A*3303, and/or A*3401. In some cases, the epitope present in the pHLA on the surface of a cancer cell is bound to an HLA complex comprising an HLA heavy chain such as HLA-B*0702, B*0801, B*1502, B*3802, B*4001, B*4601, and/or B*5301. In some cases, the epitope present in the pHLA on the surface of a cancer cell is bound to an HLA complex comprising an HLA heavy chain such as C*0102, C*0303, C*0304, C*0401, C*0602, C*0701, C*702, C*0801, and/or C*1502.

In some cases, the epitope is a cancer-associated epitope of any one of the following cancer-associated antigens: a MUC1 polypeptide, an LMP2 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a claudin polypeptide (e.g., claudin-1, claudin-10, claudin-18 (e.g., claudin-18, isoform 2)), a Nectin-4 polypeptide, a melanoma antigen recognized by T-cells (melanA/MART1) polypeptide, a Ras polypeptide, a gp100 polypeptide, a proteinase3 (PR1) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFβ) polypeptide, a MAD-CT-2 polypeptide, a Fos-related antigen-1 (FOSL) polypeptide; a human papilloma virus (HPV) antigen; an alpha-feto protein (AFP) antigen; and a Wilms tumor-1 (WT1) antigen.

CTPs that are Antibodies

As noted above, in some cases, a CTP is an antibody that is specific for a cancer-associated antigen. In some cases, the CTP is an antibody specific for a peptide/HLA complex on the surface of a cancer cell, where the peptide can be a cancer-associated peptide (e.g., a peptide of a cancer-associated antigen).

Non-limiting examples of cancer-associated antigen-targeting antibodies include, but are not limited to, abituzumab (anti-CD51), LL1 (anti-CD74), LL2 or RFB4 (anti-CD22), veltuzumab (hA20, anti-CD20), rituxumab (anti-CD20), obinutuzumab (GA101, anti-CD20), daratumumab (anti-CD38), lambrolizumab (anti-PD-1 receptor), nivolumab (anti-PD-1 receptor), ipilimumab (anti-CTLA-4), RS7 (anti-TROP-2), PAM4 or KC4 (both anti-mucin), MN-14 (anti-CEA), MN-15 or MN-3 (anti-CEACAM6), Mu-9 (anti-colon-specific antigen-p), Immu 31 (anti-alpha-fetoprotein), Ri (anti-IGF-1R), A19 (anti-CD19), TAG-72 (e.g., CC49), Tn, J591 or HuJ591 (anti-PSMA), AB-PG1-XG1-026 (anti-PSMA dimer), D2/B (anti-PSMA), G250 (anti-carbonic anhydrase IX), L243 (anti-HLA-DR) alemtuzumab (anti-CD52), oportuzumab (anti-EpCAM), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (also known as clivatuzumab; anti-mucin), trastuzumab (anti-HER2), pertuzumab (anti-HER2), polatuzumab (anti-CD79b), and anetumab (anti-mesothelin).

In some cases, the cancer-targeting polypeptide is an antibody. In some cases, the cancer-targeting polypeptide is a single-chain antibody. In some cases, the cancer-targeting polypeptide is a scFv. In some cases, the cancer-targeting polypeptide is a nanobody (also referred to as a single domain antibody (sdAb)). In some cases, the cancer-targeting polypeptide is a heavy chain nanobody. In some cases, the cancer-targeting polypeptide is a light chain nanobody.

VH and VL amino acid sequences of various tumor antigen-binding antibodies are known in the art, as are the light chain and heavy chain CDRs of such antibodies. See, e.g., Ling et al. (2018) Frontiers Immunol. 9:469; WO 2005/012493; US 2019/0119375; US 2013/0066055. The following are non-limiting examples of tumor antigen-binding antibodies. Exemplary antibodies for which light- and heavy-chain sequences are known include anti-Her2 antibodies, anti-CD19 antibodies, anti-mesothelin antibodies, antibodies to trophoblast cell surface antigen 2 (Trop-2) (also known as epithelial glycoprotein-1, gastrointestinal tumor-associated antigen GA733-1, membrane component chromosome 1 surface marker-1, and tumor-associated calcium signal transducer-2), anti-BCMA (B-cell maturation antigen) antibodies, anti-MUC16 (also known as CA125), and anti-claudin-18 isoform 2 (“claudin-18.2”).

In some cases, the antibody targets human serum albumin (HSA). By binding to HSA, such anti-HSA antibodies can tether the fusion protein comprising the IL-2 variant polypeptide(s) to HSA and thereby potentially extend the serum half-life of the fusion protein. Alternatively, as discussed below, one or more IL-2 variant polypeptides may be fused directly to the heavy or light chain of an anti-HSA antibody.

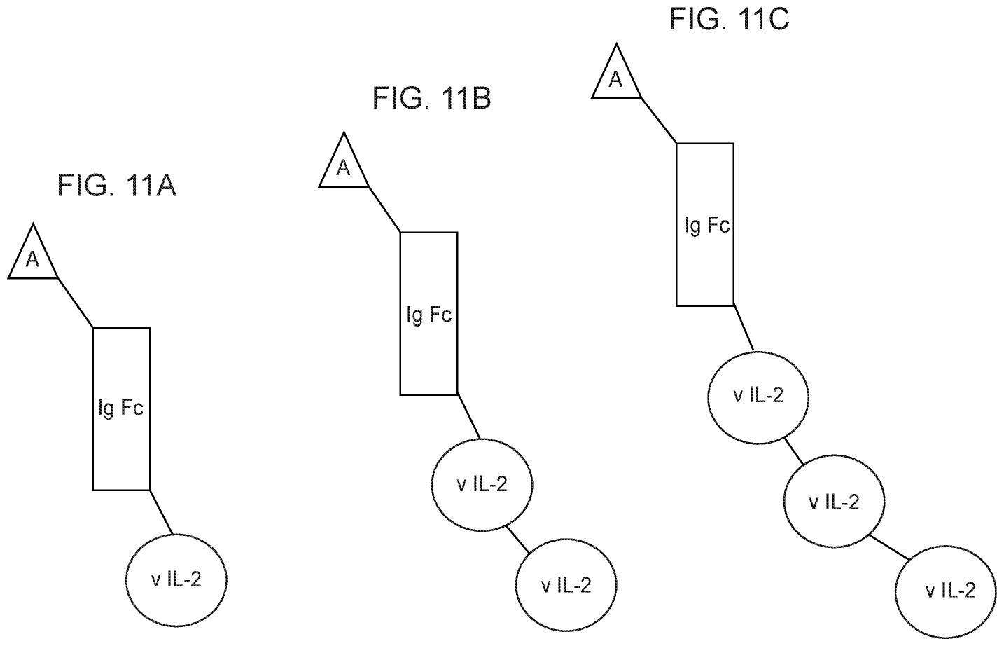

The following are non-limiting examples of cancer-associated antigen-binding antibodies that can be a CTP in an IL-2 variant fusion polypeptide of the present disclosure. Any of the CTPs described below can constitute the “A” or the “A′” in any of FIG. 10A-10C, FIG. 11A-11C, FIG. 12A-12B, and FIG. 14A-14N.

In some cases, an IL-2 variant fusion polypeptide comprises a first CTP (e.g., “A”) and a second CTP (e.g., “A′”), where the first CTP is specific for a first cancer-associated antigen, and where the second CTP is specific for a second cancer-associated antigen that is different from the first cancer-associated antigen. For example, in some cases, an IL-2 variant fusion polypeptide comprises: i) a first CTP (e.g., “A”), where the first CTP is an anti-CD19 antibody; and ii) a second CTP (e.g., “A′”), where the second CTP is an anti-CD20 antibody. As another example, in some cases, an IL-2 variant fusion polypeptide comprises: i) a first CTP (e.g., “A”), where the first CTP is an anti-Her2 antibody; and ii) a second CTP (e.g., “A′”), where the second CTP is an anti-EGFR antibody.

In some cases, an IL-2 variant fusion polypeptide comprises a first CTP (e.g., “A”) and a second CTP (e.g., “A′”), where the first CTP is specific for a first epitope on a cancer-associated antigen, and where the second CTP is specific for a second epitope on the same cancer-associated antigen. In some cases, the first epitope and the second epitope are non-identical and non-overlapping epitopes, and, as such, the cancer-associated antigen can be bound simultaneously by the first CTP and the second CTP.

### Anti-Her2

In some cases, a CTP present in an IL-2 variant fusion polypeptide of the present disclosure comprises an anti-Her2 antibody. In some cases, the anti-Her2 antibody is a scFv polypeptide. In some cases, the anti-Her2 antibody is a nanobody.

In some cases, an anti-Her2 antibody comprises: a) a light chain comprising an amino acid sequence having at least 90%, at least 95%, at least 98%, at least 99%, or 100%, amino acid sequence identity to the following amino acid sequence:

DIQMTQSPSSLSASVGDRVTITCRASQDVNTAVAWYQQKPGKAPKLLIYSASFLY SGVPSRFSGSRSGTDFTLTISSLQPEDFATYYCQQHYTTPPTFGQGTKVEIKRTVAAPSVFIFPPSD EQLKSGTASVVCLLNNFYPREAKVQWKVDNALQSGNSQESVTEQDSKDSTYSLSSTLTLSKAD YEKHKVYACEVTHQGLSSPVTKSFNRGEC (SEQ ID NO:38); and b) a heavy chain comprising an amino acid sequence having at least 90%, at least 95%, at least 98%, at least 99%, or 100%, amino acid sequence identity to the following amino acid sequence:

(SEQ ID NO: 39)
EVQLVESGGGLVQPGGSLRLSCAASGFNIKDTYIHWVRQAPGKGL
EWVARIYPTNGYTRYADSVKGRFTISADTSKNTAYLQMNSLRAED
TAVYYCSRWGGDGFYAMDYWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSREE
MTKNQVSLTCLVKGFYPSDIAVEWESNGQPENNYKTTPPVLDSDG
SFFLYSKLTVDKSRWQQGNVFSCSVMHEALHNHYTQKSLSLSPGK.

In some cases, an anti-Her2 antibody comprises a light chain variable region (VL) present in the light chain amino acid sequence provided above; and a heavy chain variable region (VH) present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence:
DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:40); and b) a VH comprising an amino acid sequence having at least 90%, at least 95%, at least 98%, at least 99%, or 100%, amino acid sequence identity to the amino acid sequence:
EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:41). In some cases, an anti-Her2 antibody comprises, in order from N-terminus to C-terminus: a) a VH comprising an amino acid sequence having at least 90%, at least 95%, at least 98%, at least 99%, or 100%, amino acid sequence identity to the amino acid sequence:
EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:41); b) a linker; and c) a VL comprising an amino acid sequence having at least 90%, at least 95%, at least 98%, at least 99%, or 100%, amino acid sequence identity to the amino acid sequence:
DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:40). Suitable linkers are described elsewhere herein and include, e.g., (GGGGS)n (SEQ ID NO:42), where n is an integer from 1 to 10 (e.g., 1, 2, 3, 4, 5, 6, 7, 8, 9, or 10).

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). In some cases, the V_{H} and V_{L} CDRs are as defined by MacCallum (see, e.g., Table 1, above; and MacCallum 1996).

For example, an anti-Her2 antibody can comprise a VL CDR1 having the amino acid sequence RASQDVNTAVA (SEQ ID NO:43); a VL CDR2 having the amino acid sequence SASFLY (SEQ ID NO:44); a VL CDR3 having the amino acid sequence QQHYTTPP (SEQ ID NO:45); a VH CDR1 having the amino acid sequence GFNIKDTY (SEQ ID NO:46); a VH CDR2 having the amino acid sequence IYPTNGYT (SEQ ID NO:47); and a VH CDR3 having the amino acid sequence SRWGGDGFYAMDY (SEQ ID NO:48).

In some cases, an anti-Her2 antibody is a scFv antibody. For example, an anti-Her2 scFv can comprise an amino acid sequence having at least 90%, at least 95%, at least 98%, at least 99%, or 100%, amino acid sequence identity to the following amino acid sequence:

(SEQ ID NO: 49)
EVQLVESGGGLVQPGGSLRLSCAASGFNIKDTYIHWVRQAPGKGL
EWVARIYPTNGYTRYADSVKGRFTISADTSKNTAYLQMNSLRAED
TAVYYCSRWGGDGFYAMDYWGQGTLVTVSSGGGGSGGGGSGGGGS
DIQMTQSPSSLSASVGDRVTITCRASQDVNTAVAWYQQKPGKAPK
LLIYSASFLYSGVPSRFSGSRSGTDFTLTISSLQPEDFATYYCQQ
HYTTPPTFGQGTKVEIK.

As another example, in some cases, an anti-Her2 antibody comprises: a) a light chain variable region (VL) comprising an amino acid sequence having at least 90%, at least 95%, at least 98%, at least 99%, or 100%, amino acid sequence identity to the following amino acid sequence:

DIQMTQSPSSLSASVGDRVTITCKASQDVSIGVAWYQQKPGKAPKLLIYSASYRY TGVPSRFSGSGSGTDFTLTISSLQPEDFATYYCQQYYIYPYTFGQGTKVEIKRTVAAPSVFIFPPSD EQLKSGTASVVCLLNNFYPREAKVQWKVDNALQSGNSQESVTEQDSKDSTYSLSSTLTLSKAD YEKHKVYACEVTHQGLSSPVTKSFNRGEC (SEQ ID NO:50); and b) a heavy chain variable region (VH) comprising an amino acid sequence having at least 90%, at least 95%, at least 98%, at least 99%, or 100%, amino acid sequence identity to the following amino acid sequence:

(SEQ ID NO: 51)
EVQLVESGGGLVQPGGSLRLSCAASGFTFTDYTMDWVRQAPGKGL
EWVADVNPNSGGSIYNQRFKGRFTLSVDRSKNTLYLQMNSLRAED
TAVYYCARNLGPSFYFDYWGQGTLVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSREEM
TKNQVSLTCLVKGFYPSDIAVEWESNGQPENNYKTTPPVLDSDGS
FFLYSKLTVDKSRWQQGNVFSCSVMHEALHNHYTQKSLSLSPG.

In some cases, an anti-Her2 antibody comprises a VL present in the light chain amino acid sequence provided above; and a VH present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence:
DIQMTQSPSSLSASVGDRVTITCKASQDVSIGVAWYQQKPGKAPKLLIYSASYRYTGVPSRFSGS GSGTDFTLTISSLQPEDFATYYCQQYYIYPYTFGQGTKVEIK (SEQ ID NO:52); and b) a VH comprising an amino acid sequence having at least 90%, at least 95%, at least 98%, at least 99%, or 100%, amino acid sequence identity to the amino acid sequence:

(SEQ ID NO: 53)
EVQLVESGGGLVQPGGSLRLSCAASGFTFTDYTMDWVRQAPGKGL
EWVADVNPNSGGSIYNQRFKGRFTLSVDRSKNTLYLQMNSLRAED
TAVYYCARNLGPSFYFDYWGQGTLVTVSS.

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). In some cases, the V_{H} and V_{L} CDRs are as defined by MacCallum (see, e.g., Table 1, above; and MacCallum 1996).

For example, an anti-HER2 antibody can comprise a VL CDR1 having the amino acid sequence KASQDVSIGVA (SEQ ID NO:54); a VL CDR2 having the amino acid sequence SASYRY (SEQ ID NO:55); a VL CDR3 having the amino acid sequence QQYYIYPY (SEQ ID NO:56); a VH CDR1 having the amino acid sequence GFTFTDYTMD (SEQ ID NO:57); a VH CDR2 having the amino acid sequence ADVNPNSGGSIYNQRFKG (SEQ ID NO:58); and a VH CDR3 having the amino acid sequence ARNLGPSFYFDY (SEQ ID NO:59).

In some cases, an anti-Her2 antibody is a scFv. For example, in some cases, an anti-Her2 scFv comprises an amino acid sequence having at least 90%, at least 95%, at least 98%, at least 99%, or 100%, amino acid sequence identity to the following amino acid sequence:

(SEQ ID NO: 49)
EVQLVESGGGLVQPGGSLRLSCAASGFNIKDTYIHWVRQAPGKG
LEWVARIYPTNGYTRYADSVKGRFTISADTSKNTAYLQMNSLRAE
DTAVYYCSRWGGDGFYAMDYWGQGTLVTVSSGGGGSGGGGSGGGG
SDIQMTQSPSSLSASVGDRVTITCRASQDVNTAVAWYQQKPGKAP
KLLIYSASFLYSGVPSRFSGSRSGTDFTLTISSLQPEDFATYYCQ
QHYTTPPTFGQGTKVEIK.

### Anti-CD19

In some cases, a CTP present in an IL-2 variant fusion polypeptide of the present disclosure comprises an anti-CD19 antibody. In some cases, the anti-CD19 antibody is a scFv polypeptide. In some cases, the anti-CD19 antibody is a nanobody. Anti-CD19 antibodies are known in the art; and the VH and VL, or the VH and VL CDRs, of any anti-CD19 antibody can be used in a CTP. See e.g., WO 2005/012493.

In some cases, an anti-CD19 antibody includes a VL CDR1 comprising the amino acid sequence KASQSVDYDGDSYLN (SEQ ID NO:60); a VL CDR2 comprising the amino acid sequence DASNLVS (SEQ ID NO:61); and a VL CDR3 comprising the amino acid sequence QQSTEDPWT (SEQ ID NO:62). In some cases, an anti-CD19 antibody includes a VH CDR1 comprising the amino acid sequence SYWMN (SEQ ID NO:63); a VH CDR2 comprising the amino acid sequence QIWPGDGDTNYNGKFKG (SEQ ID NO:64); and a VH CDR3 comprising the amino acid sequence RETTTVGRYYYAMDY (SEQ ID NO:65). In some cases, an anti-CD19 antibody includes a VL CDR1 comprising the amino acid sequence KASQSVDYDGDSYLN (SEQ ID NO:60); a VL CDR2 comprising the amino acid sequence DASNLVS (SEQ ID NO:61); a VL CDR3 comprising the amino acid sequence QQSTEDPWT (SEQ ID NO:62); a VH CDR1 comprising the amino acid sequence SYWMN (SEQ ID NO:63); a VH CDR2 comprising the amino acid sequence QIWPGDGDTNYNGKFKG (SEQ ID NO:64); and a VH CDR3 comprising the amino acid sequence RETTTVGRYYYAMDY (SEQ ID NO:65).

In some cases, an anti-CD19 antibody is a scFv. For example, in some cases, an anti-CD19 scFv comprises an amino acid sequence having at least 90%, at least 95%, at least 98%, at least 99%, or 100%, amino acid sequence identity to the following amino acid sequence:

(SEQ ID NO: 66)
DIQLTQSPASLAVSLGQRATISCKASQSVDYDGDSYLNWYQQIPG
QPPKLLIYDASNLVSGIPPRFSGSGSGTDFTLNIHPVEKVDAATY
HCQQSTEDPWTFGGGTKLEIKGGGGSGGGGSGGGGSQVQLQQSGA
ELVRPGSSVKISCKASGYAFSSYWMNWVKQRPGQGLEWIGQIWPG
DGDTNYNGKFKGKATLTADESSSTAYMQLSSLASEDSAVYFCARR
ETTTVGRYYYAMDYWGQGTTVTVS.

In some cases, an anti-CD19 antibody is an scFv polypeptide comprising the following amino acid sequence:

(SEQ ID NO: 67)
DIQLTQSPASLAVSLGQRATISCKASQSVDYDGDSYLNWYQQIPG
QPPKLLIYDASNLVSGIPPRFSGSGSGTDFTLNIHPVEKVDAATY
HCQQSTEDPWTFGGGTKLEIKGGGGSGGGGSGGGGSQVQLQQSGA
ELVRPGSSVKISCKASGYAFSSYWMNWVKQRPGQGLEWIGQIWPG
DGDTNYNGKFKGKATLTADESSSTAYMQLSSLASEDSAVYFCARR
ETTTVGRYYYAMDYWGQGTTVTVSS.

### Anti-Mesothelin

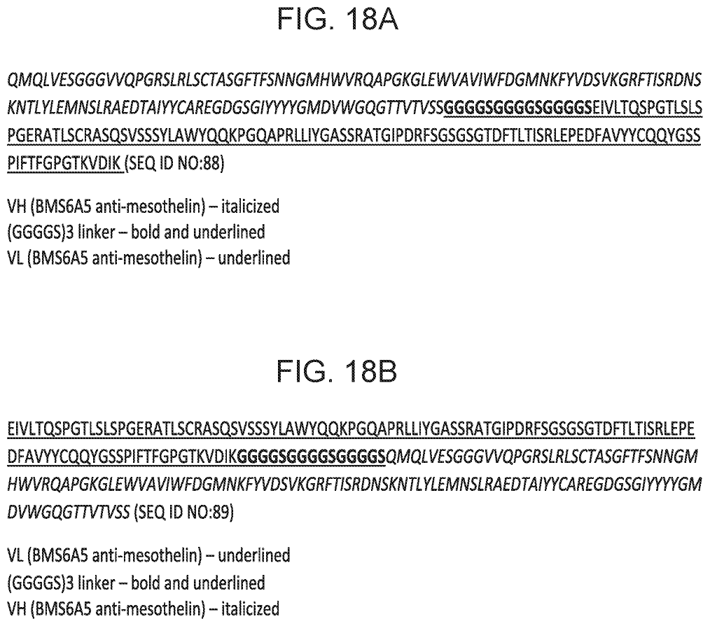

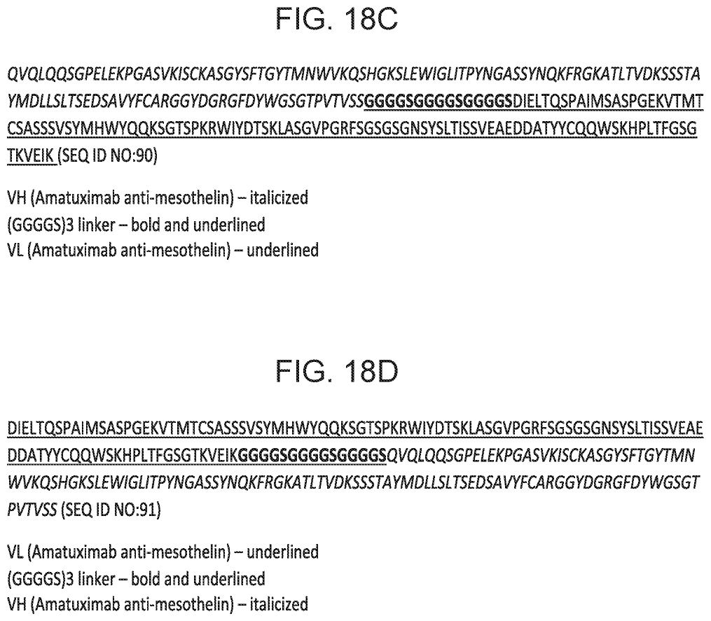

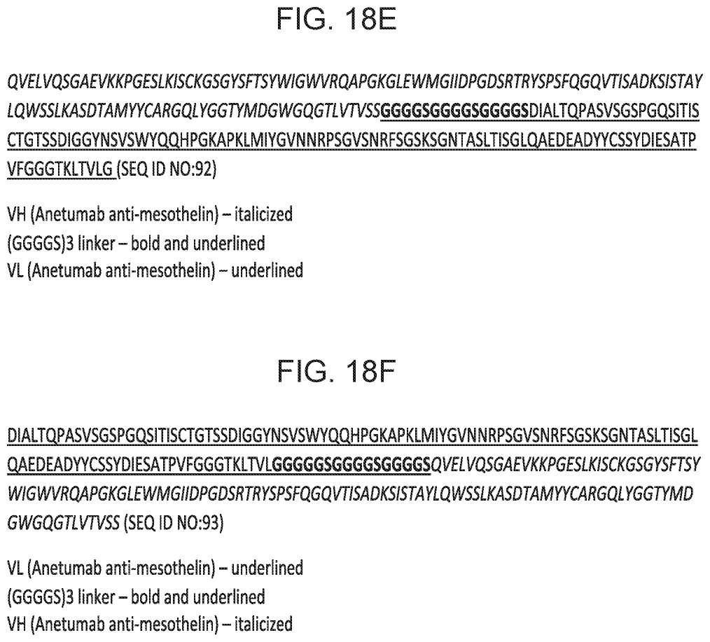

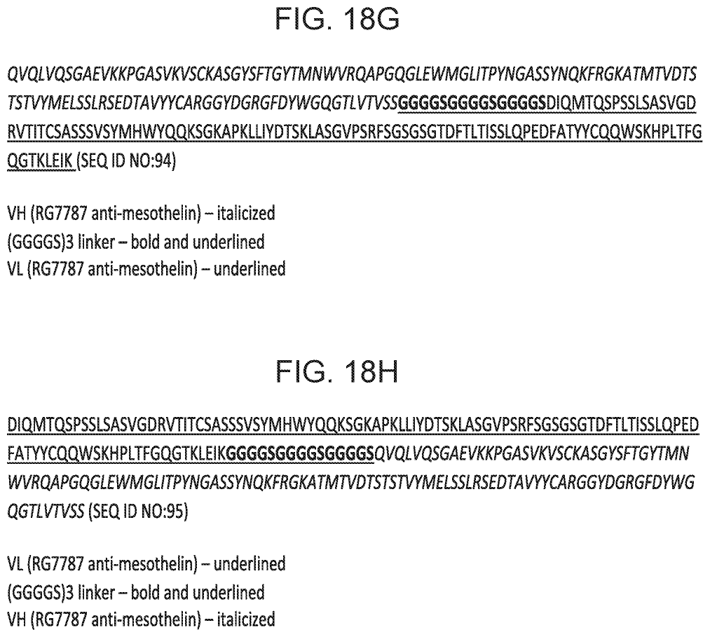

In some cases, a CTP present in an IL-2 variant fusion polypeptide of the present disclosure comprises an anti-mesothelin antibody. In some cases, the anti-mesothelin antibody is a scFv polypeptide. In some cases, the anti-mesothelin antibody is a nanobody. Anti-mesothelin antibodies are known in the art; and the VH and VL, or the VH and VL CDRs, of any anti-mesothelin antibody can be used as a CTP in an IL-2 variant fusion polypeptide. See, e.g., U.S. 2019/0000944; WO 2009/045957; WO 2014/031476; U.S. Pat. No. 8,460,660; US 2013/0066055; and WO 2009/068204. In some cases, the CTP is an anti-mesothelin scFv or an anti-mesothelin nanobody comprising VH and VL CDRs present in any one of the amino acid sequences set forth in FIG. 18A-18H. In some cases, the CTP is an anti-mesothelin scFv comprising an amino acid sequence as set forth in any one of FIG. 18A-18H.

In some cases, an anti-mesothelin antibody comprises: a) a light chain comprising an amino acid sequence having at least 90%, at least 95%, at least 98%, at least 99%, or 100%, amino acid sequence identity to the following amino acid sequence:

(SEQ ID NO: 68)
DIALTQPASVSGSPGQSITISCTGTSSDIGGYNSVSWYQQHPGKA
PKLMIYGVNNRPSGVSNRFSGSKSGNTASLTISGLQAEDEADYYC
SSYDIESATPVFGGGTKLTVLGQPKAAPSVTLFPPSSEELQANKA
TLVCLISDFYPGAVTVAWKGDSSPVKAGVETTTPSKQSNNKYAAS
SYLSLTPEQWKSHRSYSCQVTHEGSTVEKTVAPTESS;
and

b) a heavy chain comprising an amino acid sequence having at least 90%, at least 95%, at least 98%, at least 99%, or 100%, amino acid sequence identity to the following amino acid sequence:

(SEQ ID NO: 69)
QVELVQSGAEVKKPGESLKISCKGSGYSFTSYWIGWVRQAPGKGL
EWMGIIDPGDSRTRYSPSFQGQVTISADKSISTAYLQWSSLKASD
TAMYYCARGQLYGGTYMDGWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSRDE
LTKNQVSLTCLVKGFYPSDIAVEWESNGQPENNYKTTPPVLDSDG
SFFLYSKLTVDKSRWQQGNVFSCSVMHEALHNHYTQKSLSLSPGK.

In some cases, an anti-mesothelin antibody comprises a VL present in the light chain amino acid sequence provided above; and a VH present in the heavy chain amino acid sequence provided above. For example, an anti-mesothelin antibody can comprise: a) a VL comprising an amino acid sequence having at least 90%, at least 95%, at least 98%, at least 99%, or 100%, amino acid sequence identity to the amino acid sequence:
DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTK (SEQ ID NO:70); and b) a VH comprising an amino acid sequence having at least 90%, at least 95%, at least 98%, at least 99%, or 100%, amino acid sequence identity to the amino acid sequence:

(SEQ ID NO: 71)
QVELVQSGAEVKKPGESLKISCKGSGYSFTSYWIGWVRQAPGKGL
EWMGIIDPGDSRTRYSPSFQGQVTISADKSISTAYLQWSSLKASD
TAMYYCARGQLYGGTYMDGWGQGTLVTVSS.

In some cases, an anti-mesothelin antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). In some cases, the V_{H} and V_{L} CDRs are as defined by MacCallum (see, e.g., Table 1, above; and MacCallum 1996).

For example, an anti-mesothelin antibody can comprise a VL CDR1 having the amino acid sequence TGTSSDIGGYNSVS (SEQ ID NO:72); a VL CDR2 having the amino acid sequence LMIYGVNNRPS (SEQ ID NO:73); a VL CDR3 having the amino acid sequence SSYDIESATP (SEQ ID NO:74); a VH CDR1 having the amino acid sequence GYSFTSYWIG (SEQ ID NO:75); a VH CDR2 having the amino acid sequence WMGIIDPGDSRTRYSP (SEQ ID NO:76); and a VH CDR3 having the amino acid sequence GQLYGGTYMDG (SEQ ID NO:77).

An anti-mesothelin antibody can be a scFv. As one non-limiting example, an anti-mesothelin scFv can comprise the following amino acid sequence:
QVQLQQSGAEVKKPGASVKVSCKASGYTFTGYYMHWVRQAPGQGLEWMGRINPNSGGTNYA QKFQGRVTMTRDTSISTAYMELSRLRSEDTAVYYCARGRYYGMDVWGQGTMVTVSSGGGGS GGGGSGGGGSGGGGSEIVLTQSPATLSLSPGERATISCRASOSVSSNFAWYQQRPGQAPRLLIYD ASNRATGIPPRFSGSGSGTDFTLTISSLEPED FAAYYCHORSNWLYTFGQGTKVDIK (SEQ ID NO:78), where VH CDR1, CDR2, and CDR3 are underlined; and VL CDR1, CDR2, and CDR3 are bolded and underlined.

As one non-limiting example, an anti-mesothelin scFv can comprise the following amino acid sequence:
QVQLVQSGAEVKKPGASVKVSCKASGYTFTGYYMHWVRQAPGQGLEWMGWINPNSGGTNY AQKFQGRVTMTRDTSISTAYMELSRLRSDDTAVYYCARDLRRTVVTPRAYYGMDVWGQGTTV TVSSGGGGSGGGGSGGGGSGGGGSDIQLTQSPSTLSASVGDRVTITCQASQDISNSLNWYQQKA GKAPKLLIYDASTLETGVPSRFSGSGSGTDFSF
TISSLQPEDIATYYCQQHDNLPLTFGQGTKVEIK (SEQ ID NO:79), where VH CDR1, CDR2, and CDR3 are underlined; and VL CDR1, CDR2, and CDR3 are bolded and underlined.

In some cases, a CTP is an anti-mesothelin antibody suitable that comprises: a) VL CDR1, VL CDR2, and VL CDR3 present in a light chain variable region (VL) comprising the following amino acid sequence:
EIVLTQSPGTLSLSPGERATLSCRASQSVSSSYLAWYQQKPGQAPRLLIYGASSRATGIPDRFSGS GSGTDFTLTISRLEPEDFAVYYCQQYGSSPIFTFGPGTKVDIK (SEQ ID NO:80); and b) VH CDR1, CDR2, and CDR3 present in a heavy chain variable region (VH) comprising the following amino acid sequence:
QMQLVESGGGVVQPGRSLRLSCTASGFTFSNNGMHWVRQAPGKGLEWVAVIWFDGMNKFYV DSVKGRFTISRDNSKNTLYLEMNSLRAEDTAIYYCAREGDGSGIYYYYGMDVWGQGTTVTVSS (SEQ ID NO:81). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). In some cases, the V_{H} and V_{L} CDRs are as defined by MacCallum (see, e.g., Table 1, above; and MacCallum 1996).

In some cases, a CTP is an anti-mesothelin antibody that comprises: a) a VL region comprising an amino acid sequence having at least 90%, at least 95%, at least 98%, at least 99%, or 100%, amino acid sequence identity to the following amino acid sequence:
EIVLTQSPGTLSLSPGERATLSCRASQSVSSSYLAWYQQKPGQAPRLLIYGASSRATGIPDRFSGS GSGTDFTLTISRLEPEDFAVYYCQQYGSSPIFTFGPGTKVDIK (SEQ ID NO:80); and b) a VH region comprising an amino acid sequence having at least 90%, at least 95%, at least 98%, at least 99%, or 100%, amino acid sequence identity to the following amino acid sequence:

(SEQ ID NO: 81)
QMQLVESGGGVVQPGRSLRLSCTASGFTFSNNGMHWVRQAPGKGL
EWVAVIWFDGMNKFYVDSVKGRFTISRDNSKNTLYLEMNSLRAED
TAIYYCAREGDGSGIYYYYGMDVWGQGTTVTVSS

In some cases, a CTP is an anti-mesothelin scFv comprising, in order from N-terminus to C-terminus: a) a VH region comprising an amino acid sequence having at least 90%, at least 95%, at least 98%, at least 99%, or 100%, amino acid sequence identity to the following amino acid sequence:
QMQLVESGGGVVQPGRSLRLSCTASGFTFSNNGMHWVRQAPGKGLEWVAVIWFDGMNKFYV DSVKGRFTISRDNSKNTLYLEMNSLRAEDTAIYYCAREGDGSGIYYYYGMDVWGQGTTVTVSS (SEQ ID NO:81); b) a peptide linker; and c) a VL region comprising an amino acid sequence having at least 90%, at least 95%, at least 98%, at least 99%, or 100%, amino acid sequence identity to the following amino acid sequence:
EIVLTQSPGTLSLSPGERATLSCRASQSVSSSYLAWYQQKPGQAPRLLIYGASSRATGIPDRFSGS GSGTDFTLTISRLEPEDFAVYYCQQYGSSPIFTFGPGTKVDIK (SEQ ID NO:80). In some cases, the peptide linker comprises the amino acid sequence (GGGGS)n (SEQ ID NO:42), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:82) and has a length of 15 amino acids.

In some cases, a CTP is an anti-mesothelin scFv comprising, in order from N-terminus to C-terminus: a) a VL region comprising an amino acid sequence having at least 90%, at least 95%, at least 98%, at least 99%, or 100%, amino acid sequence identity to the following amino acid sequence:
EIVLTQSPGTLSLSPGERATLSCRASQSVSSSYLAWYQQKPGQAPRLLIYGASSRATGIPDRFSGS GSGTDFTLTISRLEPEDFAVYYCQQYGSSPIFTFGPGTKVDIK (SEQ ID NO:80); b) a peptide linker; and c) a VH region comprising an amino acid sequence having at least 90%, at least 95%, at least 98%, at least 99%, or 100%, amino acid sequence identity to the following amino acid sequence:
QMQLVESGGGVVQPGRSLRLSCTASGFTFSNNGMHWVRQAPGKGLEWVAVIWFDGMNKFYV DSVKGRFTISRDNSKNTLYLEMNSLRAEDTAIYYCAREGDGSGIYYYYGMDVWGQGTTVTVSS (SEQ ID NO:81). In some cases, the peptide linker comprises the amino acid sequence (GGGGS)n (SEQ ID NO:42), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:82) and has a length of 15 amino acids.

In some cases, a CTP is an anti-mesothelin antibody that comprises: a) VL CDR1, VL CDR2, and VL CDR3 present in a light chain variable region (VL) comprising the following amino acid sequence:
DIELTQSPAIMSASPGEKVTMTCSASSSVSYMHWYQQKSGTSPKRWIYDTSKLASGVPGRFSGS GSGNSYSLTISSVEAEDDATYYCQQWSKHPLTFGSGTKVEIK (SEQ ID NO:83); and b) VH CDR1, CDR2, and CDR3 present in a heavy chain variable region (VH) comprising the following amino acid sequence:
QVQLQQSGPELEKPGASVKISCKASGYSFTGYTMNWVKQSHGKSLEWIGLITPYNGASSYNQKF RGKATLTVDKSSSTAYMDLLSLTSEDSAVYFCARGGYDGRGFDYWGSGTPVTVSS (SEQ ID NO:84). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). In some cases, the V_{H} and V_{L} CDRs are as defined by MacCallum (see, e.g., Table 1, above; and MacCallum 1996). See, e.g., Amatuximab.

In some cases, a CTP is an anti-mesothelin antibody that comprises: a) a VL region comprising an amino acid sequence having at least 90%, at least 95%, at least 98%, at least 99%, or 100%, amino acid sequence identity to the following amino acid sequence:
DIELTQSPAIMSASPGEKVTMTCSASSSVSYMHWYQQKSGTSPKRWIYDTSKLASGVPGRFSGS GSGNSYSLTISSVEAEDDATYYCQQWSKHPLTFGSGTKVEIK (SEQ ID NO:83); and b) a VH region comprising an amino acid sequence having at least 90%, at least 95%, at least 98%, at least 99%, or 100%, amino acid sequence identity to the following amino acid sequence:

(SEQ ID NO: 84)
QVQLQQSGPELEKPGASVKISCKASGYSFTGYTMNWVKQSHGKSLEWIGL
ITPYNGASSYNQKFRGKATLTVDKSSSTAYMDLLSLTSEDSAVYFCARGG
YDGRGFDYWGSGTPVTVSS.

In some cases, a CTP is an anti-mesothelin scFv comprising, in order from N-terminus to C-terminus: a) a VL region comprising an amino acid sequence having at least 90%, at least 95%, at least 98%, at least 99%, or 100%, amino acid sequence identity to the following amino acid sequence:
DIELTQSPAIMSASPGEKVTMTCSASSSVSYMHWYQQKSGTSPKRWIYDTSKLASGVPGRFSGS GSGNSYSLTISSVEAEDDATYYCQQWSKHPLTFGSGTKVEIK (SEQ ID NO:83); b) a peptide linker; and c) a VH region comprising an amino acid sequence having at least 90%, at least 95%, at least 98%, at least 99%, or 100%, amino acid sequence identity to the following amino acid sequence:
QVQLQQSGPELEKPGASVKISCKASGYSFTGYTMNWVKQSHGKSLEWIGLITPYNGASSYNQKF RGKATLTVDKSSSTAYMDLLSLTSEDSAVYFCARGGYDGRGFDYWGSGTPVTVSS (SEQ ID NO:84). In some cases, the peptide linker comprises the amino acid sequence (GGGGS)n (SEQ ID NO:42), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:82) and has a length of 15 amino acids.

In some cases, a CTP is an anti-mesothelin scFv comprising, in order from N-terminus to C-terminus: a) a VH region comprising an amino acid sequence having at least 90%, at least 95%, at least 98%, at least 99%, or 100%, amino acid sequence identity to the following amino acid sequence:
QVQLQQSGPELEKPGASVKISCKASGYSFTGYTMNWVKQSHGKSLEWIGLITPYNGASSYNQKF RGKATLTVDKSSSTAYMDLLSLTSEDSAVYFCARGGYDGRGFDYWGSGTPVTVSS (SEQ ID NO:84); b) a peptide linker; and c) a VL region comprising an amino acid sequence having at least 90%, at least 95%, at least 98%, at least 99%, or 100%, amino acid sequence identity to the following amino acid sequence:
DIELTQSPAIMSASPGEKVTMTCSASSSVSYMHWYQQKSGTSPKRWIYDTSKLASGVPGRFSGS GSGNSYSLTISSVEAEDDATYYCQQWSKHPLTFGSGTKVEIK (SEQ ID NO:83). In some cases, the peptide linker comprises the amino acid sequence (GGGGS)n (SEQ ID NO:42), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:82) and has a length of 15 amino acids.

In some cases, a CTP is an anti-mesothelin antibody that comprises: a) VL CDR1, VL CDR2, and VL CDR3 present in a light chain variable region (VL) comprising the following amino acid sequence:
DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTKLTVLG (SEQ ID NO:85); and b) VH CDR1, CDR2, and CDR3 present in a heavy chain variable region (VH) comprising the following amino acid sequence:
QVELVQSGAEVKKPGESLKISCKGSGYSFTSYWIGWVRQAPGKGLEWMGIIDPGDSRTRYSPSF QGQVTISADKSISTAYLQWSSLKASDTAMYYCARGQLYGGTYMDGWGQGTLVTVSS (SEQ ID NO:71). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). In some cases, the V_{H} and V_{L} CDRs are as defined by MacCallum (see, e.g., Table 1, above; and MacCallum 1996).

In some cases, a CTP is an anti-mesothelin antibody that comprises: a) a VL region comprising an amino acid sequence having at least 90%, at least 95%, at least 98%, at least 99%, or 100%, amino acid sequence identity to the following amino acid sequence:
DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTKLTVLG (SEQ ID NO:85); and b) a VH region comprising an amino acid sequence having at least 90%, at least 95%, at least 98%, at least 99)%, or 100%, amino acid sequence identity to the following amino acid sequence:

(SEQ ID NO: 71)
QVELVQSGAEVKKPGESLKISCKGSGYSFTSYWIGWVRQAPGKGL
EWMGIIDPGDSRTRYSPSFQGQVTISADKSISTAYLQWSSLKASD
TAMYYCARGQLYGGTYMDGWGQGTLVTVSS.

In some cases, a CTP is an anti-mesothelin scFv comprising, in order from N-terminus to C-terminus: a) a VL region comprising an amino acid sequence having at least 90%, at least 95%, at least 98%, at least 99%, or 100%, amino acid sequence identity to the following amino acid sequence:
DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTKLTVLG (SEQ ID NO:85); b) a peptide linker; and c) a VH region comprising an amino acid sequence having at least 90%, at least 95%, at least 98%, at least 99%, or 100%, amino acid sequence identity to the following amino acid sequence:
QVELVQSGAEVKKPGESLKISCKGSGYSFTSYWIGWVRQAPGKGLEWMGIIDPGDSRTRYSPSF QGQVTISADKSISTAYLQWSSLKASDTAMYYCARGQLYGGTYMDGWGQGTLVTVSS (SEQ ID NO:71). In some cases, the peptide linker comprises the amino acid sequence (GGGGS)n (SEQ ID NO:42), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:82) and has a length of 15 amino acids.

In some cases, a CTP is an anti-mesothelin scFv comprising, in order from N-terminus to C-terminus: a) a VH region comprising an amino acid sequence having at least 90%, at least 95%, at least 98%, at least 99%, or 100%, amino acid sequence identity to the following amino acid sequence:
QVELVQSGAEVKKPGESLKISCKGSGYSFTSYWIGWVRQAPGKGLEWMGIIDPGDSRTRYSPSF QGQVTISADKSISTAYLQWSSLKASDTAMYYCARGQLYGGTYMDGWGQGTLVTVSS (SEQ ID NO:71); b) a peptide linker; and c) a VL region comprising an amino acid sequence having at least 90%, at least 95%, at least 98%, at least 99%, or 100%, amino acid sequence identity to the following amino acid sequence:
DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTKLTVLG (SEQ ID NO:85). In some cases, the peptide linker comprises the amino acid sequence (GGGGS)n (SEQ ID NO:42), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:82) and has a length of 15 amino acids.

In some cases, a CTP is an anti-mesothelin antibody that comprises: a) VL CDR1, VL CDR2, and VL CDR3 present in a light chain variable region (VL) comprising the following amino acid sequence:
DIQMTQSPSSLSASVGDRVTITCSASSSVSYMHWYQQKSGKAPKLLIYDTSKLASGVPSRFSGSG SGTDFTLTISSLQPEDFATYYCQQWSKHPLTFGQGTKLEIK (SEQ ID NO:86); and b) VH CDR1, CDR2, and CDR3 present in a heavy chain variable region (VH) comprising the following amino acid sequence:
QVQLVQSGAEVKKPGASVKVSCKASGYSFTGYTMNWVRQAPGQGLEWMGLITPYNGASSYN QKFRGKATMTVDTSTSTVYMELSSLRSEDTAVYYCARGGYDGRGFDYWGQGTLVTVSS (SEQ ID NO:87). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). In some cases, the V_{H} and V_{L} CDRs are as defined by MacCallum (see, e.g., Table 1, above; and MacCallum 1996). See, e.g., RG7787.

In some cases, a CTP is an anti-mesothelin antibody that comprises: a) a VL region comprising an amino acid sequence having at least 90%, at least 95%, at least 98%, at least 99%, or 100%, amino acid sequence identity to the following amino acid sequence:
DIQMTQSPSSLSASVGDRVTITCSASSSVSYMHWYQQKSGKAPKLLIYDTSKLASGVPSRFSGSG SGTDFTLTISSLQPEDFATYYCQQWSKHPLTFGQGTKLEIK (SEQ ID NO:86); and b) a VH region comprising an amino acid sequence having at least 90%, at least 95%, at least 98%, at least 99%, or 100%, amino acid sequence identity to the following amino acid sequence:

(SEQ ID NO: 87)
QVQLVQSGAEVKKPGASVKVSCKASGYSFTGYTMNWVRQAPGQGL
EWMGLITPYNGASSYNQKFRGKATMTVDTSTSTVYMELSSLRSED
TAVYYCARGGYDGRGFDYWGQGTLVTVSS.

In some cases, a CTP is an anti-mesothelin scFv comprising, in order from N-terminus to C-terminus: a) a VL region comprising an amino acid sequence having at least 90%, at least 95%, at least 98%, at least 99%, or 100%, amino acid sequence identity to the following amino acid sequence:
DIQMTQSPSSLSASVGDRVTITCSASSSVSYMHWYQQKSGKAPKLLIYDTSKLASGVPSRFSGSG SGTDFTLTISSLQPEDFATYYCQQWSKHPLTFGQGTKLEIK (SEQ ID NO:86); b) a peptide linker; and c) a VH region comprising an amino acid sequence having at least 90%, at least 95%, at least 98%, at least 99%, or 100%, amino acid sequence identity to the following amino acid sequence:
QVQLVQSGAEVKKPGASVKVSCKASGYSFTGYTMNWVRQAPGQGLEWMGLITPYNGASSYN QKFRGKATMTVDTSTSTVYMELSSLRSEDTAVYYCARGGYDGRGFDYWGQGTLVTVSS (SEQ ID NO:87). In some cases, the peptide linker comprises the amino acid sequence (GGGGS)n (SEQ ID NO:42), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:82) and has a length of 15 amino acids.

In some cases, a CTP is an anti-mesothelin scFv comprising, in order from N-terminus to C-terminus: a) a VH region comprising an amino acid sequence having at least 90%, at least 95%, at least 98%, at least 99%, or 100%, amino acid sequence identity to the following amino acid sequence:
QVQLVQSGAEVKKPGASVKVSCKASGYSFTGYTMNWVRQAPGQGLEWMGLITPYNGASSYN QKFRGKATMTVDTSTSTVYMELSSLRSEDTAVYYCARGGYDGRGFDYWGQGTLVTVSS (SEQ ID NO:87); b) a peptide linker; and c) a VL region comprising an amino acid sequence having at least 90%, at least 95%, at least 98%, at least 99%, or 100%, amino acid sequence identity to the following amino acid sequence:
DIQMTQSPSSLSASVGDRVTITCSASSSVSYMHWYQQKSGKAPKLLIYDTSKLASGVPSRFSGSG SGTDFTLTISSLQPEDFATYYCQQWSKHPLTFGQGTKLEIK (SEQ ID NO:86). In some cases, the peptide linker comprises the amino acid sequence (GGGGS)n (SEQ ID NO:42), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:82) and has a length of 15 amino acids.

In some cases, an anti-mesothelin scFv comprises the following amino acid sequence:
QMQLVESGGGVVQPGRSLRLSCTASGFTFSNNGMHWVRQAPGKGLEWVAVIWFDGMNKFYVDSVK GRFTISRDNSKNTLYLEMNSLRAEDTAIYYCAREGDGSGIYYYYGMDVWGQGTTVTVSSGGGGSGGG GSGGGGSEIVLTQSPGTLSLSPGERATLSCRASQSVSSSYLAWYQQKPGQAPRLLIYGASSRATG IPDRFSGSGSGTDFTLTISRLEPEDFAVYYCQQYGSSPIFTFGPGTKVDIK (SEQ ID NO:88), where the VH sequence is italicized, the (GGGGS)3 (SEQ ID NO:82) linker is bolded and underlined, and the VL sequence is underlined.

In some cases, an anti-mesothelin scFv comprises the following amino acid sequence: EIVLTOSPGTLSLSPGERATLSCRASQSVSSSYLAWYQQKPGOAPRLLIYGASSRATGIPDRFSGS GSGTDFTLTISRLEPEDFAVYYCQQYGSSPIFTFGPGTKVDIKGGGGSGGGGSGGGGSQMQLVE SGGGVVQPGRSLRLSCTASGFTFSNNGMHWVRQAPGKGLEWVAVIWFDGMNKFYVDSVKGRFTISR DNSKNTLYLEMNSLRAEDTAIYYCAREGDGSGIYYYYGMDVWGQGTTVTVSS (SEQ ID NO:89), where the VL sequence is underlined, the (GGGGS)3 (SEQ ID NO:82) linker is bolded and underlined, and the VL sequence is italicized.

In some cases, an anti-mesothelin scFv comprises the following amino acid sequence:
QVQLQQSGPELEKPGASVKISCKASGYSFTGYTMNWVKQSHGKSLEWIGLITPYNGASSYNQKFRGKA TLTVDKSSSTAYMDLLSLTSEDSAVYFCARGGYDGRGFDYWGSGTPVTVSSGGGGSGGGGSGGGGS DIELTOSPAIMSASPGEKVTMTCSASSSVSYMHWYOOKSGTSPKRWIYDTSKLASGVPGRFSGS GSGNSYSLTISSVEAEDDATYYCQQWSKHPLTFGSGTKVEIK (SEQ ID NO:90), where the VH sequence is italicized, the (GGGGS)3 (SEQ ID NO:82) linker is bolded and underlined, and the VL sequence is underlined.

In some cases, an anti-mesothelin scFv comprises the following amino acid sequence: DIELTOSPAIMSASPGEKVTMTCSASSSVSYMHWYOOKSGTSPKRWIYDTSKLASGVPGRFSGS GSGNSYSLTISSVEAEDDATYYCQQWSKHPLTFGSGTKVEIKGGGGSGGGGSGGGGSQVQLQ QSGPELEKPGASVKISCKASGYSFTGYTMNWVKQSHGKSLEWIGLITPYNGASSYNQKFRGKATLTVD KSSSTAYMDLLSLTSEDSAVYFCARGGYDGRGFDYWGSGTPVTVSS (SEQ ID NO:91), where the VL sequence is underlined, the (GGGGS)3 (SEQ ID NO:82) linker is bolded and underlined, and the VL sequence is italicized.

In some cases, an anti-mesothelin scFv comprises the following amino acid sequence:
QVELVQSGAEVKKPGESLKISCKGSGYSFTSYWIGWVRQAPGKGLEWMGIIDPGDSRTRYSPSFQGQV TISADKSISTAYLQWSSLKASDTAMYYCARGQLYGGTYMDGWGQGTLVTVSSGGGGSGGGGSGGG GSDIALTOPASVSGSPGOSITISCTGTSSDIGGYNSVSWYQQHPGKAPKLMIYGVNNRPSGVSNR FSGSKSGNTASLTISGLQAEDEADYYCSSYDIESATPVFGGGTKLTVLG (SEQ ID NO:92), where the VH sequence is italicized, the (GGGGS)3 (SEQ ID NO:82) linker is bolded and underlined, and the VL sequence is underlined.

In some cases, an anti-mesothelin scFv comprises the following amino acid sequence:
DIALTOPASVSGSPGOSITISCTGTSSDIGGYNSVSWYOOHPGKAPKLMIYGVNNRPSGVSNRFS GSKSGNTASLTISGLQAEDEADYYCSSYDIESATPVFGGGTKLTVLGGGGGSGGGGSGGGGSQVELVQSGAEVKKPGESLKISCKGSGYSFTSYWIGWVRQAPGKGLEWMGIIDPGDSRTRYSPSFQGQVT ISADKSISTAYLQWSSLKASDTAMYYCARGQLYGGTYMDGWGQGTLVTVSS (SEQ ID NO:93), where the VL sequence is underlined, the (GGGGS)3 (SEQ ID NO:82) linker is bolded and underlined, and the VL sequence is italicized.

In some cases, an anti-mesothelin scFv comprises the following amino acid sequence:
QVQLVQSGAEVKKPGASVKVSCKASGYSFTGYTMNWVRQAPGQGLEWMGLITPYNGASSYNQKFRG KATMTVDTSTSTVYMELSSLRSEDTAVYYCARGGYDGRGFDYWGQGTLVTVSSGGGGSGGGGSGG GGSDIQMTOSPSSLSASVGDRVTITCSASSSVSYMHWYQQKSGKAPKLLIYDTSKLASGVPSRFS GSGSGTDFTLTISSLOPEDFATYYCQQWSKHPLTFGOGTKLEIK (SEQ ID NO:94), where the VH sequence is italicized, the (GGGGS)3 (SEQ ID NO:82) linker is bolded and underlined, and the VL sequence is underlined.

In some cases, an anti-mesothelin scFv comprises the following amino acid sequence: DIQMTOSPSSLSASVGDRVTITCSASSSVSYMHWYOQKSGKAPKLLIYDTSKLASGVPSRFSGSG SGTDFTLTISSLOPEDFATYYCQQWSKHPLTFGOGTKLEIKGGGGSGGGGSGGGGSQVQLVQS GAEVKKPGASVKVSCKASGYSFTGYTMNWVRQAPGQGLEWMGLITPYNGASSYNQKFRGKATMTVD TSTSTVYMELSSLRSEDTAVYYCARGGYDGRGFDYWGQGTLVTVSS (SEQ ID NO:95), where the VL sequence is underlined, the (GGGGS)3 (SEQ ID NO:82) linker is bolded and underlined, and the VL sequence is italicized.

### Anti-PSMA

Prostate-specific membrane antigen (PSMA) (also known as folate hydrolase 1 (FOLH1); membrane glutamate carboxypeptidase, and N-Acetylated-Alpha-Linked Acidic Dipeptidase 1) that is up-regulated in cancerous cells in the prostate and is used as a diagnostic and prognostic indicator of prostate cancer. In some cases, a CTP present in an IL-2 variant fusion polypeptide of the present disclosure comprises an anti-PSMA antibody. In some cases, the anti-PSMA antibody is a scFv polypeptide. In some cases, the anti-PSMA antibody is a nanobody. Anti-PSMA antibodies are known in the art; and the VH and VL, or the VH and VL CDRs, of any anti-PSMA antibody can be used as a CTP. See, e.g., U.S. Pat. No. 10,179,819 and U.S. Patent Publication No. 2021/0277141.

### Anti-CD22

CD22 (also known as B-Lymphocyte Cell Adhesion Molecule, Sialic Acid-Binding Ig-Like Lectin 2, or SIGLEC2) is a sialic acid-binding adhesion molecule largely restricted to the B cell lineage and expressed on most B-lineage malignancies. In some cases, a CTP present in an IL-2 variant fusion polypeptide of the present disclosure comprises an anti-CD22 antibody. In some cases, the anti-CD22 antibody is a scFv polypeptide. In some cases, the anti-CD22 antibody is a nanobody.

Anti-CD22 antibodies are known in the art; and the VH and VL, or the VH and VL CDRs, of any anti-CD22 antibody can be used as a CTP. Sec, e.g., Xiao et al. (2009) Mabs 1:297 (describing the fully human anti-CD22 m971 scFv); and U.S. Patent Publication No. 2020/0147134. Examples of anti-CD22 antibodies include epratuzumab and inotuzumab. See, e.g., Lenoard et al. (2007) Oncogene 26:3704 and U.S. Pat. No. 5,789,554 (describing epratuzumab); and DiJoseph et al. (2007) Leukemia 21:2240 (describing inotuzumab).

For example, an anti-CD22 antibody can comprise: i) a heavy chain variable region (VH) CDR1 having the amino acid sequence: GDSVSSNSAA (SEQ ID NO:96); ii) a VH CDR2 having the amino acid sequence: TYYRSKWYN (SEQ ID NO:97); iii) a VH CDR3 having the amino acid sequence: AREVTGDLEDAFDI (SEQ ID NO:98); iv) a light chain variable region (VL) CDR1 having the amino acid sequence: QTIWSY (SEQ ID NO:99); v) a VL CDR2 having the amino acid sequence: AAS (Ala-Ala-Ser); and vi) a VL CDR3 having the amino acid sequence: QQSYSIPQT (SEQ ID NO:100).

### Anti-TROP-2

Trophoblast cell surface antigen 2 (Trop-2) (also known as epithelial glycoprotein-1, gastrointestinal tumor-associated antigen GA733-1, membrane component chromosome 1 surface marker-1, and tumor-associated calcium signal transducer-2) is a transmembrane glycoprotein that is upregulated in numerous cancer types, and is the protein product of the TACSTD2 gene. In some cases, a CTP present in an IL-2 variant fusion polypeptide of the present disclosure comprises an anti-TROP-2 antibody. In some cases, the anti-TROP-2 antibody is a scFv polypeptide. In some cases, the anti-TROP-2 antibody is a nanobody.

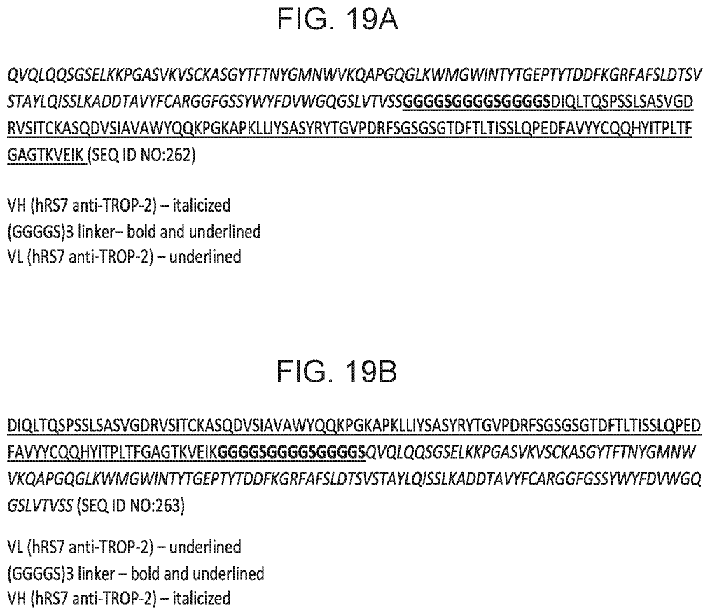

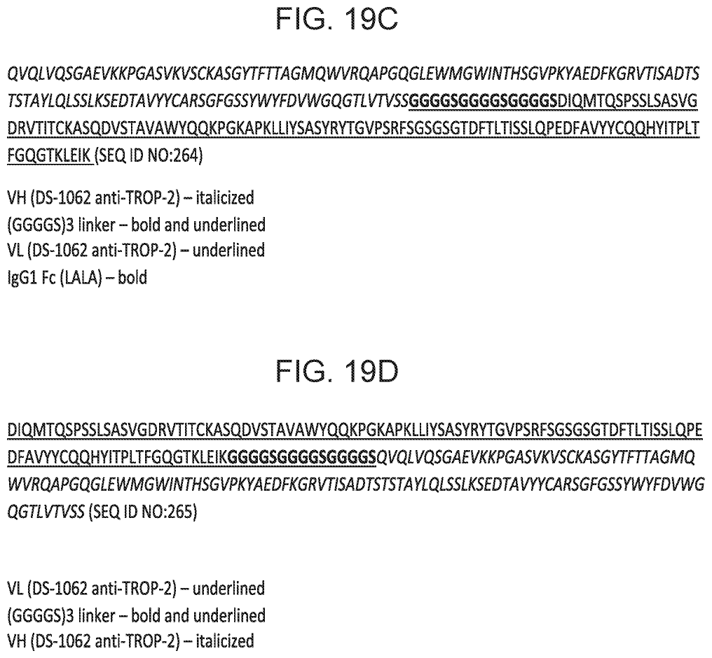

In some cases, the CTP is an anti-TROP-2 scFv or an anti-TROP-2 nanobody comprising VH and VL CDRs present in any one of the amino acid sequences set forth in FIG. 19A-19D. In some cases, the CTP is an anti-TROP-2 scFv comprising an amino acid sequence as set forth in any one of FIG. 19A-19D.

Anti-TROP-2 antibodies are known in the art; and the VH and VL, or the VH and VL CDRs, of any anti-TROP-2 antibody can be used as a CTP. See, e.g., U.S. Pat. No. 7,238,785). In some cases, an anti-TROP-2 antibody comprises: i) light chain CDR sequences CDR1 (KASQDVSIAVA; SEQ ID NO:101); CDR2 (SASYRYT; SEQ ID NO:102); and CDR3 (QQHYITPLT; SEQ ID NO:103); and ii) heavy chain CDR sequences CDR1 (NYGMN; SEQ ID NO:104); CDR2 (WINTYTGEPTYTDDFKG; SEQ ID NO:105); and CDR3 (GGFGSSYWYFDV; SEQ ID NO:106).

In some cases, an anti-TROP-2 antibody comprises: i) heavy chain CDR sequences CDR1 (TAGMQ; SEQ ID NO:107); CDR2 (WINTHSGVPKYAEDFKG (SEQ ID NO:108); and CDR3 (SGFGSSYWYFDV; SEQ ID NO:109); and ii) light chain CDR sequences CDR1 (KASQDVSTAVA; SEQ ID NO:110); CDR2 (SASYRYT; SEQ ID NO:102); and CDR3 (QQHYITPLT; SEQ ID NO:103).

In some cases, a CTP is an anti-TROP2 antibody that comprises: a) VL CDR1, VL CDR2, and VL CDR3 present in a light chain variable region (VL) comprising the following amino acid sequence:
DIQLTQSPSSLSASVGDRVSITCKASQDVSIAVAWYQQKPGKAPKLLIYSASYRYTGVPDRFSGS GSGTDFTLTISSLQPEDFAVYYCQQHYITPLTFGAGTKVEIK (SEQ ID NO:111); and b) VH CDR1, CDR2, and CDR3 present in a heavy chain variable region (VH) comprising the following amino acid sequence:
QVQLQQSGSELKKPGASVKVSCKASGYTFTNYGMNWVKQAPGQGLKWMGWINTYTGEPTYT DDFKGRFAFSLDTSVSTAYLQISSLKADDTAVYFCARGGFGSSYWYFDVWGQGSLVTVSS (SEQ ID NO:112). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). In some cases, the V_{H} and V_{L} CDRs are as defined by MacCallum (see, e.g., Table 1, above; and MacCallum 1996).

In some cases, a CTP is an anti-TROP-2 antibody that comprises: a) a VL region comprising an amino acid sequence having at least 90%, at least 95%, at least 98%, at least 99%, or 100%, amino acid sequence identity to the following amino acid sequence:
DIQLTQSPSSLSASVGDRVSITCKASQDVSIAVAWYQQKPGKAPKLLIYSASYRYTGVPDRFSGS GSGTDFTLTISSLQPEDFAVYYCQQHYITPLTFGAGTKVEIK (SEQ ID NO:111); and b) a VH region comprising an amino acid sequence having at least 90%, at least 95%, at least 98%, at least 99%, or 100%, amino acid sequence identity to the following amino acid sequence:

(SEQ ID NO: 112)
QVQLQQSGSELKKPGASVKVSCKASGYTFTNYGMNWVKQAPGQGL
KWMGWINTYTGEPTYTDDFKGRFAFSLDTSVSTAYLQISSLKADD
TAVYFCARGGFGSSYWYFDVWGQGSLVTVSS.

In some cases, a CTP is an anti-TROP-2 scFv comprising, in order from N-terminus to C-terminus: a) a VL region comprising an amino acid sequence having at least 90%, at least 95%, at least 98%, at least 99%, or 100%, amino acid sequence identity to the following amino acid sequence:
DIQLTQSPSSLSASVGDRVSITCKASQDVSIAVAWYQQKPGKAPKLLIYSASYRYTGVPDRFSGS GSGTDFTLTISSLQPEDFAVYYCQQHYITPLTFGAGTKVEIK (SEQ ID NO:111); b) a peptide linker; and c) a VH region comprising an amino acid sequence having at least 90%, at least 95%, at least 98%, at least 99%, or 100%, amino acid sequence identity to the following amino acid sequence:
QVQLQQSGSELKKPGASVKVSCKASGYTFTNYGMNWVKQAPGQGLKWMGWINTYTGEPTYT DDFKGRFAFSLDTSVSTAYLQISSLKADDTAVYFCARGGFGSSYWYFDVWGQGSLVTVSS (SEQ ID NO: 112). In some cases, the peptide linker comprises the amino acid sequence (GGGGS)n (SEQ ID NO:42), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:82) and has a length of 15 amino acids.

In some cases, a CTP is an anti-TROP-2 scFv comprising, in order from N-terminus to C-terminus: a) a VH region comprising an amino acid sequence having at least 90%, at least 95%, at least 98%, at least 99%, or 100%, amino acid sequence identity to the following amino acid sequence:
QVQLQQSGSELKKPGASVKVSCKASGYTFTNYGMNWVKQAPGQGLKWMGWINTYTGEPTYT DDFKGRFAFSLDTSVSTAYLQISSLKADDTAVYFCARGGFGSSYWYFDVWGQGSLVTVSS (SEQ ID NO:112); b) a peptide linker; and c) a VL region comprising an amino acid sequence having at least 90%, at least 95%, at least 98%, at least 99%, or 100%, amino acid sequence identity to the following amino acid sequence:
DIQLTQSPSSLSASVGDRVSITCKASQDVSIAVAWYQQKPGKAPKLLIYSASYRYTGVPDRFSGS GSGTDFTLTISSLQPEDFAVYYCQQHYITPLTFGAGTKVEIK (SEQ ID NO:111). In some cases, the peptide linker comprises the amino acid sequence (GGGGS)n (SEQ ID NO:42), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:82) and has a length of 15 amino acids.

In some cases, a CTP is an anti-TROP2 antibody that comprises: a) VL CDR1, VL CDR2, and VL CDR3 present in a light chain variable region (VL) comprising the following amino acid sequence:
DIQMTQSPSSLSASVGDRVTITCKASQDVSTAVAWYQQKPGKAPKLLIYSASYRYTGVPSRFSG SGSGTDFTLTISSLQPEDFAVYYCQQHYITPLTFGQGTKLEIK (SEQ ID NO:113); and b) VH CDR1, CDR2, and CDR3 present in a heavy chain variable region (VH) comprising the following amino acid sequence:
QVQLVQSGAEVKKPGASVKVSCKASGYTFTTAGMQWVRQAPGQGLEWMGWINTHSGVPKYA EDFKGRVTISADTSTSTAYLQLSSLKSEDTAVYYCARSGFGSSYWYFDVWGQGTLVTVSS (SEQ ID NO:114). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). In some cases, the V_{H} and V_{L} CDRs are as defined by MacCallum (see, e.g., Table 1, above; and MacCallum 1996).

In some cases, a CTP is an anti-TROP-2 antibody that comprises: a) a VL region comprising an amino acid sequence having at least 90%, at least 95%, at least 98%, at least 99%, or 100%, amino acid sequence identity to the following amino acid sequence:
DIQMTQSPSSLSASVGDRVTITCKASQDVSTAVAWYQQKPGKAPKLLIYSASYRYTGVPSRFSG SGSGTDFTLTISSLQPEDFAVYYCQQHYITPLTFGQGTKLEIK (SEQ ID NO: 113); and b) a VH region comprising an amino acid sequence having at least 90%, at least 95%, at least 98%, at least 99%, or 100%, amino acid sequence identity to the following amino acid sequence:
QVQLVQSGAEVKKPGASVKVSCKASGYTFTTAGMQWVRQAPGQGLEWMGWINTHSGVPKYA EDFKGRVTISADTSTSTAYLQLSSLKSEDTAVYYCARSGFGSSYWYFDVWGQGTLVTVSS (SEQ ID NO:114).

In some cases, a CTP is an anti-TROP-2 scFv comprising, in order from N-terminus to C-terminus: a) a VL region comprising an amino acid sequence having at least 90%, at least 95%, at least 98%, at least 99%, or 100%, amino acid sequence identity to the following amino acid sequence:
DIQMTQSPSSLSASVGDRVTITCKASQDVSTAVAWYQQKPGKAPKLLIYSASYRYTGVPSRFSG SGSGTDFTLTISSLQPEDFAVYYCQQHYITPLTFGQGTKLEIK (SEQ ID NO: 113); b) a peptide linker; and c) a VH region comprising an amino acid sequence having at least 90%, at least 95%, at least 98%, at least 99%, or 100%, amino acid sequence identity to the following amino acid sequence:
QVQLVQSGAEVKKPGASVKVSCKASGYTFTTAGMQWVRQAPGQGLEWMGWINTHSGVPKYA EDFKGRVTISADTSTSTAYLQLSSLKSEDTAVYYCARSGFGSSYWYFDVWGQGTLVTVSS (SEQ ID NO: 114). In some cases, the peptide linker comprises the amino acid sequence (GGGGS)n (SEQ ID NO:42), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:82) and has a length of 15 amino acids.

In some cases, a CTP is an anti-TROP-2 scFv comprising, in order from N-terminus to C-terminus: a) a VH region comprising an amino acid sequence having at least 90%, at least 95%, at least 98%, at least 99%, or 100%, amino acid sequence identity to the following amino acid sequence:
QVQLVQSGAEVKKPGASVKVSCKASGYTFTTAGMQWVRQAPGQGLEWMGWINTHSGVPKYA EDFKGRVTISADTSTSTAYLQLSSLKSEDTAVYYCARSGFGSSYWYFDVWGQGTLVTVSS (SEQ ID NO:114); b) a peptide linker; and c) a VL region comprising an amino acid sequence having at least 90%, at least 95%, at least 98%, at least 99%, or 100%, amino acid sequence identity to the following amino acid sequence:
DIQMTQSPSSLSASVGDRVTITCKASQDVSTAVAWYQQKPGKAPKLLIYSASYRYTGVPSRFSG SGSGTDFTLTISSLQPEDFAVYYCQQHYITPLTFGQGTKLEIK (SEQ ID NO: 113). In some cases, the peptide linker comprises the amino acid sequence (GGGGS)n (SEQ ID NO:42), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:82) and has a length of 15 amino acids. Anti-BCMA

In some cases, a CTP present in an IL-2 variant fusion polypeptide of the present disclosure comprises an anti-BCMA antibody. In some cases, the anti-BCMA antibody is a scFv polypeptide. In some cases, the anti-BCMA antibody is a nanobody. Anti-BCMA (B-cell maturation antigen) antibodies are known in the art; and the VH and VL, or the VH and VL CDRs, of any anti-BCMA antibody can be used as a CTP. See, e.g., WO 2014/089335; US 2019/0153061; and WO 2017/093942.

In some cases, an anti-BCMA antibody comprises: a) a light chain comprising an amino acid sequence having at least 90%, at least 95%, at least 98%, at least 99%, or 100%, amino acid sequence identity to the following amino acid sequence:

(SEQ ID NO: 115)
QSVLTQPPSASGTPGQRVTISCSGSSSNIGSNTVNWYQQLPGTAP
KLLIFNYHQRPSGVPDRFSGSKSGSSASLAISGLQSEDEADYYCA
AWDDSLNGWVFGGGTKLTVLGQPKAAPSVTLFPPSSEELQANKAT
LVCLISDFYPGAVTVAWKADSSPVKAGVETTTPDSKQSNNKYAAS
SYLSLTPEQWKSHRSYSCQVTHEGSTVEKTVAPTECS;
and

b) a heavy chain comprising an amino acid sequence having at least 90%, at least 95%, at least 98%, at least 99%, or 100%, amino acid sequence identity to the following amino acid sequence:

(SEQ ID NO: 116)
EVOLVESGGGLVKPGGSLRLSCAASGFTFGDYALSWFRQAPGKGLEWVGV
SRSKAYGGTTDYAASVKGRFTISRDDSKSTAYLQMNSLKTEDTAVYYCAS
SGYSSGWTPFDYWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK.

In some cases, an anti-BCMA antibody comprises a VL present in the light chain amino acid sequence provided above; and a VH present in the heavy chain amino acid sequence provided above. For example, an anti-BCMA antibody can comprise: a) a VL comprising an amino acid sequence having at least 90%, at least 95%, at least 98%, at least 99%, or 100%, amino acid sequence identity to the amino acid sequence:

QSVLTQPPSASGTPGQRVTISCSGSSSNIGSNTVNWYQQLPGTAPKLLIFNYHQRP SGVPDRFSGSKSGSSASLAISGLQSEDEADYYCAAWDDSLNGWVFGGGTKLTVLG (SEQ ID NO:117); and b) a VH comprising an amino acid sequence having at least 90%, at least 95%, at least 98%, at least 99%, or 100%, amino acid sequence identity to the amino acid sequence:

(SEQ ID NO: 118)
EVQLVESGGGLVKPGGSLRLSCAASGFTFGDYALSWFRQAPGKGLEWVGV
SRSKAYGGTTDYAASVKGRFTISRDDSKSTAYLQMNSLKTEDTAVYYCAS
SGYSSGWTPFDYWGQGTLVTVSSASTKGPSV.

In some cases, an anti-BCMA antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). In some cases, the V_{H} and V_{L} CDRs are as defined by MacCallum (see, e.g., Table 1, above; and MacCallum 1996).

For example, an anti-BCMA antibody can comprise a VL CDR1 having the amino acid sequence SSNIGSNT (SEQ ID NO: 119), a VL CDR2 having the amino acid sequence NYH, a VL CDR3 having the amino acid sequence AAWDDSLNGWV (SEQ ID NO:120)), a VH CDR1 having the amino acid sequence GFTFGDYA (SEQ ID NO:121), a VH CDR2 having the amino acid sequence SRSKAYGGTT (SEQ ID NO:122), and a VH CDR3 having the amino acid sequence ASSGYSSGWTPFDY (SEQ ID NO:123).

An anti-BCMA antibody can be a scFv. As one non-limiting example, an anti-BCMA scFv can comprise the following amino acid sequence:

(SEQ ID NO: 124)
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYNGYDVLDNWGQGTLVTVSSGGGGSGGGGSGGGGSGGGGSDIQMTQSPS
SLSASVGDRVTITCSASQDISNYLNWYQQKPGKAPKLLIYYTSNLHSGVP
SRFSGSGSGTDFTLTISSLQPEDFATYYCQQYRKLPWTFGQGTKLEIKR.

As another example, an anti-BCMA scFv can comprise the following amino acid sequence:

(SEQ ID NO: 125)
DIQMTQSPSSLSASVGDRVTITCSASQDISNYLNWYQQKPGKAPKLLIYY
TSNLHSGVPSRESGSGSGTDFTLTISSLQPEDFATYYCQQYRKLPWTFGQ
GTKLEIKRGGGGSGGGGSGGGGSGGGGSQVQLVQSGAEVKKPGSSVKVSC
KASGGTFSNYWMHWVRQAPGQGLEWMGATYRGHSDTYYNQKFKGRVTITA
DKSTSTAYMELSSLRSEDTAVYYCARGAIYNGYDVLDNWGQGTLVTVSS.

In some cases, an anti-BCMA antibody can comprise a VL CDR1 having the amino acid sequence SASQDISNYLN (SEQ ID NO:126); a VL CDR2 having the amino acid sequence YTSNLHS (SEQ ID NO:127); a VL CDR3 having the amino acid sequence QQYRKLPWT (SEQ ID NO:128); a VH CDR1 having the amino acid sequence NYWMH (SEQ ID NO:129); a VH CDR2 having the amino acid sequence ATYRGHSDTYYNQKFKG (SEQ ID NO:130); and a VH CDR3 having the amino acid sequence GAIYNGYDVLDN (SEQ ID NO:131).

In some cases, an anti-BCMA antibody comprises: a) a light chain comprising an amino acid sequence having at least 90%, at least 95%, at least 98%, at least 99%, or 100%, amino acid sequence identity to the following amino acid sequence:

(SEQ ID NO: 132)
DIQMTQSPSSLSASVGDRVTITCSASQDISNYLNWYQQKPGKAPKLLIYY
TSNLHSGVPSRFSGSGSGTDFTLTISSLQPEDFATYYCQQYRKLPWTFGQ
GTKLEIKR.

In some cases, an anti-BCMA antibody comprises: a) a heavy chain comprising an amino acid sequence having at least 90%, at least 95%, at least 98%, at least 99%, or 100%, amino acid sequence identity to the following amino acid sequence:

(SEQ ID NO: 133)
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYDGYDVLDNWGQGTLVTVSS.

In some cases, an anti-BCMA antibody (e.g., an antibody referred to in the literature as belantamab) comprises a light chain comprising the amino acid sequence:
DIQMTQSPSSLSASVGDRVTITCSASQDISNYLNWYQQKPGKAPKLLIYYTSNLHSGVPSRFSGS GSGTDFTLTISSLQPEDFATYYCQQYRKLPWTFGQGTKLEIKR (SEQ ID NO:132); and a heavy chain comprising the amino acid sequence:

(SEQ ID NO: 133)
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYDGYDVLDNWGQGTLVTVSS.

In some cases, the anti-BCMA antibody has a cancer chemotherapeutic agent linked to the antibody. For example, in some cases, the anti-BCMA antibody is GSK2857916 (belantamab-mafodotin), where monomethyl auristatin F (MMAF) is linked via a maleimidocaproyl linker to the anti-BCMA antibody belantamab.

### Anti-MUC1

In some cases, a CTP present in an IL-2 variant fusion polypeptide of the present disclosure comprises an anti-MUC1 antibody. In some cases, the anti-MUC1 antibody is a scFv polypeptide. In some cases, the anti-MUC1 antibody is a nanobody. For example, a CTP can be specific for a MUC1 polypeptide present on a cancer cell. In some cases, the CTP is specific for the cleaved form of MUC1; see, e.g., Fessler et al. (2009) Breast Cancer Res. Treat. 118:113. In some cases, the CTP is an antibody specific for a glycosylated MUC1 peptide; see, e.g., Naito et al. (2017) ACS Omega 2:7493; and U.S. Pat. No. 10,017,580.

As one non-limiting example, a CTP can be a single-chain Fv specific for MUC1. See, e.g., Singh et al. (2007) Mol. Cancer Ther. 6:562; Thie et al. (2011) PloSOne 6:e15921; Imai et al. (2004) Leukemia 18:676; Posey et al. (2016) Immunity 44:1444; EP3130607; EP3164418; WO 2002/044217; and US 2018/0112007. In some cases, a CTP is a scFv specific for the MUC1 peptide VTSAPDTRPAPGSTAPPAHG (SEQ ID NO:134). In some cases, a CTP is a scFv specific for the MUC1 peptide SNIKFRPGSVVVQLTLAFREGTINVHDVETQFNQYKTEAASRY (SEQ ID NO:135).

In some cases, a CTP is a scFv specific for the MUC1 peptide SVVVQLTLAFREGTINVHDVETQFNQYKTEAASRY (SEQ ID NO:136). In some cases, a CTP is a scFv specific for the MUC1 peptide LAFREGTINVHDVETQFNQY (SEQ ID NO:137). In some cases, a CTP is a scFv specific for the MUC1 peptide SNIKFRPGSVVVQLTLAAFREGTIN (SEQ ID NO:138).

As an example, an anti-MUC1 antibody can comprise: a VH CDR1 having the amino acid sequence RYGMS (SEQ ID NO:139); a VH CDR2 having the amino acid sequence TISGGGTYIYYPDSVKG (SEQ ID NO:140); a VH CDR3 having the amino acid sequence DNYGRNYDYGMDY (SEQ ID NO:141); a VL CDR1 having the amino acid sequence SATSSVSYIH (SEQ ID NO:142); a VL CDR2 having the amino acid sequence STSNLAS (SEQ ID NO:143); and a VL CDR3 having the amino acid sequence QQRSSSPFT (SEQ ID NO:144). See, e.g., US 2018/0112007.

As another example, an anti-MUC1 antibody can comprise a VH CDR1 having the amino acid sequence GYAMS (SEQ ID NO:145); a VH CDR2 having the amino acid sequence TISSGGTYIYYPDSVKG (SEQ ID NO:146); a VH CDR3 having the amino acid sequence LGGDNYYEYFDV (SEQ ID NO:147); a VL CDR1 having the amino acid sequence RASKSVSTSGYSYMH (SEQ ID NO:148); a VL CDR2 having the amino acid sequence LASNLES (SEQ ID NO:149); and a VL CDR3 having the amino acid sequence QHSRELPFT (SEQ ID NO:150). See, e.g., US 2018/0112007.

As another example, an anti-MUC1 antibody can comprise a VH CDR1 having the amino acid sequence DYAMN (SEQ ID NO:151); a VH CDR2 having the amino acid sequence VISTFSGNINFNQKFKG (SEQ ID NO:152); a VH CDR3 having the amino acid sequence SDYYGPYFDY (SEQ ID NO:153); a VL CDR1 having the amino acid sequence RSSQTIVHSNGNTYLE (SEQ ID NO:154); a VL CDR2 having the amino acid sequence KVSNRFS (SEQ ID NO:155); and a VL CDR3 having the amino acid sequence (FQGSHVPFT (SEQ ID NO:156). See, e.g., US 2018/0112007.

As another example, an anti-MUC1 antibody can comprise a VH CDR1 having the amino acid sequence GYAMS (SEQ ID NO:145); a VH CDR2 having the amino acid sequence TISSGGTYIYYPDSVKG (SEQ ID NO:146); a VH CDR3 having the amino acid sequence LGGDNYYEY (SEQ ID NO:157); a VL CDR1 having the amino acid sequence TASKSVSTSGYSYMH (SEQ ID NO:158); a VL CDR2 having the amino acid sequence LVSNLES (SEQ ID NO:159); and a VL CDR3 having the amino acid sequence QHIRELTRSE (SEQ ID NO:160). See, e.g., US 2018/0112007.

### Anti-MUC16

In some cases, a CTP present in an IL-2 variant fusion polypeptide of the present disclosure comprises an anti-MUC16 antibody. MUC16 is also known as CA125. In some cases, the anti-MUC16 antibody is a seFv polypeptide. In some cases, the anti-MUC16 antibody is a nanobody. See, e.g., Yin et al. (2002) Int. J. Cancer 98:737. For example, a CTP can be specific for a MUC16 polypeptide present on a cancer cell. See, e.g., US 2018/0118848; and US 2018/0112008.

As one example, an anti-MUC16 antibody can comprise a VH CDR1 having the amino acid sequence GFTFSNYY (SEQ ID NO:161); a VH CDR2 having the amino acid sequence ISGRGSTI (SEQ ID NO:162); a VH CDR3 having the amino acid sequence VKDRGGYSPY (SEQ ID NO:163); a VL CDR1 having the amino acid sequence QSISTY (SEQ ID NO:164); a VL CDR2 having the amino acid sequence TAS; and a VL CDR3 having the amino acid sequence QQSYSTPPIT (SEQ ID NO:165). See, e.g., US 2018/0118848.

### Anti-Claudin-18.2

In some cases, a CTP present in an IL-2 variant fusion polypeptide of the present disclosure comprises an anti-claudin-18 isoform 2 (“claudin-18.2”) antibody. In some cases, the anti-claudin 18.2 antibody is a scFv polypeptide. In some cases, the anti-claudin 18.2 antibody is a nanobody. See, e.g., WO 2013/167259. In some cases, a CTP is an antibody specific for TEDEVQSYPSKHDYV (SEQ ID NO:166) or EVQSYPSKHDYV (SEQ ID NO:167).

As one example, an anti-claudin-18.2 antibody can comprise a VH CDR1 having the amino acid sequence GYTFTDYS (SEQ ID NO:168); a VH CDR2 having the amino acid sequence INTETGVP (SEQ ID NO:169); a VH CDR3 having the amino acid sequence ARRTGFDY (SEQ ID NO:170); a VL CDR1 having the amino acid sequence KNLLHSDGITY (SEQ ID NO:171); a VL CDR2 having the amino acid sequence RVS; and a VL CDR3 having the amino acid sequence VQVLELPFT (SEQ ID NO:172).

As another example, an anti-claudin-18.2 antibody can comprise a VH CDR1 having the amino acid sequence GFTFSSYA (SEQ ID NO:173); a VH CDR2 having the amino acid sequence ISDGGSYS (SEQ ID NO:174); a VH CDR3 having the amino acid sequence ARDSYYDNSYVRDY (SEQ ID NO:175); a VL CDR1 having the amino acid sequence QDINTF (SEQ ID NO:176); a VL CDR2 having the amino acid sequence RTN; and a VL CDR3 having the amino acid sequence LQYDEFPLT (SEQ ID NO:177).

### Anti-EGFR

In some cases, a CTP present in an IL-2 variant fusion polypeptide of the present disclosure is an antibody specific for epidermal growth factor receptor (EGFR) See, e.g., U.S. Pat. No. 6,217,866 and US 2015/0141620. In some cases, an EGFR-specific CTP is a scFv. In some cases, an EGFR-specific CTP is a nanobody. In some cases, an EGFR-specific CTP comprises VL and VH CDRs present in Cetuximab.

In some cases, a suitable anti-EGFR antibody comprises VL CDRs present in the following amino acid sequence:

(SEQ ID NO: 178)
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGA.
See, e.g., WO 2020/225552.

In some cases, a suitable anti-EGFR antibody comprises VH CDRs present in the following amino acid sequence:

(SEQ ID NO: 179)
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK.
See, e.g., WO 2020/225552.

In some cases, an anti-EGFR antibody comprises: a) a heavy chain comprising an amino acid sequence having at least 90%, at least 95%, at least 98%, at least 99%, or 100%, amino acid sequence identity to the following amino acid sequence:
QVQLKQSGPGLVQPSQSLSITCTVSGFSLTNYGVHWVRQSPGKGLEWLGVIWSGGNTDYNTPF TSRLSINKDNSKSQVFFKMNSLQSNDTAIYYCARALTYYDYEFAYWGQGTLVTVSAASTKGPS VFPLAPSSKSTSGGTAALGCLVKDYFPEPVTVSWNSGALTSGVHTFPAVLQSSGLYSLSSVVTVP SSSLGTQTYICNVNHKPSNTKVDKRVEPKSCDKTHTCPPCPAPELLGGPSVFLFPPKPKDTLMISR TPEVTCVVVDVSHEDPEVKFNWYVDGVEVHNAKTKPREEQYNSTYRVVSVLTVLHQDWLNG KEYKCKVSNKALPAPIEKTISKAKGQPREPQVYTLPPSREEMTKNQVSLTCLVKGFYPSDIAVE WESNGQPENNYKTTPPVLDSDGSFFLYSKLTVDKSRWQQGNVFSCSVMHEALHNHYTQKSLSL SPGK (SEQ ID NO:179); and b) a light chain comprising an amino acid sequence having at least 90%, at least 95%, at least 98%, at least 99%, or 100%, amino acid sequence identity to the following amino acid sequence:

(SEQ ID NO: 178)
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGA.

As one example, an anti-EGFR antibody can comprise a VH CDR1 having the amino acid sequence NYGVH (SEQ ID NO:180), a VH CDR2 having the amino acid sequence VIWSGGNTDYNTPFTS (SEQ ID NO:181), a VH CDR3 having the amino acid sequence ALTYYDYEFAY (SEQ ID NO:182), a VL CDR1 having the amino acid sequence RASQSIGTNIH (SEQ ID NO:183), a VL CDR2 having the amino acid sequence YASESIS (SEQ ID NO:184), and a VL CDR3 having the amino acid sequence QQNNNQPTT (SEQ ID NO:185). See, e.g., WO 2020/225552.

As another example, an anti-EGFR antibody can comprise a VH CDR1 having the amino acid sequence SYWIE (SEQ ID NO:186), a VH CDR2 having the amino acid sequence EILPGSKKTNYNEKFKG (SEQ ID NO:187), a VH CDR3 having the amino acid sequence YYYRNDDYGMDT (SEQ ID NO:188), a VL CDR1 having the amino acid sequence SASQDIRNYLN (SEQ ID NO:189), a VL CDR2 having the amino acid sequence YTSTLHS (SEQ ID NO:190, and a VL CDR3 having the amino acid sequence QQYSKIPYT (SEQ ID NO:191). See, e.g., U.S. Pat. No. 6,217,866.

As another example, an anti-EGFR antibody can comprise a VH CDR1 having the amino acid sequence NYDMS (SEQ ID NO:192), a VH CDR2 having the amino acid sequence YIGNGGNTYSPDTVKG (SEQ ID NO:193), a VH CDR3 having the amino acid sequence HYGYDGRF (SEQ ID NO:194), a VL CDR1 having the amino acid sequence RSSQSLEHSNGDTYLH (SEQ ID NO:195), a VL CDR2 having the amino acid sequence KVSNRFS (SEQ ID NO:196), and a VL CDR3 having the amino acid sequence CQSTHVPWT (SEQ ID NO:197). See, e.g., U.S. Pat. No. 6,217,866.

### Anti-CD20

In some cases, a CTP present in an IL-2 variant fusion polypeptide of the present disclosure comprises an anti-CD20 antibody. Anti-CD20 antibodies are known in the art; see, e.g., US 2015/0141620. In some cases, a CD20-specific CTP comprises VL and VH CDRs present in Rituximab. In some cases, a CD20-specific CTP comprises VL and VH present in Rituximab. In some cases, the anti-CD20 antibody is a scFv polypeptide. In some cases, the anti-CD20 antibody is a nanobody.

In some cases, a suitable anti-CD20 antibody comprises VH CDRs present in the following amino acid sequence:

(SEQ ID NO: 198)
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVASASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKAEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K.

In some cases, a suitable anti-CD20 antibody comprises VL CDRs present in the following amino acid sequence:

(SEQ ID NO: 199)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

In some cases, a suitable anti-CD20 antibody comprises: a) a VH comprising an amino acid sequence having at least 90%, at least 95%, at least 98%, at least 99%, or 100%, amino acid sequence identity to the following amino acid sequence:
QVQLQQPGAELVKPGASVKMSCKASGYTFTSYNMHWVKQTPGRGLEWIGAIYPGNGDTSYNQ KFKGKATLTADKSSSTAYMQLSSLTSEDSAVYYCARSTYYGGDWYFNVWGAGTTVTVASAST KGPSVFPLAPSSKSTSGGTAALGCLVKDYFPEPVTVSWNSGALTSGVHTFPAVLQSSGLYSLSSV VTVPSSSLGTQTYICNVNHKPSNTKVDKKAEPKSCDKTHTCPPCPAPELLGGPSVFLFPPKPKDT LMISRTPEVTCVVVDVSHEDPEVKFNWYVDGVEVHNAKTKPREEQYNSTYRVVSVLTVLHQD WLNGKEYKCKVSNKALPAPIEKTISKAKGQPREPQVYTLPPSRDELTKNQVSLTCLVKGFYPSDI AVEWESNGQPENNYKTTPPVLDSDGSFFLYSKLTVDKSRWQQGNVFSCSVMHEALHNHYTQK SLSLSPGK (SEQ ID NO:198); and b) a VL comprising an amino acid sequence having at least 90%, at least 95%, at least 98%, at least 99%, or 100%, amino acid sequence identity to the following amino acid sequence:

(SEQ ID NO: 199)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

CTPs that are mAb Fusion Polypeptides

In embodiments, one or more IL-2 variant polypeptides can be part of a mAb fusion polypeptide (not shown in the figures). The mAb fusion polypeptide can comprise a heterodimer, wherein the first polypeptide of the heterodimer comprises an antibody heavy chain polypeptide, and the second polypeptide of the heterodimer comprises an antibody light chain polypeptide, and wherein either the first polypeptide or the second polypeptide comprises one or more variant IL-2 polypeptides, e.g., on the N-terminus of the antibody light chain polypeptide, on the C-terminus of the antibody light chain polypeptide, or on the C-terminus of the antibody heavy chain polypeptide. Such fusion polypeptides are referred to herein as “monoclonal antibody fusions” or “mAb fusions.” The VH polypeptide and the VL polypeptide are on separate polypeptides of the heterodimer; together, the VH polypeptide and the VL polypeptide bind to a cancer epitope and therefore together constitute a CTP. Such mAbs can be directed against any of the above cancer targets or HSA.

### T Cell Receptors (TCRs)

As noted above, in some cases, a functional protein is a TCR such as single-chain T cell receptor or “scTCR”. A functional protein can be a scTCR that is specific for a peptide/HLA complex on the surface of a cancer cell, where the peptide can be a cancer-associated peptide (e.g., a peptide of a cancer-associated antigen). Amino acid sequences of scTCRs specific for cancer-associated peptides bound to an HLA complex are known in the art. See, e.g., US 2019/0135914; US 2019/0062398; and US 2018/0371049.

A scTCR includes an alpha chain variable region (Vα) and a beta chain variable region (Vβ) covalently linked through a suitable peptide linker sequence. For example, the Vα can be covalently linked to the Vβ through a suitable peptide linker (L) sequence fused to the C-terminus of the Vα and the N-terminus of the Vβ. An scTCR can have the structure Vα-L-Vβ. An scTCR can have the structure Vβ-L-Vα. An scTCR can also comprise a constant domain (also referred to as constant region). In some cases, an scTCR comprises, in order from N-terminus to C-terminus: i) a TCR α chain variable domain polypeptide; ii) a peptide linker; iii) a TCR β chain variable domain polypeptide; and iv) a TCR β chain constant region extracellular domain polypeptide. In some cases, an scTCR comprises, in order from N-terminus to C-terminus: i) a TCR β chain variable domain polypeptide; ii) a peptide linker; iii) a TCR a chain variable domain polypeptide; and iv) a TCR a chain constant region extracellular domain polypeptide.

Amino acid sequences of scTCRs specific for peptide/HLA complexes, where the peptide is a cancer-associated peptide, are known in the art. See, e.g., US 2019/0135914; US 2019/0062398; US 2018/0371049; US 2019/0144563; and US 2019/0119350.

Alternatively, as with the mAb Fusion Polypeptides described above, the one or more IL-2 variant polypeptides can be part of a TCR fusion polypeptide (also not shown in the figures). Such TCR fusion polypeptides can comprise a heterodimer, wherein the first polypeptide of the heterodimer comprises at least the variable region of an a chain of a TCR (and optionally also the constant region), and the second polypeptide of the heterodimer comprises at least the variable region of the β chain of the TCR (and optionally also the constant region), and wherein either the first polypeptide or the second polypeptide comprises one or more variant IL-2 polypeptides, e.g., on the C-terminus of the variable region of the polypeptide comprising the a chain polypeptide(s), and/or on the C-terminus of the variable region of the polypeptide comprising the β chain polypeptide(s). Such fusion polypeptides are referred to herein as “TCR fusion polypeptides”. The a chain polypeptide(s) and β chain polypeptide(s) are on separate polypeptides of the heterodimer; together, they bind to a pHLA complex on the surface of a cancer cell therefore together constitute a CTP.

### Immunomodulatory Polypeptides

The functional protein also may comprise a wild-type or variant immunomodulatory polypeptide, e.g., a wild type or variant immunostimulatory polypeptide such as B7 family of costimulatory receptors, e.g., CD80, CD86, a cytokine such as IL-7, IL-12, IL-15 or IL-21, a TNF superfamily member such as CD-40, 4-1BBL and OX40, or a chemokine such as CCL19, CCL21, CXCL9/10/11, or CXCL12.

### Linkers

An IL-2 variant polypeptide or fusion polypeptide of the present disclosure can include one or more independently selected linkers, i.e., a contiguous stretch of two or more amino acids that join one or more IL-2 variant polypeptides of a higher order IL-2 variant polypeptide such as a dimer, trimer, etc., or that join one or more components of a fusion polypeptide as described herein.

Suitable linkers (also referred to as “spacers”) can be readily selected and can be of any of a number of suitable lengths, such as from 1 amino acid to 25 amino acids, from 25 amino acids to 50 amino acids, or greater than 50 amino acids in length.

Generally speaking, linkers can be flexible linkers or rigid linkers. Linkers also can comprise a cysteine residue in instances where it is desired to join the linker to another part of the polypeptide through a disulfide bond. Such linkers typically are not cleavable linkers, i.e., they are not designed to be cleaved in vivo.

### Flexible Peptide Linkers

Exemplary flexible linkers include glycine polymers (G)ₙ, glycine-serine polymers (including, for example, (GS)ₙ, (GSGGS)ₙ (SEQ ID NO:200), (GGGGS)ₙ (SEQ ID NO:201), and (GGGS)ₙ (SEQ ID NO:202), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:203), GGSGG (SEQ ID NO:204), GSGSG (SEQ ID NO:205), GSGGG (SEQ ID NO:206), GGGSG (SEQ ID NO:207), GSSSG (SEQ ID NO:208), and the like. Exemplary linkers can include, e.g., Gly(Ser₄)n (SEQ ID NO:209), where n is 1, 2, 3, 4, 5, 6, 7, 8, 9, or 10. In some cases, a linker comprises the amino acid sequence (GSSSS)n (SEQ ID NO:210), where n is 4. In some cases, a linker comprises the amino acid sequence (GSSSS)n (SEQ ID NO:211), where n is 5. Exemplary linkers can include, e.g., (GGGGS)ₙ (SEQ ID NO:42), where n is 1, 2, 3, 4, 5, 6, 7, 8, 9, or 10. In some cases, a linker comprises the amino acid sequence (GGGGS)n (SEQ ID NO:212), where n is 2. In some cases, a linker comprises the amino acid sequence (GGGGS)n (SEQ ID NO:82), where n is 3. In some cases, a linker comprises the amino acid sequence (GGGGS)n (SEQ ID NO:213), where n is 4. In some cases, a linker comprises the amino acid sequence (GGGGS)n (SEQ ID NO:214), where n is 5.

Exemplary flexible peptide linkers include, e.g., (GGGGS)n (SEQ ID NO:42); also referred to as a “G4S” linker), where n is 1, 2, 3, 4, 5, 6, 7, 8, 9, or 10. In some cases, a linker comprises the amino acid sequence AAAGG (SEQ ID NO:215. Also suitable is a linker having the amino acid sequence AAAGG (SEQ ID NO:215).

As used in this disclosure, a “short flexible peptide linker” means a flexible peptide linker that comprises fewer than 15 amino acids, i.e., from 2-14 amino acids. For example, a short flexible peptide linker can comprise from 2-4, 2-5, or 3-6 amino acids (e.g., a GGS linker), or from 4-8, 5-10 or from 10-14 amino acids. Within this range includes flexible peptide linkers comprising 2, 3, 4, 5, 6, 7, 8, 9, 10, 11, 12, 13 or 14 amino acids.

### Rigid Peptide Linkers

In some cases, a peptide linker is a rigid peptide linker. As used herein, the term “rigid peptide linker” refers to a linker comprising a contiguous stretch of two or more amino acids that effectively separates protein domains by maintaining a substantially fixed distance/spatial separation between the domains, thereby reducing or substantially eliminating unfavorable interactions between such domains. Rigid peptide linkers are known in the art and generally adopt a relatively well-defined conformation when in solution. Rigid peptide linkers include those which have a particular secondary and/or tertiary structure in solution; and are typically of a length sufficient to confer secondary or tertiary structure to the linker. Rigid peptide linkers include peptide linkers rich in proline, and peptide linkers having an inflexible helical structure, such as an a-helical structure. Rigid peptide linkers are described in, for example, Chen et al. (2013) Adv. Drug Deliv. Rev. 65:1357; and Klein et al. (2014) Protein Engineering, Design & Selection 27:325.

Examples of rigid peptide linkers include, e.g., (EAAAK)n (SEQ ID NO:216), A(EAAAK)n (SEQ ID NO:217), A(EAAAK)nA (SEQ ID NO:218), A(EAAAK)nALEA(EAAAK)nA (SEQ ID NO:219), (Lys-Pro)n (SEQ ID NO:220), (Glu-Pro)n (SEQ ID NO:221), (Thr-Pro-Arg)n (SEQ ID NO:222), and (Ala-Pro)n (SEQ ID NO:223) where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid peptide linkers comprising EAAAK (SEQ ID NO:224) include EAAAK (SEQ ID NO:224), (EAAAK)₂ (SEQ ID NO:225), (EAAAK)₃ (SEQ ID NO:226), A(EAAAK)₄ALEA(EAAAK)₄A (SEQ ID NO:227), and AEAAAKEAAAKA (SEQ ID NO:228). Non-limiting examples of suitable rigid peptide linkers comprising (AP)n include PAPAP (SEQ ID NO:229); also referred to herein as “(AP)2”); APAPAPAP (SEQ ID NO:230); also referred to herein as “(AP)4”); APAPAPAPAPAP (SEQ ID NO:231); also referred to herein as “(AP)6”); APAPAPAPAPAPAPAP (SEQ ID NO:232); also referred to herein as “(AP)8”); and APAPAPAPAPAPAPAPAPAP (SEQ ID NO:233); also referred to herein as “(AP)10”). Non-limiting examples of suitable rigid peptide linkers comprising (KP)n include KPKP (SEQ ID NO:234); also referred to herein as “(KP)2”); KPKPKPKP (SEQ ID NO:235); also referred to herein as “(KP)4”); KPKPKPKPKPKP (SEQ ID NO:236); also referred to herein as “(KP)6”); KPKPKPKPKPKPKPKP (SEQ ID NO:237); also referred to herein as “(KP)8”); and KPKPKPKPKPKPKPKPKPKP (SEQ ID NO:238); also referred to herein as “(KP)10”). Non-limiting examples of suitable rigid peptide linkers comprising (EP)n include EPEP (SEQ ID NO:239); also referred to herein as “(EP)2”); EPEPEPEP (SEQ ID NO:240); also referred to herein as “(EP)4”); EPEPEPEPEPEP (SEQ ID NO:241); also referred to herein as “(EP)6”); EPEPEPEPEPEPEPEP (SEQ ID NO:242); also referred to herein as “(EP)8”); and EPEPEPEPEPEPEPEPEPEP (SEQ ID NO:243); also referred to herein as “(EP)10”).

Generally speaking, where a higher order IL-2 variant polypeptide or fusion polypeptide comprises a rigid peptide linker and/or a short flexible peptide linker, the rigid peptide linker and/or short flexible peptide linker may be positioned between any two of the components. In some cases, the use of a rigid peptide linker or short flexible peptide linker may increase the thermal stability of the resulting polypeptide.

### Cysteine-Containing Linkers

As noted above, in some cases, a linker peptide includes a cysteine residue that can form an intrachain disulfide bond with a cysteine residue present elsewhere in the polypeptide chain. Exemplary cysteine-containing linkers include GCGGS (SEQ ID NO:244), CGGGS (SEQ ID NO:245), CGGGS(GGGGS)n (SEQ ID NO:246), where n is 1, 2, 3, 4, 5, 6, 7, 8, 9, or 10; e.g., 1, 2, or 3, GGCGS (SEQ ID NO:247), GGCGS(GGGGS)n (SEQ ID NO:248), where n is 1, 2, 3, 4, 5, 6, 7, 8, 9 or 10, e.g., 1, 2, or 3, GGGCS (SEQ ID NO:249), GGGCS(GGGGS)n (SEQ ID NO:250), where n is 1, 2, 3, 4, 5, 6, 7, 8, 9, or 10, e.g., 1, 2, or 3, GGGGC (SEQ ID NO:251), and GGGGC(GGGGS)n (SEQ ID NO:252), where n is 1, 2, 3, 4, 5, 6, 7, 8, 9, or 10, e.g., 1, 2, or 3.

### Exemplary Polypeptides

Exemplary polypeptides are shown in the Figures. Exemplary IL-2 variant polypeptides include those where an F42 substitution (e.g., with Ala or Lys) is provided in order to substantially reduce the binding of the IL-2 variant to IL-2Rα, e.g., F42A or F42K, e.g., by at least about 100-fold. As discussed above, IL-2 variant polypeptides having F42A or F42K substitutions also will have one or more substitutions that decrease the binding of the variant to IL-2Rβ, e.g., substitutions at E15, H16 or N88. Of these, H16 substitutions have been shown to reduce the binding to IL-2Rβ by about 3-fold. H16 substitutions include, e.g., Ala, Glu, Thr, or Asp. Exemplary variants thus can comprise any one of the following pairs of substitutions:
H16A, F42A;
H16T, F42A;
H16E, F42A;
H16D, F42A;
H16A, F42K;
H16T, F42K;
H16E, F42K; or
H16D, F42K

Thus, for example, the higher order forms of IL-2 variant polypeptides illustrated in FIGS. 1A-IC may comprise IL-2 variants that comprise any one of the combination of substitutions set forth in Table 3, including, e.g., one of the following pairs of substitutions: H16A, F42A; H16T, F42A; H16E, F42A; H16D, F42A; H16A, F42K; H16T, F42K; H16E, F42K; or H16D, F42K. The IL-2 variant polypeptides may have one or more additional substitutions, and may be joined by linkers as described above.

Likewise, the fusion polypeptides illustrated in FIGS. 2A-2C, 3A-3C, 4A-4C, 5A-5B, 6A-6B, 7A-7B, 8A-8F, 10A-10C, 11A-11C, 12A-12B, 13A-13B, 14A-14N, and 30A-30G all may comprise variant IL-2 polypeptides that comprise any one of the combination of substitutions set forth in Table 3, including, e.g., one of the following pairs of substitutions: H16A, F42A; H16T, F42A; H16E, F42A; H16D, F42A; H16A, F42K; H16T, F42K; H16E, F42K; or H16D, F42K. The IL-2 variant polypeptides may have one or more additional substitutions, and one or more of the components of the fusion polypeptides may be joined by linkers as described above. Exemplary fusion proteins may employ any one of the Ig Fc polypeptides in FIGS. 9A-9M such as a human IgG1 Fc variant polypeptide that has a substantially reduced ability to effect complement-dependent cytotoxicity (CDC) or antibody-dependent cell cytotoxicity (ADCC), e.g., a variant polypeptide having at least 85%, at least 90%, at least 95%, at least 98%, at least 99%, or 100%, amino acid sequence identity to the human the variant human IgG1 Fc polypeptides of FIG. 9B (SEQ ID NO:24) and FIG. 9D (SEQ ID NO: 26).

As one non-limiting example, a fusion polypeptide (e.g., as depicted schematically in FIG. 11B and FIG. 13B), can comprise, in order from N-terminus to C-terminus:
i) an anti-CD19 scFv comprising an amino acid sequence having at least 85%, at least 90%, at least 95%, at least 97%, at least 98%, at least 99%, or 100%, amino acid sequence identity to the following amino acid sequence:

(SEQ ID NO: 67)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIKGGGGSGGGGSGGGGSQVQLQQSGAELVRPGSSVKISCKA
SGYAFSSYWMNWVKQRPGQGLEWIGQIWPGDGDTNYNGKFKGKATLTADE
SSSTAYMQLSSLASEDSAVYFCARRETTTVGRYYYAMDYWGQGTTVTVSS;

ii) a peptide linker;
iii) an Ig Fe polypeptide comprising an amino acid sequence having at least 85%, at least 90%, at least 95%, at least 97%, at least 98%, at least 99%, or 100%, amino acid sequence identity to the following amino acid sequence:
DKTHTCPPCPAPEAAGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLT VDKSRWQQGNVFSCSVMHEALHNHYTQKSLSLSPG (SEQ ID NO:253), where amino acid 14 is Ala and amino acid 15 is Ala;
iv) a peptide linker;
v) a first copy of a variant IL-2 polypeptide, where the variant IL-2 polypeptide comprises an amino acid sequence having at least 85%, at least 90%, at least 95%, at least 97%, at least 98%, at least 99%, or 100%, amino acid sequence identity to the following amino acid sequence:
APTSSSTKKTQLQLEALLLDLQMILNGINNYKNPKLTRMLTAKFYMPKKATELKHLQCLEEELK PLEEVLNLAQSKNFHLRPRDLISNINVIVLELKGSETTFMCEYADETATIVEFLNRWITFCQSIIST LT (SEQ ID NO:254), where amino acid 16 is other than His and where amino acid 42 is other than Phe;
vi) a peptide linker; and
vii) a second copy of a variant IL-2 polypeptide, where the variant IL-2 polypeptide comprises an amino acid sequence having at least 85%, at least 90%, at least 95%, at least 97%, at least 98%, at least 99%, or 100%, amino acid sequence identity to the following amino acid sequence:
APTSSSTKKTQLQLEALLLDLQMILNGINNYKNPKLTRMLTAKFYMPKKATELKHLQCLEEELK PLEEVLNLAQSKNFHLRPRDLISNINVIVLELKGSETTFMCEYADETATIVEFLNRWITFCQSIIST LT (SEQ ID NO:254), where amino acid 16 is other than His and where amino acid 42 is other than Phe.

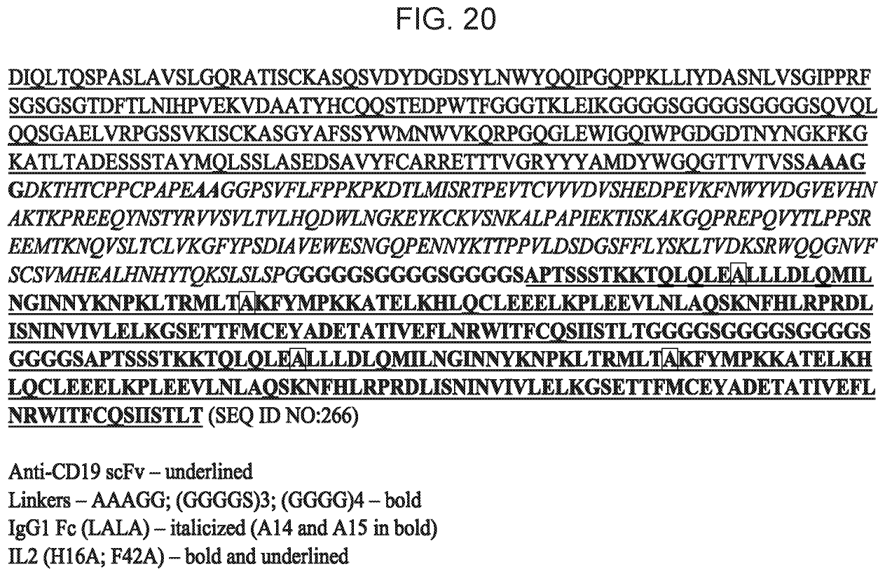

In some cases, the fusion polypeptide comprises an amino acid sequence having at least 85%, at least 90%, at least 95%, at least 97%, at least 98%, at least 99%, or 100%, amino acid sequence identity to the amino acid sequence depicted in FIG. 20. In some cases, the fusion polypeptide comprises the amino acid sequence depicted in FIG. 20.

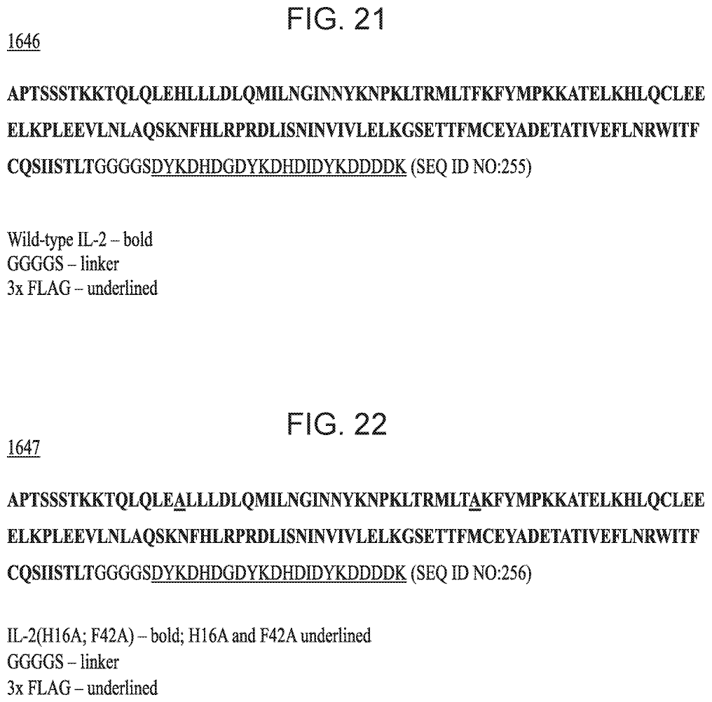

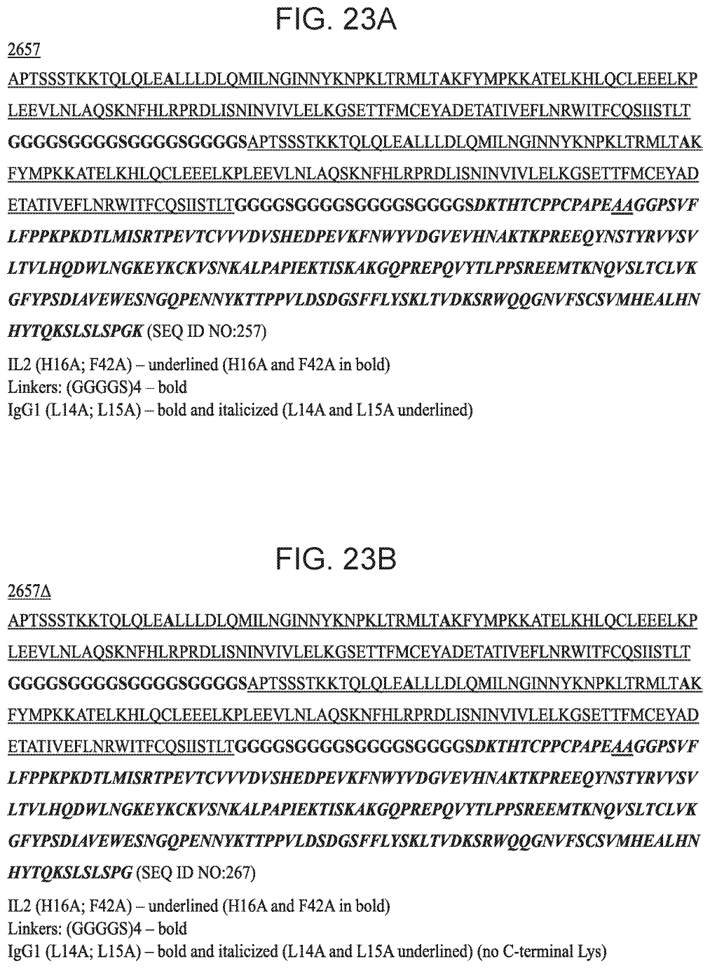

In some cases, the fusion polypeptide comprises, consists essentially of, or consists of, an amino acid sequence having at least 85%, at least 90%, at least 95%, at least 97%, at least 98%, at least 99%, or 100%, amino acid sequence identity to any one of the amino acid sequences depicted in FIGS. 21-26. In some cases, the fusion polypeptide comprises, consists essentially of, or consists of, a homodimer having two copies of an immunomodulatory protein, each copy having an amino acid sequence having at least 85%, at least 90%, at least 95%, at least 97%, at least 98%, at least 99%, or 100%, amino acid sequence identity to the “2657” amino acid sequence depicted in FIG. 23A (and set forth in SEQ ID NO:257). Polypeptide 2657 spontaneously forms a homodimer with a second copy of 2657, wherein the two copies are joined by two disulfide bonds that link the Ig Fc polypeptides in each copy of 2657. FIG. 5B illustrates the structure of a homodimer comprising two polypeptides like 2657. In some cases, the fusion polypeptide comprises, consists essentially of, or consists of, a homodimer having two copies of an immunomodulatory protein, each copy having an amino acid sequence having at least 85%, at least 90%, at least 95%, at least 97%, at least 98%, at least 99%, or 100%, amino acid sequence identity to the “2657A” amino acid sequence depicted in FIG. 23B (and set forth in SEQ ID NO:267). Polypeptide 2657A spontaneously forms a homodimer with a second copy of 2657A, wherein the two copies are joined by two disulfide bonds that link the Ig Fc polypeptides in each copy of 2657A. The fusion polypeptide designated 2657A and shown in FIG. 23B lacks the C-terminal Lys present in 2657.

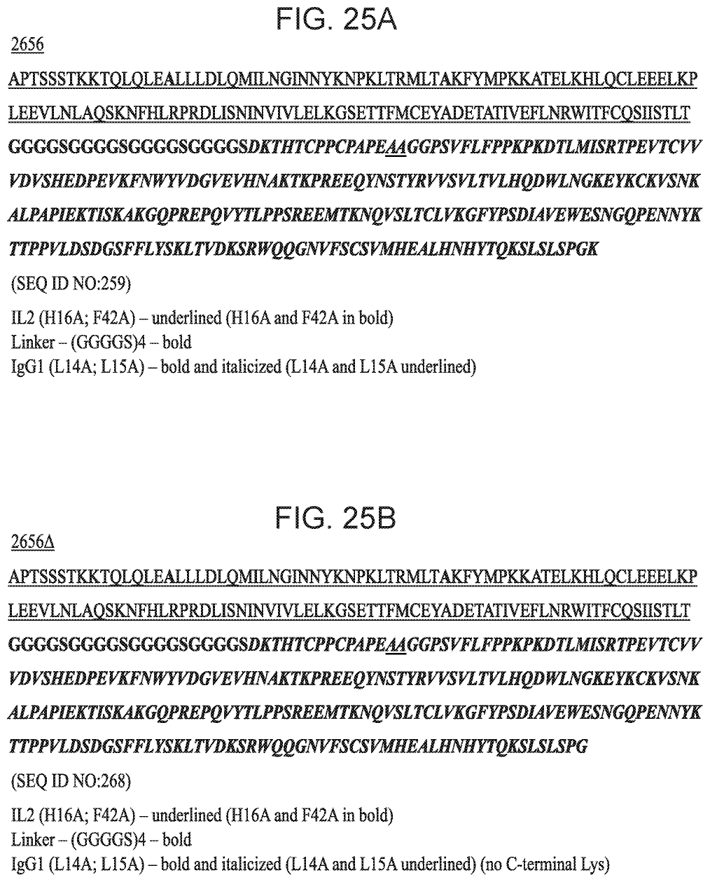

In some cases, the fusion polypeptide comprises, consists essentially of, or consists of, a homodimer having two copies of an immunomodulatory protein, each copy having an amino acid sequence having at least 85%, at least 90%, at least 95%, at least 97%, at least 98%, at least 99%, or 100%, amino acid sequence identity to the “2656” amino acid sequence depicted in FIG. 25A (and set forth in SEQ ID NO:259). Polypeptide 2656 also spontaneously forms a homodimer with a second copy of 2656, wherein the two copies are joined by two disulfide bonds that link the Ig Fe polypeptides in each copy of 2656. FIG. 5A illustrates the structure of a homodimer comprising two polypeptides like 2656. In some cases, the fusion polypeptide comprises, consists essentially of, or consists of, a homodimer having two copies of an immunomodulatory protein, each copy having an amino acid sequence having at least 85%, at least 90%, at least 95%, at least 97%, at least 98%, at least 99%, or 100%, amino acid sequence identity to the “2656A” amino acid sequence depicted in FIG. 25B (and set forth in SEQ ID NO:268). Polypeptide 2656A also spontaneously forms a homodimer with a second copy of 2656A, wherein the two copies are joined by two disulfide bonds that link the Ig Fc polypeptides in each copy of 2656A. The fusion polypeptide designated 2656A and shown in FIG. 25B lacks the C-terminal Lys present in 2656.

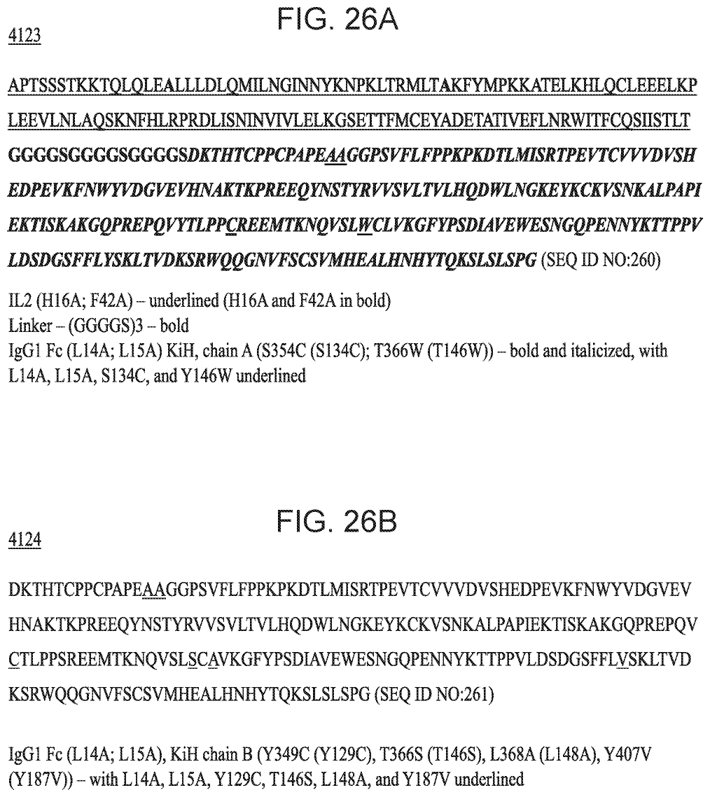

In some cases, a heterodimeric fusion polypeptide comprises, consists essentially of, or consists of: a) a first polypeptide comprising an amino acid sequence having at least 85%, at least 90%, at least 95%, at least 97%, at least 98%, at least 99%, or 100%, amino acid sequence identity to the “4123” amino acid sequence depicted in FIG. 26A (and set forth in SEQ ID NO:260); and b) a second polypeptide comprising an amino acid sequence having at least 85%, at least 90%, at least 95%, at least 97%, at least 98%, at least 99%, or 100%, amino acid sequence identity to the “4124” amino acid sequence depicted in FIG. 26B (and set forth in SEQ ID NO:261). FIG. 30A illustrates the structure of a heterodimer comprising two polypeptides like 4123 and 4124.

### Methods of Generating IL-2 Variant Polypeptides or Fusion Polypeptides

This disclosure provides methods of obtaining an IL-2 variant polypeptide or fusion polypeptide as described herein.

### Nucleic Acids

This disclosure provides a nucleic acid comprising a nucleotide sequence encoding an IL-2 variant polypeptide or fusion polypeptide as described herein. Where the IL-2 variant polypeptide or fusion polypeptide comprises a single chain, or where the fusion polypeptide comprises a homodimer of two single-chain polypeptides, the single-chain polypeptides may be encoded in a single nucleic acid.

Where the fusion polypeptide is heterodimeric, the fusion polypeptide may be encoded by a single nucleic acid or two separate nucleic acids. That is, in some cases, the individual polypeptide chains of a heterodimeric fusion polypeptide described herein are encoded in separate nucleic acids, and may be operably linked to transcriptional control elements, e.g., promoters, such as promoters that are functional in a eukaryotic cell, where the promoter can be a constitutive promoter or an inducible promoter. Where the individual polypeptide chains of a heterodimeric fusion polypeptide are encoded in a single nucleic acid, the nucleic acid can include, e.g., a proteolytically cleavable linker interposed between the nucleotide sequence encoding the first polypeptide and the nucleotide sequence, an internal ribosome entry site (IRES) interposed between the nucleotide sequence encoding the first polypeptide and the nucleotide sequence encoding the second polypeptide, or a ribosome skipping signal (or cis-acting hydrolase element, CHYSEL) interposed between the nucleotide sequence encoding the first polypeptide and the nucleotide sequence encoding the second polypeptide. In some cases, the nucleotide sequence encoding the first polypeptide, and the second nucleotide sequence encoding the second polypeptide, are each operably linked to transcriptional control elements, e.g., promoters, such as promoters that are functional in a eukaryotic cell, where the promoter can be a constitutive promoter or an inducible promoter.

### Recombinant Expression Vectors

The present disclosure provides recombinant expression vectors comprising nucleic acids of the present disclosure. In some cases, the recombinant expression vector is a non-viral vector. In some cases, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors are known in the art and include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXTI, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control element, including constitutive and inducible promoters, transcription enhancer elements, transcription terminators, etc. may be used in the expression vector (see e.g., Bitter et al. (1987) Methods in Enzymology, 153:516-544).

In some cases, a nucleotide sequence encoding IL-2 variant polypeptide or fusion polypeptide is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some cases, a nucleotide sequence encoding an IL-2 variant polypeptide or fusion polypeptide is operably linked to multiple control elements that allow expression of the nucleotide sequence encoding an IL-2 variant polypeptide or fusion polypeptide in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-1. Selection of the appropriate vector and promoter is well within the level of ordinary skill in the art. The expression vector may also contain a ribosome binding site for translation initiation and a transcription terminator. The expression vector may also include appropriate sequences for amplifying expression.

### Genetically Modified Host Cells

The present disclosure provides a genetically modified host cell, where the host cell is genetically modified with one or more nucleic acids of the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize endogenous MHC 02-M.

In some cases, the host cell is a mammalian cell that has been genetically modified such that it does not synthesize endogenous MHC Class I heavy chain. In some cases, the host cell is a mammalian cell that has been genetically modified such that it does not synthesize endogenous MHC P2-M and such that it does not synthesize endogenous MHC Class I heavy chain.

The host cells are then cultured under conditions such that the host cells produce the desired IL-2 variant polypeptide or fusion polypeptide, after which the product is recovered and purified.

### Pharmaceutical Compositions

The present disclosure provides pharmaceutical compositions comprising an IL-2 variant polypeptide or a composition comprising one or more IL-2 variant polypeptide, e.g., fusion polypeptide. Such pharmaceutical compositions may comprise one or more pharmaceutically acceptable additives, a variety of which are known in the art and need not be discussed in detail herein. See, for example, the ninth (or latest) edition of Sheskey et al., “Handbook of Pharmaceutical Excipients” (2020), and/or the 23^{rd} (or latest) edition of “Remington: The Science and Practice of Pharmacy”, 23rd Ed. (2020).

In some cases, a subject pharmaceutical composition will be suitable for administration to a subject, e.g., will be sterile. For example, in some cases, a subject pharmaceutical composition will be suitable for administration to a human subject, e.g., where the composition is sterile and is substantially free of detectable pyrogens and/or other toxins, or where such detectable pyrogens and/or other toxins are present at a level within acceptable limits set by an applicable regulatory agency, e.g., the USF&DA.

For example, compositions may include aqueous solution, powder form, granules, tablets, pills, suppositories, capsules, suspensions, sprays, and the like. The composition may be formulated according to the various routes of administration described below.

Where a pharmaceutical composition is administered as an injectable (e.g. subcutaneously, intraperitoneally, intramuscularly, and/or intravenously) directly into a tissue, a formulation can be provided as a ready-to-use dosage form, or as non-aqueous form (e.g. a reconstitutable storage-stable powder) or aqueous form, such as liquid composed of pharmaceutically acceptable carriers and excipients. The protein-containing formulations may also be provided so as to enhance serum half-life of the IL-2 variant polypeptide or fusion protein administration. For example, the IL-2 variant polypeptide or fusion protein may be provided in a liposome formulation, prepared as a colloid, or other conventional techniques for extending serum half-life. The preparations may also be provided in controlled release or slow-release forms.

In some cases, the pharmaceutical composition is a liquid composition that comprises saline (e.g., 0.9% NaCl).

### Methods of Use

As noted above, it has been discovered that the IL-2 variants and compositions comprising one or more IL-2 variant polypeptides, e.g., fusion polypeptides, described herein do not systemically activate multiple immune cell subsets, as native IL-2 delivered in high doses would, but rather predominantly or preferentially activate only T cells whose T cell receptors (TCRs) are engaged with a peptide-MHC complex (pMHC) presented by an antigen presenting cell. Pharmaceutical compositions comprising such proteins thus can possess a useful therapeutic index, i.e., the blood concentration of drug that is required for efficacy is significantly less than the concentration at which the drug produces unacceptable adverse events or becomes toxic, such that the drug can be both efficacious and reasonably well tolerated by a patient. Further, it has been discovered that such IL-2 variants and compositions comprising one or more IL-2 variant polypeptides, e.g., fusion polypeptides, when engaged with IL-2 receptors on CAR-T cells and other cytotoxic cells (e.g., macrophages and NK cells) that have exogenous activation receptors such as CARs and TCRs, can provide homeostatic signals that can prolong the survival of such cells and/or provide activating signals to the cells that cause them to proliferate and retain their cytotoxic function. The IL-2 variants and compositions comprising one or more IL-2 variant polypeptide, e.g., fusion polypeptides, are thus suitable to be used in combination with certain medical treatments such as cancer vaccines, cell therapies and immune checkpoint inhibitors.

One such method of treatment involves administering a therapeutically effective amount of a pharmaceutical composition comprising an IL-2 variant polypeptide or a composition comprising one or more IL-2 variant polypeptides, e.g., a fusion polypeptide, to a patient who has received, is receiving, or will receive modified or unmodified T cells having a T cell receptor (TCR), e.g., where the TCR is specific for the patient's cancer. Such therapies include, e.g., (i) TCR-T cell therapy in which the patient's T cell are removed and modified to express a TCR specific for a cancer antigen expressed on a specific human leukocyte antigen (HLA), and (ii) TIL therapy in which tumor infiltrating lymphocytes (TILs) are removed from the patient, grown in large numbers in vitro, and then the TILs are administered to the patient. When co-administered (at the same or different times), the IL-2 variant polypeptide or composition comprising one or more IL-2 variant polypeptides, e.g., a fusion polypeptide, does not systemically activate multiple immune cell subsets, as native IL-2 delivered in high doses would, but rather can engage with IL-2 receptors on such modified or unmodified T cells to provide homeostatic signals that can prolong the survival of such cells and/or provide activating signals to the cells that cause them to proliferate and retain their cytotoxic function.

Another method of treatment involves administering a therapeutically effective amount of a pharmaceutical composition comprising an IL-2 variant polypeptide or composition comprising one or more IL-2 variant polypeptides, e.g., a fusion polypeptide, to a patient who has received, is receiving, or will receive a product (e.g., a vaccine such as a polypeptide cancer vaccine) that can engage with the TCR of a T cell or be processed by an immune system to be presented via an antigen presenting cell (APC) by a major histocompatibility complex (MHC) to the TCR of a T cell, or a precursor of a product that can engage with the TCR of a T cell or be processed by an immune system to be presented by an MHC to the TCR of a T cell, e.g., an mRNA cancer vaccine that produces a protein that is processed by the immune system to be presented via an antigen presenting cell APC by an MHC to the TCR of a T cell. Such cancer vaccines have been known to generate T cells in patients, but such T cells may not be activated to destroy cancer cells. When co-administered (at the same or different times) the IL-2 variant polypeptide or composition comprising one or more IL-2 variant polypeptides, e.g., a fusion polypeptide, substantially does not systemically activate multiple immune cell subsets, as native IL-2 delivered in high doses would, but rather predominantly or preferentially activates only those T cells whose TCRs are engaged with a pMHC presented by an APC. When co-administered with a cancer vaccine, therefore, IL-2 variant polypeptide or composition comprising one or more IL-2 variant polypeptides, e.g., a fusion polypeptide, can provide IL-2 activation signals to T cells that are engaged by with an APC that is presenting the antigen of the cancer vaccine. As a result, the T cells can become cytotoxic to cancer cells bearing a pMHC presenting the antigen of the cancer vaccine. Numerous types of vaccines are known for inducing a T cell response against an antigen (e.g., a cancer-associated antigen), which T cell response may be enhanced when the vaccine is used in combination with fusion polypeptides described herein. Examples of such vaccines include, but are not limited to, inactivated vaccines that employ a killed version of a pathogen that causes a disease, a live-attenuated vaccine that employs a weakened form of a pathogen that causes a disease, messenger RNA (mRNA) vaccines that make proteins in order to trigger an immune response, subunit, recombinant, polysaccharide, and conjugate vaccines that employ specific pieces of an antigen, such as its protein, sugar, or capsid (a casing around the germ), as well as toxoid vaccines and viral vector vaccines. As referred to herein, the term “cancer vaccine” refers to any vaccine that can elicit an immune response that produces T cells that then can attack cancer cells. Such cancer vaccines can either be a preventative vaccine that can reduce the likelihood that the individual will develop a cancer associated with the protein present in the cancer vaccine or reduce the likelihood that the individual will develop a cancer associated with the protein encoded by a nucleic acid present in the cancer vaccine), or a therapeutic vaccine that can induce T cells that can attack and kill cancer cells.

Another method of treatment involves administering a therapeutically effective amount of one or more IL-2 variant polypeptides or a composition comprising one or more IL-2 variant polypeptides, e.g., a fusion polypeptide, to a patient who has received, is receiving, or will receive modified cells (e.g., T cells, macrophages or NK cells) that comprise a chimeric antigen receptor (CAR), wherein the CAR binds to a target antigen and the modified cell comprises an intracellular signaling domain that is activated by interaction of the modified cell with IL-2. When co-administered (at the same or different times) the IL-2 variant polypeptide or composition comprising one or more IL-2 variant polypeptides, e.g., a fusion polypeptide, substantially does not systemically activate multiple immune cell subsets, as native IL-2 delivered in high doses would, but rather can engage with IL-2 receptors on such cells to provide homeostatic signals that can prolong the survival of such cells and/or provide activating signals to the cells that cause them to proliferate and retain their cytotoxic function.

Another method of treatment involves administering a therapeutically effective amount of one or more IL-2 variant polypeptides or composition comprising one or more IL-2 variant polypeptides, e.g., a fusion polypeptide, to a patient who has received, is receiving, or will receive cells (e.g., macrophages or NK cells) that have been modified to express one or more exogenous activation receptors (e.g., a CAR or TCR). When co-administered (at the same or different times) the IL-2 variant polypeptide or composition comprising one or more IL-2 variant polypeptides, e.g., a fusion polypeptide, substantially does not systemically activate multiple immune cell subsets, as native IL-2 delivered in high doses would, but rather can engage with and activate the modified cells and/or provide homeostatic signals that can prolong the survival of such cells and/or provide activating signals to the cells that cause them to proliferate and retain their cytotoxic function.

Another method of treatment involves administering a therapeutically effective amount of one or more IL-2 variant polypeptides or composition comprising one or more IL-2 variant polypeptides, e.g., a fusion polypeptide, to a cancer patient, either as a monotherapy, or in combination with another therapy such as an immune-checkpoint inhibitor (CPI). For example, where a CPI has demonstrated a therapeutic benefit in the treatment of a particular cancer (whether alone or in combination with another agent), such therapeutic benefit is likely due, at least in part, to the CPI's ability to enhance a patient's T cell response to that cancer. In such cases, administering a composition comprising one or more IL-2 variant polypeptides, e.g., a fusion polypeptide, may enhance the efficacy of the treatment by enhancing the activation and/or proliferation of the patient's cancer-specific T cells. Administering a composition comprising one or more IL-2 variant polypeptides, e.g., a fusion polypeptide, also may enhance the efficacy of the CPI treatment by enhancing the activation and/or proliferation of other immune cells such as NK cells.

As noted above, the IL-2 variant polypeptide(s) or composition comprising one or more IL-2 variant polypeptides, e.g., a fusion polypeptide, substantially do not systemically activate multiple immune cell subsets, as native IL-2 delivered in high doses would, but rather predominantly or preferentially activate only T cells whose TCRs are engaged with a peptide-MHC complex (pMHC) presented by an APC. If, for example, such T cells are specific to an antigen expressed on the surface of a cancer cell, then the IL-2 polypeptide(s) will activate these T cells for potential killing of the cancer cells. In this way, such polypeptides can provide immune stimulation in a manner that provides a therapeutic index that permits administration of an active amount of the IL-2 variant polypeptide without the resulting toxicity that is observed native IL-2 or other IL-2 variants.

Cancers that can be treated with a method of the present disclosure include carcinomas, sarcomas, melanoma, leukemias, and lymphomas. Cancers that can be treated with a method of the present disclosure include solid tumors. Cancers that can be treated with a method of the present disclosure include metastatic cancers.

Carcinomas that can treated by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be treated by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated by a method disclosed herein include, but are not limited to, glioma, astrocytoma, medulloblastoma, craniopharyngioma, ependymoma, pincaloma, hemangioblastoma, acoustic neuroma, oligodendroglioma, menangioma, melanoma, neuroblastoma, and retinoblastoma.

Leukemias that can be amenable to therapy by a method disclosed herein include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; non-Hodgkin's lymphoma, and the like.

Other cancers that can be treated according to the methods disclosed herein include atypical meningioma, islet cell carcinoma, medullary carcinoma of the thyroid, mesenchymoma, hepatocellular carcinoma, hepatoblastoma, clear cell carcinoma of the kidney, and neurofibroma mediastinum.

### Dosages

A suitable dosage can be determined by an attending physician or by other qualified medical personnel, based on various clinical factors. As is well known in the medical arts, dosages for any one patient depend upon many factors, including the patient's size, body surface area, age, the amount of IL-2 variant polypeptide to be administered, e.g., whether a fusion polypeptide comprises 1, 2, 4 or more variant IL-2 polypeptides, the sex of the patient, time, and route of administration, general health, and other drugs being administered concurrently. Depending on these factors, the IL-2 variant polypeptide or composition comprising one or more IL-2 variant polypeptides, e.g., a fusion polypeptide, may be administered in amounts between 0.1 mg/kg body weight and 20 mg/kg body weight per dose, e.g. between 0.1 mg/kg body weight and 0.5 mg/kg body weight, between 0.5 mg/kg body weight and 1 mg/kg body weight, between 1 mg/kg body weight and 5 mg/kg body weight, between 5 mg/kg body weight and 10 mg/kg body weight, between 10 mg/kg body weight and 15 mg/kg body weight, and between 15 mg/kg body weight and 20 mg/kg body weight.

Persons of ordinary skill in the art can readily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a pharmaceutical composition of the present disclosure is administered in maintenance doses within the above-mentioned ranges.

Those of skill will readily appreciate that dose levels can vary as a function of the specific IL-2 variant polypeptide or fusion protein, the severity of the symptoms and the susceptibility of the subject to side effects. Preferred dosages for a given compound are readily determinable by those of skill in the art by a variety of means.

In some cases, the administration of multiple doses of one or more IL-2 variant polypeptides or composition comprising one or more IL-2 variant polypeptides, e.g., a fusion polypeptide, can vary depending on any of a variety of factors, e.g., severity of the symptoms, etc. For example, in some cases, administration is once per month, approximately every three weeks, twice per month, three times per month, every other week (qow), once per week (qw), or more often than once per week.

The duration of administration times of one or more IL-2 variant polypeptides or composition comprising one or more IL-2 variant polypeptides, e.g., a fusion polypeptide, can vary, depending on any of a variety of factors, e.g., patient response, etc. For example, administration can be a one-time event, or can comprise multiple administrations that occur over a period of time ranging from less than one month, e.g., one day to one week, two weeks to four weeks, or longer than a month, e.g., from about one month to about two months, from about two months to about four months, from about four months to about six months, from about six months to about eight months, from about eight months to about 1 year, from about 1 year to about 2 years, or from about 2 years to about 4 years, or more.

### Routes of Administration

A pharmaceutical composition comprising an IL-2 variant polypeptide or composition comprising one or more IL-2 variant polypeptides, e.g., a fusion polypeptide, is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intralymphatic, intratracheal, intracranial, subcutaneous, intradermal, topical application, intravenous, intraarterial, rectal, nasal, oral, and other enteral and parenteral routes of administration. Routes of administration may be combined, if desired, or adjusted depending upon the IL-2 variant polypeptide or fusion protein and/or the desired effect.

In some cases, the pharmaceutical composition is administered intravenously, intramuscularly, or subcutaneously. In some cases, the pharmaceutical composition administered locally. In some cases, the pharmaceutical composition is administered intratumorally. In some cases, the pharmaceutical composition is administered peritumorally. In some cases, the pharmaceutical composition is administered intracranially, or intra-lymphatically.

### Combination Therapy

As noted above, pharmaceutical composition comprising an IL-2 variant polypeptide or composition comprising one or more IL-2 variant polypeptides, e.g., a fusion polypeptide, can be co-administered with modified or unmodified T cells (e.g., in TCR-T or TIL therapy); vaccines, including cancer vaccines; modified cells (e.g., T cells, macrophages or NK cells) that comprise a chimeric antigen receptor (CAR); or modified cells (e.g., macrophages and NK cells) comprising one or more exogenous activation receptors. Such pharmaceutical compositions also can be co-administered to an individual in need thereof in combination with one or more additional therapeutic agents or therapeutic treatments, including, e.g., immune checkpoint inhibitors.

By “co-administration” is meant that both an IL-2 variant polypeptide or fusion polypeptide of this disclosure and at least one additional therapeutic agent are administered to an individual, although not necessarily at the same time, in order to achieve a therapeutic effect that is the result of having administered both the pharmaceutical composition comprising an IL-2 variant polypeptide or composition comprising one or more IL-2 variant polypeptides, e.g., a fusion polypeptide, and the at least one additional therapeutic agent. The administration of the pharmaceutical composition and additional therapeutic agent can be substantially simultaneous, e.g., they can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the at least one additional therapeutic agent. In some cases, a pharmaceutical composition comprising IL-2 variant polypeptide or composition comprising one or more IL-2 variant polypeptides, e.g., a fusion polypeptide, is administered to an individual who will undergo treatment with, is undergoing treatment with, or who has undergone treatment with, the at least one additional therapeutic agent. The administration of the pharmaceutical composition comprising IL-2 variant polypeptide or composition comprising one or more IL-2 variant polypeptides, e.g., a fusion polypeptide, can occur at different times and/or at different frequencies.

As another example, a treatment method of the present disclosure can comprise co-administration of pharmaceutical composition comprising IL-2 variant polypeptide or composition comprising one or more IL-2 variant polypeptides, e.g., a fusion polypeptide, and an immune checkpoint inhibitor such as an antibody specific for an immune checkpoint. By “co-administration” is meant that both an pharmaceutical composition and an antibody specific for an immune checkpoint are administered to an individual, although not necessarily at the same time, in order to achieve a therapeutic effect that is the result of having administered both the pharmaceutical composition comprising IL-2 variant polypeptide or composition comprising one or more IL-2 variant polypeptides, e.g., a fusion polypeptide, and the immune checkpoint inhibitor. The administration of the pharmaceutical composition comprising IL-2 variant polypeptide or composition comprising one or more IL-2 variant polypeptides, e.g., a fusion polypeptide, and the antibody specific for an immune checkpoint can be substantially simultaneous, e.g., within about 1 minute to about 24 hours (e.g., within about 1 minute, within about 5 minutes, within about 15 minutes, within about 30 minutes, within about 1 hour, within about 2 hours, within about 4 hours, within about 8 hours, within about 12 hours, or within about 24 hours) of administration of the antibody specific for an immune checkpoint. In some cases, a pharmaceutical composition comprising IL-2 variant polypeptide or composition comprising one or more IL-2 variant polypeptides, e.g., a fusion polypeptide, is administered to an individual who is undergoing treatment with, or who has undergone treatment with, an antibody specific for an immune checkpoint. The administration of the pharmaceutical composition comprising IL-2 variant polypeptide or composition comprising one or more IL-2 variant polypeptides, e.g., a fusion polypeptide, and the antibody specific for an immune checkpoint can occur at different times and/or at different frequencies.

Exemplary immune checkpoint inhibitors include inhibitors that target immune checkpoint polypeptides such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122, and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT, and VISTA.

In some cases, the immune checkpoint inhibitor is an antibody specific for an immune checkpoint. Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (MedImmune/AZ), CP-870,893 (Genentech), Mogamulizumab (Kyowa Hakko Kirin), Varlilumab (CelIDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035; and combinations thereof. For example, in some cases, the immune checkpoint inhibitor is an anti-PD-1 antibody. Suitable anti-PD-1 antibodies include, e.g., nivolumab, cemiplimab, pembrolizumab (also known as MK-3475), pidilizumab, SHR-1210, PDR001, and AMP-224. In some cases, the anti-PD-1 monoclonal antibody is nivolumab, cemiplimab, pembrolizumab, or PDR001. Suitable anti-PD1 antibodies are described in U.S. Patent Publication No. 2017/0044259. For pidilizumab, see, e.g., Rosenblatt et al. (2011) J. Immunother. 34:409-18. In some cases, the immune checkpoint inhibitor is an anti-CTLA-4 antibody. In some cases, the anti-CTLA-4 antibody is ipilimumab or tremelimumab. For tremelimumab, see, e.g., Ribas et al. (2013) J. Clin. Oncol. 31:616-22. In some cases, the immune checkpoint inhibitor is an anti-PD-L1 antibody. In some cases, the anti-PD-L1 monoclonal antibody is BMS-935559, MED14736, MPDL3280A (also known as RG7446), KN035, or MSB0010718C. In some embodiments, the anti-PD-L1 monoclonal antibody is MPDL3280A (atezolizumab) or MED14736 (durvalumab). For durvalumab, see, e.g., WO 2011/066389. For atezolizumab, see, e.g., U.S. Pat. No. 8,217,149. In some cases, the anti-TIGIT antibody is Tiragolumab (RG6058; MTIG7192A) (see US 2018/0186875. In some cases, the anti-TIGIT antibody is Vibostolimab (MK-7684) (see US 2018/0066055). In some cases, the anti-TIGIT antibody is Etigilimab (OMP-313M32).

### Subjects Suitable for Treatment

Subjects suitable for treatment with a method of the present disclosure include individuals who may develop cancer (either for the first time or a recurring cancer), have cancer, including individuals who have been diagnosed as having cancer, individuals who have been treated for cancer but who failed to respond to the treatment, and individuals who have been treated for cancer and who initially responded but subsequently became refractory to the treatment.

### Examples of Non-Limiting Aspects of the Disclosure

### Aspects Set A

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method comprising administering to an individual
(i) a first composition comprising:
   (a) modified or unmodified T cells having a T cell receptor (TCR), a product that can engage with the TCR of a T cell or be processed by an immune system to be presented by a major histocompatibility complex (MHC) to the TCR of a T cell, or a precursor of a product that can engage with the TCR of a T cell or be processed by an immune system to be presented by an MHC to the TCR of a T cell, or
   (b) modified cells that comprise a chimeric antigen receptor (CAR), wherein the CAR binds to a target antigen and the modified cell comprises an intracellular signaling domain that is activated by interaction of the modified cell with IL-2, or
   (c) modified cells comprising one or more exogenous activation receptors; and
(ii) a second composition comprising an immunomodulatory protein,
   wherein the immunomodulatory protein comprises one or more variant IL-2 polypeptides that have at least two amino acid substitutions relative to set forth in SEQ ID NO:1,
   wherein the one or more variant IL-2 polypeptides bind to an IL-2R alpha chain (IL-2Rα), and wherein the binding affinity to IL-2Rα is less than the affinity of a wild-type IL-2 polypeptide for IL-2Rα when assayed under the same conditions, wherein IL-2Rα has the amino acid sequence set forth in SEQ ID NO:2, and
   wherein the one or more variant IL-2 polypeptides bind to an IL-2R beta chain (IL-2Rβ), and wherein the binding affinity to IL-2Rβ is less than the affinity of a wild-type IL-2 polypeptide for IL-2Rβ when assayed under the same conditions, wherein the IL-2Rβ has the amino acid sequence set forth in SEQ ID NO:3, wherein the second composition preferentially activates T cells whose TCRs are engaged with an antigen presented by an MHC, as compared to T cells whose TCRs are not engaged with an antigen presented by an MHC, and wherein the first and second compositions are administered at the same time or at different times.

2. A method according to Aspect 1, wherein the at least one variant IL-2 polypeptide exhibits at least a two-fold decrease in binding affinity to IL-2R9 compared to the binding affinity of a wild-type IL-2 polypeptide for IL-2Rβ, and exhibits at least a fifty-fold decrease in binding affinity to IL-2Rα compared to the binding affinity of a wild-type IL-2 polypeptide for IL-2Rα.

3. A method according to Aspect 1 or 2, wherein the one or more variant IL-2 polypeptides comprise at least one substitution that decreases the affinity of the variant IL-2 polypeptide for IL-2Rα, optionally wherein the at least one substitution is selected from a substitution at R38, F42, K43, Y45, E62, P65, E68, V69, L72, and combinations thereof.

4. A method according to any one of Aspects 1-3, wherein the at least one variant IL-2 polypeptide comprises a substitution of the phenylalanine amino acid F42, optionally wherein the phenylalanine is substituted with Ala, Gly, Val, Ile, or Len.

5. A method according to any of Aspects 1-4, wherein the one or more variant IL-2 polypeptides comprise at least one substitution that decreases the affinity of the variant IL-2 polypeptide for IL-2Rβ, optionally wherein the at least one substitution is selected from a substitution at E15, H16, L19, D20, D84, 587, N88, V91, I92, and combinations thereof.

6. A method according to any one of Aspects 1-5, wherein the at least one variant IL-2 polypeptide comprises a substitution of the histidine amino acid H16, optionally wherein the histidine is substituted with Ala, Gly, Val, Len, Thr, Ile, Asp, or Glu.

7. A method according to any one of Aspects 1-6, wherein the at least one variant IL-2 polypeptide comprises a substitution of the asparagine amino acid N88, optionally wherein the asparagine is substituted with Gly, Ala, Ser, Thr, Arg or Asp.

8. A method according to any one of Aspects 1-7, wherein the at least one variant IL-2 polypeptide comprises substitutions of amino acids F42 and H16, optionally wherein the phenylalanine is substituted with Ala, and wherein the histidine is substituted with Ala, Thr, Asp, or Glu.

9. A method according to any one of Aspects 1-8, wherein the at least one variant IL-2 polypeptide comprises: (i) an H16A substitution and an F42A substitution; (ii) an H16T substitution and an F42A substitution, (iii) an H16E substitution and an F42A substitution, and (iv) an H16D substitution and an F42A substitution.

10. A method according to any one of Aspects 1-9, wherein the at least one variant IL-2 polypeptide comprises substitutions at F42, H16, and N88.

11. A method according to Aspect 10, wherein the asparagine amino acid N88 is substituted with Gly, Ala, Ser, Thr, Arg or Asp.

12. A method according to any one of Aspects 1-11, wherein the immunomodulatory protein comprises two or more variant IL-2 polypeptides, wherein each variant polypeptide comprises the same amino acid sequence.

13. A method according to Aspect 12, wherein the immunomodulatory protein comprises two variant IL-2 polypeptides that are in tandem and joined by an independently selected linker, optionally wherein the linker comprises glycine and serine.

14. A method according to any of Aspects 1-13, wherein the immunomodulatory protein further comprises a carrier.

15. A method according to Aspect 14, wherein the carrier is a lipid vesicle (e.g., a liposome) or micelle, a nanoparticle, a PEGylated protein, a fibronectin-based scaffold protein, or an artificial antigen presenting cell such as engineered erythroid cell or enucleated cell (e.g., a platelet).

16. A method according to any of Aspects 1-13, wherein the immunomodulatory protein further comprises an immunoglobulin (Ig) scaffold polypeptide or a non-Ig scaffold polypeptide.

17. A method according to Aspect 16, wherein the immunomodulatory protein comprises a non-Ig scaffold chosen from an XTEN polypeptide, a transferrin polypeptide, an elastin-like polypeptide, a silk-like polypeptide, or a silk-elastin-like polypeptide.

18. A method according to Aspect 16, wherein the wherein immunomodulatory protein comprises an Fe polypeptide, and wherein the Ig Fc polypeptide is an IgG1 Fe polypeptide, an IgG2 Fc polypeptide, an IgG3 Fc polypeptide, an IgG4 Fc polypeptide, an IgA Fc polypeptide, or an IgM Fc polypeptide.

19. A method according to Aspect 18, wherein the Ig Fc polypeptide is a variant that has a substantially reduced effector function, e.g., a substantially reduced ability to effect complement-dependent cytotoxicity (CDC) and/or antibody-dependent cell cytotoxicity (ADCC).

20. A method according to Aspect 18 or 19, wherein the Ig Fc polypeptide comprises one or more amino acid substitutions chosen from N297A, L234A, L235A, L234F, L235E, G237A and P331S, wherein N297, L234, L235, G237 and P331 correspond to N77, L14, L15, G17 and P111, respectively, of the amino acid sequences depicted in FIG. 9A, optionally wherein the Ig Fc polypeptide comprises amino acid substitutions L234A and L235A.

21. A method according to Aspect 18-20, wherein the Ig Fc polypeptide is an IgG1 Fc polypeptide that comprises an amino acid sequence having at least 95% amino acid sequence identity to the amino acid sequence depicted in any one of FIGS. 9A-9M.

22. A method according to any one of Aspects 18-21, wherein the immunomodulatory protein comprises a homodimer of two immunomodulatory proteins, each of which comprises an Ig Fc polypeptide, and wherein the Ig Fc of one immunomodulatory protein is joined by one or more disulfide bonds to the Ig Fc of the other immunomodulatory protein.

23. A method according to Aspect 22, wherein each of the immunomodulatory proteins in the homodimer comprises two variant IL-2 polypeptides in tandem, and wherein the variant IL-2 polypeptides are joined by an independently selected linker.

24. A method according to Aspect 22, wherein each of the immunomodulatory proteins in the homodimer comprises, from N-terminus to C-terminus:
(i) a variant IL-2 polypeptide;
(ii) an independently selected linker;
(iii) a variant IL-2 polypeptide;
(iv) an independently selected linker; and
(v) an Ig Fc polypeptide.

25. A method according to Aspect 22, wherein each of the immunomodulatory proteins in the homodimer comprises, from N-terminus to C-terminus:
(i) a variant IL-2 polypeptide;
(ii) an independently selected linker;
(iii) a variant IL-2 polypeptide;
(iv) an independently selected linker;
(v) an Ig Fc polypeptide;
(vi) an independently selected linker;
(vii) a variant IL-2 polypeptide;
(viii) an independently selected linker; and
(ix) a variant IL-2 polypeptide.

26. A method according to Aspect 22, wherein each of the immunomodulatory proteins in the homodimer comprises, from N-terminus to C-terminus:
(i) a variant IL-2 polypeptide;
(ii) an independently selected linker;
(iii) an Ig Fc polypeptide;
(iv) an independently selected linker; and
(v) a variant IL-2 polypeptide;
27. A method according to Aspect 22, wherein each of the immunomodulatory proteins in the homodimer comprises, from N-terminus to C-terminus:
(i) an Ig Fc polypeptide;
(ii) an independently selected linker;
(iii) a variant IL-2 polypeptide;
(iv) an independently selected linker; and
(v) a variant IL-2 polypeptide.

28. A method according to any one of Aspects 18-21, wherein the immunomodulatory protein comprises a heterodimer of two immunomodulatory proteins, wherein one of the immunomodulatory proteins comprises an Ig Fc polypeptide comprising an interspecific dimerization sequence and the other immunomodulatory protein comprises an Ig Fe polypeptide comprising a counterpart interspecific sequence.

29. A method according to Aspect 28, wherein one of the immunomodulatory proteins in the heterodimer comprises, from N-terminus to C-terminus:
(i) a variant IL-2 polypeptide;
(ii) an independently selected linker;
(iii) a variant IL-2 polypeptide;
(iv) an independently selected linker; and
(v) an Ig Fc polypeptide comprising an interspecific binding sequence, and wherein the other immunomodulatory protein in the heterodimer comprises, from N-terminus to C-terminus:
(i) an Ig Fc polypeptide comprising a counterpart interspecific binding sequence;
(ii) an independently selected linker;
(iii) a variant IL-2 polypeptide;
(iv) an independently selected linker; and
(v) a variant IL-2 polypeptide.

30. A method according to Aspect 28, wherein one of the immunomodulatory proteins in the heterodimer comprises, from N-terminus to C-terminus:
(i) a variant IL-2 polypeptide;
(ii) an independently selected linker; and
(iii) an Ig Fc polypeptide comprising an interspecific binding sequence, and wherein the other immunomodulatory protein in the heterodimer comprises, from N-terminus to C-terminus:
(i) an Ig Fc polypeptide comprising a counterpart interspecific binding sequence;
(ii) an independently selected linker; and
(iii) a variant IL-2 polypeptide.

31. A method according to any one of Aspects 1-30, wherein the first composition comprises one or more products that can engage with the TCR of a T cell or be processed by an immune system into one or more antigens that can be presented by a major histocompatibility complex (MHC) to the TCR of a T cell, optionally wherein the one or more antigens are cancer-associated antigens.

32. A method according to any one of Aspects 1-30, wherein the first composition comprises one or more nucleic acids encoding one or more polypeptides that can engage with the TCR of a T cell or can be processed by an immune system into one or more antigens that can be presented by a major histocompatibility complex (MHC) to the TCR of a T cell, optionally wherein the one or more antigens are cancer-associated antigens.

33. A method according to Aspect 31 or 32, wherein the first composition comprises an anti-cancer vaccine.

34. The method of any one of Aspects 1-30, wherein the first composition comprises a TCR-T cell that comprises an exogenous TCR, optionally wherein the TCR binds a cancer-associated antigen when the cancer-associated antigen is presented by an MHC to the TCR.

35. A method according to any one of Aspects 1-30, wherein the first composition comprises tumor infiltrating lymphocytes (TILS), optionally wherein the TILS have been modified to reduce the susceptibility of the TILS to T-cell suppressive signals.

36. A method according to any one of Aspects 31-35, wherein the cancer-associated antigen is an antigen chosen from alpha-feto protein, Wilms-tumor-1 (WT-1), a mutant KRAS, e.g., comprising a G12C or G12D mutation, melanoma antigen recognized by T cells 1 (MART-I), melanoma-associated antigen (MAGE), MAGE-A1, MAGE-A3, MAGE-A4, MAGEA4/A8, human papillomavirus (HPV) antigen E6, HPV antigen E7, New York esophageal squamous cell carcinoma 1 (NY-ESO-1), MUC-1 (mucin-1, cell surface associated), mesothelin, survivin, prostate stem cell antigen (PSCA), prostate-specific membrane antigen (PSMA), prostate-specific antigen (PSA), a mutant p53 polypeptide, a Ras polypeptide, nuclear factor erythroid 2-related factor 2 (NFE2L2), beta-catenin, PIK3CA (phosphatidylinositol-4,5-bisphosphate 3-kinase catalytic subunit alpha), and BRAF.

37. A method according to any one of Aspects 1-30, wherein the first composition comprises a CAR having an antigen-binding domain that is specific for a cancer-associated antigen, optionally wherein the cell is a T cell, a macrophage, or an NK cell, optionally wherein
(i) when the CAR is not bound to a cancer-associated antigen, the second composition provides homeostatic signals to the cell for survival, and/or
(ii) when the CAR is not bound to a cancer-associated antigen, the second composition provides activating signals to the cell that cause the cell to proliferate and retain its cytotoxic function.

38. The method of Aspect 37, wherein the antigen-binding domain is a single-chain Fv polypeptide or a nanobody.

39. The method of Aspect 37 or 38, wherein the cancer-associated antigen is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, glypican-3, GPC3, HER-2, kappa immunoglobulin, LcY, LMP1, mesothelin, MG7, MUC1, NKG2D ligand, PD-L1, PSCA, PSMA, ROR1, ROR1R, TACI, and VEGFR2, e.g., BCMA and CD19, optionally wherein the first composition comprises TECARTUS®, KYMRIAH®, ABECMA®, BREYANZI® or YESCARTA®.

40. The method of any one of Aspects 1-39, wherein the method is for the treatment of a cancer in the individual.

41. The method of Aspect 40, further comprising administering at least one immune checkpoint inhibitor (CPI) to the individual, wherein the CPI, first composition, and second composition are administered at the same time or at different times.

42. The method of Aspect 41, wherein the at least one immune checkpoint inhibitor comprises an antibody specific for the immune checkpoint inhibitor.

43. The method of Aspect 42, wherein the antibody is specific for an immune checkpoint inhibitor chosen from CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2, optionally wherein the immune checkpoint inhibitor is an antibody specific for PD-1, PD-L1, CTLA-4, TIGIT or LAG3.

44. The method of any one of Aspects 1-36, wherein the method is for the prevention of a cancer in the individual.

45. The method of any one of Aspects 1-36, wherein the method is for the treatment of a cancer in the individual.

46. A method according to any one of Aspects 1-30, wherein the individual is administered a CAR-T therapy product, a TCR-T therapy product, or a CAR-NK therapy product.

47. The method of Aspect 46, wherein the individual is administered a CAR-T therapy product, wherein the CAR-T cell therapy product comprises a population of modified autologous T cells comprising a CAR or allogeneic T cells comprising a CAR, wherein the CAR comprises an antigen-binding domain specific for a cancer-associated antigen.

48. The method of Aspect 47, wherein the antigen-binding domain is a single-chain Fv polypeptide or a nanobody.

49. The method of Aspect 47 or 48, wherein the cancer-associated antigen is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2.

50. The method of any one of Aspects 1-49, further comprising administering to the individual an immune checkpoint inhibitor.

51. The method of Aspect 50, wherein the immune checkpoint inhibitor is an antibody specific for an immune checkpoint inhibitor selected from CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-113, B7-14, BTLA, CTLA-4, LAG3, TIM3, VISTA, CD96, TIGIT, CD122, PD-1, PD-L1 and PD-L2.

52. A method according to any one of Aspects 1-30, wherein the first composition is a vaccine comprising one or more products that can engage with the TCR of a T cell or be processed by an immune system into one or more antigens that can be presented by a major histocompatibility complex (MHC) to the TCR of a T cell.

53. A method according to any one of Aspects 1-30, wherein the first composition is a vaccine comprising a nucleic acid comprising a nucleotide sequence encoding a polypeptide that is capable of being processed by an immune system into one or more antigens that can be presented by a major histocompatibility complex (MHC) to the TCR of a T cell.

54. A method according to Aspect 52 or 53, wherein the one or more antigens are cancer-associated antigens.

55. The method of Aspect 52, wherein the vaccine comprises one or more products that can engage with the TCR of a T cell or be processed by an immune system into more than one cancer-associated antigens that can be presented by a major histocompatibility complex (MHC) to the TCRs of T cells.

56. The method of Aspect 52, wherein the vaccine comprises one or more nucleic acids comprising one or more nucleotide sequences that encode a plurality of polypeptides, wherein the polypeptides can engage with the TCR of a T cell or be processed by an immune system into more than one cancer-associated antigens that can be presented by a major histocompatibility complex (MHC) to the TCRs of T cells.

57. A fusion polypeptide as shown in any one of FIGS. 2A-C, 3A-C, 4A-C, 5A-B, 6A-B, 7A-B, 8A-F, 10A-C, 11A-C, 12A-B, 13A-B and 14A-N, wherein the immunomodulatory protein comprises one or more variant IL-2 polypeptides that have at least two amino acid substitutions relative to set forth in SEQ ID NO:1, wherein the one or more variant IL-2 polypeptides bind to an IL-2R alpha chain (IL-2Rα), and
wherein the binding affinity to IL-2Rα is less than the affinity of a wild-type IL-2 polypeptide for IL-2Rα when assayed under the same conditions, wherein IL-2Rα has the amino acid sequence set forth in SEQ ID NO:2, and wherein the one or more variant IL-2 polypeptides bind to an IL-2R beta chain (IL-2Rβ), and
wherein the binding affinity to IL-2Rβ is less than the affinity of a wild-type IL-2 polypeptide for IL-2Rβ when assayed under the same conditions, wherein the IL-2Rβ has the amino acid sequence set forth in SEQ ID NO:3.

58. A fusion polypeptide according to Aspect 57, wherein the fusion protein comprises one or more mutations that can reduce binding of IL-2 to IL-2Rα, wherein the one or more mutations are chosen from substitutions at one or more of amino acids R38, F42, K43, Y45, E62, P65, E68, V69, and L72.

59. A fusion polypeptide according to Aspect 58, wherein the fusion protein comprises a substitution of amino acid F42, optionally wherein the Phe is substituted with Ala or Lys.

60. A fusion polypeptide according to any one of Aspects 57-59, wherein the fusion protein comprises one or more mutations that can reduce binding of IL-2 to IL-2Rβ, wherein the one or more mutations are chosen from substitutions at one or more of amino acids E15, H16, L19, D20, D84, S87, N88, V91, I92.

61. A fusion polypeptide according to Aspect 60, wherein the fusion protein comprises a substitution of amino acid H16, optionally wherein the His is substituted with Ala, Glu, Thr, or Asp.

62. A fusion polypeptide according to Aspect 57, wherein the fusion protein comprises substitutions chosen from E15A with R38A, R38D or R38E.

63. A fusion polypeptide according to Aspect 57, wherein the fusion protein comprises substitutions chosen from: H16A with R38A, R38D or R38E; H16T with R38A, R38D or R38E; H16E with R38A, R38D or R38E; and H16D with R38A, R38D or R38E.

64. A fusion polypeptide according to Aspect 57, wherein the fusion protein comprises substitutions chosen from: D84H with R38A, R38D or R38E; D84K with R38A, R38D or R38E; and D84R with R38A, R38D or R38E.

65. A fusion polypeptide according to Aspect 57, wherein the fusion protein comprises substitutions chosen from: R38A with N88S, N88A, N88G, N88R, N88T, or N88D; R38D with N88S, N88A, N88G, N88R, N88T, or N88D; and R38E with N88S, N88A, N88G, N88R, N88T, or N88D.

66. A fusion polypeptide according to Aspect 57, wherein the fusion protein comprises substitutions chosen from: R38A with V91E, V91A or V91T, R38D with V91E, V91A or V91T; and R38E with V91E, V91A or V91T.

67. A fusion polypeptide according to Aspect 57, wherein the fusion protein comprises substitutions chosen from R38A, 192A, R38D, 192A and R38E, 192A.

68. A fusion polypeptide according to Aspect 57, wherein the fusion protein comprises substitutions chosen from E15A, F42A and E15A, F42K.

69. A fusion polypeptide according to Aspect 57, wherein the fusion protein comprises substitutions chosen from: H16A, F42A; H16T, F42A; H16E, F42A; H16D, F42A; H16A, F42K; H16T, F42K; and H16E, F42K; H16D, F42K.

70. A fusion polypeptide according to Aspect 57, wherein the fusion protein comprises substitutions chosen from: F42A with N88S, N88A, N88G, N88R, N88T, or N88D; and F42K with N88S, N88A, N88G, N88R, N88T, or N88D.

71. A fusion polypeptide according to Aspect 57, wherein the fusion protein comprises substitutions chosen from: F42A with V91E, V91A, or V91T; and F42K with V91E, V91A, or V91T

72. A fusion polypeptide according to Aspect 57, wherein the fusion protein comprises substitutions chosen from: F42A with I92A; and F42K with 192A.

73. A fusion polypeptide according to Aspect 57, wherein the fusion protein comprises substitutions of E15A and K43E.

74. A fusion polypeptide according to Aspect 57, wherein the fusion protein comprises substitutions chosen from: H16A, K43E; H16T, K43E; H16E, K43E; and H16D, K43E.

75. A fusion polypeptide according to Aspect 57, wherein the fusion protein comprises substitutions chosen from K43E with D84H, D84K or D84R.

76. A fusion polypeptide according to Aspect 57, wherein the fusion protein comprises substitutions chosen from K43E with N88S, N88A, N88G, N88R, N88T, or N88D.

77. A fusion polypeptide according to Aspect 57, wherein the fusion protein comprises substitutions chosen from K43E with V91E, V91A, or V91T.

78. A fusion polypeptide according to Aspect 57, wherein the fusion protein comprises substitutions of K43E and 192A or E15A, E62Q.

79. A fusion polypeptide according to Aspect 57, wherein the fusion protein comprises substitutions chosen from: H16A, E62Q; H16T, E62Q; H16E, E62Q; and H16D, E62Q.

80. A fusion polypeptide according to Aspect 57, wherein the fusion protein comprises substitutions chosen from E62Q with D84H, D84K or D84R.

81. A fusion polypeptide according to Aspect 57, wherein the fusion protein comprises substitutions chosen from E62Q with N88S, N88A, N88G, N88R, N88T, or N88D.

82. A fusion polypeptide according to Aspect 57, wherein the fusion protein comprises substitutions chosen from E62Q with V91E, V91A, or V91T.

83. A fusion polypeptide according to Aspect 57, wherein the fusion protein comprises substitutions chosen from E62Q and 192A.

84. A fusion polypeptide according to Aspect 57, wherein the fusion protein comprises substitutions chosen from E62Q with V91E, V91A, or V91T.

85. A fusion polypeptide according to Aspect 68, wherein the fusion protein comprises substitutions at F42, E15 and N88.

86. A fusion polypeptide according to Aspect 68, wherein the fusion protein comprises substitutions at F42, E15 and V91.

87. A fusion polypeptide according to Aspect 69, wherein the fusion protein comprises substitutions at F42, H16 and D84.

88. A fusion polypeptide according to Aspect 69, wherein the fusion protein comprises substitutions at F42, H16 and N88.

89. A fusion polypeptide according to Aspect 69, wherein the fusion protein comprises substitutions at F42, H16 and V91.

90. A fusion polypeptide according to Aspect 69, wherein the fusion protein comprises substitutions at F42, H16 and 192.

91. A fusion polypeptide according to any one of Aspects 57-90, wherein the fusion protein is as shown in any one of FIGS. 10A-C, FIGS. 11A-C, FIGS. 12A-B and FIGS. 13A-B, and FIGS. 14A-N, and wherein the fusion polypeptide comprises a functional protein that is a cancer-targeting polypeptide (CTP).

92. A fusion polypeptide according to Aspect 91, wherein the target of the cancer-targeting polypeptide is a peptide-HLA complex on the surface of a cancer cell.

93. A fusion polypeptide according to Aspect 91, wherein the target of the CTP is a cancer-associated epitope.

94. A fusion polypeptide according to Aspect 91, wherein the CTP is an antibody that is specific for a cancer-associated antigen.

95. A fusion polypeptide according to Aspect 91, wherein the CTP is an antibody that is specific for a peptide/HLA complex on the surface of a cancer cell, wherein the peptide can be a cancer-associated peptide (e.g., a peptide of a cancer-associated antigen).

96. A fusion polypeptide according to Aspect 91, wherein the functional protein is a TCR such as single-chain T cell receptor of “scTCR” that is specific for a peptide/HLA complex on the surface of a cancer cell, wherein the peptide can be a cancer-associated peptide (e.g., a peptide of a cancer-associated antigen).

97. A fusion polypeptide according to Aspect 91, wherein the functional protein comprises a wild-type or variant immunomodulatory polypeptide, e.g., a wild type or variant immunostimulatory polypeptide such as B7 family of costimulatory receptors, e.g., CD80, CD86, a cytokine such as IL-7, IL-12, IL-15 or IL-21, a TNF superfamily member such as CD-40, 4-1BBL and OX40, or a chemokine such as CCL19, CCL21, CXCL9/10/11, or CXCL12.

98. A fusion polypeptide according to any one of Aspects 57-97, wherein the fusion polypeptide comprises one or more independently selected linkers.

99. A fusion polypeptide according to any one of Aspects 57-97, wherein the fusion polypeptide comprises a variant Ig Fe polypeptide that has a substantially reduced effector function, e.g. a substantially reduced ability to effect complement-dependent cytotoxicity (CDC) and/or antibody-dependent cell cytotoxicity (ADCC).

100. A fusion polypeptide according to Aspect 99, wherein the Ig Fc polypeptide comprises one or more amino acid substitutions selected from N297A, L234A, L235A, L234F, L235E, G237A and P331S, wherein N297, L234, L235, G237 and P331 correspond to N77, L14, L15, G17 and P111, respectively, of the amino acid sequences depicted in FIG. 9A.

101. A fusion polypeptide according to Aspect 99 or 100, wherein the Ig Fc polypeptide is an IgG1 Fe polypeptide that comprises an amino acid sequence having at least 95% amino acid sequence identity to the amino acid sequence depicted in any one of FIGS. 9A-9M.

102. A fusion polypeptide according to any one of Aspects 57-101, wherein the fusion polypeptide comprises a homodimer of two fusion polypeptides, each of which comprises an Ig Fc polypeptide, and wherein the Ig Fe of one immunomodulatory protein is joined by one or more disulfide bonds to the Ig Fe of the other immunomodulatory protein.

103. A fusion polypeptide according to Aspect 102, wherein each of the immunomodulatory proteins in the homodimer comprises two variant IL-2 polypeptides in tandem, and wherein the variant IL-2 polypeptides are joined by an independently selected linker.

104. A method of treating cancer comprising administering to a patient a pharmaceutical composition comprising an effective amount of a fusion polypeptide according to any one of Aspects 57-103, wherein the pharmaceutical composition is co-administered with a second pharmaceutical composition that is a cancer vaccine.

105. A method of treating cancer comprising administering to a patient a pharmaceutical composition comprising an effective amount of a fusion polypeptide according to any one of Aspects 57-103, wherein the pharmaceutical composition is co-administered with a second pharmaceutical composition that is a TCR-T cell therapy.

106. A method of treating cancer comprising administering to a patient a pharmaceutical composition comprising an effective amount of a fusion polypeptide according to any one of Aspects 57-103, wherein the pharmaceutical composition is co-administered with a second pharmaceutical composition that is a CAR-T cell therapy.

107. A method of treating cancer comprising administering to a patient a pharmaceutical composition comprising an effective amount of a fusion polypeptide according to any one of Aspects 57-103, wherein the pharmaceutical composition is co-administered with a second pharmaceutical composition that is a modified cell (e.g., a macrophage or NK cell) comprising one or more exogenous activation receptors (e.g., a CAR or a TCR).

108. A method of treating cancer comprising administering to a patient a pharmaceutical composition comprising an effective amount of a fusion polypeptide according to any one of Aspects 57-103, wherein the pharmaceutical composition is co-administered with a second pharmaceutical composition that is an immune checkpoint inhibitor (e.g., an anti-PD1 antibody).

109. A method of treating cancer according to any of Aspects 104-108, wherein the fusion polypeptide is administered before, at the same time, or after administration of the second pharmaceutical composition.

110. Use of fusion polypeptide according to any of Aspects 57-103 to prepare a medicament for treating cancer to be administered before, at the same time, or after administration of a cancer vaccine.

111. Use of fusion polypeptide according to any of Aspects 57-103 to prepare a medicament for treating cancer to be administered before, at the same time, or after administration of a pharmaceutical composition that is a TCR-T cell therapy.

112. Use of fusion polypeptide according to any of Aspects 57-103 to prepare a medicament for treating cancer to be administered before, at the same time, or after administration of a pharmaceutical composition that is a CAR-T cell therapy.

113. Use of fusion polypeptide according to any of Aspects 57-103 to prepare a medicament for treating cancer to be administered before, at the same time, or after administration of a pharmaceutical composition that is a modified cell other than a T cell (e.g., a macrophage or NK cell) comprising one or more exogenous activation receptors (e.g., a CAR or a TCR).

114. Use of fusion polypeptide according to any of Aspects 57-103 to prepare a medicament for treating cancer to be administered before, at the same time, or after administration of a pharmaceutical composition that is an immune checkpoint inhibitor (e.g., an anti-PD1 antibody).

115. A fusion polypeptide according to any one of Aspects 94-103, wherein the CTP is an antibody specific for a cancer-associated antigen selected from the group consisting of epidermal growth factor receptor (EGFR), Her2, CD19, mesothelin, prostate-specific membrane antigen (PSMA), CD22, TROP-2, B-cell maturation antigen (BCMA), mucin-1 (MUC1), claudin 18.2, and CD20.

116. A fusion polypeptide according to any one of Aspects 94-103 and 115, wherein the fusion polypeptide comprises a first CTP and a second CTP, wherein the first CTP is a first antibody that is specific for a first cancer-associated antigen, and wherein the second CTP is an antibody that specific for a second cancer-associated antigen.

117. A fusion polypeptide according to Aspect 116, wherein the first antibody is specific for CD19 and wherein the second antibody is specific for CD20.

118. A fusion polypeptide according to Aspect 116, wherein the first antibody is specific for Her2 and wherein the second antibody is specific for EGFR.

119. A method of treating cancer comprising administering to a patient a pharmaceutical composition comprising an effective amount of a fusion polypeptide according to any one of Aspects 115-118, wherein the pharmaceutical composition is co-administered with a second pharmaceutical composition that is a cancer vaccine.

120. A method of treating cancer comprising administering to a patient a pharmaceutical composition comprising an effective amount of a fusion polypeptide according to any one of Aspects 115-118, wherein the pharmaceutical composition is co-administered with a second pharmaceutical composition that is a TCR-T cell therapy.

121. A method of treating cancer comprising administering to a patient a pharmaceutical composition comprising an effective amount of a fusion polypeptide according to any one of Aspects 115-118, wherein the pharmaceutical composition is co-administered with a second pharmaceutical composition that is a CAR-T cell therapy.

122. A method of treating cancer comprising administering to a patient a pharmaceutical composition comprising an effective amount of a fusion polypeptide according to any one of Aspects 115-118, wherein the pharmaceutical composition is co-administered with a second pharmaceutical composition that is a modified cell (e.g., a macrophage or NK cell) comprising one or more exogenous activation receptors (e.g., a CAR or a TCR).

123. A method of treating cancer comprising administering to a patient a pharmaceutical composition comprising an effective amount of a fusion polypeptide according to any one of Aspects 115-118, wherein the pharmaceutical composition is co-administered with a second pharmaceutical composition that is an immune checkpoint inhibitor (e.g., an anti-PD1 antibody).

124. A method of treating cancer according to any of Aspects 119-124, wherein the fusion polypeptide is administered before, at the same time, or after administration of the second pharmaceutical composition.

### Aspects Set B

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method comprising administering to an individual:
(i) a first composition comprising:
(a) modified or unmodified T cells having a T cell receptor (TCR); or
(b) modified cells that comprise a chimeric antigen receptor (CAR), wherein the CAR binds to a target antigen and the modified cell comprises an intracellular signaling domain that is activated by interaction of the modified cell with IL-2; or
(c) modified cells comprising one or more exogenous activation receptors; or
(d) a product that can engage with the TCR of a T cell or be processed by an immune system into one or more antigens that can be presented by a major histocompatibility complex (MHC) to the TCR of a T cell, optionally wherein the one or more antigens are cancer-associated antigens; or
(e) one or more nucleic acids encoding one or more polypeptides that can engage with the TCR of a T cell or can be processed by an immune system into one or more antigens that can be presented by a major histocompatibility complex (MHC) to the TCR of a T cell, optionally wherein the one or more antigens are cancer-associated antigens; or
(f) at least one immune checkpoint inhibitor (CPI), and
(ii) a second composition comprising an immunomodulatory protein,
wherein the immunomodulatory protein comprises one or more variant IL-2 polypeptides that have at least two amino acid substitutions relative to set forth in SEQ ID NO:1, wherein the one or more variant IL-2 polypeptides bind to an IL-2R alpha chain (IL-2Rα), and wherein the binding affinity to IL-2Rα is less than the affinity of a wild-type IL-2 polypeptide for IL-2Rα when assayed under the same conditions, wherein IL-2Rα has the amino acid sequence set forth in SEQ ID NO:2, and wherein the one or more variant IL-2 polypeptides bind to an IL-2R beta chain (IL-2Rβ), and wherein the binding affinity to IL-2Rβ is less than the affinity of a wild-type IL-2 polypeptide for IL-2Rβ when assayed under the same conditions, wherein the IL-2Rβ has the amino acid sequence set forth in SEQ ID NO:3, wherein the second composition preferentially activates T cells whose TCRs are engaged with an antigen presented by an MHC, as compared to T cells whose TCRs are not engaged with an antigen presented by an MHC, wherein the first and second compositions are administered at the same time or at different times, and wherein when the individual is administered a first composition comprising (a), (b), (c), (d) or (e), the individual also may be administered a CPI.

Aspect 2. A method according to aspect 1, wherein at least one of the one or more variant IL-2 polypeptides exhibits at least a two-fold decrease in binding affinity to IL-2Rβ compared to the binding affinity of a wild-type IL-2 polypeptide for IL-2Rβ, and exhibits at least a fifty-fold decrease in binding affinity to IL-2Rα compared to the binding affinity of a wild-type IL-2 polypeptide for IL-2Rα.

Aspect 3. A method according to aspect 1 or 2, wherein at least one of the one or more variant IL-2 polypeptides comprise at least one substitution that decreases the affinity of the variant IL-2 polypeptide for IL-2Rα, optionally wherein the at least one substitution is selected from a substitution at R38, F42, K43, Y45, E62, P65, E68, V69, L72, and combinations thereof.

Aspect 4. A method according to any one of aspects 1-3, wherein at least one of the one or more variant IL-2 polypeptides comprises a substitution of the phenylalanine amino acid F42, optionally wherein the phenylalanine is substituted with Ala, Gly, Val, Ile, or Leu.

Aspect 5. A method according to any one of aspects 1-4, wherein at least one of the one or more variant IL-2 polypeptides comprise at least one substitution that decreases the affinity of the variant IL-2 polypeptide for IL-2Rβ, optionally wherein the at least one substitution is selected from a substitution at E15, H16, L19, D20, D84, S87, N88, V91, I92, and combinations thereof.

Aspect 6. A method according to any one of aspects 1-5, wherein at least one of the one or more variant IL-2 polypeptides comprises a substitution of the histidine amino acid H16, optionally wherein the histidine is substituted with Ala, Gly, Val, Leu, Thr, Ile, Asp, or Glu.

Aspect 7. A method according to any one of aspects 1-6, wherein at least one of the one or more variant IL-2 polypeptides comprises a substitution of the asparagine amino acid N88, optionally wherein the asparagine is substituted with Gly, Ala, Ser, Thr, Arg or Asp.

Aspect 8. A method according to any one of aspects 1-7, wherein at least one of the one or more variant IL-2 polypeptides comprises substitutions of amino acids F42 and H16, optionally wherein the phenylalanine is substituted with Ala, and wherein the histidine is substituted with Ala, Thr, Asp, or Glu.

Aspect 9. A method according to any one of aspects 1-8, wherein at least one of the one or more variant IL-2 polypeptides comprises: (i) an H16A substitution and an F42A substitution; (ii) an H16T substitution and an F42A substitution, (iii) an H16E substitution and an F42A substitution, and (iv) an H16D substitution and an F42A substitution.

Aspect 10. A method according to any one of aspects 1-9, wherein at least one of the one or more variant IL-2 polypeptides comprises substitutions at F42, H16, and N88.

Aspect 11. A method according to aspect 10, wherein the asparagine amino acid N88 is substituted with Gly, Ala, Ser, Thr, Arg or Asp.

Aspect 12. A method according to any one of aspects 1-11, wherein the immunomodulatory protein comprises two or more variant IL-2 polypeptides, wherein each variant IL-2 polypeptide comprises the same amino acid sequence.

Aspect 13. A method according to aspect 12, wherein the immunomodulatory protein comprises two variant IL-2 polypeptides that are in tandem and joined by an independently selected linker, optionally wherein the linker comprises glycine and serine.

Aspect 14. A method according to any of aspects 1-13, wherein the immunomodulatory protein further comprises a carrier.

Aspect 15. A method according to aspect 14, wherein the carrier is a lipid vesicle (e.g., a liposome) or micelle, a nanoparticle, a PEGylated protein, or an artificial antigen presenting cell such as engineered erythroid cell or enucleated cell (e.g., a platelet).

Aspect 16. A method according to any of aspects 1-13, wherein the immunomodulatory protein further comprises an immunoglobulin (Ig) scaffold polypeptide or a non-Ig scaffold polypeptide.

Aspect 17. A method according to aspect 16, wherein the immunomodulatory protein comprises a non-Ig scaffold chosen from an XTEN polypeptide, a transferrin polypeptide, an elastin-like polypeptide, a silk-like polypeptide, a fibronectin-based scaffold protein, or a silk-elastin-like polypeptide.

Aspect 18. A method according to aspect 16, wherein the wherein immunomodulatory protein is a fusion polypeptide that comprises: a) the one or more variant IL-2 polypeptides; and b) an Fc polypeptide, and wherein the Ig Fe polypeptide is an IgG1 Fe polypeptide, an IgG2 Fe polypeptide, an IgG3 Fc polypeptide, an IgG4 Fc polypeptide, an IgA Fc polypeptide, or an IgM Fc polypeptide.

Aspect 19. A method according to aspect 18, wherein the Ig Fc polypeptide is a variant that has a substantially reduced effector function, e.g. a substantially reduced ability to effect complement-dependent cytotoxicity (CDC) and/or antibody-dependent cell cytotoxicity (ADCC).

Aspect 20. A method according to aspect 18 or 19, wherein the Ig Fe polypeptide comprises one or more amino acid substitutions selected from N297A, L234A, L235A, L234F, L235E, G237A and P331S, wherein N297, L234, L235, G237 and P331 correspond to N77, L14, L15, G17 and P111, respectively, of the amino acid sequences depicted in FIG. 9A.

Aspect 21. A method according to aspect 18-20, wherein the Ig Fc polypeptide is an IgG1 Fc polypeptide that comprises an amino acid sequence having at least 95% amino acid sequence identity to the amino acid sequence depicted in any one of FIGS. 9A-9M.

Aspect 22. A method according to any one of aspects 18-21, wherein the immunomodulatory protein comprises a homodimer of two immunomodulatory proteins, each of which comprises an Ig Fe polypeptide, and wherein the Ig Fe of one immunomodulatory protein is joined by one or more disulfide bonds to the Ig Fc of the other immunomodulatory protein.

Aspect 23. A method according to aspect 22, wherein each of the immunomodulatory proteins in the homodimer comprises two variant IL-2 polypeptides in tandem, and wherein the variant IL-2 polypeptides are joined by an independently selected linker.

Aspect 24. A method according to aspect 22, wherein each of the immunomodulatory proteins in the homodimer comprises, from N-terminus to C-terminus:
(i) a variant IL-2 polypeptide;
(ii) an independently selected linker;
(iii) a variant IL-2 polypeptide;
(iv) an independently selected linker; and
(v) an Ig Fe polypeptide.

Aspect 25. A method according to aspect 22, wherein each of the immunomodulatory proteins in the homodimer comprises, from N-terminus to C-terminus:
(i) a variant IL-2 polypeptide;
(ii) an independently selected linker;
(iii) a variant IL-2 polypeptide;
(iv) an independently selected linker;
(v) an Ig Fe polypeptide;
(vi) an independently selected linker;
(vii) a variant IL-2 polypeptide;
(viii) an independently selected linker; and
(ix) a variant IL-2 polypeptide.

Aspect 26. A method according to aspect 22, wherein each of the immunomodulatory proteins in the homodimer comprises, from N-terminus to C-terminus:
(i) a variant IL-2 polypeptide;
(ii) an independently selected linker;
(iii) an Ig Fe polypeptide;
(iv) an independently selected linker; and
(v) a variant IL-2 polypeptide;
Aspect 27. A method according to aspect 22, wherein each of the immunomodulatory proteins in the homodimer comprises, from N-terminus to C-terminus:
(i) an Ig Fe polypeptide;
(ii) an independently selected linker;
(iii) a variant IL-2 polypeptide;
(iv) an independently selected linker; and
(v) a variant IL-2 polypeptide.

Aspect 28. A method according to any one of aspects 18-21, wherein the immunomodulatory protein comprises a heterodimer of two immunomodulatory proteins, wherein one of the immunomodulatory proteins comprises an Ig Fc polypeptide comprising an interspecific dimerization sequence and the other immunomodulatory protein comprises an Ig Fe polypeptide comprising a counterpart interspecific sequence, or wherein the immunomodulatory protein comprises a heterodimer comprising first and second polypeptides, wherein the first polypeptide comprises an Ig Fe polypeptide comprising an interspecific dimerization sequence, wherein the second polypeptide comprises an Ig Fc polypeptide comprising a counterpart interspecific sequence, and wherein one of either the first or second polypeptide comprises the one or more variant IL-2 polypeptides.

Aspect 29. A method according to aspect 28, wherein
(A) one of the immunomodulatory proteins in the heterodimer comprises, from N-terminus to C-terminus:
(i) a variant IL-2 polypeptide;
(ii) an independently selected linker;
(iii) a variant IL-2 polypeptide;
(iv) an independently selected linker; and
(v) an Ig Fc polypeptide comprising an interspecific binding sequence, and
wherein the other immunomodulatory protein in the heterodimer comprises, from N-terminus to C-terminus:
(i) an Ig Fc polypeptide comprising a counterpart interspecific binding sequence;
(ii) an independently selected linker;
(iii) a variant IL-2 polypeptide;
(iv) an independently selected linker; and
(v) a variant IL-2 polypeptide, or
(B) the first polypeptide in the heterodimer comprises, from N-terminus to C-terminus:
(i) a variant IL-2 polypeptide;
(ii) an independently selected linker;
(iii) a variant IL-2 polypeptide;
(iv) an independently selected linker; and
(v) an Ig Fe polypeptide comprising an interspecific binding sequence, and
the second polypeptide in the heterodimer comprises an Ig Fc polypeptide comprising a counterpart interspecific binding sequence, but does not comprise a variant IL-2 polypeptide, or
(C) the first polypeptide in the heterodimer comprises, from N-terminus to C-terminus:
(i) an Ig Fe polypeptide comprising an interspecific binding sequence,
(ii) an independently selected linker;
(iii) a variant IL-2 polypeptide;
(iv) an independently selected linker;
(v) a variant IL-2 polypeptide, and
the second polypeptide in the heterodimer comprises an Ig Fc polypeptide comprising a counterpart interspecific binding sequence, but does not comprise a variant IL-2 polypeptide.

Aspect 30. A method according to aspect 28, wherein one of the immunomodulatory proteins in the heterodimer comprises, from N-terminus to C-terminus:
(i) a variant IL-2 polypeptide;
(ii) an independently selected linker; and
(iii) an Ig Fe polypeptide comprising an interspecific binding sequence, and
wherein the other immunomodulatory protein in the heterodimer comprises, from N-terminus to C-terminus:
(i) an Ig Fe polypeptide comprising a counterpart interspecific binding sequence;
(ii) an independently selected linker; and
(iii) a variant IL-2 polypeptide.

Aspect 31. A method according to aspect 28, wherein
(A) the first polypeptide in the heterodimer comprises, from N-terminus to C-terminus:
(i) a variant IL-2 polypeptide;
(ii) an independently selected linker; and
(iii) an Ig Fe polypeptide comprising an interspecific binding sequence, and
the second polypeptide in the heterodimer comprises an Ig Fe polypeptide comprising a counterpart interspecific binding sequence, but does not comprise a variant IL-2 polypeptide, or
(B) the first polypeptide in the heterodimer comprises, from N-terminus to C-terminus:
(i) an Ig Fe polypeptide comprising an interspecific binding sequence;
(ii) an independently selected linker; and
(iii) a variant IL-2 polypeptide, and
wherein the second polypeptide in the heterodimer comprises an Ig Fc polypeptide comprising a counterpart interspecific binding sequence, but does not comprise a variant IL-2 polypeptide.

Aspect 32. A method according to any one of aspects 1-31, wherein the first composition comprises one or more products that can engage with the TCR of a T cell or be processed by an immune system into one or more antigens that can be presented by a major histocompatibility complex (MHC) to the TCR of a T cell, optionally wherein the one or more antigens are cancer-associated antigens.

Aspect 33. A method according to any one of aspects 1-31, wherein the first composition comprises one or more nucleic acids encoding one or more polypeptides that can engage with the TCR of a T cell or can be processed by an immune system into one or more antigens that can be presented by a major histocompatibility complex (MHC) to the TCR of a T cell, optionally wherein the one or more antigens are cancer-associated antigens.

Aspect 34. The method of any one of aspects 1-31, wherein the first composition comprises a TCR-T cell that comprises an exogenous TCR, optionally wherein the TCR binds a cancer-associated antigen when the cancer-associated antigen is presented by an MHC to the TCR.

Aspect 35. A method according to any one of aspects 1-31, wherein the first composition comprises tumor infiltrating lymphocytes (TILS), optionally wherein the TILS have been modified to reduce the susceptibility of the TILS to T-cell suppressive signals.

Aspect 36. A method according to any one of aspects 31-35, wherein the cancer-associated antigen is an antigen chosen from alpha-feto protein, Wilms-tumor-1 (WT-1), a mutant KRAS, e.g., comprising a G12C or G12D mutation, melanoma antigen recognized by T cells 1 (MART-I), melanoma-associated antigen (MAGE), MAGE-A1, MAGE-A3, MAGE-A4, human papillomavirus (HPV) antigen E6, HPV antigen E7, New York esophageal squamous cell carcinoma 1 (NY-ESO-1), MUC-1 (mucin-1, cell surface associated), mesothelin, survivin, prostate stem cell antigen (PSCA), prostate-specific membrane antigen (PSMA), prostate-specific antigen (PSA), a mutant p53 polypeptide, a Ras polypeptide, nuclear factor erythroid 2-related factor 2 (NFE2L2), beta-catenin, PIK3CA (phosphatidylinositol-4,5-bisphosphate 3-kinase catalytic subunit alpha), and BRAF.

Aspect 37. A method according to any one of aspects 1-31, wherein the first composition comprises a CAR having an antigen-binding domain that is specific for a cancer-associated antigen, optionally wherein the cell is a T cell, a macrophage, or an NK cell, optionally wherein: (i) when the CAR is not bound to a cancer-associated antigen, the second composition provides homeostatic signals to the cell for survival, and/or (ii) when the CAR is not bound to a cancer-associated antigen, the second composition provides activating signals to the cell that cause the cell to proliferate and retain its cytotoxic function.

Aspect 38. The method of aspect 37, wherein the antigen-binding domain is a single-chain Fv polypeptide or a nanobody.

Aspect 39. The method of aspect 37 or 38, wherein the cancer-associated antigen is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2.

Aspect 40. The method of any one of aspects 1-39, wherein the method is for the treatment of a cancer in the individual.

Aspect 41. The method of aspect 40, wherein the first composition comprises (a), (b), (c), (d) or (e), and further comprising administering at least one immune checkpoint inhibitor (CPI) to the individual, wherein the CPI, first composition, and second composition are administered at the same time or at different times.

Aspect 42. The method of aspect 41, wherein the at least one immune checkpoint inhibitor comprises an antibody specific for the immune checkpoint.

Aspect 43. The method of aspect 42, wherein the antibody is specific for an immune checkpoint chosen from CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2, optionally wherein the immune checkpoint inhibitor is an antibody specific for PD-1, PD-L1, CTLA-4, TIGIT and LAG3.

Aspect 44. The method of any one of aspects 1-36, wherein the method is for the prevention of a cancer in the individual.

Aspect 45. The method of any one of aspects 1-36, wherein the method is for the treatment of a cancer in the individual.

Aspect 46. A method according to any one of aspects 1-30, wherein the individual is administered a CAR-T therapy product, a TCR-T therapy product, or a CAR-NK therapy product.

Aspect 47. The method of aspect 46, wherein the individual is administered a CAR-T therapy product, wherein the CAR-T cell therapy product comprises a population of modified autologous T cells comprising a CAR or allogeneic T cells comprising a CAR, wherein the CAR comprises an antigen-binding domain specific for a cancer-associated antigen.

Aspect 48. The method of aspect 47, wherein the antigen-binding domain is a single-chain Fv polypeptide or a nanobody.

Aspect 49. The method of aspect 47 or 48, wherein the cancer-associated antigen is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2.

Aspect 50. The method of any one of aspects 44-49, further comprising administering to the individual an immune checkpoint inhibitor.

Aspect 51. The method of aspect 50, wherein the immune checkpoint inhibitor is an antibody specific for an immune checkpoint selected from CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2, optionally wherein the CPI is an antibody specific for an immune checkpoint selected from CTLA-4, TIGIT, PD-1 and PD-L1, or from PD-1 or PD-L1.

Aspect 52. A method according to any one of aspects 1-31, wherein the first composition is a vaccine comprising one or more products that can engage with the TCR of a T cell or be processed by an immune system into one or more antigens that can be presented by a major histocompatibility complex (MHC) to the TCR of a T cell, optionally wherein the one or more antigens are cancer-associated antigens.

Aspect 53. A method according to any one of aspects 1-31, wherein the first composition is a vaccine comprising a nucleic acid comprising a nucleotide sequence encoding a polypeptide that is capable of being processed by an immune system into one or more antigens that can be presented by a major histocompatibility complex (MHC) to the TCR of a T cell, optionally wherein the one or more antigens are cancer-associated antigens.

Aspect 54. The method of aspect 52, wherein the vaccine comprises one or more products that can engage with the TCR of a T cell or be processed by an immune system into more than one cancer-associated antigens that can be presented by a major histocompatibility complex (MHC) to the TCRs of T cells.

Aspect 55. The method of aspect 53, wherein the vaccine comprises one or more nucleic acids comprising one or more nucleotide sequences that encode a plurality of polypeptides, wherein the polypeptides can engage with the TCR of a T cell or be processed by an immune system into more than one cancer-associated antigens that can be presented by a major histocompatibility complex (MHC) to the TCRs of T cells.

Aspect 56. A method according to any one of claims 1-55, wherein the immunomodulatory protein is selected from the group of: i) an immunomodulatory protein comprising, consisting essentially of, or consisting of a homodimer of the 2657 protein depicted in FIG. 23A, wherein the two copies of the 2657 protein are joined by two disulfide bonds that link the Ig Fc polypeptides in each copy of 2657; ii) an immunomodulatory protein comprising, consisting essentially of, or consisting of a homodimer of the 2656 protein depicted in FIG. 25A, wherein the two copies of the 2656 protein are joined by two disulfide bonds that link the Ig Fc polypeptides in each copy of 2656; iii) an immunomodulatory protein comprising, consisting essentially of, or consisting of a homodimer of the 2657A protein depicted in FIG. 23B, wherein the two copies of the 2657A protein are joined by two disulfide bonds that link the Ig Fe polypeptides in each copy of 2657A; iv) an immunomodulatory protein comprising, consisting essentially of, or consisting of a homodimer of the 2656A protein depicted in FIG. 25B, wherein the two copies of the 2656A protein are joined by two disulfide bonds that link the Ig Fc polypeptides in each copy of 2656A; and v) a heterodimer comprising the 4123 protein depicted in FIG. 26A and the 4124 protein depicted in FIG. 26B. A fusion polypeptide as shown in any one of FIGS. 2A-2C, 3A-3C, 4A-4C, 5A-5B, 6A-6B, 7A-7B, 8A-8F, 10A-10C, 11A-11C, 12A-12B, 13A-14B, 14A-14N, and 30A-30G wherein the fusion polypeptide comprises one or more variant IL-2 polypeptides that have at least two amino acid substitutions relative to set forth in SEQ ID NO:1, wherein the one or more variant IL-2 polypeptides bind to an IL-2R alpha chain (IL-2Rα), and wherein the binding affinity to IL-2Rα is less than the affinity of a wild-type IL-2 polypeptide for IL-2Rα when assayed under the same conditions, wherein IL-2Rα has the amino acid sequence set forth in SEQ ID NO:2, and wherein the one or more variant IL-2 polypeptides bind to an IL-2R beta chain (IL-2Rβ), and wherein the binding affinity to IL-2Rβ is less than the affinity of a wild-type IL-2 polypeptide for IL-2Rβ when assayed under the same conditions, wherein the IL-2Rβ has the amino acid sequence set forth in SEQ ID NO:3.

Aspect 57. A fusion polypeptide as shown in any one of FIGS. 2A-2C, 3A-3C, 4A-4C, 5A-5B, 6A-6B, 7A-7B, 8A-8F, 10A-10C, 11A-11C, 12A-12B, 13A-14B, 14A-14N, and 30A-30G wherein the fusion polypeptide comprises one or more variant IL-2 polypeptides that have at least two amino acid substitutions relative to set forth in SEQ ID NO:1, wherein the one or more variant IL-2 polypeptides bind to an IL-2R alpha chain (IL-2Rα), and wherein the binding affinity to IL-2Rα is less than the affinity of a wild-type IL-2 polypeptide for IL-2Rα when assayed under the same conditions, wherein IL-2Rα has the amino acid sequence set forth in SEQ ID NO:2, and wherein the one or more variant IL-2 polypeptides bind to an IL-2R beta chain (IL-2Rβ), and wherein the binding affinity to IL-2Rβ is less than the affinity of a wild-type IL-2 polypeptide for IL-2Rβ when assayed under the same conditions, wherein the IL-2Rβ has the amino acid sequence set forth in SEQ ID NO:3.

Aspect 58. A fusion polypeptide according to aspect 57, wherein at least one of the one or more variant IL-2 polypeptides comprises one or more mutations that can reduce binding of IL-2 to IL-2Rα, wherein the one or more mutations are chosen from substitutions at one or more of amino acids R38, F42, K43, Y45, E62, P65, E68, V69, and L72.

Aspect 59. A fusion polypeptide according to aspect 58, wherein at least one of the one or more variant IL-2 polypeptides comprises a substitution of amino acid F42, optionally wherein the Phe is substituted with Ala or Lys.

Aspect 60. A fusion polypeptide according to any one of aspects 57-59, wherein at least one of the one or more variant IL-2 polypeptides comprises one or more mutations that can reduce binding of IL-2 to IL-2Rβ, wherein the one or more mutations are chosen from substitutions at one or more of amino acids E15, H16, L19, D20, D84, S87, N88, V91, I92.

Aspect 61. A fusion polypeptide according to aspect 60, wherein at least one of the one or more variant IL-2 polypeptides comprises a substitution of amino acid H16, optionally wherein the His is substituted with Ala, Glu, Thr, or Asp.

Aspect 62. A fusion polypeptide according to aspect 57, wherein at least one of the one or more variant IL-2 polypeptides comprises substitutions chosen from E15A with R38A, R38D or R38E.

Aspect 63. A fusion polypeptide according to aspect 57, wherein at least one of the one or more variant IL-2 polypeptides comprises substitutions chosen from: H16A with R38A, R38D or R38E; H16T with R38A, R38D or R38E; H16E with R38A, R38D or R38E; and H16D with R38A, R38D or R38E.

Aspect 64. A fusion polypeptide according to aspect 57, wherein at least one of the one or more variant IL-2 polypeptides comprises substitutions chosen from: D84H with R38A, R38D or R38E; D84K with R38A, R38D or R38E; and D84R with R38A, R38D or R38E.

Aspect 65. A fusion polypeptide according to aspect 57, wherein at least one of the one or more variant IL-2 polypeptides comprises substitutions chosen from: R38A with N88S, N88A, N88G, N88R, N88T, or N88D; R38D with N88S, N88A, N88G, N88R, N88T, or N88D; and R38E with N88S, N88A, N88G, N88R, N88T, or N88D.

Aspect 66. A fusion polypeptide according to aspect 57, wherein at least one of the one or more variant IL-2 polypeptides comprises substitutions chosen from: R38A with V91E, V91A or V91T, R38D with V91E, V91A or V91T; and R38E with V91E, V91A or V91T.

Aspect 67. A fusion polypeptide according to aspect 57, wherein at least one of the one or more variant IL-2 polypeptides comprises substitutions chosen from R38A, 192A, R38D, 192A and R38E, I92A.

Aspect 68. A fusion polypeptide according to aspect 57, wherein at least one of the one or more variant IL-2 polypeptides comprises substitutions chosen from E15A, F42A and E15A, F42K.

Aspect 69. A fusion polypeptide according to aspect 57, wherein at least one of the one or more variant IL-2 polypeptides comprises substitutions chosen from: H16A, F42A; H16T, F42A; H16E, F42A; H16D, F42A; H16A, F42K; H16T, F42K; and H16E, F42K; H16D, F42K.

Aspect 70. A fusion polypeptide according to aspect 57, wherein at least one of the one or more variant IL-2 polypeptides comprises substitutions chosen from: F42A with N88S, N88A, N88G, N88R, N88T, or N88D; and F42K with N88S, N88A, N88G, N88R, N88T, or N88D.

Aspect 71. A fusion polypeptide according to aspect 57, wherein at least one of the one or more variant IL-2 polypeptides comprises substitutions chosen from: F42A with V91E, V91A, or V91T; and F42K with V91E, V91A, or V91T

Aspect 72. A fusion polypeptide according to aspect 57, wherein at least one of the one or more variant IL-2 polypeptides comprises substitutions chosen from: F42A with I92A; and F42K with I92A.

Aspect 73. A fusion polypeptide according to aspect 57, wherein at least one of the one or more variant IL-2 polypeptides comprises substitutions of E15A and K43E.

Aspect 74. A fusion polypeptide according to aspect 57, wherein at least one of the one or more variant IL-2 polypeptides comprises substitutions chosen from: H16A, K43E; H16T, K43E; H16E, K43E; and H16D, K43E.

Aspect 75. A fusion polypeptide according to aspect 57, wherein at least one of the one or more variant IL-2 polypeptides comprises substitutions chosen from K43E with D84H, D84K or D84R.

Aspect 76. A fusion polypeptide according to aspect 57, wherein at least one of the one or more variant IL-2 polypeptides comprises substitutions chosen from K43E with N88S, N88A, N88G, N88R, N88T, or N88D.

Aspect 77. A fusion polypeptide according to aspect 57, wherein at least one of the one or more variant IL-2 polypeptides comprises substitutions chosen from K43E with V91E, V91A, or V91 T.

Aspect 78. A fusion polypeptide according to aspect 57, wherein at least one of the one or more variant IL-2 polypeptides comprises substitutions of K43E and I92A or E15A, E62Q.

Aspect 79. A fusion polypeptide according to aspect 57, wherein at least one of the one or more variant IL-2 polypeptides comprises substitutions chosen from: H16A, E62Q; H16T, E62Q; H16E, E62Q; and H16D, E62Q.

Aspect 80. A fusion polypeptide according to aspect 57, wherein at least one of the one or more variant IL-2 polypeptides comprises substitutions chosen from E62Q with D84H, D84K or D84R.

Aspect 81. A fusion polypeptide according to aspect 57, wherein at least one of the one or more variant IL-2 polypeptides comprises substitutions chosen from E62Q with N88S, N88A, N88G, N88R, N88T, or N88D.

Aspect 82. A fusion polypeptide according to aspect 57, wherein at least one of the one or more variant IL-2 polypeptides comprises substitutions chosen from E62Q with V91E, V91A, or V91T.

Aspect 83. A fusion polypeptide according to aspect 57, wherein at least one of the one or more variant IL-2 polypeptides comprises substitutions chosen from E62Q and I92A.

Aspect 84. A fusion polypeptide according to aspect 57, wherein at least one of the one or more variant IL-2 polypeptides comprises substitutions chosen from E62Q with V91E, V91A, or V91T.

Aspect 85. A fusion polypeptide according to aspect 68, wherein at least one of the one or more variant IL-2 polypeptides comprises substitutions at F42, E15 and N88.

Aspect 86. A fusion polypeptide according to aspect 68, wherein at least one of the one or more variant IL-2 polypeptides comprises substitutions at F42, E15 and V91.

Aspect 87. A fusion polypeptide according to aspect 69, wherein at least one of the one or more variant IL-2 polypeptides comprises substitutions at F42, H16 and D84.

Aspect 88. A fusion polypeptide according to aspect 69, wherein at least one of the one or more variant IL-2 polypeptides comprises substitutions at F42, H16 and N88.

Aspect 89. A fusion polypeptide according to aspect 69, wherein at least one of the one or more variant IL-2 polypeptides comprises substitutions at F42, H16 and V91.

Aspect 90. A fusion polypeptide according to aspect 69, wherein at least one of the one or more variant IL-2 polypeptides comprises substitutions at F42, H16 and 192.

Aspect 91. A fusion polypeptide according to any one of aspects 57-90, wherein the fusion protein is as shown in any one of FIGS. 10A-10C, FIGS. 11A-11C, FIGS. 12A-12B and FIGS. 13A-13B, FIGS. 14A-14N, and FIG. 30A-30G and wherein the fusion polypeptide comprises a functional protein that is a cancer-targeting polypeptide (CTP).

Aspect 92. A fusion polypeptide according to aspect 91, wherein the target of the cancer-targeting polypeptide is a peptide-HLA complex on the surface of a cancer cell.

Aspect 93. A fusion polypeptide according to aspect 91, wherein the target of the CTP is a cancer-associated epitope.

Aspect 94. A fusion polypeptide according to aspect 91, wherein the CTP is an antibody that is specific for a cancer-associated antigen.

Aspect 95. A fusion polypeptide according to aspect 91, wherein the CTP is an antibody that is specific for a peptide/HLA complex on the surface of a cancer cell, wherein the peptide can be a cancer-associated peptide (e.g., a peptide of a cancer-associated antigen).

Aspect 96. A fusion polypeptide according to aspect 91, wherein the functional protein is a TCR such as single-chain T cell receptor of “scTCR” that is specific for a peptide/HLA complex on the surface of a cancer cell, wherein the peptide can be a cancer-associated peptide (e.g., a peptide of a cancer-associated antigen).

Aspect 97. A fusion polypeptide according to aspect 91, wherein the functional protein comprises a wild-type or variant immunomodulatory polypeptide, e.g., a wild type or variant immunostimulatory polypeptide such as B7 family of costimulatory receptors, e.g., CD80, CD86, a cytokine such as IL-7, IL-12, IL-15 or IL-21, a TNF superfamily member such as CD-40, 4-1BBL and OX40, or a chemokine such as CCL19, CCL21, CXCL9/10/11, or CXCL12.

Aspect 98. A fusion polypeptide according to any one of aspects 57-97, wherein the fusion polypeptide comprises one or more independently selected linkers.

Aspect 99. A method comprising administering to an individual:
(i) a first composition comprising:
(a) modified or unmodified T cells having a T cell receptor (TCR), or
(b) modified cells that comprise a chimeric antigen receptor (CAR), wherein the CAR binds to a target antigen and the modified cell comprises an intracellular signaling domain that is activated by interaction of the modified cell with IL-2, or
(c) modified cells comprising one or more exogenous activation receptors; or
(d) one or more products that can engage with the TCR of a T cell or be processed by an immune system into one or more antigens that can be presented by a major histocompatibility complex (MHC) to the TCR of a T cell, optionally wherein the one or more antigens are cancer-associated antigens; or
(e) one or more nucleic acids encoding one or more polypeptides that can engage with the TCR of a T cell or can be processed by an immune system into one or more antigens that can be presented by a major histocompatibility complex (MHC) to the TCR of a T cell, optionally wherein the one or more antigens are cancer-associated antigens; or
(f) at least one immune checkpoint inhibitor (CPI), and
and
(ii) a second composition comprising a fusion polypeptide of any one of aspects 57-98,
wherein when the individual is administered a first composition comprising (a), (b), (c), (d) or (e), the individual also may be administered a CPI, and
   wherein the first and second compositions are administered at the same time or at different times.

Aspect 100. A method according to aspect 99, wherein the second composition preferentially activates T cells whose TCRs are engaged with an antigen presented by an MHC, as compared to T cells whose TCRs are not engaged with an antigen presented by an MHC,

Aspect 101. A method according to aspect 99 or 100, wherein the first composition comprises modified or unmodified T cells having a T cell receptor (TCR).

Aspect 102. A method according to aspect 99 or 100, wherein the first composition comprises modified cells that comprise a chimeric antigen receptor (CAR), wherein the CAR binds to a target antigen and the modified cell comprises an intracellular signaling domain that is activated by interaction of the modified cell with IL-2.

Aspect 103. A method according to aspect 99 or 100, wherein the first composition comprises modified cells comprising one or more exogenous activation receptors.

Aspect 104. A method according to aspect 99 or 100, wherein the first composition comprises one or more products that can engage with the TCR of a T cell or be processed by an immune system into one or more antigens that can be presented by a major histocompatibility complex (MHC) to the TCR of a T cell, optionally wherein the one or more antigens are cancer-associated antigens.

Aspect 105. A method according to aspect 99 or 100, wherein the first composition comprises one or more nucleic acids encoding one or more polypeptides that can engage with the TCR of a T cell or can be processed by an immune system into one or more antigens that can be presented by a major histocompatibility complex (MHC) to the TCR of a T cell, optionally wherein the one or more antigens are cancer-associated antigens.

Aspect 106. A method according to aspect 99 or 100, wherein the first composition comprises a CPI.

Aspect 107. A method according to any of aspects 101-105, wherein the method further comprises administering a CPI.

Aspect 108. A method according to any one of aspects 99-107, wherein the CPI is an antibody specific for an immune checkpoint selected from CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2.

Aspect 109. A method according to any one of aspects 99-107, wherein the CPI is an antibody specific for an immune checkpoint selected from CTLA-4, TIGIT. PD-1, and PD-L1.

Aspect 110. A method according to any one of aspects 99-107, wherein the CPI is an antibody specific for an immune checkpoint selected from PD-1 and PD-L1.

### Example

The following example is put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1—Determining Binding Affinities to IL-2Rα and IL-2Rβ

Kinetic measurements were performed using bio-layer interferometry on ForteBio's Octet Red96e using anti-Human IgG Fc capture (AHC) for human IL-2R. All experiments were performed at 30° C. with the orbital shake speed of 1000 rpm. Samples were diluted in assay buffer of 25 mM HEPES, pH 6.8, 50 mM KCl, 5% polyethylene glycol (PEG) 6000, 0.1% bovine serum albumin (BSA), 0.02% Tween20 in a 96-well non-binding black plate (Greiner 655900).

All proteins were diluted in assay buffer containing 25 mM HEPES, pH 6.8, 50 mM KCl, 5% PEG 6000, 0.1% BSA, 0.02% Tween20. The receptor ligands were prepared to a final concentration of 20 nM for IL-2Rα-Fc, and 25 nM for IL-2Rβ-Fc, and dispensed into a column of a 96-well non-binding plate at a volume of 200 μl/well. The analyte solutions were serially diluted 2-fold for 7 concentrations, starting from 1000 nM for CUE-1646 (WT-IL-2-FLAG-) or 3000 nM for CUE-1647 (H16A; F42A mutant IL-2-FLAG) for human IL-2R. The analytes were added to the 96-well plate at 200 μl/well. Kinetic measurements were made with appropriate concentration ranges depending on signal and robustness of fit.

AHC or HIS1K biosensors were pre-hydrated for 10 minutes and normalized in the assay buffer for 60 seconds (see). For each run a column of 8 biosensors were simultaneously submerged in the IL-2R ligand solution for 300 s. Immobilization of ligands onto the biosensors ranged from 1.6-1.4 nm for human IL-2Rα, and 2.0-1.7 nm for human IL-2Rβ. A 120 s baseline step in assay buffer alone allowed for any non-bound ligand to be washed off and stabilization of the signal before analyte association. The kinetics of the interactions were measured by submerging the 8 loaded biosensors into analyte solutions of either CUE-1646 (amino acid sequence depicted in FIG. 21) or CUE-1647 (amino acid sequence depicted in FIG. 22) at varying concentrations for 300 seconds; a reference well with buffer alone was included for each run to compensate for any baseline drift of the immobilized IL-2R. This was followed by a 300 second dissociation period into wells containing only assay buffer and monitoring the dissociation of the analytes from the immobilized receptor ligands. Empty biosensors were also subjected to the analyte dilutions to check for non-specific binding on the sensors. Fresh biosensors were used for each binding experiment. The binding sensorgrams were collected using the standard kinetics acquisition rate.

Raw data was processed and analyzed with ForteBio's Data Analysis 11.0 software. For each run the varying analyte concentrations were subtracted from their corresponding reference well to compensate for any background drift. The sensorgrams Y-axis were aligned to the baseline for a common reference point, and the inter-step correction was aligned to dissociation to correct for any misalignments between the association and dissociation steps. The sensorgrams were processed with a Savitzky-Golay Filtering to remove any high-frequency noise from the curves. The processed curves were globally fit to a 1:1 binding model [Hahnefeld et al. (2004) Methods in Molecular Medicine 94:299]. Robustness of fits were evaluated based on visual alignment of raw data to calculated fits, as well as reasonable statistical measurements (i.e., equilibrium dissociation constant [KD] Error <10%, χ²<1, and R²>0.95). Typically, KD error was <10% except for CUE-1647 binding to human IL-2Rβ, where KD error was ˜20%. This may be due to low signal amplitude to noise. Binding experiments for IL-2Rα-Fc and IL-2Rβ-Fc on CUE-1646 and CUE-1647 were performed in triplicate. Rat IL-2Rα-his binding experiments were performed in duplicate.

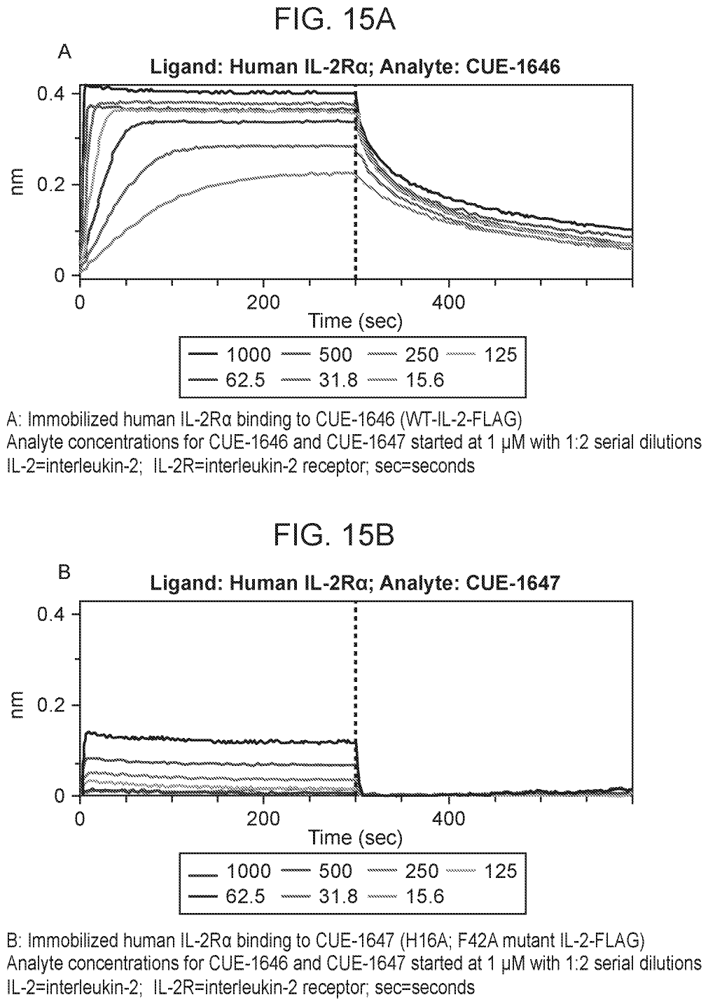

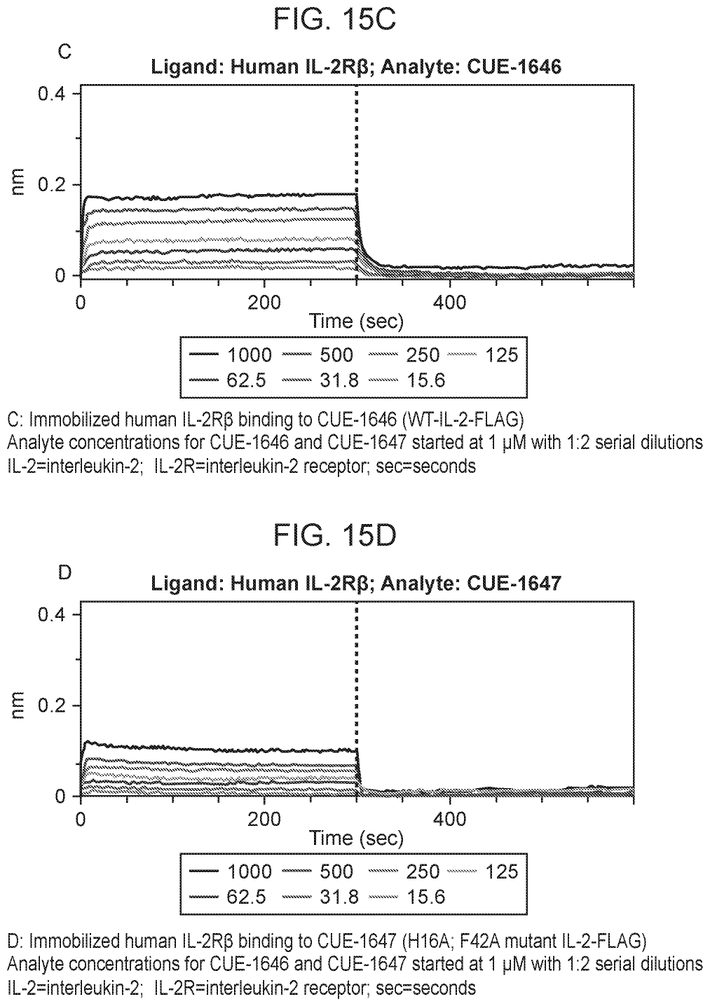

The differences in binding between CUE-1646 and CUE-1647 to human IL-2Rα and β subunits were studied using the Forte Bio Octet RED96e system. The binding signal of CUE-1647 was lower for both human IL-2Rα and β compared with CUE-1646 (see FIGS. 15A-D). CUE-1646 and CUE-1647 were run at the same concentrations to compare the binding signal on each receptor. Significant binding signal was observed even at the lowest concentration of 15 nM for CUE-1646 with IL-2Rα, whereas signal is barely detectable at 250 nM with CUE-1647 (FIGS. 15A-B). Compared to CUE-1646, CUE-1647 demonstrates a reduced binding signal with human IL-2Rβ (FIGS. 15C-D).

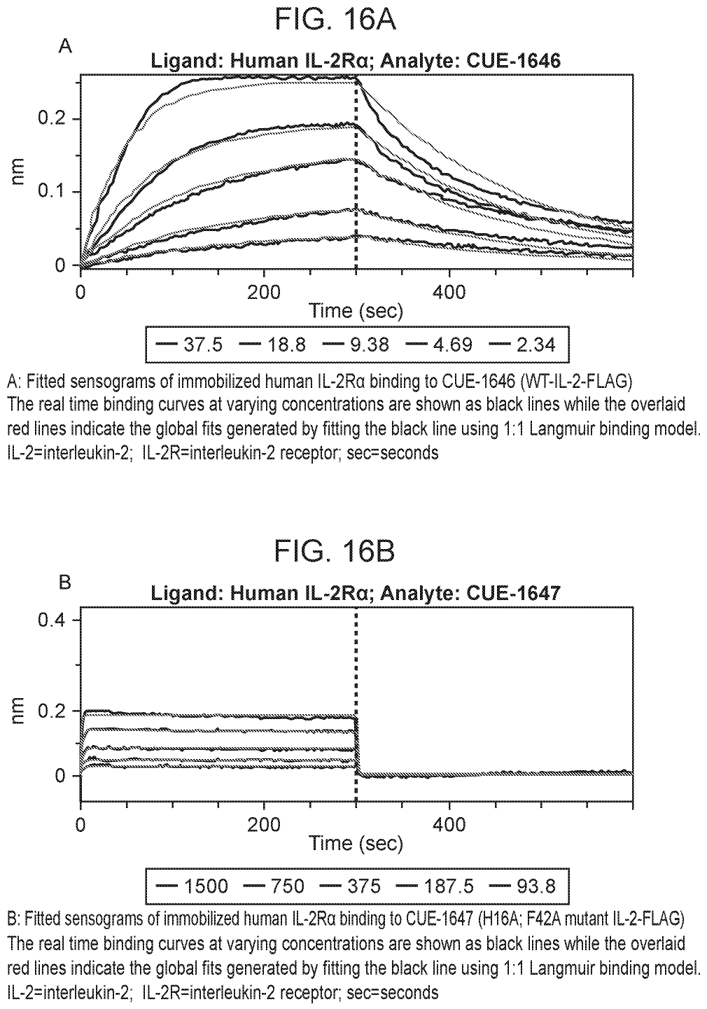

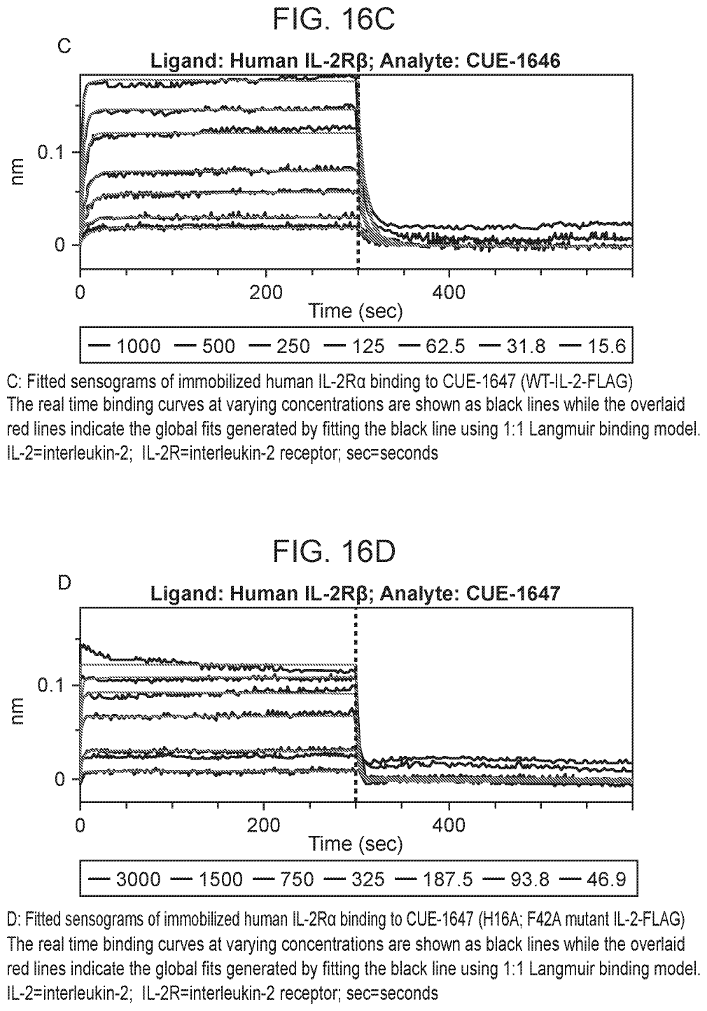

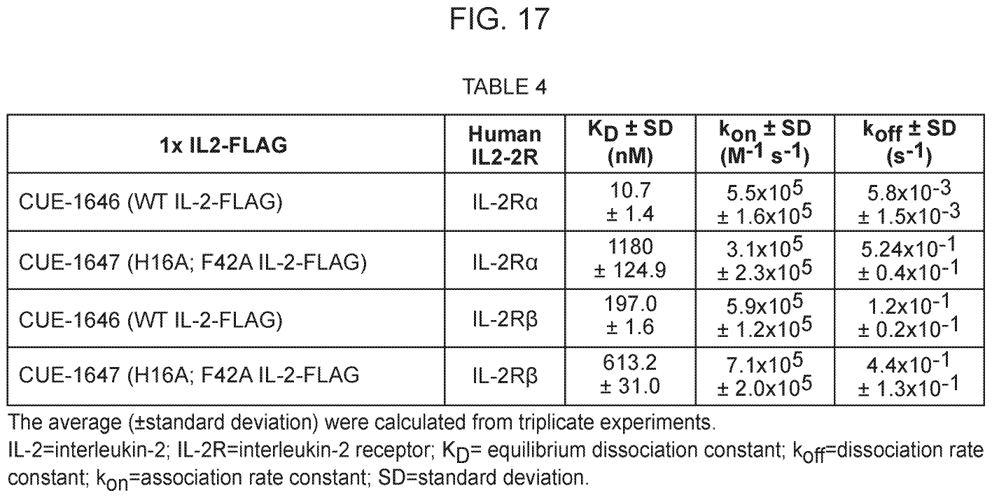

To calculate affinity and kinetic measurements, concentrations were expanded to cover appropriate ranges for each receptor and construct tested and fit to a global 1:1 binding model (FIGS. 16A-D). The binding experiments were repeated in triplicate; affinity and kinetics were averaged, and standard deviations calculated (see Table 4 provided as FIG. 17). The fits for CUE-1646 did not visually align as well to the raw data, however calculation of the KD from steady-state analysis gave similar KD values. The KD values of human IL-2Rα binding to CUE-1647 (H16A; F42A mutant IL-2-FLAG) and CUE-1646 (WT IL-2-FLAG) were ˜1000 nM and 10 nM, respectively, indicating an ˜100-fold reduction in binding affinity to the IL-2Rα subunit. CUE-1647 also showed a decrease in affinity to human IL-2Rβ with a KD of ˜600 nM compared with ˜200 nM for CUE-1646, indicating a ˜3-fold reduction in binding affinity to the IL-2Rβ subunit. These binding affinities (KD) obtained in the Octet system are similar to those previously reported by Levin et al. 2012 using surface plasmon resonance to quantify binding of human IL-2Rα with wild type (WT) IL-2 (6.6 nM) and an F42A IL-2 mutant (786 nM), and binding of human IL-2Rβ to WT IL-2 (280 nM).

The equilibrium dissociation constant (KD) is measured by the ratio between the dissociation rate constant (koff) and association rate constant (kon). The large difference in affinity of CUE-1646 vs CUE-1647 for human IL-2Rα is primarily due to the faster off rate of the double mutant with IL-2Rα compared with CUE-1646, whereas the association rate remains similar for both constructs (see Table 4 provided as FIG. 17). The association rate on human IL-2Rβ is similar for both WT and mutant IL-2 constructs, but slightly faster dissociation is observed with CUE-1647. This translates into a weaker affinity, as reported by the CUE-1647 KD for the IL-2Rβ subunit.

### Example 2: Proliferation of NK Cells

The effect of an IL-2/Fc fusion polypeptide on frequency in vivo of NK cells was evaluated. BALB/c mice were administered with a single intravenous dose of 10 mg/kg of the “Rgt 2657” construct produced by Chinese hamster ovary (CHO) cells. After 96 hours, flow cytometry was used to detect NK cells in the peripheral blood. The amino acid sequence of the “Rgt 2657” construct is provided in FIG. 23, and as noted above, polypeptide 2657 spontaneously forms a homodimer with a second copy of polypeptide 2657.

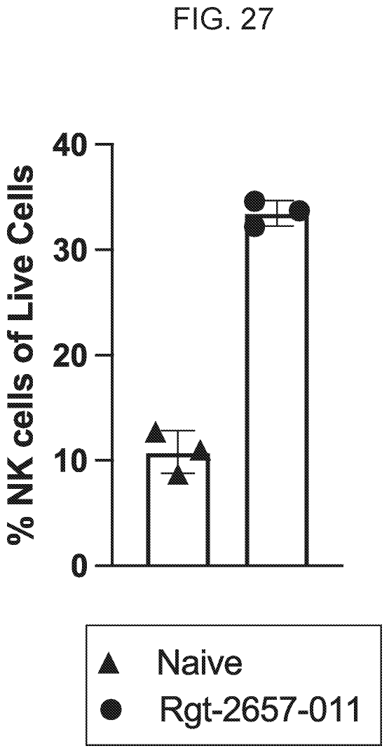

The data are shown in FIG. 27.

In FIG. 27, the right-hand bar represents data with Rgt-2657 and the left-hand bar represent data from naïve mice (i.e., mice not administered with an IL-2/Fc fusion polypeptide). The data in FIG. 27 show, inter alia, that NK cells are increased by treatment with the IL-2/Fc fusion polypeptide, compared to naïve mice.

### Example 3: Effect of IL-2/Fe Fusion Polypeptide on Antigen-Specific CD8⁺ T Cells In Vivo

The effect of an IL-2/Fc fusion polypeptide on proliferation of antigen-specific CD8′ T cells was assessed. The IL-2/Fc fusion polypeptide was a 4× Fc-IL2 referred to as “Rgt-2657.” This IL-2 fusion polypeptide comprises 4 copies of a variant IL-2 polypeptide having an Ala at amino acid 16 and an Ala at amino acid 42.

BALB/c mice were implanted with CT26 tumor cells. CT26 is a colorectal cancer cell line that expresses murine leukemia virus (MuLV) envelope glycoprotein 70 (gp70).

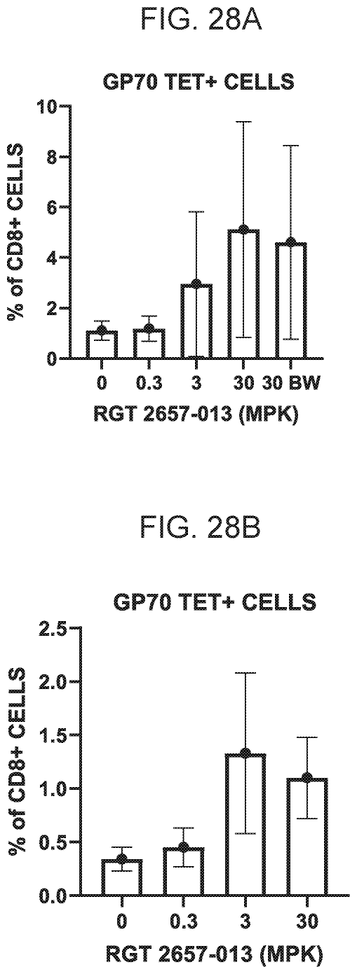

The Rgt-2657 polypeptide was administered to the mice 2 weeks after the implantation of the CT26 cells. Rgt-2657 was administered at Day 1 only, or for the group designated “BW” at Day 1 (first dose) and at Day 3 (second dose). Rgt-2657 was administered at 0, 0.3, 3, or 30 mg/kg body weight. At Day 6, blood samples were taken from all mice, and total CD8⁺ T cells and gp70-specific CD8⁺ T cells in the blood samples were quantified by flow cytometry (data shown in FIG. 28A). On Day 7, the mice who received Rgt-2657 only on Day 1 were given a repeat dose, and blood samples from those mice were taken on Day13 (data shown in FIG. 28B).

As shown in FIG. 28A, animals that received a higher dose of Rgt-2657 generally had a higher frequency of tumor antigen-specific CD8⁺ T cells, suggesting that the tumor antigen-specific CD8⁺ T cells expanded faster than all CD8⁺ T cells. As shown in FIG. 28B, at higher dose levels, an increased percentage of tumor antigen specific CD8⁺ T cells was also observed relative to all CD8⁺ T cells following repeated doses of Rgt-2657.

### Example 4: Effect of IL-2 Valency on CTLL-2 Proliferation

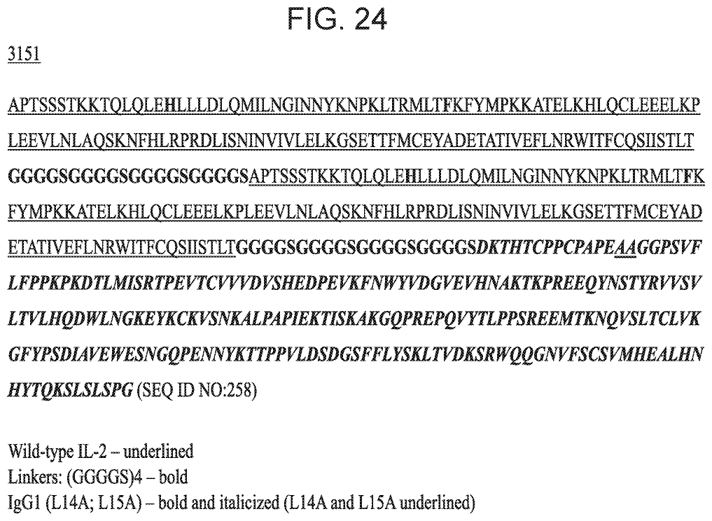

The effect of IL-2 valency on proliferation of CTLL-2 cells (American Type Culture Collection TIB-214) was assessed. CTLL-2 cells depend on IL-2 for growth; thus, these cells are used to assay for IL-2 activity. CTLL-2 cells were cultured for 24 hours in culture medium without IL-2. After the 24-hour period, IL-2/Fc fusion polypeptide was added to the cells (5×10³ CTLL-2 cells/well). The IL-2/Fc fusion polypeptides that were tested were: i) Rgt-3151 (4× wild-type IL-2; amino acid sequence provided in FIG. 24, which spontaneously forms a homodimer with a second copy of 3151); ii) Rgt-2657 (4× IL-2 (H16A; F42A); amino acid sequence provided in FIG. 23, which spontaneously forms a homodimer with a second copy of 2657); iii) Rgt-2656 (2× IL-2(H16A; F42A); amino acid sequence provided in FIG. 25, which spontaneously forms a homodimer with a second copy of 2656); and iv) Rgt-4123-4124 (1× IL-2(H16A; F42A); amino acid sequences provided in FIG. 26A and FIG. 26B). For comparison, cells were also incubated with: a) recombinant human IL-2 (rhIL-2); or b) a T-cell modulatory multimeric polypeptide (TMMP) that is referred to as “IST-1715-1717”. IST-1715-1717 is a homodimer of two heterodimers. Each heterodimer comprises: a) a first polypeptide comprising: i) two copies of IL-2(H16A; F42A); ii) a major histocompatibility complex (MHC) heavy chain polypeptide that is an HLA-A0201 polypeptide comprising a Cys at amino acid 84 and a Cys at amino acid 236; and iii) an IgG Fc polypeptide comprising an Ala at amino acid 234 and an Ala at amino acid 235; and b) a second polypeptide comprising: i) a cytomegalovirus (CMV) peptide NLVPMVATV (SEQ ID NO:269); ii) a linker comprising a Cys at amino acid 2; and iii) a beta-2 microglobulin (02M) polypeptide comprising a Cys at amino acid 12. The first and second polypeptides are joined by a first disulfide bond formed between the Cys in the linker and a Cys at amino acid 84 of the MHC heavy chain polypeptide and a second disulfide bond formed between the Cys at amino acid 12 of the P2M polypeptide and the Cys at amino acid 236 of the MHC heavy chain polypeptide. Thus, IST-1715-1717 has an IL-2 valency of 4 (2× IL-2 per heterodimer; two heterodimers per homodimer).

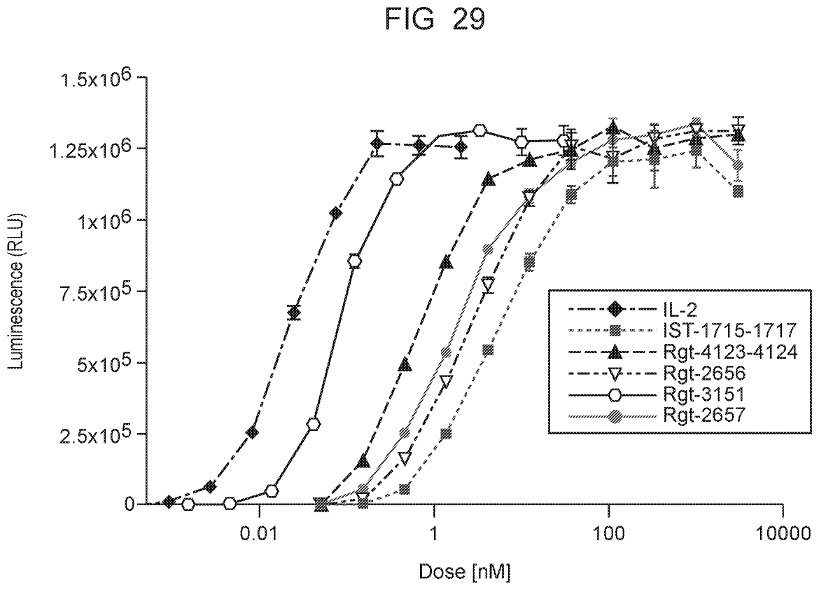

After a 72-hour incubation period with the various IL-2/Fc fusion polypeptides, the wild-type IL-2, or the TMMP, the relative cell proliferation was determined by a cell viability assay where increased number of viable cells is directly proportional to increased relative luminescence units (RLU). The data are shown in FIG. 29.

As shown in FIG. 29, CTLL-2 cells proliferated in response to all the IL-2/Fc fusion polypeptides. Rgt-2657, Rgt-2656 and Rgt-4123-4124 (IL-2 with H16A and F42A mutations) triggered more attenuated proliferation than Rgt-3151 (wild-type IL-2) or recombinant human IL-2. Differences were observed between the proliferation induced by IL-2/Fc fusion polypeptides with different valency of IL-2 (i.e. 1x, 2x, or 4× IL-2(H16A; F42A)), suggesting that IL-2 valency of the IL-2/Fc fusion polypeptides influences the proliferation of CTLL-2 cells.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the claims appended hereto.

What is claimed is:

1. A method comprising administering to an individual:
(i) a first composition comprising:
(a) modified or unmodified T cells having a T cell receptor (TCR); or
(b) modified cells that comprise a chimeric antigen receptor (CAR), wherein the CAR binds to a target antigen and the modified cell comprises an intracellular signaling domain that is activated by interaction of the modified cell with IL-2; or
(c) modified cells comprising one or more exogenous activation receptors;
or
(d) a product that can engage with the TCR of a T cell or be processed by an immune system into one or more antigens that can be presented by a major histocompatibility complex (MHC) to the TCR of a T cell, optionally wherein the one or more antigens are cancer-associated antigens; or
(e) one or more nucleic acids encoding one or more polypeptides that can engage with the TCR of a T cell or can be processed by an immune system into one or more antigens that can be presented by a major histocompatibility complex (MHC) to the TCR of a T cell, optionally wherein the one or more antigens are cancer-associated antigens; or
(f) at least one immune checkpoint inhibitor (CPI), and
(ii) a second composition comprising an immunomodulatory protein,
wherein the immunomodulatory protein comprises one or more variant IL-2 polypeptides that have at least two amino acid substitutions relative to set forth in SEQ ID NO:1,
wherein the one or more variant IL-2 polypeptides bind to an IL-2R alpha chain (IL-2Rα), and wherein the binding affinity to IL-2Rα is less than the affinity of a wild-type IL-2 polypeptide for IL-2Rα when assayed under the same conditions, wherein IL-2Rα has the amino acid sequence set forth in SEQ ID NO:2, and
wherein the one or more variant IL-2 polypeptides bind to an IL-2R beta chain (IL-2R$), and wherein the binding affinity to IL-2Rβ is less than the affinity of a wild-type IL-2 polypeptide for IL-2Rβ when assayed under the same conditions, wherein the IL-2Rβ has the amino acid sequence set forth in SEQ ID NO:3,
wherein the second composition preferentially activates T cells whose TCRs are engaged with an antigen presented by an MHC, as compared to T cells whose TCRs are not engaged with an antigen presented by an MHC,
wherein the first and second compositions are administered at the same time or at different times, and
wherein when the individual is administered a first composition comprising (a), (b), (c), (d) or (e), the individual also may be administered a CPI.

2. A method according to claim 1, wherein at least one of the one or more variant IL-2 polypeptides exhibits at least a two-fold decrease in binding affinity to IL-2Rβ compared to the binding affinity of a wild-type IL-2 polypeptide for IL-2Rβ, and exhibits at least a fifty-fold decrease in binding affinity to IL-2Rα compared to the binding affinity of a wild-type IL-2 polypeptide for IL-2Rα.

3. A method according to claim 1 or 2, wherein at least one of the one or more variant IL-2 polypeptides comprise at least one substitution that decreases the affinity of the variant IL-2 polypeptide for IL-2Rα, optionally wherein the at least one substitution is selected from a substitution at R38, F42, K43, Y45, E62, P65, E68, V69, L72, and combinations thereof.

4. A method according to any one of claims 1-3, wherein at least one of the one or more variant IL-2 polypeptides comprises a substitution of the phenylalanine amino acid F42, optionally wherein the phenylalanine is substituted with Ala, Gly, Val, Ile, or Leu.

5. A method according to any of claims 1-4, wherein at least one of the one or more variant IL-2 polypeptides comprise at least one substitution that decreases the affinity of the variant IL-2 polypeptide for IL-2Rβ, optionally wherein the at least one substitution is selected from a substitution at E15, H16, L19, D20, D84, S87, N88, V91, I92, and combinations thereof.

6. A method according to any one of claims 1-5, wherein at least one of the one or more variant IL-2 polypeptides comprises a substitution of the histidine amino acid H16, optionally wherein the histidine is substituted with Ala, Gly, Val, Leu, Thr, Ile, Asp, or Glu.

7. A method according to any one of claims 1-6, wherein at least one of the one or more variant IL-2 polypeptides comprises a substitution of the asparagine amino acid N88, optionally wherein the asparagine is substituted with Gly, Ala, Ser, Thr, Arg or Asp.

8. A method according to any one of claims 1-7, wherein at least one of the one or more variant IL-2 polypeptides comprises substitutions of amino acids F42 and H16, optionally wherein the phenylalanine is substituted with Ala, and wherein the histidine is substituted with Ala, Thr, Asp, or Glu.

9. A method according to any one of claims 1-8, wherein at least one of the one or more variant IL-2 polypeptides comprises: (i) an H16A substitution and an F42A substitution; (ii) an H16T substitution and an F42A substitution, (iii) an H16E substitution and an F42A substitution, and (iv) an H16D substitution and an F42A substitution.

10. A method according to any one of claims 1-9, wherein at least one of the one or more variant IL-2 polypeptides comprises substitutions at F42, H16, and N88.

11. A method according to claim 10, wherein the asparagine amino acid N88 is substituted with Gly, Ala, Ser, Thr, Arg or Asp.

12. A method according to any one of claims 1-11, wherein the immunomodulatory protein comprises two or more variant IL-2 polypeptides, wherein each variant IL-2 polypeptide comprises the same amino acid sequence.

13. A method according to claim 12, wherein the immunomodulatory protein comprises two variant IL-2 polypeptides that are in tandem and joined by an independently selected linker, optionally wherein the linker comprises glycine and serine.

14. A method according to any of claims 1-13, wherein the immunomodulatory protein further comprises a carrier.

15. A method according to claim 14, wherein the carrier is a lipid vesicle (e.g., a liposome) or micelle, a nanoparticle, a PEGylated protein, or an artificial antigen presenting cell such as engineered erythroid cell or enucleated cell (e.g., a platelet).

16. A method according to any of claims 1-13, wherein the immunomodulatory protein further comprises an immunoglobulin (Ig) scaffold polypeptide or a non-Ig scaffold polypeptide.

17. A method according to claim 16, wherein the immunomodulatory protein comprises a non-Ig scaffold chosen from an XTEN polypeptide, a transferrin polypeptide, an elastin-like polypeptide, a silk-like polypeptide, a fibronectin-based scaffold protein, or a silk-elastin-like polypeptide.

18. A method according to claim 16, wherein the wherein immunomodulatory protein is a fusion polypeptide that comprises: a) the one or more variant IL-2 polypeptides; and b) an Fc polypeptide, and wherein the Ig Fe polypeptide is an IgG1 Fc polypeptide, an IgG2 Fe polypeptide, an IgG3 Fc polypeptide, an IgG4 Fc polypeptide, an IgA Fc polypeptide, or an IgM Fc polypeptide.

19. A method according to claim 18, wherein the Ig Fc polypeptide is a variant that has a substantially reduced effector function, e.g. a substantially reduced ability to effect complement-dependent cytotoxicity (CDC) and/or antibody-dependent cell cytotoxicity (ADCC).

20. A method according to claim 18 or 19, wherein the Ig Fc polypeptide comprises one or more amino acid substitutions selected from N297A, L234A, L235A, L234F, L235E, G237A and P331S, wherein N297, L234, L235, G237 and P331 correspond to N77, L14, L15, G17 and P111, respectively, of the amino acid sequences depicted in FIG. 9A.

21. A method according to claim 18-20, wherein the Ig Fc polypeptide is an IgG1 Fc polypeptide that comprises an amino acid sequence having at least 95% amino acid sequence identity to the amino acid sequence depicted in any one of FIGS. 9A-9M.

22. A method according to any one of claims 18-21, wherein the immunomodulatory protein comprises a homodimer of two immunomodulatory proteins, each of which comprises an Ig Fc polypeptide, and wherein the Ig Fe of one immunomodulatory protein is joined by one or more disulfide bonds to the Ig Fc of the other immunomodulatory protein.

23. A method according to claim 22, wherein each of the immunomodulatory proteins in the homodimer comprises two variant IL-2 polypeptides in tandem, and wherein the variant IL-2 polypeptides are joined by an independently selected linker.

24. A method according to claim 22, wherein each of the immunomodulatory proteins in the homodimer comprises, from N-terminus to C-terminus:
(i) a variant IL-2 polypeptide;
(ii) an independently selected linker;
(iii) a variant IL-2 polypeptide;
(iv) an independently selected linker; and
(v) an Ig Fc polypeptide.

25. A method according to claim 22, wherein each of the immunomodulatory proteins in the homodimer comprises, from N-terminus to C-terminus:
(i) a variant IL-2 polypeptide;
(ii) an independently selected linker;
(iii) a variant IL-2 polypeptide;
(iv) an independently selected linker;
(v) an Ig Fc polypeptide;
(vi) an independently selected linker;
(vii) a variant IL-2 polypeptide;
(viii) an independently selected linker; and
(ix) a variant IL-2 polypeptide.

26. A method according to claim 22, wherein each of the immunomodulatory proteins in the homodimer comprises, from N-terminus to C-terminus:
(i) a variant IL-2 polypeptide;
(ii) an independently selected linker;
(iii) an Ig Fe polypeptide;
(iv) an independently selected linker; and
(v) a variant IL-2 polypeptide;

27. A method according to claim 22, wherein each of the immunomodulatory proteins in the homodimer comprises, from N-terminus to C-terminus:
(i) an Ig Fc polypeptide;
(ii) an independently selected linker;
(iii) a variant IL-2 polypeptide;
(iv) an independently selected linker; and
(v) a variant IL-2 polypeptide.

28. A method according to any one of claims 18-21, wherein the immunomodulatory protein comprises a heterodimer of two immunomodulatory proteins, wherein one of the immunomodulatory proteins comprises an Ig Fc polypeptide comprising an interspecific dimerization sequence and the other immunomodulatory protein comprises an Ig Fc polypeptide comprising a counterpart interspecific sequence, or
wherein the immunomodulatory protein comprises a heterodimer comprising first and second polypeptides, wherein the first polypeptide comprises an Ig Fc polypeptide comprising an interspecific dimerization sequence,
wherein the second polypeptide comprises an Ig Fc polypeptide comprising a counterpart interspecific sequence, and
wherein one of either the first or second polypeptide comprises the one or more variant IL-2 polypeptides.

29. A method according to claim 28, wherein
(A) one of the immunomodulatory proteins in the heterodimer comprises, from N-terminus to C-terminus:
(i) a variant IL-2 polypeptide;
(ii) an independently selected linker;
(iii) a variant IL-2 polypeptide;
(iv) an independently selected linker; and
(v) an Ig Fc polypeptide comprising an interspecific binding sequence, and
wherein the other immunomodulatory protein in the heterodimer comprises, from N-terminus to C-terminus:
(i) an Ig Fc polypeptide comprising a counterpart interspecific binding sequence;
(ii) an independently selected linker;
(iii) a variant IL-2 polypeptide;
(iv) an independently selected linker; and
(v) a variant IL-2 polypeptide, or
(B) the first polypeptide in the heterodimer comprises, from N-terminus to C-terminus:
(i) a variant IL-2 polypeptide;
(ii) an independently selected linker;
(iii) a variant IL-2 polypeptide;
(iv) an independently selected linker; and
(v) an Ig Fc polypeptide comprising an interspecific binding sequence, and
the second polypeptide in the heterodimer comprises an Ig Fc polypeptide comprising a counterpart interspecific binding sequence, but does not comprise a variant IL-2 polypeptide, or
(C) the first polypeptide in the heterodimer comprises, from N-terminus to C-terminus:
(i) an Ig Fc polypeptide comprising an interspecific binding sequence,
(ii) an independently selected linker;
(iii) a variant IL-2 polypeptide;
(iv) an independently selected linker;
(v) a variant IL-2 polypeptide, and
the second polypeptide in the heterodimer comprises an Ig Fc polypeptide comprising a counterpart interspecific binding sequence, but does not comprise a variant IL-2 polypeptide.

30. A method according to claim 28, wherein one of the immunomodulatory proteins in the heterodimer comprises, from N-terminus to C-terminus:
(i) a variant IL-2 polypeptide;
(ii) an independently selected linker; and
(iii) an Ig Fe polypeptide comprising an interspecific binding sequence, and wherein the other immunomodulatory protein in the heterodimer comprises, from N-terminus to C-terminus:
(i) an Ig Fc polypeptide comprising a counterpart interspecific binding sequence;
(ii) an independently selected linker; and
(iii) a variant IL-2 polypeptide.

31. A method according to claim 28, wherein
(A) the first polypeptide in the heterodimer comprises, from N-terminus to C-terminus:
(i) a variant IL-2 polypeptide;
(ii) an independently selected linker; and
(iii) an Ig Fc polypeptide comprising an interspecific binding sequence, and
the second polypeptide in the heterodimer comprises an Ig Fc polypeptide comprising a counterpart interspecific binding sequence, but does not comprise a variant IL-2 polypeptide, or
(B) the first polypeptide in the heterodimer comprises, from N-terminus to C-terminus:
(i) an Ig Fc polypeptide comprising an interspecific binding sequence;
(ii) an independently selected linker; and
(iii) a variant IL-2 polypeptide, and
wherein the second polypeptide in the heterodimer comprises an Ig Fc polypeptide comprising a counterpart interspecific binding sequence, but does not comprise a variant IL-2 polypeptide.

32. A method according to any one of claims 1-30, wherein the first composition comprises one or more products that can engage with the TCR of a T cell or be processed by an immune system into one or more antigens that can be presented by a major histocompatibility complex (MHC) to the TCR of a T cell, optionally wherein the one or more antigens are cancer-associated antigens.

33. A method according to any one of claims 1-30, wherein the first composition comprises one or more nucleic acids encoding one or more polypeptides that can engage with the TCR of a T cell or can be processed by an immune system into one or more antigens that can be presented by a major histocompatibility complex (MHC) to the TCR of a T cell, optionally wherein the one or more antigens are cancer-associated antigens.

34. The method of any one of claims 1-30, wherein the first composition comprises a TCR-T cell that comprises an exogenous TCR, optionally wherein the TCR binds a cancer-associated antigen when the cancer-associated antigen is presented by an MHC to the TCR.

35. A method according to any one of claims 1-30, wherein the first composition comprises tumor infiltrating lymphocytes (TILS), optionally wherein the TILS have been modified to reduce the susceptibility of the TILS to T-cell suppressive signals.

36. A method according to any one of claims 31-35, wherein the cancer-associated antigen is an antigen chosen from alpha-feto protein, Wilms-tumor-1 (WT-1), a mutant KRAS, e.g., comprising a G12C or G12D mutation, melanoma antigen recognized by T cells 1 (MART-I), melanoma-associated antigen (MAGE), MAGE-A1, MAGE-A3, MAGE-A4, human papillomavirus (HPV) antigen E6, HPV antigen E7, New York esophageal squamous cell carcinoma 1 (NY-ESO-1), MUC-1 (mucin-1, cell surface associated), mesothelin, survivin, prostate stem cell antigen (PSCA), prostate-specific membrane antigen (PSMA), prostate-specific antigen (PSA), a mutant p53 polypeptide, a Ras polypeptide, nuclear factor erythroid 2-related factor 2 (NFE2L2), beta-catenin, PIK3CA (phosphatidylinositol-4,5-bisphosphate 3-kinase catalytic subunit alpha), and BRAF.

37. A method according to any one of claims 1-30, wherein the first composition comprises a CAR having an antigen-binding domain that is specific for a cancer-associated antigen, optionally wherein the cell is a T cell, a macrophage, or an NK cell, optionally wherein
i) when the CAR is not bound to a cancer-associated antigen, the second composition provides homeostatic signals to the cell for survival, and/or
ii) when the CAR is not bound to a cancer-associated antigen, the second composition provides activating signals to the cell that cause the cell to proliferate and retain its cytotoxic function.

38. The method of claim 37, wherein the antigen-binding domain is a single-chain Fv polypeptide or a nanobody.

39. The method of claim 37 or 38, wherein the cancer-associated antigen is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2.

40. The method of any one of claims 1-39, wherein the method is for the treatment of a cancer in the individual.

41. The method of claim 40, wherein the first composition comprises (a), (b), (c), (d) or (e), and further comprising administering at least one immune checkpoint inhibitor (CPI) to the individual, wherein the CPI, first composition, and second composition are administered at the same time or at different times.

42. The method of claim 41, wherein the at least one immune checkpoint inhibitor comprises an antibody specific for the immune checkpoint.

43. The method of claim 42, wherein the antibody is specific for an immune checkpoint chosen from CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2, optionally wherein the immune checkpoint inhibitor is an antibody specific for PD-1, PD-L1, CTLA-4, TIGIT and LAG3.

44. The method of any one of claims 1-36, wherein the method is for the prevention of a cancer in the individual.

45. The method of any one of claims 1-36, wherein the method is for the treatment of a cancer in the individual.

46. A method according to any one of claims 1-30, wherein the individual is administered a CAR-T therapy product, a TCR-T therapy product, or a CAR-NK therapy product.

47. The method of claim 46, wherein the individual is administered a CAR-T therapy product, wherein the CAR-T cell therapy product comprises a population of modified autologous T cells comprising a CAR or allogeneic T cells comprising a CAR, wherein the CAR comprises an antigen-binding domain specific for a cancer-associated antigen.

48. The method of claim 47, wherein the antigen-binding domain is a single-chain Fv polypeptide or a nanobody.

49. The method of claim 47 or 48, wherein the cancer-associated antigen is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2.

50. The method of any one of claims 44-49, further comprising administering to the individual an immune checkpoint inhibitor.

51. The method of claim 50, wherein the immune checkpoint inhibitor is an antibody specific for an immune checkpoint selected from CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2, optionally wherein the CPI is an antibody specific for an immune checkpoint selected from CTLA-4, TIGIT, PD-1 and PD-L1, or from PD-1 or PD-L1.

52. A method according to any one of claims 1-30, wherein the first composition is a vaccine comprising one or more products that can engage with the TCR of a T cell or be processed by an immune system into one or more antigens that can be presented by a major histocompatibility complex (MHC) to the TCR of a T cell, optionally wherein the one or more antigens are cancer-associated antigens.

53. A method according to any one of claims 1-30, wherein the first composition is a vaccine comprising a nucleic acid comprising a nucleotide sequence encoding a polypeptide that is capable of being processed by an immune system into one or more antigens that can be presented by a major histocompatibility complex (MHC) to the TCR of a T cell, optionally wherein the one or more antigens are cancer-associated antigens.

54. The method of claim 52, wherein the vaccine comprises one or more products that can engage with the TCR of a T cell or be processed by an immune system into more than one cancer-associated antigens that can be presented by a major histocompatibility complex (MHC) to the TCRs of T cells.

55. The method of claim 53, wherein the vaccine comprises one or more nucleic acids comprising one or more nucleotide sequences that encode a plurality of polypeptides, wherein the polypeptides can engage with the TCR of a T cell or be processed by an immune system into more than one cancer-associated antigens that can be presented by a major histocompatibility complex (MHC) to the TCRs of T cells.

56. A method according to any one of claims 1-55, wherein the immunomodulatory protein is selected from the group of: i) an immunomodulatory protein comprising, consisting essentially of, or consisting of a homodimer of the 2657 protein depicted in FIG. 23A, wherein the two copies of the 2657 protein are joined by two disulfide bonds that link the Ig Fc polypeptides in each copy of 2657; ii) an immunomodulatory protein comprising, consisting essentially of, or consisting of a homodimer of the 2656 protein depicted in FIG. 25A, wherein the two copies of the 2656 protein are joined by two disulfide bonds that link the Ig Fe polypeptides in each copy of 2656; iii) an immunomodulatory protein comprising, consisting essentially of, or consisting of a homodimer of the 2657Δ protein depicted in FIG. 23B, wherein the two copies of the 2657Δ protein are joined by two disulfide bonds that link the Ig Fc polypeptides in each copy of 2657Δ; iv) an immunomodulatory protein comprising, consisting essentially of, or consisting of a homodimer of the 2656A protein depicted in FIG. 25B, wherein the two copies of the 2656Δ protein are joined by two disulfide bonds that link the Ig Fc polypeptides in each copy of 2656Δ; and v) a heterodimer comprising the 4123 protein depicted in FIG. 26A and the 4124 protein depicted in FIG. 26B.

57. A fusion polypeptide as shown in any one of FIGS. 2A-2C, 3A-3C, 4A-4C, 5A-5B, 6A-6B, 7A-7B, 8A-8F, 10A-10C, 11A-11C, 12A-12B, 13A-14B, 14A-14N, and 30A-30G wherein the fusion polypeptide comprises one or more variant IL-2 polypeptides that have at least two amino acid substitutions relative to set forth in SEQ ID NO:1,
wherein the one or more variant IL-2 polypeptides bind to an IL-2R alpha chain (IL-2Rα), and wherein the binding affinity to IL-2Rα is less than the affinity of a wild-type IL-2 polypeptide for IL-2Rα when assayed under the same conditions, wherein IL-2Rα has the amino acid sequence set forth in SEQ ID NO:2, and
wherein the one or more variant IL-2 polypeptides bind to an IL-2R beta chain (IL-2Rβ), and wherein the binding affinity to IL-2Rβ is less than the affinity of a wild-type IL-2 polypeptide for IL-2Rβ when assayed under the same conditions, wherein the IL-2Rβ has the amino acid sequence set forth in SEQ ID NO:3.

58. A fusion polypeptide according to claim 57, wherein at least one of the one or more variant IL-2 polypeptides comprises one or more mutations that can reduce binding of IL-2 to IL-2Rα, wherein the one or more mutations are chosen from substitutions at one or more of amino acids R38, F42, K43, Y45, E62, P65, E68, V69, and L72.

59. A fusion polypeptide according to claim 58, wherein at least one of the one or more variant IL-2 polypeptides comprises a substitution of amino acid F42, optionally wherein the Phe is substituted with Ala or Lys.

60. A fusion polypeptide according to any one of claims 57-59, wherein at least one of the one or more variant IL-2 polypeptides comprises one or more mutations that can reduce binding of IL-2 to IL-2Rβ, wherein the one or more mutations are chosen from substitutions at one or more of amino acids E15, H16, L19, D20, D84, S87, N88, V91, I92.

61. A fusion polypeptide according to claim 60, wherein at least one of the one or more variant IL-2 polypeptides comprises a substitution of amino acid H16, optionally wherein the His is substituted with Ala, Glu, Thr, or Asp.

62. A fusion polypeptide according to claim 57, wherein at least one of the one or more variant IL-2 polypeptides comprises substitutions chosen from E15A with R38A, R38D or R38E.

63. A fusion polypeptide according to claim 57, wherein at least one of the one or more variant IL-2 polypeptides comprises substitutions chosen from: H16A with R38A, R38D or R38E; H16T with R38A, R38D or R38E; H16E with R38A, R38D or R38E; and H16D with R38A, R38D or R38E.

64. A fusion polypeptide according to claim 57, wherein at least one of the one or more variant IL-2 polypeptides comprises substitutions chosen from: D84H with R38A, R38D or R38E; D84K with R38A, R38D or R38E; and D84R with R38A, R38D or R38E.

65. A fusion polypeptide according to claim 57, wherein at least one of the one or more variant IL-2 polypeptides comprises substitutions chosen from: R38A with N88S, N88A, N88G, N88R, N88T, or N88D; R38D with N88S, N88A, N88G, N88R, N88T, or N88D; and R38E with N88S, N88A, N88G, N88R, N88T, or N88D.

66. A fusion polypeptide according to claim 57, wherein at least one of the one or more variant IL-2 polypeptides comprises substitutions chosen from: R38A with V91E, V91A or V91T, R38D with V91E, V91A or V91T; and R38E with V91E, V91A or V91T.

67. A fusion polypeptide according to claim 57, wherein at least one of the one or more variant IL-2 polypeptides comprises substitutions chosen from R38A, I92A, R38D, I92A and R38E, I92A.

68. A fusion polypeptide according to claim 57, wherein at least one of the one or more variant IL-2 polypeptides comprises substitutions chosen from E15A, F42A and E15A, F42K.

69. A fusion polypeptide according to claim 57, wherein at least one of the one or more variant IL-2 polypeptides comprises substitutions chosen from: H16A, F42A; H16T, F42A; H16E, F42A; H16D, F42A; H16A, F42K; H16T, F42K; and H16E, F42K; H16D, F42K.

70. A fusion polypeptide according to claim 57, wherein at least one of the one or more variant IL-2 polypeptides comprises substitutions chosen from: F42A with N88S, N88A, N88G, N88R, N88T, or N88D; and F42K with N88S, N88A, N88G, N88R, N88T, or N88D.

71. A fusion polypeptide according to claim 57, wherein at least one of the one or more variant IL-2 polypeptides comprises substitutions chosen from: F42A with V91E, V91A, or V91T; and F42K with V91E, V91A, or V91T

72. A fusion polypeptide according to claim 57, wherein at least one of the one or more variant IL-2 polypeptides comprises substitutions chosen from: F42A with I92A; and F42K with I92A.

73. A fusion polypeptide according to claim 57, wherein at least one of the one or more variant IL-2 polypeptides comprises substitutions of E15A and K43E.

74. A fusion polypeptide according to claim 57, wherein at least one of the one or more variant IL-2 polypeptides comprises substitutions chosen from: H16A, K43E; H16T, K43E; H16E, K43E; and H16D, K43E

75. A fusion polypeptide according to claim 57, wherein at least one of the one or more variant IL-2 polypeptides comprises substitutions chosen from K43E with D84H, D84K or D84R

76. A fusion polypeptide according to claim 57, wherein at least one of the one or more variant IL-2 polypeptides comprises substitutions chosen from K43E with N88S, N88A, N88G, N88R, N88T, or N88D.

77. A fusion polypeptide according to claim 57, wherein at least one of the one or more variant IL-2 polypeptides comprises substitutions chosen from K43E with V91E, V91A, or V91T

78. A fusion polypeptide according to claim 57, wherein at least one of the one or more variant IL-2 polypeptides comprises substitutions of K43E and 192A or E15A, E62Q.

79. A fusion polypeptide according to claim 57, wherein at least one of the one or more variant IL-2 polypeptides comprises substitutions chosen from: H16A, E62Q; H16T, E62Q; H16E, E62Q; and H16D, E62Q

80. A fusion polypeptide according to claim 57, wherein at least one of the one or more variant IL-2 polypeptides comprises substitutions chosen from E62Q with D84H, D84K or D84R.

81. A fusion polypeptide according to claim 57, wherein at least one of the one or more variant IL-2 polypeptides comprises substitutions chosen from E62Q with N88S, N88A, N88G, N88R, N88T, or N88D.

82. A fusion polypeptide according to claim 57, wherein at least one of the one or more variant IL-2 polypeptides comprises substitutions chosen from E62Q with V91E, V91A, or V91T.

83. A fusion polypeptide according to claim 57, wherein at least one of the one or more variant IL-2 polypeptides comprises substitutions chosen from E62Q and I92A.

84. A fusion polypeptide according to claim 57, wherein at least one of the one or more variant IL-2 polypeptides comprises substitutions chosen from E62Q with V91E, V91A, or V91T.

85. A fusion polypeptide according to claim 68, wherein at least one of the one or more variant IL-2 polypeptides comprises substitutions at F42, E15 and N88.

86. A fusion polypeptide according to claim 68, wherein at least one of the one or more variant IL-2 polypeptides comprises substitutions at F42, E15 and V91.

87. A fusion polypeptide according to claim 69, wherein at least one of the one or more variant IL-2 polypeptides comprises substitutions at F42, H16 and D84.

88. A fusion polypeptide according to claim 69, wherein at least one of the one or more variant IL-2 polypeptides comprises substitutions at F42, H16 and N88.

89. A fusion polypeptide according to claim 69, wherein at least one of the one or more variant IL-2 polypeptides comprises substitutions at F42, H16 and V91.

90. A fusion polypeptide according to claim 69, wherein at least one of the one or more variant IL-2 polypeptides comprises substitutions at F42, H16 and 192.

91. A fusion polypeptide according to any one of claims 57-90, wherein the fusion protein is as shown in any one of FIGS. 10A-10C, FIGS. 11A-11C, FIGS. 12A-12B and FIGS. 13A-13B, FIGS. 14A-14N, and FIG. 30A-30G and wherein the fusion polypeptide comprises a functional protein that is a cancer-targeting polypeptide (CTP).

92. A fusion polypeptide according to claim 91, wherein the target of the cancer-targeting polypeptide is a peptide-HLA complex on the surface of a cancer cell.

93. A fusion polypeptide according to claim 91, wherein the target of the CTP is a cancer-associated epitope.

94. A fusion polypeptide according to claim 91, wherein the CTP is an antibody that is specific for a cancer-associated antigen.

95. A fusion polypeptide according to claim 91, wherein the CTP is an antibody that is specific for a peptide/HLA complex on the surface of a cancer cell, wherein the peptide can be a cancer-associated peptide (e.g., a peptide of a cancer-associated antigen).

96. A fusion polypeptide according to claim 91, wherein the functional protein is a TCR such as single-chain T cell receptor of “scTCR” that is specific for a peptide/HLA complex on the surface of a cancer cell, wherein the peptide can be a cancer-associated peptide (e.g., a peptide of a cancer-associated antigen).

97. A fusion polypeptide according to claim 91, wherein the functional protein comprises a wild-type or variant immunomodulatory polypeptide, e.g., a wild type or variant immunostimulatory polypeptide such as B7 family of costimulatory receptors, e.g., CD80, CD86, a cytokine such as IL-7, IL-12, IL-15 or IL-21, a TNF superfamily member such as CD-40, 4-1BBL and OX40, or a chemokine such as CCL19, CCL21, CXCL9/10/11, or CXCL12.

98. A fusion polypeptide according to any one of claims 57-97, wherein the fusion polypeptide comprises one or more independently selected linkers.

99. A method comprising administering to an individual:
(i) a first composition comprising:
(a) modified or unmodified T cells having a T cell receptor (TCR), or
(b) modified cells that comprise a chimeric antigen receptor (CAR), wherein the CAR binds to a target antigen and the modified cell comprises an intracellular signaling domain that is activated by interaction of the modified cell with IL-2, or
(c) modified cells comprising one or more exogenous activation receptors;
or
(d) one or more products that can engage with the TCR of a T cell or be processed by an immune system into one or more antigens that can be presented by a major histocompatibility complex (MHC) to the TCR of a T cell, optionally wherein the one or more antigens are cancer-associated antigens; or
(e) one or more nucleic acids encoding one or more polypeptides that can engage with the TCR of a T cell or can be processed by an immune system into one or more antigens that can be presented by a major histocompatibility complex (MHC) to the TCR of a T cell, optionally wherein the one or more antigens are cancer-associated antigens; or
(f) at least one immune checkpoint inhibitor (CPI), and
and
(ii) a second composition comprising a fusion polypeptide of any one of claims 57-98,
wherein when the individual is administered a first composition comprising (a), (b), (c), (d) or (e), the individual also may be administered a CPI, and
wherein the first and second compositions are administered at the same time or at different times.

100. A method according to claim 99, wherein the second composition preferentially activates T cells whose TCRs are engaged with an antigen presented by an MHC, as compared to T cells whose TCRs are not engaged with an antigen presented by an MHC.

101. A method according to claim 99 or 100, wherein the first composition comprises modified or unmodified T cells having a T cell receptor (TCR).

102. A method according to claim 99 or 100, wherein the first composition comprises modified cells that comprise a chimeric antigen receptor (CAR), wherein the CAR binds to a target antigen and the modified cell comprises an intracellular signaling domain that is activated by interaction of the modified cell with IL-2.

103. A method according to claim 99 or 100, wherein the first composition comprises modified cells comprising one or more exogenous activation receptors.

104. A method according to claim 99 or 100, wherein the first composition comprises one or more products that can engage with the TCR of a T cell or be processed by an immune system into one or more antigens that can be presented by a major histocompatibility complex (MHC) to the TCR of a T cell, optionally wherein the one or more antigens are cancer-associated antigens.

105. A method according to claim 99 or 100, wherein the first composition comprises one or more nucleic acids encoding one or more polypeptides that can engage with the TCR of a T cell or can be processed by an immune system into one or more antigens that can be presented by a major histocompatibility complex (MHC) to the TCR of a T cell, optionally wherein the one or more antigens are cancer-associated antigens.

106. A method according to claim 99 or 100, wherein the first composition comprises a CPI.

107. A method according to any of claims 101-105, wherein the method further comprises administering a CPI.

108. A method according to any one of claims 99-107, wherein the CPI is an antibody specific for an immune checkpoint selected from CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2.

109. A method according to any one of claims 99-107, wherein the CPI is an antibody specific for an immune checkpoint selected from CTLA-4, TIGIT, PD-1, and PD-L1.

110. A method according to any one of claims 99-107, wherein the CPI is an antibody specific for an immune checkpoint selected from PD-1 and PD-L1.
